(19) United States
(12) Patent Application Publication
(10) Pub. No.: US 2026/0356809 A1
(43) Pub. Date: Nov. 12, 2026

(54) DOPAMINE D3/D2 RECEPTOR PARTIAL AGONISTS FOR THE TREATMENT OF NEUROPSYCHIATRIC DISORDERS

(71) Applicant: The United States of America, as represented by the Secretary, Department of Health & Human Services, Bethesda, MD (US)

(72) Inventors: Amy Hauck Newman, Baltimore, MD (US); Emma S. Gogarnoiu, Hoboken, NJ (US); Caleb David Vogt, Baltimore, MD (US); Anver Basha Shaik, Columbus, OH (US); Omar Soler-Cedeno, Baltimore, MD (US); Zheng-Xiong Xi, Baltimore, MD (US)

(73) Assignee: The United States of America, as represented by the Secretary, Department of Health & Human Services, Bethesda, MD (US)

(21) Appl. No.: 19/116,951

(22) PCT Filed: Sep. 28, 2023
(86) PCT No.: PCT/US2023/033932

§ 371 Date: Mar. 28, 2025

Related U.S. Application Data
(60) Provisional application No. 63/377,893, filed on Sep. 30, 2022.

(51) Int. Cl. C07D 295/135; A61K 31/454; A61K 31/495; A61K 31/496; A61K 31/506; A61K 31/513; A61K 31/5355; A61K 45/06; A61P 25/30; C07D 207/12; C07D 207/27; C07D 209/42; C07D 211/26; C07D 211/48; C07D 211/76; C07D 213/56; C07D 213/74; C07D 215/50; C07D 217/06; C07D 233/36; C07D 239/10; C07D 239/42; C07D 265/10; C07D 275/04; C07D 307/85; C07D 401/12; C07D 413/04

(52) U.S. Cl. CPC C07D 295/135; A61K 31/454; A61K 31/495; A61K 31/496; A61K 31/506; A61K 31/513; A61K 31/5355; A61K 45/06; A61P 25/30; C07D 207/12; C07D 207/27; C07D 209/42; C07D 211/26; C07D 211/48; C07D 211/76; C07D 213/56; C07D 213/74; C07D 215/50; C07D 217/06; C07D 233/36; C07D 239/10; C07D 239/42; C07D 265/10; C07D 275/04; C07D 307/85; C07D 401/12; C07D 413/04

(57) ABSTRACT

D₃R selective partial agonists and their use in treating substance use disorders alone or in combination with affective disorders are disclosed. These substance use disorders include psychostimulant use disorder (PSUD). Compounds include, for example, compounds having D₃R/D₂R selectivities while maintaining high (low nM to pM) binding affinities at D₃R. These ligands are considered bitopic, meaning they include a primary pharmacophore (PP), which binds to the orthosteric binding site, that is then linked to a secondary pharmacophore (SP), which binds to D₃R-unique secondary binding pocket.

### CROSS-REFERENCE TO RELATED APPLICATIONS

This application claims priority to U.S. Provisional Patent Application No. 63/377,893, filed Sep. 30, 2022, which is incorporated by reference herein in its entirety.

### STATEMENT REGARDING FEDERALLY SPONSORED RESEARCH OR DEVELOPMENT

This invention was made with Government support under Grant Number ZIA DA 000424 awarded by the National Institute on Drug Abuse (NIDA) Intramural Research Program. The Government has certain rights in this invention.

### BACKGROUND OF THE INVENTION

### Field

The present disclosure relates to dopamine D3/D2 receptor partial agonists for the treatment of neuropsychiatric disorders.

### Description of Related Art

During the COVID-19 pandemic, drug overdose fatalities soared to a record in 2021. While opioids are involved with a majority of these deaths, polysubstance use is another contributing factor, leading to increases in morbidity that poses further challenges for prevention and treatment. To further complicate matters, substance use disorders (SUD) frequently occur with affective disorders, including anxiety, major depressive disorder, and bipolar disorder. Such dual disorders have also increased during the COVID-19 pandemic and are particularly difficult to treat when comorbid with psychostimulant use disorder (PSUD), for which there is currently no FDA-approved pharmacotherapeutic.

Development of highly selective D₃R antagonists/partial agonists for the treatment of SUD has been of great interest due to their ability to attenuate drug reinforcement as well as inhibit cue- and stress-induced reinstatement for psychostimulants and opioids in animal models. D₃R-selective antagonists, specifically, have been shown to block the expression of cocaine- or heroin-induced conditioned place preference and inhibit the rewarding effects of these drugs. In addition to promising preclinical work, gaining D₃R selectivity over D₂R can avoid the extrapyramidal side effects, weight gain, metabolic disorders, and motor coordination deficits associated with D₂R antagonism. Psychostimulants, such as cocaine, are drugs of high misuse potential that contribute to death by overdose. Selective dopamine D3 receptor (D₃R) partial agonists/antagonists have been developed for the treatment of psychostimulant use disorders (PSUD). However, none have reached the clinic due to insufficient potency/efficacy or potential cardiotoxicity.

While highly D₃R-preferential antagonists and low-efficacy partial agonists demonstrate potential as treatments for SUD, there are two challenges that still exist. The first is that these compounds generally show favorable results in animal models of opioid use disorder but are often not as effective in mitigating cocaine or methamphetamine self-administration, especially at low fixed-ratio schedules (e.g., FR1 or FR2) of reinforcement. Secondly, relatively high doses (e.g., 10-56 mg/kg, i.p.) are used to observe reductions in drug-seeking behaviors despite having very high affinities (Kᵢ values in the low nM to pM range) for the desired target, D₃R. The solution to this technical problem is provided by the embodiments characterized in the claims and is otherwise described herein.

### BRIEF SUMMARY OF THE INVENTION

The present disclosure is directed to D₃R selective partial agonists and their use in treating substance use disorders alone or in combination with affective disorders.

In an aspect, the invention provides a compound having Formula (I), an enantiomer thereof, a racemate thereof, a salt thereof, or any combination thereof:

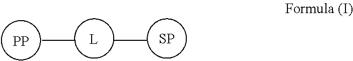

wherein
   PP is primary pharmacophore of formula PP¹:

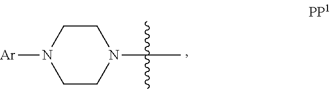

wherein Ar is a substituted or unsubstituted aromatic or heteroaromatic substituent;
L is a linker, covalently bound to the primary pharmacophore, of formula L¹, L^{2a}, L^{2b}, L^{2c}, or L^{2d}:

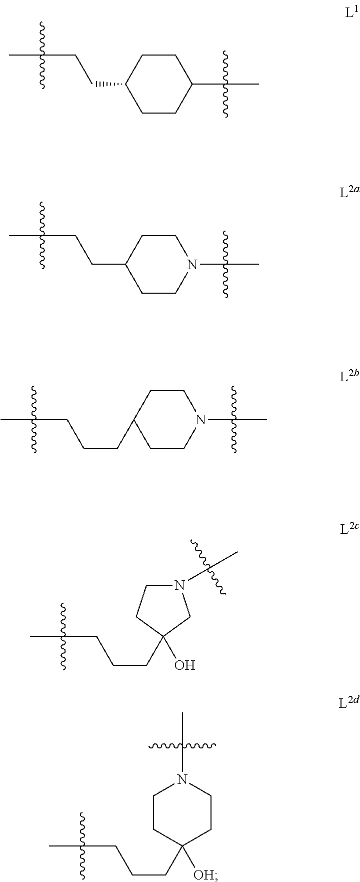

and
SP is a secondary pharmacophore, covalently bound to the linker, of formula SP¹ when L is L¹, or of formula SP² when L is L^{2a}, L^{2b}, L^{2c}, or L^{2d}.

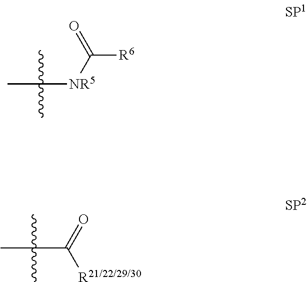

wherein R⁵ is hydrogen or C₁₋₈ alkyl,
   R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
   R⁵ and R⁶ combine to form a 5-, 6-, or 7-membered cyclic amide, cyclic carbamate, or cyclic urea, and
   R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent; and
wherein the compound is a selective dopamine D₃ receptor partial agonist.

In another aspect, the invention provides a compound having Formula (I), an enantiomer thereof, a racemate thereof, a salt thereof, or any combination thereof:

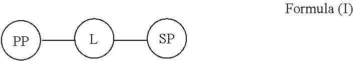

wherein PP is a primary pharmacophore, L is a linker covalently bound to the primary pharmacophore, and SP is a secondary pharmacophore covalently bound to the linker; wherein the compound is a selective dopamine D₃ receptor partial agonist; wherein the compound is represented by a first primary pharmacophore PP¹, a first linker L¹, and a first secondary pharmacophore SP¹:

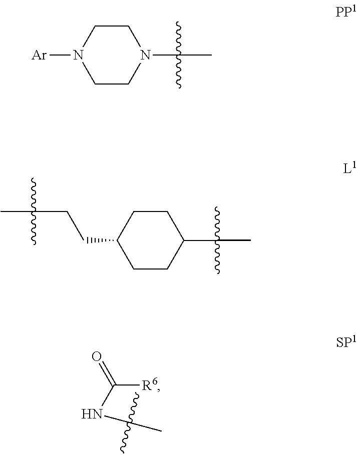

wherein Ar is a substituted or unsubstituted aromatic substituent, and R⁶ is an amine, a heterocycle, or an ether-containing alkyl substituent, or wherein the compound is represented by a second primary pharmacophore PP², a second linker L², and a second secondary pharmacophore SP²:

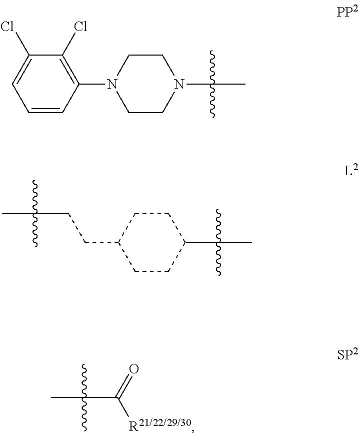

and
wherein R^{21/22/29/30} is an amine or an ether-containing alkyl.

In another aspect, the invention provides a pharmaceutical composition comprising a therapeutically effective amount of a compound having Formula (I) disclosed herein, an enantiomer thereof, a racemate thereof, a salt thereof, or any combination thereof, together with a pharmaceutically acceptable carrier.

In another aspect, the invention provides a method of treating drug misuse or drug addiction, comprising administering to a patient in need thereof a composition comprising a therapeutically effective amount of a compound having Formula (I) disclosed herein, an enantiomer thereof, a racemate thereof, a salt thereof, or any combination thereof, optionally in combination with one or more additional active ingredients.

In another aspect, the invention provides a method of treating a substance use disorder, comprising administering to a patient in need thereof a composition comprising a therapeutically effective amount of a compound having Formula (I) disclosed herein, an enantiomer thereof, a racemate thereof, a salt thereof, or any combination thereof, optionally in combination with one or more additional active ingredients.

In a further aspect, the invention provides a method of treating one or more affective disorders, schizophrenia, or a combination thereof, comprising administering to a patient in need thereof a composition comprising a therapeutically effective amount of a compound having Formula (I) disclosed herein, an enantiomer thereof, a racemate thereof, a salt thereof, or any combination thereof, optionally in combination with one or more additional active ingredients.

### BRIEF DESCRIPTION OF THE SEVERAL VIEWS OF THE DRAWINGS

For a further understanding of the nature, objects, and advantages of the present disclosure, reference should be had to the following detailed description, read in conjunction with the following drawings, wherein like reference numerals denote like elements.

FIG. 1 shows chemical formulae and structures in accordance with the present disclosure.

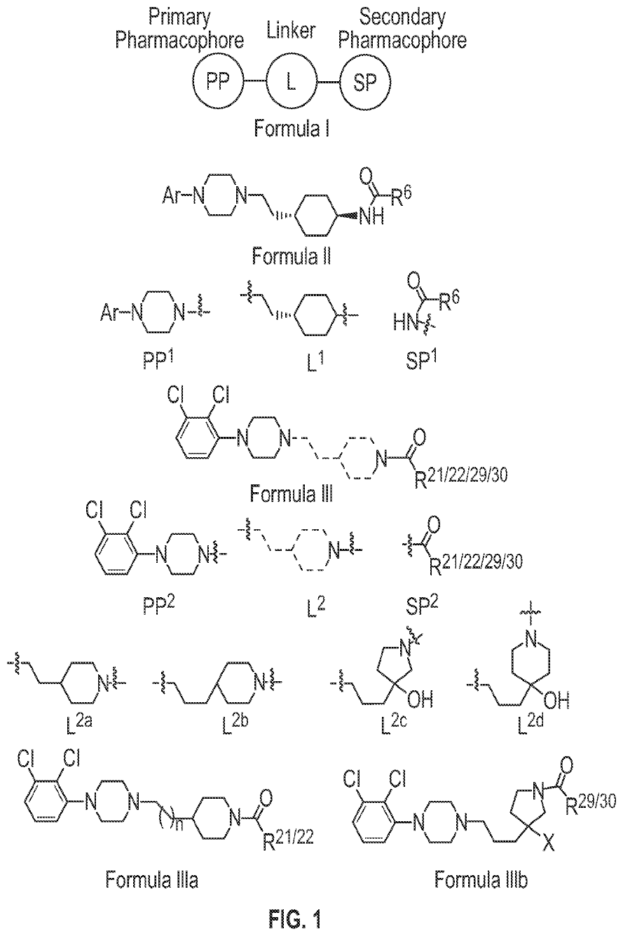

FIG. 2 shows a first synthetic scheme (Scheme 1) in accordance with the present disclosure.

FIG. 3 shows a second synthetic scheme (Scheme 2) in accordance with the present disclosure.

FIG. 4 shows a third synthetic scheme (Scheme 3) in accordance with the present disclosure.

FIG. 5 shows a fourth synthetic scheme (Scheme 4) in accordance with the present disclosure.

FIG. 6 shows, in panels A-F, agonism profiles at D₂R. Two differents bioluminescence resonance energy transfer (BRET) assays were used to assess D₂R activation: an amplified Gα_{oA} G protein (GPA Gα_{oA}) activation assay, shown on the left panels, and a less amplified miniG recruitment assay shown on the right panels. Panels A-F show concentration response curves for a subset of the 14 selected ligands: 2,3-dichorophenyl, 2-chloro-3-ethylphenyl and 2-fluoro-3-methoxyphenyl compounds—Panel A, GPA Gα_{oA}, and Panel B, miniGₛᵢ; 2-trifluoromethyl substituted pyridine and 2-methoxy-3-chloro-5-ethylphenyl compounds—Panel C, GPA Gαo_{A}, and Panel D, miniGₛᵢ; and linker modified compounds—Panel E, GPA Gα_{oA}, and Panel F, miniGₛᵢ. Data points represent the mean±SEM of three independent experiments performed in duplicate.

FIG. 7 shows, in panels A-F, functional profiles at the D₃R. Using the Gα_{oA} assay, all ligands were tested for D₃R agonism and are shown in Panels A, C, and E. 11 out of the 14 selected ligands were tested for D₃R antagonism (compounds were added to cells together with and EC₅₀ concentration (3 nM) of quinpirole) and are shown in Panels B, D, and F. Compounds 6c, 13c, and 21a signaled as robust D₃R agonists and were thus not tested as antagonists as they would not displace quinpirole signal. Each panel shows concentration response curves for a subset of the selected ligands: 2,3-dichorophenyl, 2-chloro-3-ethylphenyl and 2-fluoro-3-methoxyphenyl compounds—Panel A, D₃R agonism, and Panel B, D₃R antagonism; 2-trifluoromethyl substituted pyridine and 2-methoxy-3-chloro-5-ethylphenyl compounds—Panel C, D₃R agonism, and Panel D, D₃R antagonism; linker modified compounds—Panel E, D₃R agonism and Panel F, D₃R antagonism. Data points represent the mean±SEM of three independent experiments performed in duplicate.

FIG. 8, panels A-C, shows dose-dependent effects of cariprazine (compound 6a) on cocaine self administration in rats. Panel A shows the dose-dependent effects of cariprazine on cocaine self-administration (infusions). Panel B shows effects of cariprazine on inactive lever presses. Panel C shows representative cocaine self-administration (infusions) records, illustrating the typical extinction-like patterns of drug taking and drug seeking—lower doses of cariprazine (0.3 mg/kg) increased, while at higher doses it produced an initial burst-like drug infusions followed by cessation of drug self-administration. *p<0.05, **p<0.01, compared to vehicle (0 mg/kg).

FIG. 9, panels A-C, shows dose-dependent effects of compound 13a on cocaine self administration. Panel A shows mean numbers after different doses of 13a administration. Panel B shows mean numbers of inactive lever presses after different doses of 13a pretreatments. Panel C shows representative cocaine infusions behavior indicating an extinction-like pattern of cocaine self-administration after 13a administration in a way similar to compound 6a. (*p<0.05, **p<0.01, ***p<0.001, compared to the vehicle control group).

FIG. 10 shows dose-dependent effects of 13e on cocaine self administration. Panel A shows mean number of self-infusions after different doses of 13e. Panel B shows mean numbers of inactive lever presses 13e pretreatments. Panel C shows representative cocaine infusions illustrating the patterns of cocaine self-administration after vehicle or 13e administration. (*p<0.05; **p<0.01, compared to the vehicle control group).

FIGS. 11A-11C show the results of the multiple dose analysis of cocaine under fixed-ratio (FR2), as determined for mice treated with cariprazine (FIG. 11A), compound 13a (FIG. 11B), and compound 13e (FIG. 11C), as described in Example 5. (*p<0.05, **p<0.01, ***p<0.001, compared to the vehicle control group).

FIGS. 12A and 12B show the results of the self-administration under progressive ratio (PR) break point analysis, as determined for mice treated with cariprazine and compound 13a (FIG. 12A) and compound 13e (FIG. 12B), as described in Example 5. (*p<0.05; **p<0.01, compared to the vehicle control group).

FIGS. 13A-13C show the results of the drug-induced reinstatement analysis of cocaine seeking behavior, as determined for mice treated with cariprazine (FIG. 13A), compound 13a (FIG. 13B), and compound 13e (FIG. 13C), as described in Example 5. (*p<0.05, **p<0.01, ***p<0.001, compared to the vehicle control group).

### DETAILED DESCRIPTION OF THE INVENTION

Compounds are described using standard nomenclature. Unless defined otherwise, all technical and scientific terms used herein have the same meaning as is commonly understood by one of skill in the art to which this disclosure belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (for example, “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, for example, in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein can include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I, and ¹³¹I. Formulae, subformulae thereof, and compounds thereof include all pharmaceutically acceptable salts of the same.

The term “substituted” means that any one or more hydrogens on the designated atom or group is replaced with a selection from the indicated group, provided that the designated atom's normal valence is not exceeded. Combinations of substituents and/or variables are permissible, for example, if such combinations result in stable compounds or useful synthetic intermediates. A stable compound or stable structure can be a compound that is sufficiently robust to survive isolation from a reaction mixture, and subsequent formulation into an effective therapeutic agent. A substituent or combination of substituents described with respect to one formula, subformula, or compound, can also be used in any other formula, subformula, or compound where consistent with valence, polarity, size, structure, and other parameters, unless otherwise indicated.

A dash (“-”) that is not between two letters or symbols is used to indicate a point of attachment for a substituent.

“Alkyl” refers to a group derived from a straight or branched chain saturated aliphatic hydrocarbon having the specified number of carbon atoms and having a valence of one, optionally substituted with one or more substituents where indicated, provided that the valence of the alkyl group is not exceeded. If the number of carbon atoms is not specified, the number of carbon atoms is 1 to 8.

“Cycloalkyl” refers to a group that comprises one or more saturated and/or partially saturated rings in which all ring members are carbon, the group having the specified number of carbon atoms. Cycloalkyl groups do not include an aromatic ring or a heterocyclic ring. If the number of carbon atoms is not specified, the number of carbon atoms is 4 to 9.

“Heterocycl” or “heterocycle” refers to a group that comprises one or more saturated and/or partially saturated rings in which all ring members are carbon, in which at least one ring member (for example, one, two or three ring members) is a heteroatom selected from nitrogen (N), oxygen (O), sulfur (S), and phosphorus (P), the group having the specified number of carbon atoms. Cycloalkyl groups do not include an aromatic ring or a heterocyclic ring. If the number of carbon atoms is not specified, the number of carbon atoms is 3 to 8. For example, the heterocycl or heterocycle can be morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, piperzinyl, or dihydropyrimidinyl.

“Alkanoyl” refers to a group having formula “alkyl-C(═O)-”, wherein “alkyl” is the same as defined above.

“Cycloalkanoyl” refers to a group having formula “cycloalkyl-C(═O)-”, wherein “cycloalkyl” is the same as defined above.

“Aryl” or “aromatic” refers to a cyclic group in which all ring members are carbon and all rings are aromatic, the group having the specified number of carbon atoms, and having a valence of one, optionally substituted with one or more substituents where indicated, provided that the valence of the aryl group is not exceeded. More than one ring can be present, and any additional rings can be fused, pendant (i.e., bridged), spirocyclic, or a combination thereof.

“Heteroaryl” or “heteroaromatic” means a monovalent carbocyclic ring group that includes one or more aromatic rings, in which at least one ring member (for example, one, two or three ring members) is a heteroatom selected from nitrogen (N), oxygen (O), sulfur (S), and phosphorus (P), the group having the specified number of carbon atoms. When more than one ring is present, the additional rings can be fused, pendant (i.e., bridged), spirocyclic, or a combination thereof. In addition, the additional ring can be “aryl” or “aromatic” so long as at least one of the more than one rings is “heteroaryl” or “heteroaromatic”. For example, the heteroaryl or heteroaromatic group can be furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, quinazolinyl, benzoisothiazolyl, or benzoisoxazolyl.

“Halogen” means fluoro, chloro, bromo, or iodo, and are defined herein to include all isotopes of the same, including heavy isotopes and radioactive isotopes. Examples of useful halo isotopes include ¹⁸F, ⁷⁶Br, and ¹³¹I. Additional isotopes will be readily appreciated by one of skill in the art.

“Substituted” means that the compound or group is substituted with at least one (for example, 1, 2, 3, or 4) substituent independently selected from a halogen (—F, —Cl, —Br, —I), a hydroxyl (—OH), a C₁—C alkoxy, a C₁-C₉ haloalkoxy, an oxo (═O), a nitro (—NO₂), a cyano (—CN), an amino (—NR₂, wherein each R is independently hydrogen or C₁-C₁₀ alkyl), an azido (—N₃), an amidino (—C(═NH)NH₂), a hydrazino (—NHNH₂), a hydrazono (—C(═NNH₂)—), a carbonyl (—C(═O)—), a carbamoyl group (—C(O)NH₂), a sulfonyl (—S(═O)₂—), a thiol (—SH), a thiocyano (—SCN), a tosyl (CH₃C₆H₄SO₂—), a carboxylic acid (—C(═O)OH), a carboxylic C₁-C₆ alkyl ester (—C(═O)OR wherein R is C₁-C₁₀ alkyl), a carboxylic acid salt (—C(═O)OM) wherein M is an organic or inorganic anion, a sulfonic acid (—SO₃H₂), a sulfonic mono- or dibasic salt (—SO₃MH or —SO₃M₂ wherein M is an organic or inorganic anion), a phosphoric acid (—PO₃H₂), a phosphoric acid mono- or dibasic salt (—PO₃MH or —PO₃M₂ wherein M is an organic or inorganic anion), a C₁-C₁₂ alkyl, a C₃-C₁₂ cycloalkyl, a C₂-C₁₂ alkenyl, a C₅-C₁₂ cycloalkenyl, a C₂-C₁₂ alkynyl, a C₆-C₁₂aryl, a C₇-C₁₃ arylalkylene, a C₄-C₁₂ heterocycloalkyl, and a C₃-C₁₂ heteroaryl instead of hydrogen, provided that the substituted atom's normal valence is not exceeded. Alternatively, or additionally, when referring to a substituted aryl (aromatic) or heteroaryl (heteroaromatic), the term “substituted” can refer to a fused or bridged aryl, heteroaryl, or C₂₋₆ heterocyclyl.

“Pharmaceutical composition” means a composition comprising at least one active agent, such as a compound or salt of Formula (I), and at least one other substance, such as a carrier. Pharmaceutical compositions can meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, for example, excipient, diluent, or vehicle, that is useful in preparing a pharmaceutical composition that is generally safe, non-toxic and neither biologically nor otherwise undesirable, and can include a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. For example, the patient can be a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. Treatment of the disease can be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

Compounds of formulae can contain one or more asymmetric elements such as stereogenic centers, stereogenic axes and the like, for example, asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent can be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, for example, melting points, boiling points, spectral properties, and reactivities. Mixtures of diastereomers can separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which can occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture can occur where there has been no stereoselection or stereospecificity in a chemical reaction or process. Combinations of two enantiomeric species other than 50:50 racemic mixtures are also provided by the present disclosure, for example, 1:10,000, 1:1,000, 1:100, 1:10, 1:9, 1:7.5, 1:5, 1:3, 1:2.5, 1:2, or 1:1.5, or any opposite ratio, or any intervening ratio.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions can be carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids, for example, hydrochloric, hydrobromic, sulfuric, sulfamic, phosphoric, nitric and the like; and the salts prepared from organic acids, for example, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Any suitable pharmaceutical salt can be used.

Abbreviations used in present disclosure include the following: D₂R, dopamine D₂ receptor; D₃R, dopamine D₃ receptor; BD-1, bipolar 1 disorder; BMS, borane-dimethyl sulfide; BRET, bioluminescence resonance energy transfer; DIPEA, N,N-diisopropylethylamine; EDCI, 1-ethyl-3-(3-dimethylaminopropyl)carbodiimide·HCl; GPA, G-protein Activation; HCTU, O-(1H-6-chlorobenzotriazole-1-yl)-1,1,3,3-tetramethyluronium hexafluorophosphate; HEK 293 cells, human embryonic kidney 293 cells; HOBt, hydroxybenzotriazole; MPO, multiparameter optimization, PP, primary pharmacophore; PSUD, psychostimulant use disorders; SP, secondary pharmacophore; SUD, substance use disorders.

Compounds of the present disclosure include, for example, compounds having D₃R/D₂R selectivities while maintaining high (low nM to pM) binding affinities at D₃R. These ligands are considered bitopic, meaning they include a primary pharmacophore (PP), which binds to the orthosteric binding site, that is then linked to a secondary pharmacophore (SP), which binds to D₃R-unique secondary binding pocket.

Accordingly, the present disclosure provides a compound having Formula (I), an enantiomer thereof, a racemate thereof, a salt thereof, or any combination thereof:

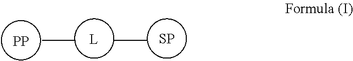

wherein
   PP is primary pharmacophore of formula PP¹:

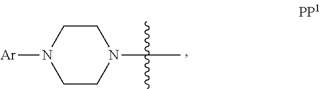

wherein Ar is a substituted or unsubstituted aromatic or heteroaromatic substituent;
L is a linker, covalently bound to the primary pharmacophore, of formula L¹, L^{2a}, L^{2b}, L^{2c}, or L^{2d}:

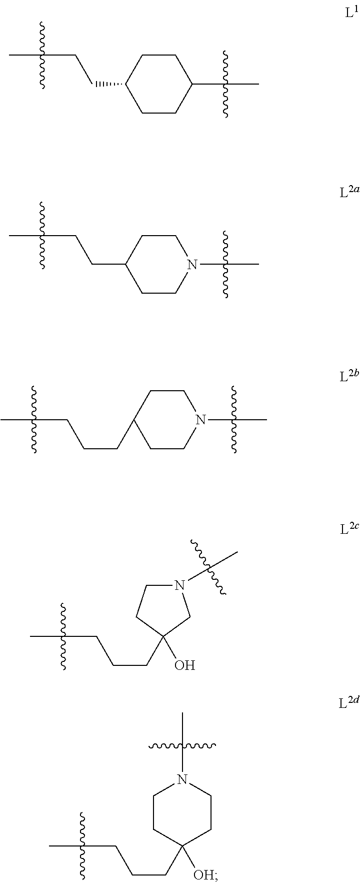

and
SP is a secondary pharmacophore, covalently bound to the linker, of formula SP¹ when L is L¹, or of formula SP² when L is L^{2a}, L^{2b}, L^{2c}, or L^{2d}:

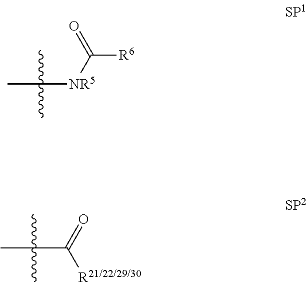

wherein R⁵ is hydrogen or C₁₋₈ alkyl,
R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
R⁵ and R⁶ combine to form a 5-, 6-, or 7-membered cyclic amide, cyclic carbamate, or cyclic urea, and
R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent; and
wherein the compound is a selective dopamine D₃ receptor partial agonist.

In any of the embodiments of the compound of Formula (I), disclosed herein, Ar is a substituted or unsubstituted aromatic or heteroaromatic substituent. In some embodiments Ar is a substituted or unsubstituted aromatic substituent (e.g., a substituted or unsubstituted phenyl substituent). In other embodiments, Ar is a substituted or unsubstituted heteroaromatic substituent (e.g., substituted or unsubstituted pyridyl). In some embodiments, Ar is

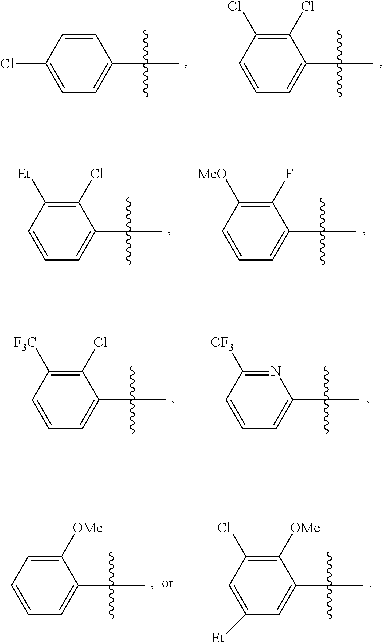

In certain embodiments, Ar is

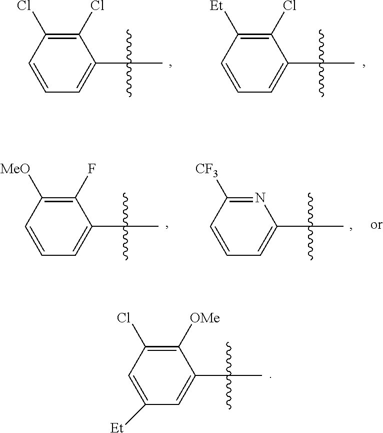 [SMILES: *c1cccc(Cl)c1Cl ; *c1cccc(CC)c1Cl ; *c1cccc(OC)c1F ; *c1cccc(C(F)(F)F)n1 ; *c1cc(CC)cc(Cl)c1OC]

In any of the embodiments of the compound of Formula (I), L is a linker, covalently bound to the primary pharmacophore, of formula L¹, L^{2a}, L^{2b}, L^{2c}, or L^{2d}:

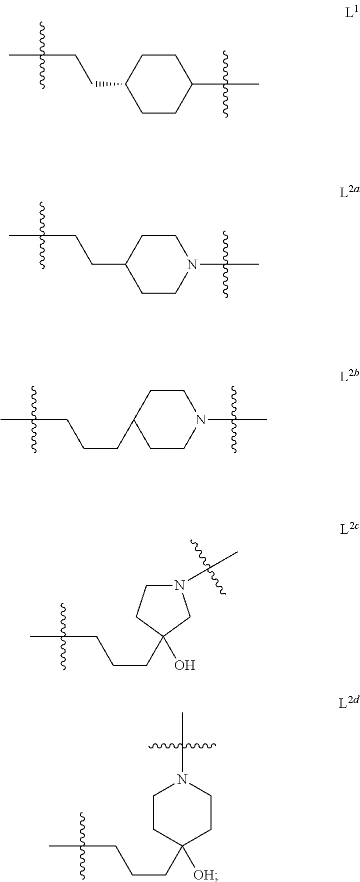

and SP is a secondary pharmacophore, covalently bound to the linker. Generally, SP (i.e., the secondary pharmacophore) is (i) of formula SP¹ when L is L¹ or (ii) of formula SP² when L is L^{2a}, L^{2b}, L^{2c}, or L^{2d}:

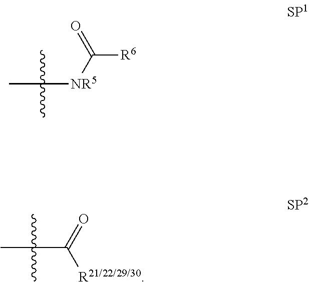

In any of the embodiments of the compound of Formula (I), R⁵ is hydrogen or C₁₋₈ alkyl (e.g., methyl, ethyl, propyl, or butyl) and R⁶ is an amine, C₁₋₈ alkyl amine, C₁₋₃ alkyl, C₁₋₈ alkoxy, a substituted or unsubstituted heterocycle, a substituted or unsubstituted aromatic, a substituted or unsubstituted heteroaromatic, or an ether-containing alkyl substituent, or R⁵ and R⁶ combine to form a 5-, 6-, or 7-membered cyclic amide, cyclic carbamate, or cyclic urea.

In some embodiments, R⁵ is hydrogen and R⁶ is an amine, C₁₋₈ alkyl amine, C₁₋₃ alkyl, C₁₋₈ alkoxy, a substituted or unsubstituted heterocycle, a substituted or unsubstituted aromatic, a substituted or unsubstituted heteroaromatic, or an ether-containing alkyl substituent. In other embodiments, R⁵ is C₁₋₈ alkyl (e.g., methyl, ethyl, propyl, or butyl) and R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent. For example, R⁶ can be

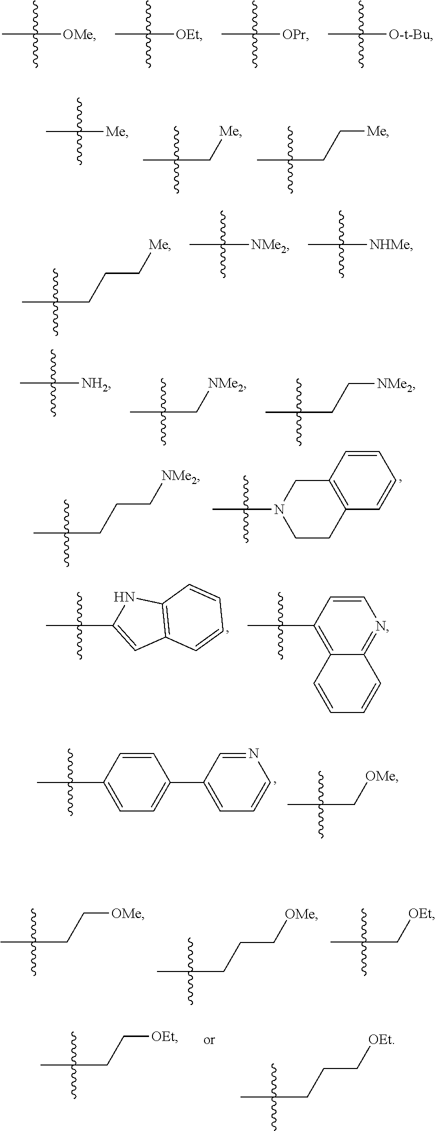

In certain embodiments, R⁵ and R⁶ combine to form a 5-, 6-, or 7-membered cyclic amide, cyclic carbamate, or cyclic urea. For example, R⁵ and R⁶ can combine to form

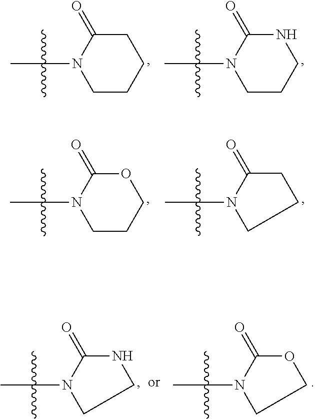 [SMILES: *N1CCCCC1=O ; *N1CCCNC1=O ; *N1CCCOC1=O ; *N1CCCC1=O ; *N1CCNC1=O ; *N1CCOC1=O]

In other words, SP¹ can be a 5-, 6-, or 7-membered cyclic amide, cyclic carbamate, or cyclic urea such as, for example, a group selected from

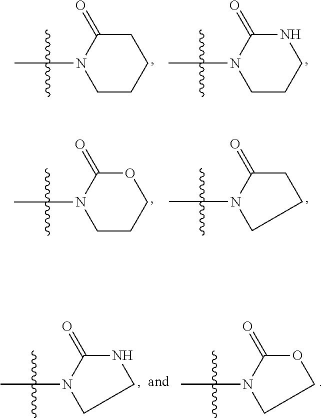 [SMILES: *N1CCCCC1=O ; *N1CCCNC1=O ; *N1CCCOC1=O ; *N1CCCC1=O ; *N1CCNC1=O ; *N1CCOC1=O]

In any of the embodiments of the compound of Formula (I), R^{21/22/29/30} is an amine, C₁₋₈ alkyl amine, C₁₋₃ alkyl, C₁₋₈ alkoxy, a substituted or unsubstituted heterocycle, a substituted or unsubstituted aromatic, a substituted or unsubstituted heteroaromatic, or an ether-containing alkyl substituent. For example, R^{21/22/29/30} can be

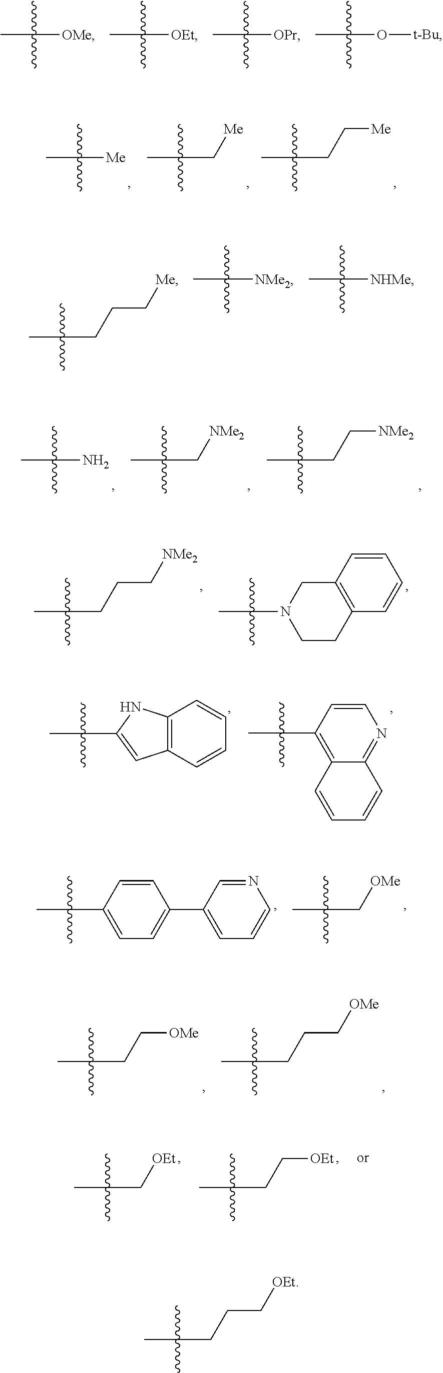

In some embodiments, the present disclosure provides a compound having Formula (I), an enantiomer thereof, a racemate thereof, a salt thereof, or any combination thereof, wherein the compound is represented by primary pharmacophore PP¹, linker L¹, and secondary pharmacophore SP¹:

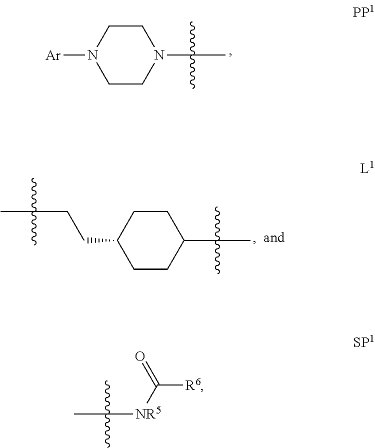

wherein
   Ar is a substituted or unsubstituted aromatic or heteroaromatic substituent,
   R⁵ is hydrogen or C₁₋₈ alkyl, and
   R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or R⁵ and R⁶ combine to form a 5-, 6-, or 7-membered cyclic amide, cyclic carbamate, or cyclic urea.

In some embodiments, the present disclosure provides a compound having Formula (I), an enantiomer thereof, a racemate thereof, a salt thereof, or any combination thereof, wherein the compound is represented by primary pharmacophore PP¹, linker L^{2a}, L^{2b}, L^{2c}, or L^{2d}, and secondary pharmacophore SP²:

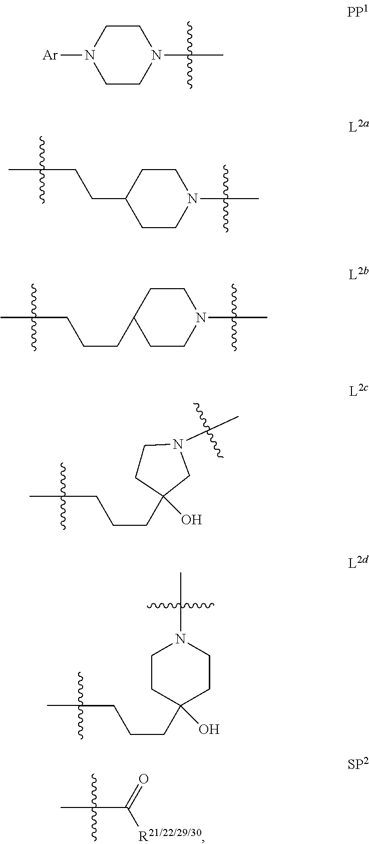

wherein
   Ar is a substituted or unsubstituted aromatic or heteroaromatic substituent, and
   R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent. For example, the compound having Formula (I), an enantiomer thereof, a racemate thereof, a salt thereof, or any combination thereof, can be represented by primary pharmacophore PP², linker L^{2a}, L^{2b}, L^{2c}, or L^{2d}, and secondary pharmacophore SP²:

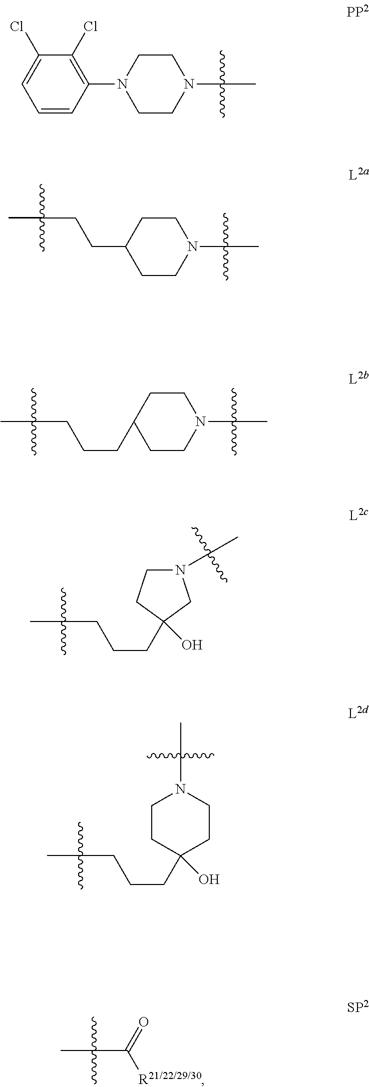

wherein R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent. In certain embodiments, R^{21/22/29/30} is an amine or an ether-containing alkyl.

Compounds of the present disclosure can exclude one or more substituents of the formulae described herein. Additionally, or alternatively, compounds of the present disclosure can exclude one or more indicated compounds. For example, one or more of the following can be excluded:

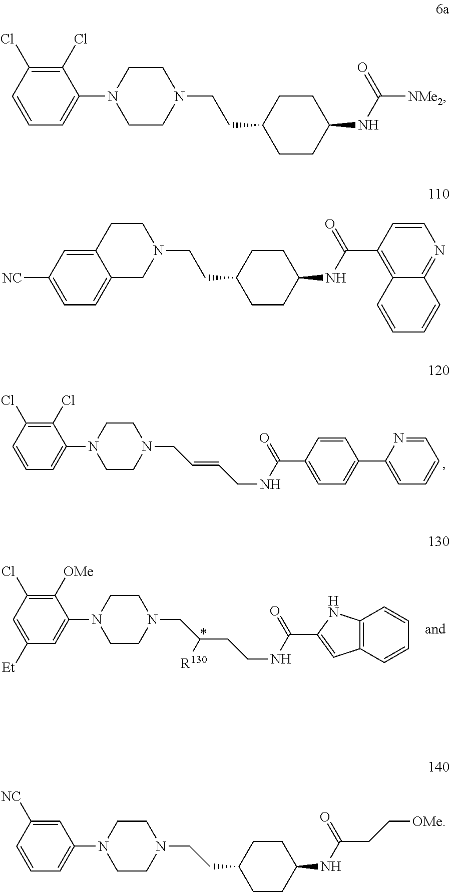

In compound 130, R¹³⁰ can be a hydrogen, hydroxyl, or a fluoride.

In some embodiments, the present disclosure provides a compound having Formula (I), its enantiomer, a racemate thereof, a salt thereof, or any combination thereof:

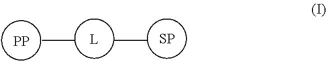

wherein PP is a primary pharmacophore, L is a linker covalently bound to the primary pharmacophore, and SP is a secondary pharmacophore covalently bound to the linker. The compound can be a selective dopamine D₃ receptor partial agonist. The compound can be represented by a first primary pharmacophore PP¹, a first linker L¹, and a first secondary pharmacophore SP¹:

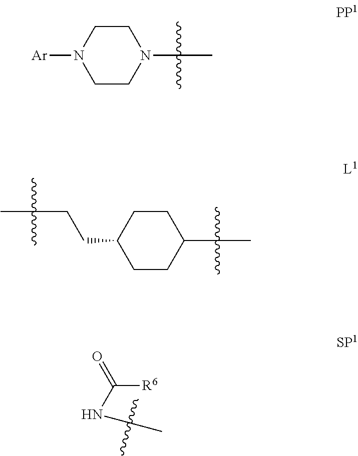

wherein Ar can be a substituted or unsubstituted aromatic substituent, and R⁶ can be an amine, a heterocycle, or an ether-containing alkyl substituent. The heterocycle can be a cycloalkane, or aromatic, or both. Alternatively, the compound can be represented by a second primary pharmacophore PP², a second linker L², and a second secondary pharmacophore SP²:

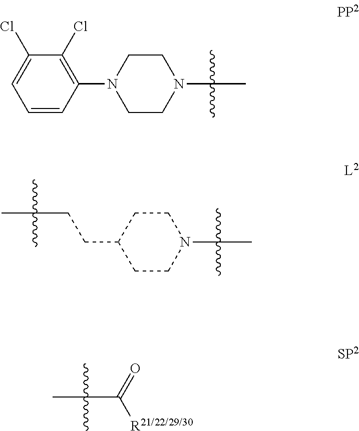

wherein R^{21/22/29/30} can be an amine or an ether-containing alkyl.

In some embodiments, the compound having Formula (I) is represented by Formula (II):

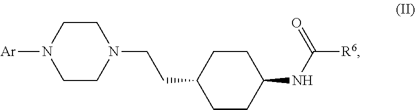

an enantiomer thereof, a racemate thereof, a salt thereof, or any combination thereof,
wherein
   Ar is a substituted or unsubstituted aromatic or heteroaromatic substituent and
   R⁵ is hydrogen or C₁₋₈ alkyl,
   R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
   R⁵ and R⁶ combine to form a 5-, 6-, or 7-membered cyclic amide, cyclic carbamate, or cyclic urea.

In certain embodiments, the compound having Formula (I) is represented by Formula (II):

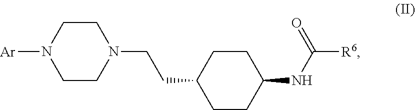

an enantiomer thereof, a racemate thereof, a salt thereof, or any combination thereof,
wherein
   Ar is a substituted or unsubstituted aromatic or heteroaromatic substituent and
   R⁶ is an amine, a heterocycle, or an ether-containing alkyl substituent.

In certain embodiments, the compound having Formula (I) is represented by Formula (II):

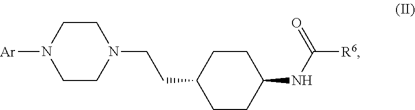

an enantiomer thereof, a racemate thereof, a salt thereof, or any combination thereof,
wherein
   Ar is a substituted or unsubstituted aromatic or heteroaromatic substituent and
   R⁶ is

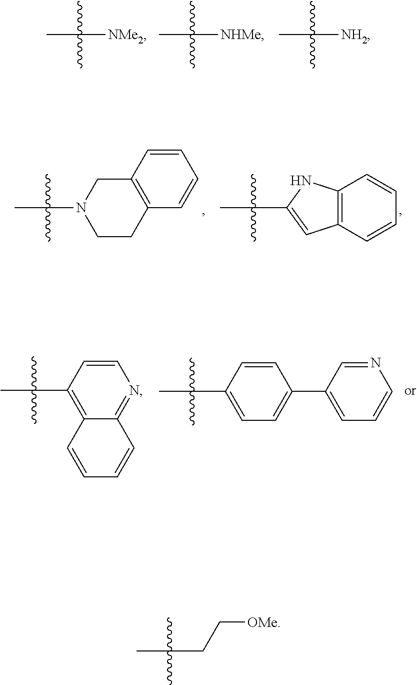

In certain embodiments, the compound having Formula (I) is represented by Formula (IIIa):

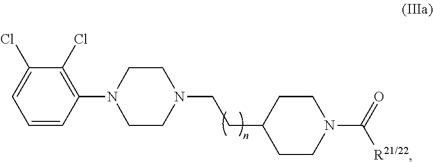

an enantiomer thereof, a racemate thereof, a salt thereof, or any combination thereof,
wherein
   n is 1 or 2, and

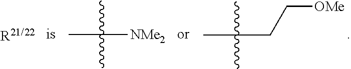

In certain embodiments, the compound having Formula (I) is represented by Formula (IIIb):

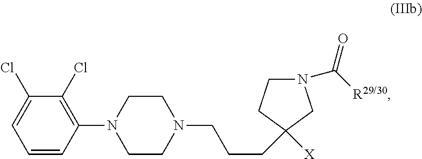

an enantiomer thereof, a racemate thereof, a salt thereof, or any combination thereof,
wherein
   X is OH, and

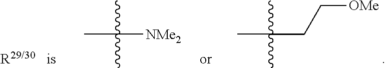

In some embodiments, the compound is of formula:

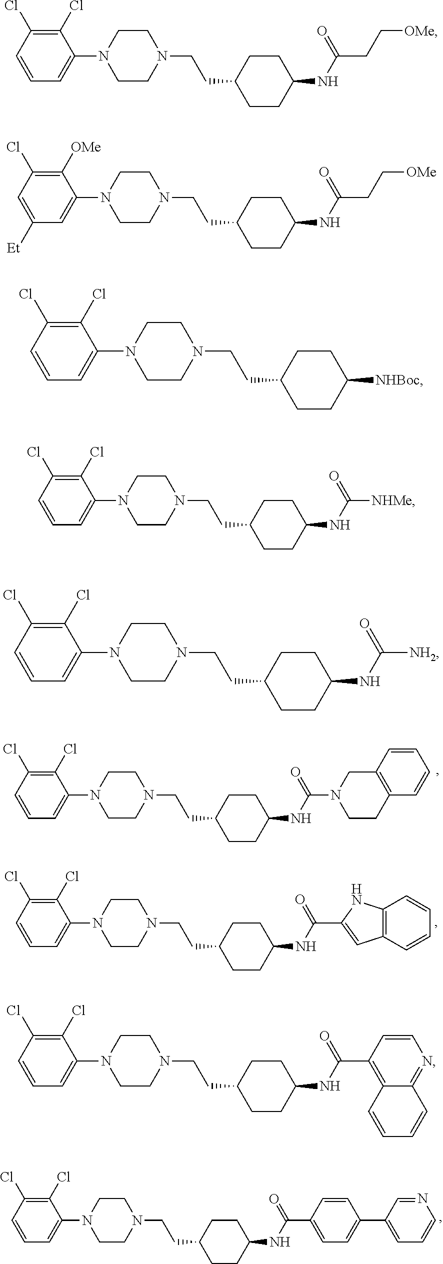

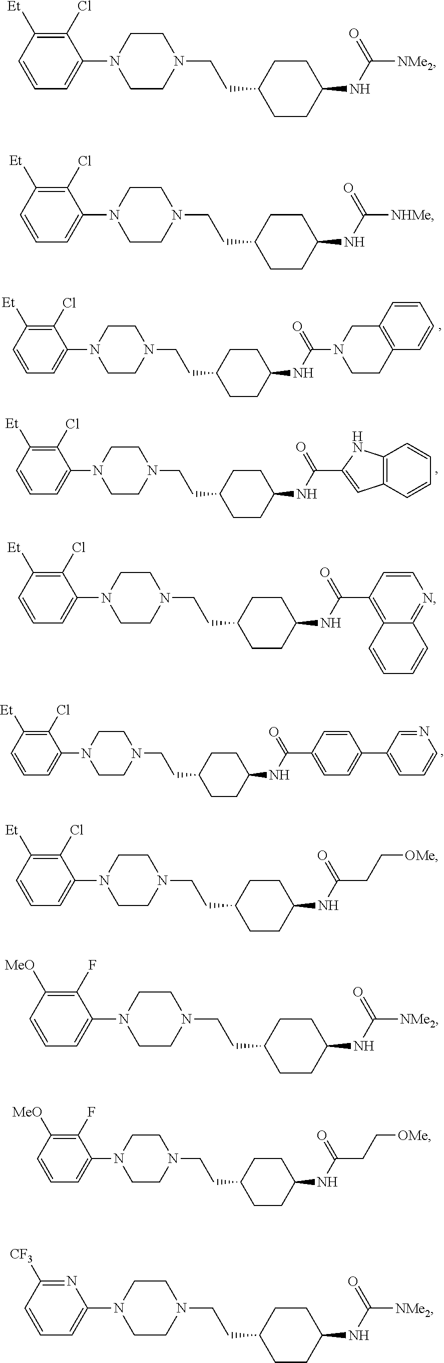

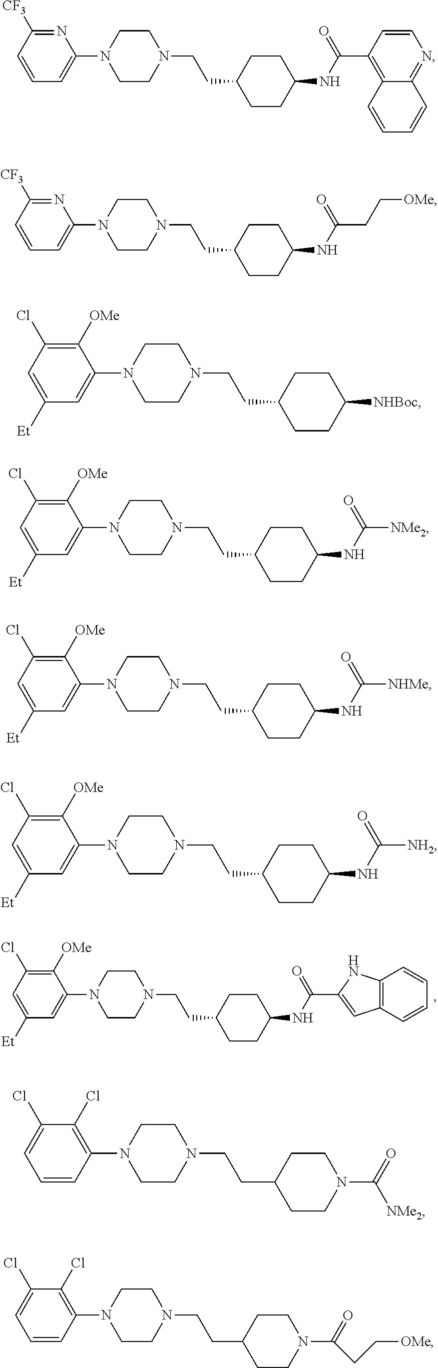

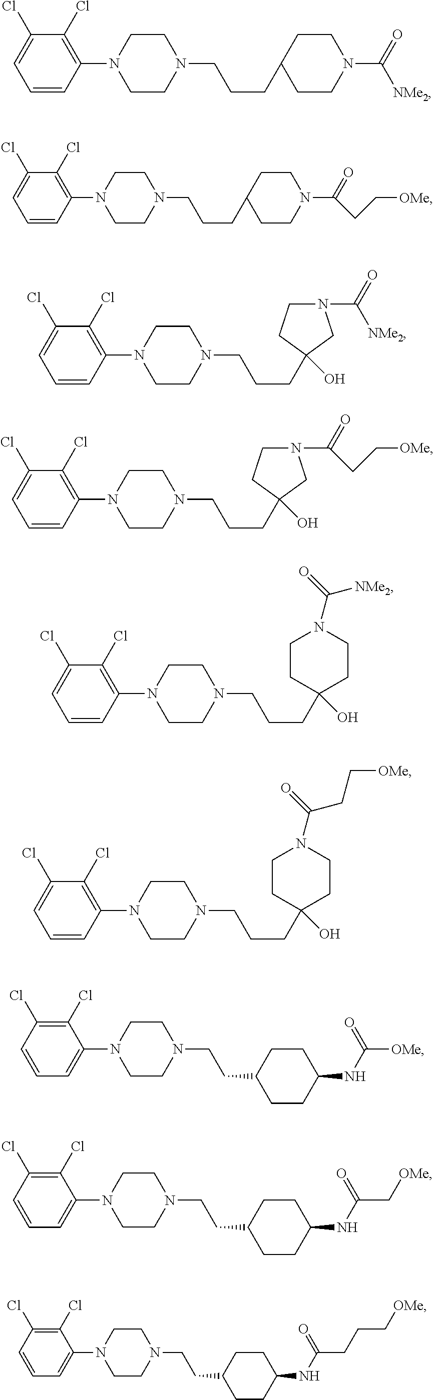

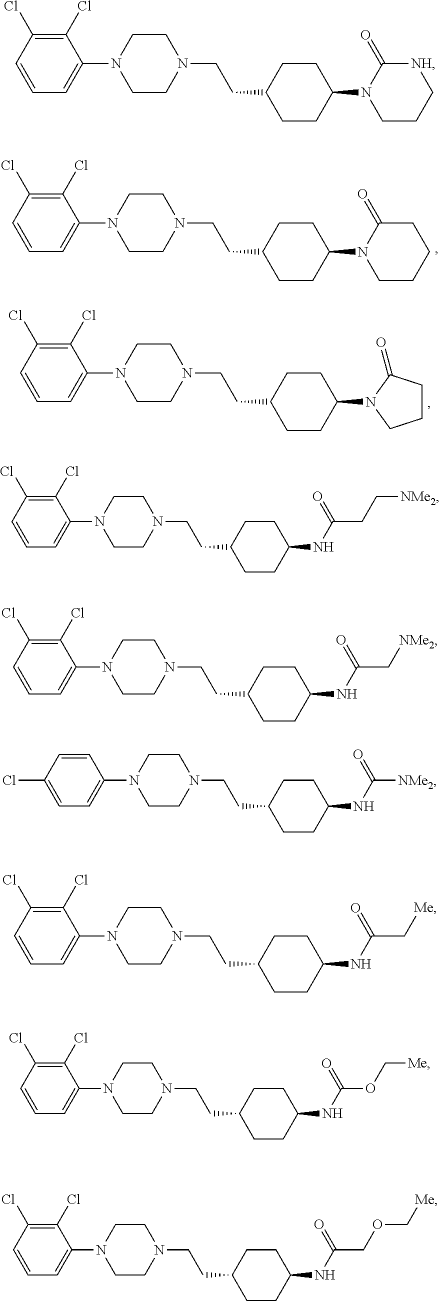

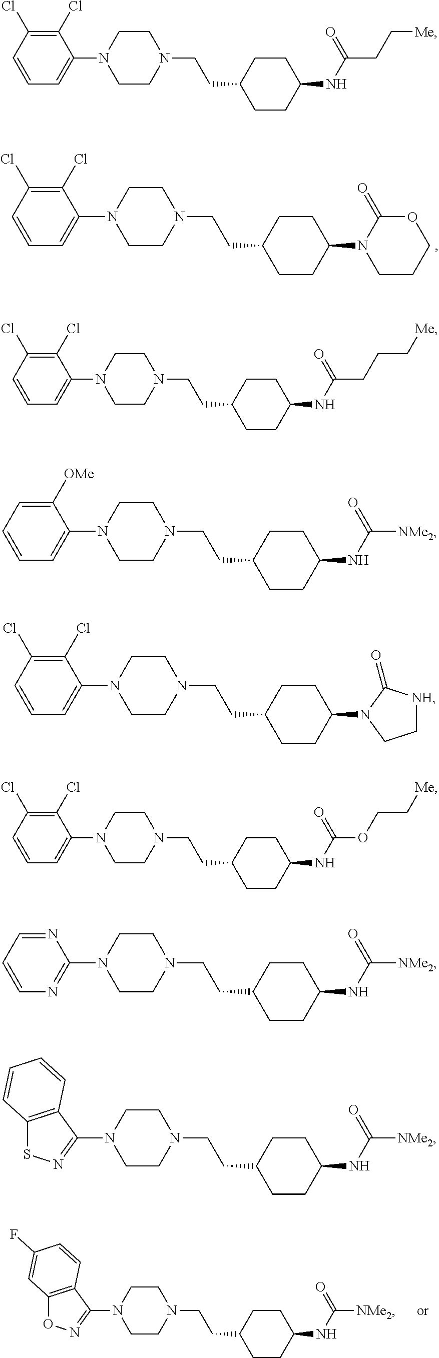

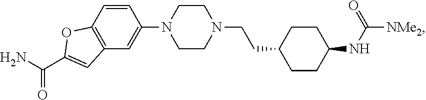 [SMILES: CN(C)C(=O)N[C@H]1CC[C@H](CCN2CCN(c3ccc4oc(C(N)=O)cc4c3)CC2)CC1]

or an enantiomer thereof, a racemate thereof, a salt thereof, or any combination thereof.

In certain embodiments, the compound is of formula:

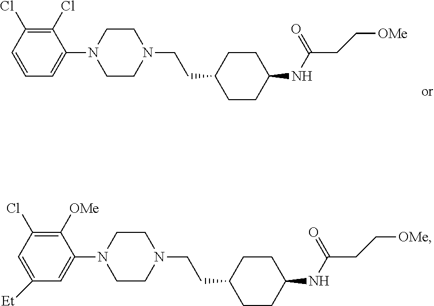 [SMILES: COCCC(=O)N[C@H]1CC[C@H](CCN2CCN(c3cccc(Cl)c3Cl)CC2)CC1 ; CCc1cc(Cl)c(OC)c(N2CCN(CC[C@H]3CC[C@H](NC(=O)CCOC)CC3)CC2)c1]

or a salt thereof.

Pharmaceutical compositions are provided by the present disclosure. For example, a pharmaceutical composition is provided that comprises a therapeutically effective amount of one or more compounds disclosed herein, their enantiomers, racemates thereof, salts thereof, or any combination thereof, together with a pharmaceutically acceptable carrier. The pharmaceutically acceptable carrier can be, for example, selected from the group consisting of binders, buffering agents, coloring agents, diluents, disintegrants, emulsifiers, flavorants, glidants, lubricants, preservatives, stabilizers, surfactants, tableting agents, and wetting agents, and combinations thereof. The pharmaceutical composition can provide an additional therapeutic compound (e.g., a second therapeutic compound) in addition to the one or more compounds disclosed herein. In other words, the compound of the pharmaceutical composition can be a first therapeutic compound and the pharmaceutical composition can further comprise a second therapeutic compound. The first and second therapeutic compounds can act additively or synergistically.

The second therapeutic compound can comprise, for example, an antidepressant, an antipsychotic, or a combination thereof. Antidepressants can include one or more of a selective serotonin reuptake inhibitor (SSRI), serotonin-norepinephrine uptake inhibitor (SNRI), a 5-HT modulator, a unicyclic antidepressant, a tricyclic antidepressant (TCA), a tetracyclic antidepressant, a mixed norepinephrine/serotonin reuptake inhibitor or receptor blocker, a NMDA antagonist, a GABA modulator, a mixed-action drug, and a monoamine oxidase inhibitor. For example, the antidepressant can include one or more of fluoxetine, sertraline, paroxetine, fluvoxamine, citalopram, escitalopram, amitriptyline, nortriptyline, protriptyline, imipramine, desipramine, doxepin, clomipramine, trimipramine, venlafaxine, desvenlafaxine, duloxetine, maprotiline, milnacipran, mirtazapine, vilazodone, bupropion, trazodone, nefazodone, vortioxetine, amoxapine, phenelzine, tranylcypromine, isocarboxid, selegiline, esketamine, and brexanolone. Antipsychotics can include, for example, a first-generation antipsychotic agent, or a second antipsychotic agent, or both. Antipsychotics can include, for example, one or more of a phenothiazine, a thioxanthene, a butyrophenone, a dibenzodiazepine, a benzisoxazole, a thienobenzodiazepine, a dibenzothiazepine, a dihydroindolone, and a dihydrocarbostyril. For example, the antipsychotic can include one or more chlorpromazine, thioridazine, perphenazine, loxapine, molindone, pimoizide, loxapine, haloperidol, fluphenazine, trifluoperazine, thiothixene, clozapine, risperidone, olanzapine, quetiapine, ziprasidone, aripiprazole, paliperidone, iloperidone, asenapine, and lurasidone. The second therapeutic compound can include an anxiolytic, for example, one or more of diazepam, flurazepam, triazolam, lorazepam, alprazolam, chlordiazepoxide, oxazepam, temazepam, clonazepam, and buspirone. The anxiolytic can be a benzodiazepine, or a non-bezodiazepine. The second therapeutic compound can include a mood stabilizer. The mood stabilizer can include, for example, one or more of lithium, valproic acid, carbamazepine, oxcarbazepine, and lamotrigine. In certain embodiments, the second therapeutic compound comprises an antidepressant such as, for example, a selective serotonin reuptake inhibitor (SSRI). For methods of treatment, a second or further therapeutic compound can be administered separately or in combination with the first therapeutic agent (i.e., a compound disclosed herein).

Methods of treatment are provided by the present disclosure that employ one or more compounds disclosed herein. The patient (subject) can be a mammal, and more specifically, a human, or non-human patients such as companion animals, for example, cats, dogs, and livestock animals. A method of treating drug misuse or drug addiction, for example, is provided. The method can comprise administering to a patient in need thereof a composition comprising a therapeutically effective amount of one or more compounds described herein, their enantiomers, racemates thereof, salts thereof, or any combination thereof optionally in combination with one or more additional active ingredients. The patient treated can have a history of misusing, for example, a stimulant, a depressant, an opioid, a cannabinoid, lysergic acid diethylamide (LSD), mescaline, psilocybin, nicotine, ethanol, a benzodiazepine, phencyclidine, ketamine, cocaine, or an amphetamine, or any combination thereof.

A method of treating a substance use disorder is also provided. The method can comprise administering to a patient in need thereof a composition comprising a therapeutically effective amount of one or more compounds described herein, their enantiomers, racemates thereof, salts thereof, or any combination thereof optionally in combination with one or more additional active ingredients. The patient treated can have one or more affective disorders, schizophrenia, or a combination thereof. Affective disorders can include, for example, depression, anxiety, major depressive disorder, mood disorders, bipolar disorders, a mania, panic disorders, phobias, stress disorders, obsessive-compulsive disorder. The substance use disorder can comprise, for example, a psychostimulant use disorder. The patient can have a history of misusing, for example, a stimulant comprising cocaine, an amphetamine, a methamphetamine, dextroamphetamine, levoamphetamine, methylenedioxymethamphetamine (MDMA), methylphenidate, modafinil, armodafinil, midodrine, oxymetazoline, dobutamine, ephedrine, pseudoephedrine, or phenylephrine, or any combination thereof.

A method of treating one or more affective disorders, schizophrenia, or a combination thereof. The method can comprise administering to a patient in need thereof a composition comprising a therapeutically effective amount of one or more compounds described herein, their enantiomers, racemates thereof, salts thereof, or any combination thereof optionally in combination with one or more additional active ingredients. Affective disorders can include, for example, depression, anxiety, major depressive disorder, mood disorders, bipolar disorders, a mania, panic disorders, phobias, stress disorders, obsessive-compulsive disorder. In some embodiments, the patient with one or more affective disorders, schizophrenia, or a combination thereof further has a substance use disorder.

Compounds disclosed herein can be administered as the neat chemical, or can be administered as a pharmaceutical composition. Accordingly, the disclosure encompasses pharmaceutical compositions comprising a therapeutically effective amount of a compound or pharmaceutically acceptable salt of a compound, together with at least one pharmaceutically acceptable carrier. The pharmaceutical composition can contain a therapeutically effective amount of the compound or salt as the only active agent, and can contain at least one additional active agent. The pharmaceutical composition can be in a dosage form that contains from about 0.1 mg to about 2000 mg, from about 10 mg to about 1000 mg, from about 100 mg to about 800 mg, or from about 200 mg to about 600 mg of a compound, and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition can be in a dosage form that contains about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100,150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of a compound, and optionally about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of an additional active agent in a unit dosage form. The pharmaceutical composition can also include a molar ratio of a compound and an additional active agent. For example, the pharmaceutical composition can contain a molar ratio of about 0.5:1, about 1:1, about 2:1, about 3:1 or from about 1.5:1 to about 4:1 of an additional active agent to a compound.

Compounds disclosed herein can be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition can be formulated as any pharmaceutically useful form, for example, as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, for example, a therapeutically effective amount to achieve the desired purpose.

Carriers include excipients and diluents of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers can be listed in more than one class, for example vegetable oil can be used as a lubricant in some formulations and a diluent in others. Exemplary pharmaceutically acceptable carriers include sugars, starches, celluloses, powdered tragacanth, malt, gelatin, talc, and vegetable oils. Optional active agents can be included in a pharmaceutical composition, which do not substantially interfere with the activity of the compound of the present disclosure.

The pharmaceutical compositions/combinations can be formulated for oral administration. These compositions contain between 0.1 and 99 weight % (wt %) of a compound and usually at least about 5 wt % of a compound of Formula (I). Compositions can contain from about 25 wt % to about 50 wt % or from about 5 wt % to about 75 wt % of the compound. Compositions can contain about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, or 95 wt % of the compound.

The methods of treatment disclosed herein include providing any suitable dosage amounts of a compound to a patient. Dosage levels of each compound of from about 0.1 mg to about 140 mg per kilogram of body weight per day are useful in the treatment of the above-indicated conditions (about 0.5 mg to about 7 g per patient per day). The amount of compound that can be combined with the carrier materials to produce a single dosage form will vary depending upon the patient treated and the particular mode of administration. Dosage unit forms can contain, for example, between from about 1 mg to about 500 mg of each active compound. For example, 25 mg to 500 mg, or 25 mg to 200 mg of a compound can be provided daily to a patient. Frequency of dosage can also vary. A dosage regimen of 4 times daily or less can be used, or a dosage regimen of 1 or 2 or 3 times daily can be used. The specific dose level for any particular patient can depend upon a variety of factors including the activity of the specific compound employed, the age, body weight, general health, sex, diet, time of administration, route of administration, and rate of excretion, drug combination, and the severity of the particular disease undergoing therapy.

The compounds employed in the methods of the present disclosure can exist in prodrug form. As used herein, “prodrug” is intended to include any covalently bonded carriers which release the active parent drug, for example, as according to a formula described herein, or other formulas or compounds employed in the methods of the present disclosure in vivo when such prodrug is administered to a mammalian subject. Because prodrugs are known to enhance numerous desirable qualities of pharmaceuticals (for example, solubility, bioavailability, manufacturing, etc.) the compounds employed in the present methods can, if desired, be delivered in prodrug form. Thus, the present disclosure contemplates methods of delivering prodrugs. Prodrugs of the compounds employed in the present disclosure can be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups, and alkyl, carbocyclic, aryl, and alkylaryl esters such as methyl, ethyl, propyl, iso-propyl, butyl, iso-butyl, sec-butyl, tert-butyl, cyclopropyl, phenyl, benzyl, and phenethyl esters, and the like.

### ASPECTS OF THE DISCLOSURE

Aspects, including embodiments, of the invention described herein may be beneficial alone or in combination, with one or more other aspects or embodiments. Without limiting the foregoing description, certain non-limiting aspects of the disclosure numbered 1-34 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
(1) In aspect (1) is provided a compound having Formula (I), an enantiomer thereof, a racemate thereof, a salt thereof, or any combination thereof:

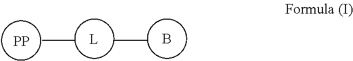

wherein
   PP is primary pharmacophore of formula PP¹:

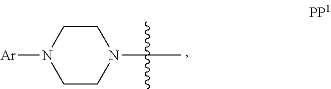

wherein Ar is a substituted or unsubstituted aromatic or heteroaromatic substituent;
L is a linker, covalently bound to the primary pharmacophore, of formula L¹, L^{2a}, L^{2b}, L^{2c}, or L^{2d}:

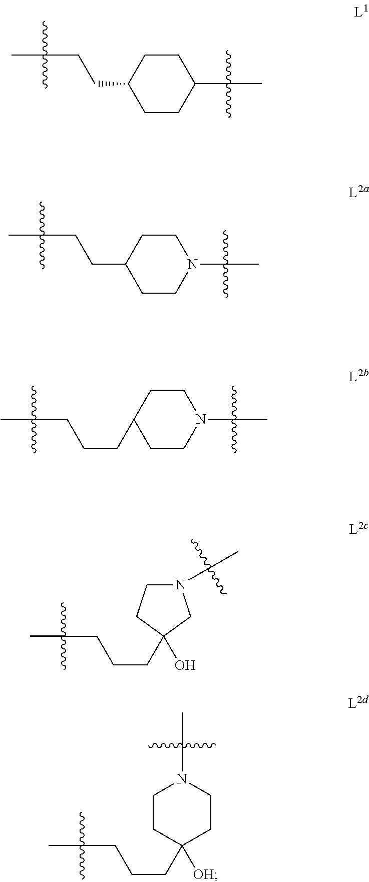

and
SP is a secondary pharmacophore, covalently bound to the linker, of formula SP¹ when L is L¹, or of formula SP² when L is L^{2a}, L^{2b}, L²C, or L^{2d}:

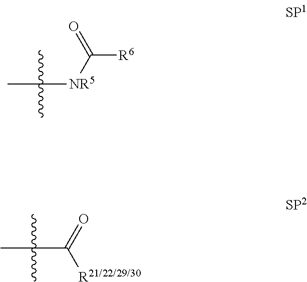

wherein R⁵ is hydrogen or C₁₋₈ alkyl,
R⁶ is an amine, C₁₋₈ alkyl amine, C₁₋₃ alkyl, C₁₋₈ alkoxy, a substituted or unsubstituted heterocycle, a substituted or unsubstituted aromatic, a substituted or unsubstituted heteroaromatic, or an ether-containing alkyl substituent, or
R⁵ and R⁶ combine to form a 5-, 6-, or 7-membered cyclic amide, cyclic carbamate, or cyclic urea, and
R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent; and
wherein the compound is a selective dopamine D₃ receptor partial agonist.
(2) In aspect (2) is provided the compound of aspect 1, an enantiomer thereof, a racemate thereof, a salt thereof, or any combination thereof, wherein the compound is represented by primary pharmacophore PP¹, linker L¹, and secondary pharmacophore SP¹:

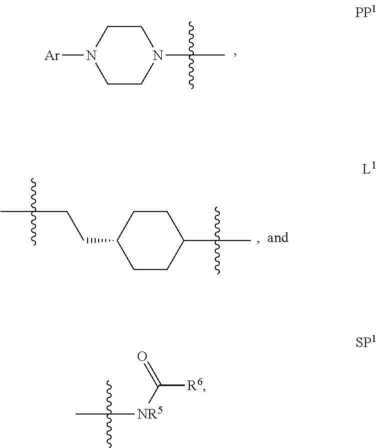

wherein
   Ar is a substituted or unsubstituted aromatic or heteroaromatic substituent,
   R⁵ is hydrogen or C₁₋₈ alkyl, and
   R⁶ is an amine, C₁₋₃ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
   R⁵ and R⁶ combine to form a 5-, 6-, or 7-membered cyclic amide, cyclic carbamate, or cyclic urea.
(3) In aspect (3) is provided the compound of aspect 1 or 2, an enantiomer thereof, a racemate thereof, a salt thereof, or any combination thereof, wherein R⁵ is hydrogen.
(4) In aspect (4) is provided the compound of any one of aspects 1-3, an enantiomer thereof, a racemate thereof, a salt thereof, or any combination thereof, wherein R⁶ is

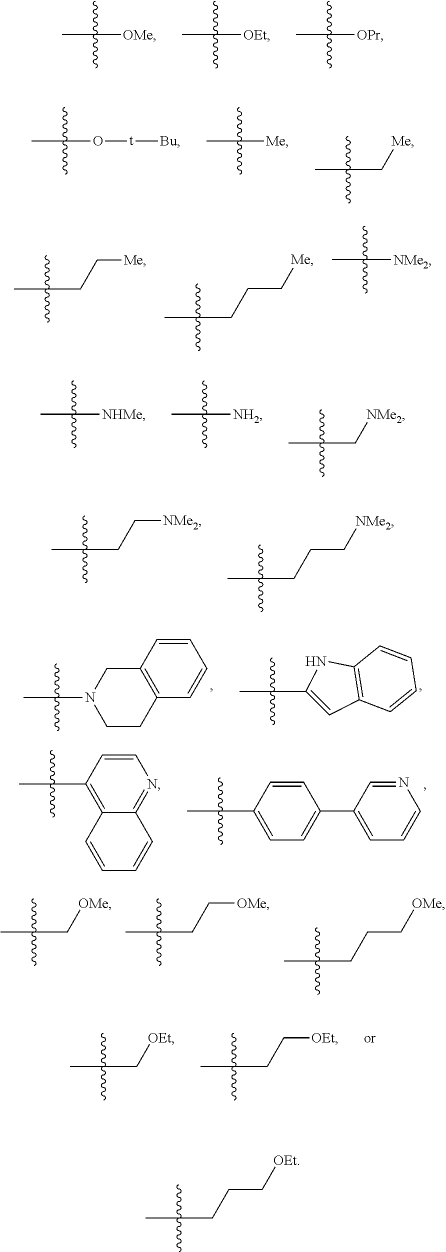

(5) In aspect (5) is provided the compound of aspect 1 or 2, an enantiomer thereof, a racemate thereof, a salt thereof, or any combination thereof, wherein R⁵ and R⁶ combine to form a 5-, 6-, or 7-membered cyclic amide, cyclic carbamate, or cyclic urea.
(6) In aspect (6) is provided the compound of aspect 5, an enantiomer thereof, a racemate thereof, a salt thereof, or any combination thereof, wherein SP¹ is

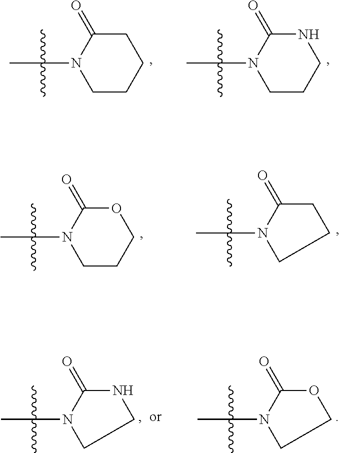 [SMILES: *N1CCCCC1=O ; *N1CCCNC1=O ; *N1CCCOC1=O ; *N1CCCC1=O ; *N1CCNC1=O ; *N1CCOC1=O]

(7) In aspect (7) is provided the compound of aspect 1 or 2, wherein Formula (I) is represented by Formula (II):

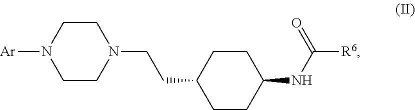

an enantiomer thereof, a racemate thereof, a salt thereof, or any combination thereof,
wherein
   Ar is a substituted or unsubstituted aromatic or heteroaromatic substituent and
   R⁶ is an amine, a heterocycle, or an ether-containing alkyl substituent.
(8) In aspect (8) is provided the compound of aspect 1 or 2, wherein Formula (I) is represented by Formula (II):

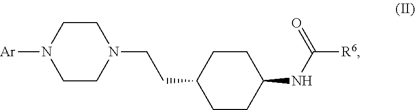

an enantiomer thereof, a racemate thereof, a salt thereof, or any combination thereof,
wherein
   Ar is a substituted or unsubstituted aromatic or heteroaromatic substituent and
   R⁶ is

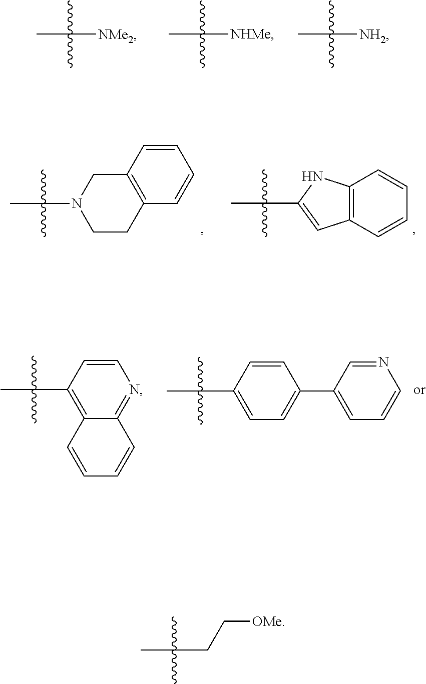

(9) In aspect (9) is provided the compound of aspect 1, an enantiomer thereof, a racemate thereof, a salt thereof, or any combination thereof, wherein the compound is represented by primary pharmacophore PP¹, linker L^{2a}, L^{2b}, L^{2c}, or L^{2d}, and secondary pharmacophore SP²:

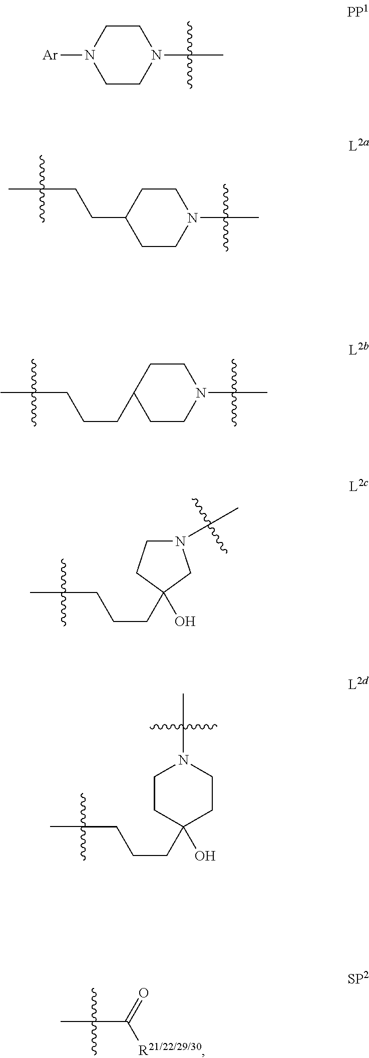

wherein Ar is a substituted or unsubstituted aromatic or heteroaromatic substituent, and
   R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent.
(10) In aspect (10) is provided the compound of aspect 9, an enantiomer thereof, a racemate thereof, a salt thereof, or any combination thereof, wherein R^{21/22/29/30} is

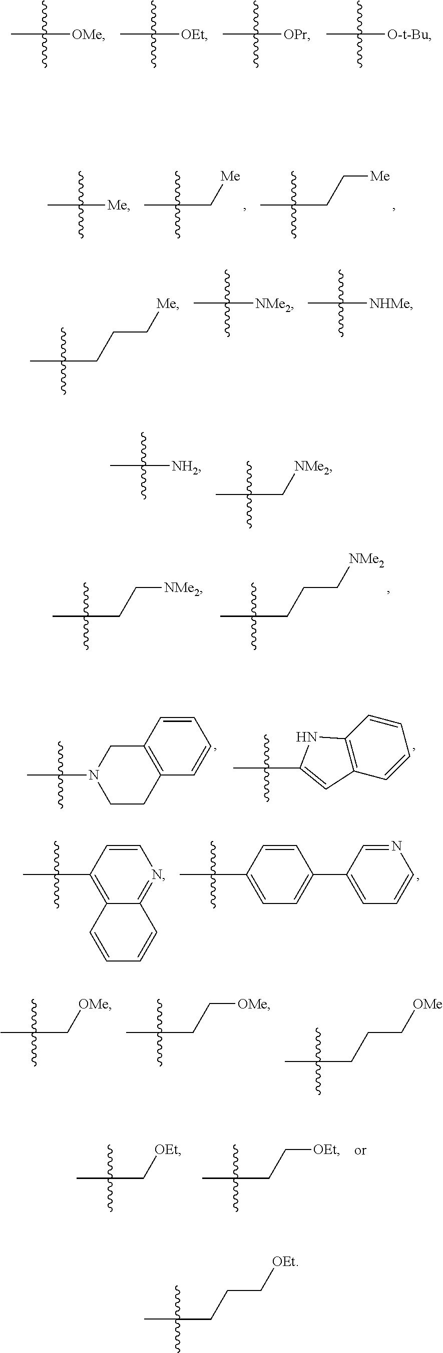

(11) In aspect (11) is provided the compound of any one of aspects 1-10, an enantiomer thereof, a racemate thereof, a salt thereof, or any combination thereof, wherein Ar is

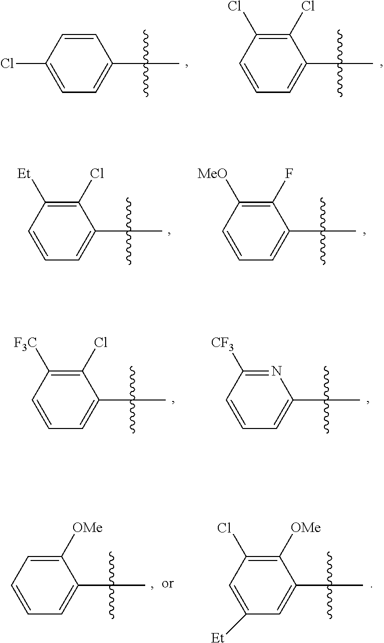

(12) In aspect (12) is provided the compound of any one of aspects 1-10, an enantiomer thereof, a racemate thereof, a salt thereof, or any combination thereof, wherein Ar is

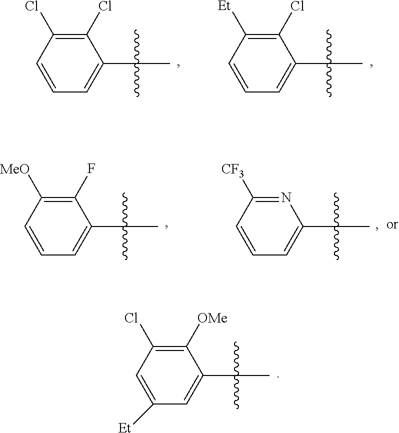 [SMILES: *c1cccc(Cl)c1Cl ; *c1cccc(CC)c1Cl ; *c1cccc(OC)c1F ; *c1cccc(C(F)(F)F)n1 ; *c1cc(CC)cc(Cl)c1OC]

(13) In aspect (13) is provided the compound of aspect 1, an enantiomer thereof, a racemate thereof, a salt thereof, or any combination thereof, wherein the compound is represented by primary pharmacophore PP², linker L^{2a}, L^{2b}, L^{2c}, or L^{2d}, and secondary pharmacophore SP²:

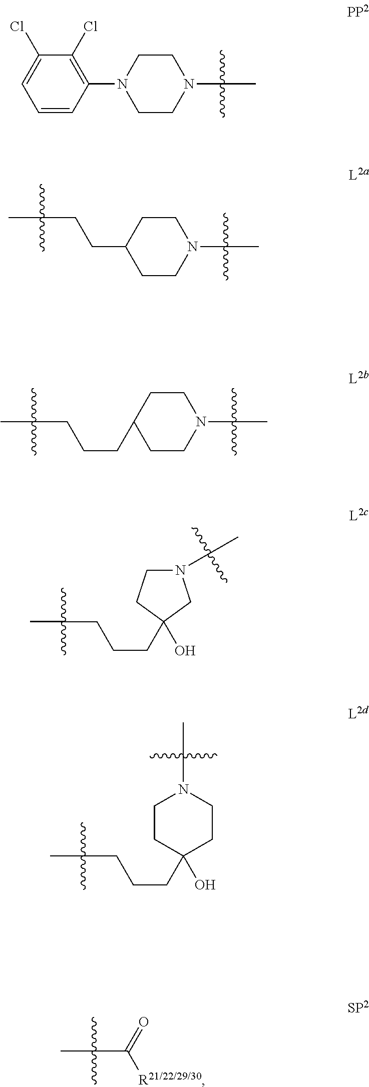

wherein R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent.
(14) In aspect (14) is provided the compound of aspect 1, an enantiomer thereof, a racemate thereof, a salt thereof, or any combination thereof, wherein the compound is represented by primary pharmacophore PP², linker L^{2a}, L^{2b}, |^{2c}, or L^{2d}, and secondary pharmacophore SP²:

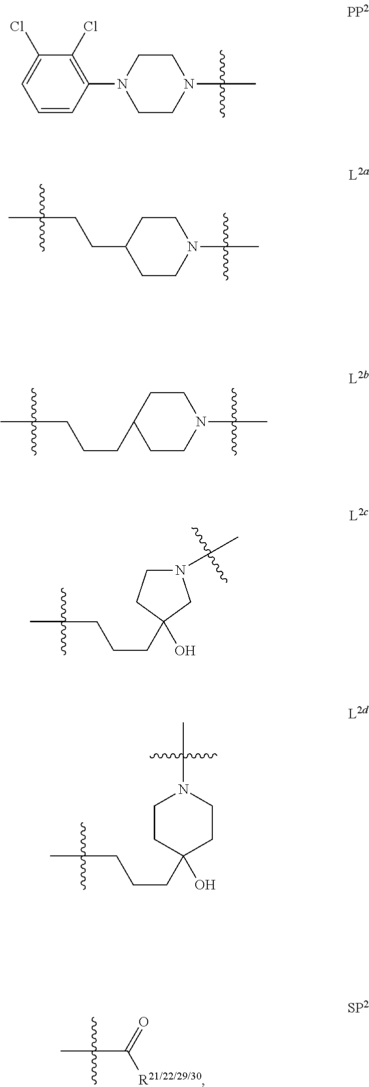

wherein R^{21/22/29/30} is an amine or an ether-containing alkyl.
(15) In aspect (15) is provided the compound of any one of aspects 1-14 or a salt thereof, wherein Ar is a substituted or unsubstituted aromatic substituent.
(16) In aspect (16) is provided the compound of aspect 1, wherein the compound of Formula (I) is

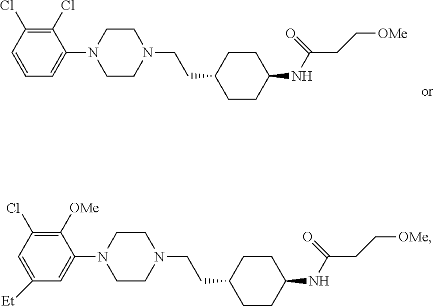 [SMILES: COCCC(=O)N[C@H]1CC[C@H](CCN2CCN(c3cccc(Cl)c3Cl)CC2)CC1 ; CCc1cc(Cl)c(OC)c(N2CCN(CC[C@H]3CC[C@H](NC(=O)CCOC)CC3)CC2)c1]

or a salt thereof.
(17) In aspect (17) is provided the compound of aspect 1, wherein the compound of Formula (I) is

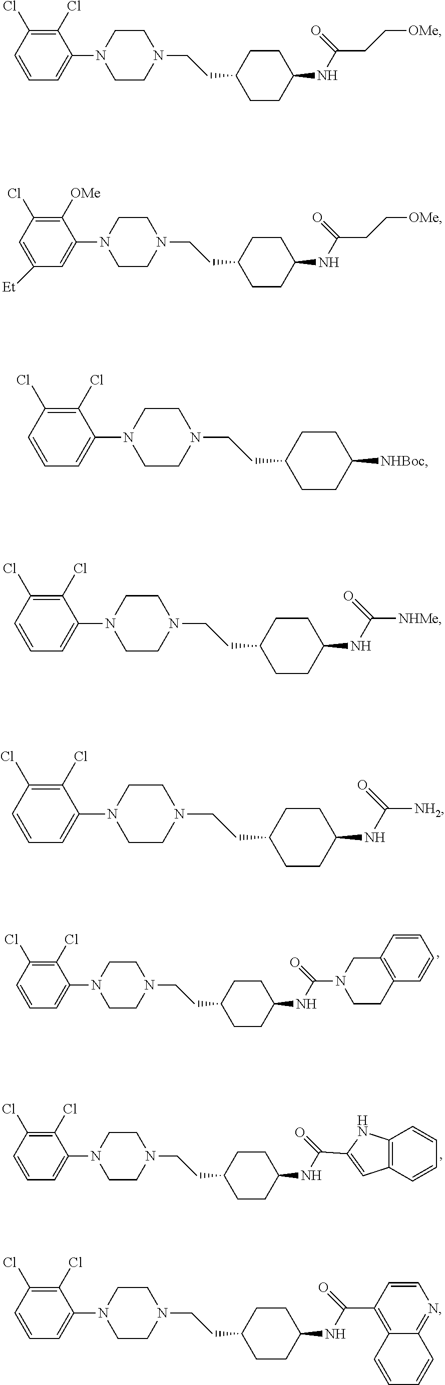

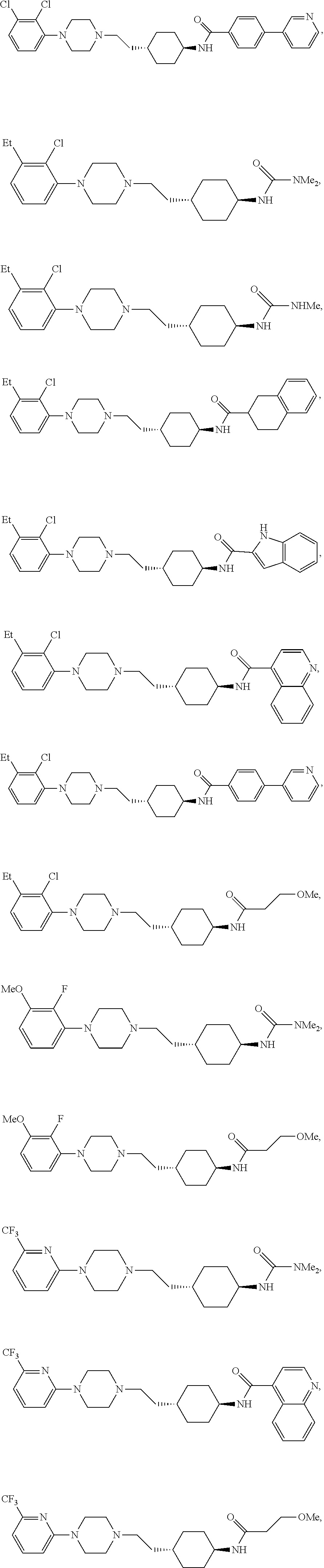

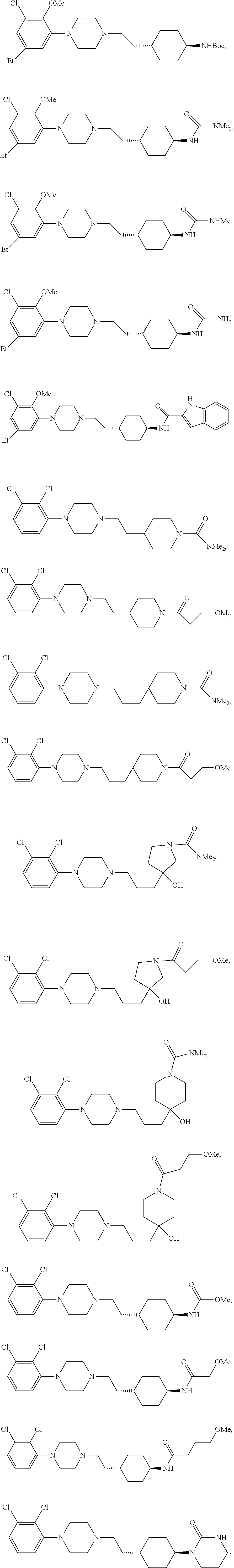

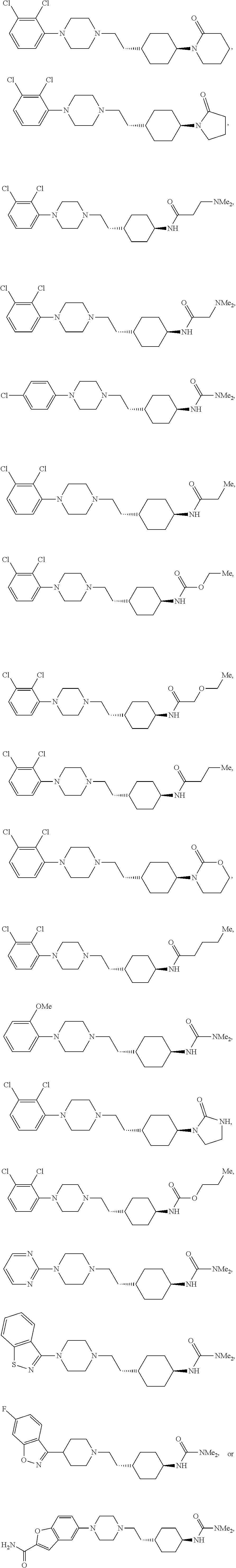

or an enantiomer thereof, a racemate thereof, a salt thereof, or any combination thereof.
(18) In aspect (18) is provided the compound of aspect 14, wherein Formula (I) is represented by Formula (IIIa):

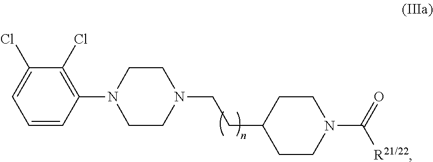

an enantiomer thereof, a racemate thereof, a salt thereof, or any combination thereof,
wherein
   n is 1 or 2, and
   R^{21/22} is

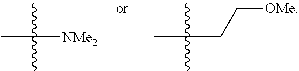

(19) In aspect (19) is provided the compound of aspect 14, wherein Formula (I) is represented by Formula (IIIb):

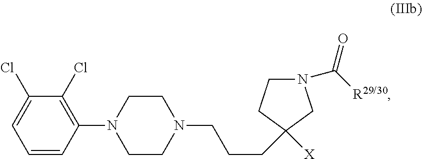

an enantiomer thereof, a racemate thereof, a salt thereof, or any combination thereof,
wherein
   X is OH, and
   R^{29/30} is

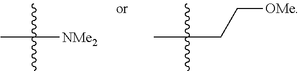

(20) In aspect (20) is provided the compound of aspect 1, wherein the compound excludes

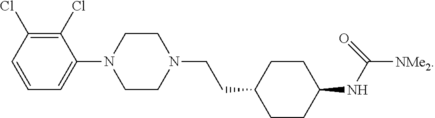 [SMILES: CN(C)C(=O)N[C@H]1CC[C@H](CCN2CCN(c3cccc(Cl)c3Cl)CC2)CC1]

(21) In aspect (21) is provided the compound of aspect 1, wherein the compound excludes

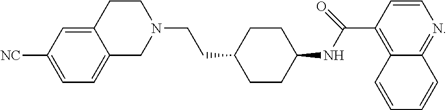 [SMILES: N#Cc1ccc2c(c1)CCN(CC[C@H]1CC[C@H](NC(=O)c3ccnc4ccccc34)CC1)C2]

(22) In aspect (22) is provided the compound of aspect 1, wherein the compound excludes

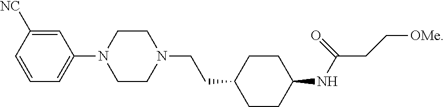 [SMILES: COCCC(=O)N[C@H]1CC[C@H](CCN2CCN(c3cccc(C#N)c3)CC2)CC1]

(23) In aspect (23) is provided a pharmaceutical composition comprising a therapeutically effective amount of a compound of any one of aspects 1-22, an enantiomer thereof, a racemate thereof, a salt thereof, or any combination thereof, together with a pharmaceutically acceptable carrier.
(24) In aspect (24) is provided the pharmaceutical composition of aspect 23, wherein the compound is a first therapeutic compound, and the pharmaceutical composition further comprises a second therapeutic compound.
(25) In aspect (25) is provided the pharmaceutical composition of aspect 24, wherein the second therapeutic compound comprises an antidepressant, an antipsychotic, or a combination thereof.
(26) In aspect (26) is provided a method of treating drug misuse or drug addiction, comprising administering to a patient in need thereof a composition comprising a therapeutically effective amount of a compound of any one of aspects 1-22, an enantiomer thereof, a racemate thereof, a salt thereof, or any combination thereof, optionally in combination with one or more additional active ingredients.
(27) In aspect (27) is provided the method of aspect 26, wherein the patient has a history of misusing a stimulant, a depressant, an opioid, a cannabinoid, lysergic acid diethylamide (LSD), mescaline, psilocybin, nicotine, ethanol, a benzodiazepine, phencyclidine, ketamine, cocaine, an amphetamine, or any combination thereof.
(28) In aspect (28) is provided a method of treating a substance use disorder, comprising administering to a patient in need thereof a composition comprising a therapeutically effective amount of a compound of any one of aspects 1-22, an enantiomer thereof, a racemate thereof, a salt thereof, or any combination thereof, optionally in combination with one or more additional active ingredients.
(29) In aspect (29) is provided the method of aspect 28, wherein the patient has one or more affective disorders, schizophrenia, or a combination thereof.
(30) In aspect (30) is provided the method of aspect 28 or aspect 29, wherein the substance use disorder comprises a psychostimulant use disorder.
(31) In aspect (31) is provided the method of any one of aspects 28-30, wherein the patient has a history of misusing a stimulant comprising cocaine, an amphetamine, a methamphetamine, dextroamphetamine, levoamphetamine, methylenedioxymethamphetamine (MDMA), methylphenidate, modafinil, armodafinil, midodrine, oxymetazoline, dobutamine, ephedrine, pseudoephedrine, phenylephrine, or any combination thereof.
(32) In aspect (32) is provided a method of treating one or more affective disorders, schizophrenia, or a combination thereof, comprising administering to a patient in need thereof a composition comprising a therapeutically effective amount of a compound of any one of aspects 1-22, an enantiomer thereof, a racemate thereof, a salt thereof, or any combination thereof, optionally in combination with one or more additional active ingredients.
(33) In aspect (33) is provided the method of aspect 32, wherein the patient has a history of misusing a stimulant, a depressant, an opioid, a cannabinoid, lysergic acid diethylamide (LSD), mescaline, psilocybin, nicotine, ethanol, a benzodiazepine, phencyclidine, ketamine, cocaine, an amphetamine, or any combination thereof.
(34) In aspect (34) is provided the method of aspect 32 or aspect 33, wherein the affective disorder is biopolar disorder, depression, or a combination thereof.

### Chemistry

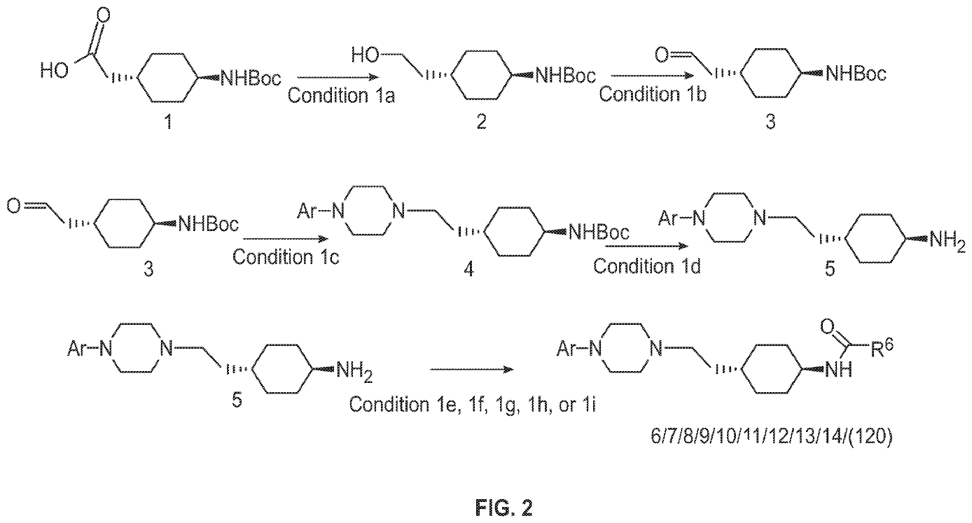

FIG. 2 shows the synthetic routes of Scheme 1. Initially, 2-(trans-4-((tert-butoxycarbonyl)amino)cyclohexyl)acetic acid (1) was reduced with borane-dimethyl sulfide (BMS) complex, and the resulting alcohol (2) was converted to aldehyde 3 via Swern oxidation. In the next step, primary pharmacophores were installed by reductive amination using various aryl piperazines (compounds 4a-e). Deprotection of these intermediates with trifluoroacetic acid (TFA) gave amine compounds 5a-e, which were then functionalized with different SPs. To that end, compound 6a (cariprazine) and substituted urea compounds 6b-7e were prepared from either N,N-dimethyl- or N-methylcarbamoyl chloride. Other urea-containing compounds were accessed by treating a selection of the aforementioned amine compounds (5a and 5b) with potassium cyanate under acidic conditions (compounds 8a and 8e) or phosgene followed by 5,6,7,8-tetrahydro-1,6-napthyridine (compounds 9a and 9b). Lastly, amide compounds 10a-13e were synthesized from a variety of carboxylic acids with either 1-ethyl-3-(3-dimethylaminopropyl)carbodiimide·HCl (EDCI) or O-(1H-6-chlorobenzotriazole-1-yl)-1,1,3,3-tetramethyluronium hexafluorophosphate (HCTU) serving as the coupling reagent. Reagents and Conditions for FIG. 2—Scheme 1: (a) BMS, THF, 0 °C to rt; (b) DMSO, (COCl)₂, NEt₃, DCM, −78 °C to rt; (c) aryl piperazine, NaBH(OAc)₃, DCE, rt; (d) TFA, DCM, rt; (e) carbamoyl chloride (e.g., N,N-dimethyl- or N-methylcarbomyl chloride) or chloroformate, DIPEA, DCM, rt; (f) KOCN, HCl, H₂O, THF, rt; (g) COCl₂, NEt₃, toluene, THF, 0 °C, then 5,6,7,8-tetrahydro-1,6-napthyridine, THF, 0 °C to rt; (h) carboxylic acid, EDCI, HOBt, DIPEA, CHCl₃, rt; (i) carboxylic acid (e.g., 3-methoxyproponic acid), HCTU, DIPEA, DCM, rt.

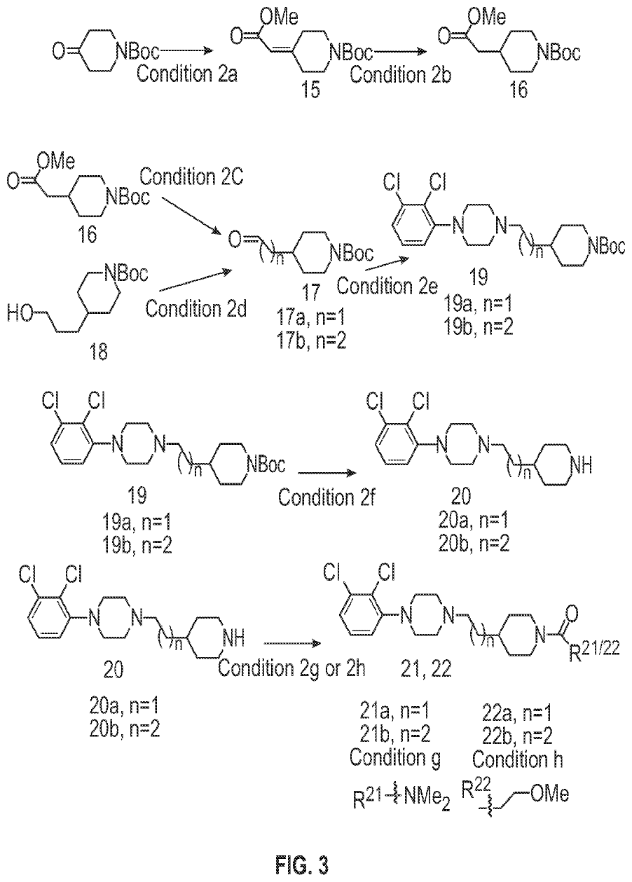

Manipulations to the linker between the primary and secondary pharmacophores of compound 6a were additionally explored. As shown in FIG. 3 (Scheme 2), the cyclohexylamine was replaced with a piperidine ring using one of two aldehydes. The first (compound 17a) retained the two-carbon chain found in compound 6a and was prepared from tert-butyl 4-oxopiperidine-1-carboxylate (compound 14). Briefly, a Horner-Wadsworth-Emmons reaction furnished α,β-unsaturated ester compound 15, which was sequentially reduced by hydrogenation over palladium on carbon (compound 16) and DIBALH to afford compound 17a. The second aldehyde (compound 17b), possessing an extended carbon chain, was obtained using a Swern oxidation on commercially available tert-butyl 4-(3-hydroxypropyl)piperidine-1-carboxylate (compound 18). The same steps described in Scheme 1 (FIG. 2) were used to prepare compounds 21a-22b (i.e., reductive amination, Boc-deprotection of compounds 19a and 19b, followed by functionalization of amine compounds 20a and 20b). Reagents and Conditions for FIG. 3—Scheme 2: (a) NaH, methyl 2-(dimethoxyphosphoryl)acetate, THF, 0 °C to rt; (b) Pd/C, H₂ (50 psi), MeOH, rt; (c) DIBALH, toluene, DCM, −78 °C to rt; (d) DMSO, (COCl)₂, NEt₃, DCM, −78 °C to rt; (e) 1-(2,3-dichlorophenyl)piperazine·HCl, NaBH(OAc)₃, DCE, rt; (f) TFA, DCM, rt; (g) N,N-dimethylcarbomyl chloride, DIPEA, DCM, rt; (h) 3-methoxyproponic acid, HCTU, DIPEA, DCM, rt.

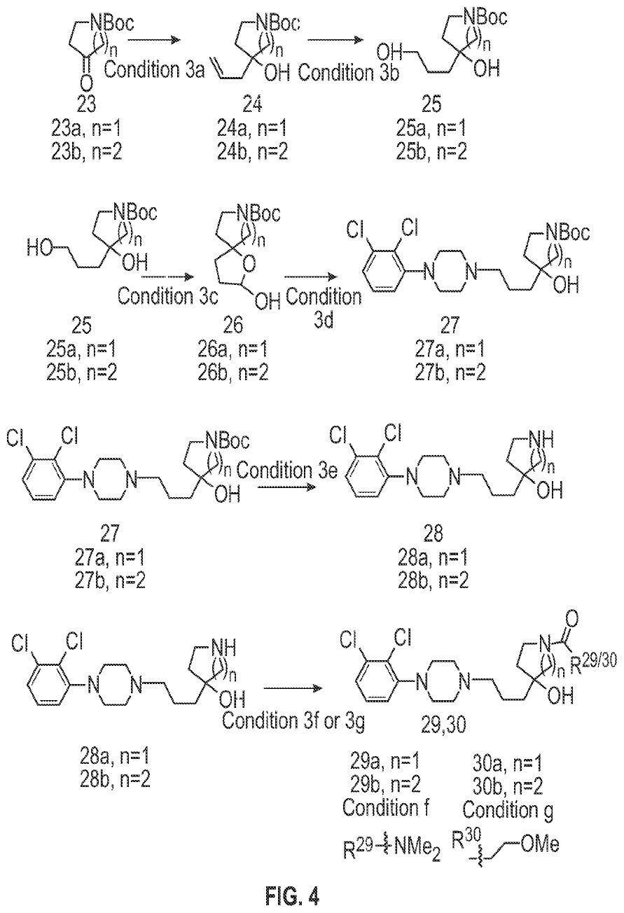

Expanding on the previous set of compounds, a hydroxyl group was also introduced in the linker, while simultaneously adjusting the ring size (Scheme 3, shown by FIG. 4). In addition to compound 6a, compounds were constructed from building block compounds 26a and 26b, which were accessed using a parallel sequence of transformations. Specifically, a three-carbon chain was introduced onto tert-butyl 3-oxopyrrolidine-1-carboxylate (compound 23a) and tert-butyl 4-oxopiperidine-1-carboxylate (compound 23b) through separate Grignard reactions with allyl magnesium bromide. The terminal alkenes of compound 24a and 24b were then subjected to hydroboration-oxidation reactions, and the corresponding alcohols (compounds 25a and 25b) were converted to hemiacetals 26a and 26b. Like before (see Schemes 1 and 2, FIGS. 2 & 3, respectively), the synthesis of compounds 29a-30b proceeded through intermediates 27a-28b. Reagents and Conditions for FIG. 4—Scheme 3: (a) allyl magnesium bromide, Et₂O, 0 °C to rt; (b) BH₃·THF, THF, 0 °C to rt, then NaOH, H₂O₂, H₂O, 0 °C to rt; (c) DMSO, (COCl)₂, NEt₃, DCM, −78 °C to rt; (d) 1-(2,3-dichlorophenyl)piperazine·HCl, NaBH(OAc)₃, DCE, rt; (e) TFA, DCM, rt; (f) N,N-dimethylcarbomyl chloride, DIPEA, DCM, rt; (g) 3-methoxyproponic acid, HCTU, DIPEA, DCM, rt.

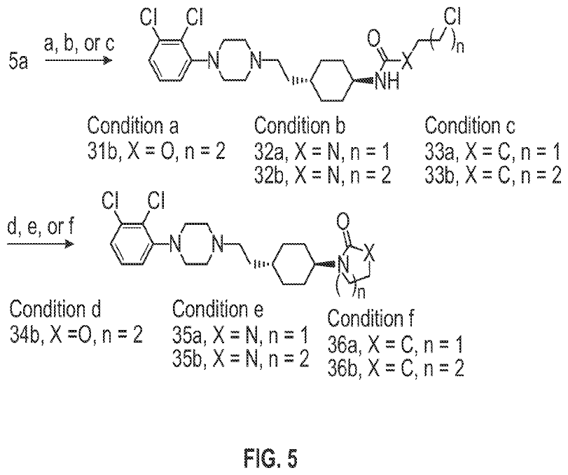

FIG. 5 shows the synthetic routes of Scheme 4. Cyclic amide, carbamate, and urea compounds 34b, 35a, 35b, 36a, and 36b were synthesized by cyclization of chlorides 31b, 32a, 32b, 33a, and 33b, respectively. Reagents and Conditions for FIG. 5—Scheme 4: (a) chloroformate, DIPEA, DCM, rt; (b) isocyanate, DCM, 0 °C to rt; (c) acid chloride, TEA, DCM, rt; (d) K2CO3, MeCN, 100 °C; (e) NaH, THF, rt; (f) NaH, THF, 100 °C.

Chemicals and solvents were purchased from commercial suppliers and used as received. Unless stated otherwise, reactions were performed under ambient conditions and monitored by thin-layer chromatography using Analtech silica gel GHLF (250 microns) coated glass plates, which were visualized with either phosphomolybdic acid, potassium permanganate, or vanillin stain. Normal phase column chromatography was conducted with a Teledyne Isco Combiflash Rf or EZ-Prep purification system (ELS detector associated). All nuclear magnetic resonance (NMR) spectra (¹H and ¹³C) were acquired in deuterated solvents (CDCl₃, CD₃OD, or CD₃CO₂D) on a Varian Mercury Plus 400 spectrometer. Chemical shifts are reported in parts per million (ppm) and were adjusted using the residual undeuterated solvents (CDCl₃: 7.26 ppm for ¹H NMR, 77.2 ppm for ¹³C NMR; CD₃OD: 3.31 ppm for ¹H NMR, 49.0 ppm for ¹³C NMR; CD₃CO₂D: 2.03 ppm for ¹H NMR, 20.0 ppm for ¹³C NMR) as an internal reference. Coupling constants are reported in Hertz (Hz) and peak multiplicities as either a singlet (s), doublet (d), triplet (t), quartet (q), or multiplet (m). Infrared (IR) spectra were acquired on a Perkin Elmer Spectrum Two FT-IR spectrometer. Melting points were determined on an Optimelt MPA100 instrument and are uncorrected. High-resolution mass spectrometry (HRMS) data were collected on a Thermo Scientific LTQ-Orbitrap Velos spectrometer using either electrospray ionization (ESI) or matrix-assisted laser desorption/ionization (MALDI). High-performance liquid chromatography (HPLC) was conducted on an Agilent Technologies 1260 Infinity system coupled to a diode-array detector. A Phenonmenex Gemini C18 100 Å LC column (50×4.6 mm, 3 μm particle size) was used as the stationary phase. For basic conditions, the mobile phase consisted of H₂O with 0.1% diethylamine (solvent A) and MeCN with 0.1% diethylamine (solvent B). For acidic conditions, solvents contained 0.1% TFA instead of diethylamine. All samples were prepared at a concentration of ca. 1 mg/mL in MeOH, and 20 μL of each solution was injected onto the column, which was maintained at 40 °C. Using a flow rate of 1.0 mL/min, the solvent gradient was as follows: 10% B held for 10 min, 10-40% B ramped over 10 min, 40% B held for 10 min, 40-80% B over 10 min, 80% B held for 20 min. Compound purity was determined based on peak integration (area under the curve) of the absorbance signals at 254 and 214 nm. Elemental analysis was performed by Atlantic Microlab, Inc. (Norcross, GA). All tested compounds were >95% pure by either HPLC or elemental analysis.

### General Procedure A.

Using oven-dried glassware under an argon atmosphere, a 0.60 M solution of (COCl)₂ in anhydrous DCM (1.5 equiv) was cooled to −78 °C. Then, anhydrous DMSO (3.0 equiv) was added over ca. 5 min, and the reaction was stirred for 30 min. Next, a 0.30 M solution of the alcohol in anhydrous DCM (1.0 equiv) was added over ca. 10 min, and the reaction was stirred for an additional 30 min. Finally, anhydrous NEt₃ (6.0 equiv) was added over ca. 5 min, and the mixture was allowed to warm to rt. When TLC analysis suggested the complete disappearance of starting material (ca. 2 h), the reaction was quenched with a 1.0 M aqueous solution of HCl, and the aqueous layer was extracted with DCM (2×). The combined organic layers were dried over anhydrous Na₂SO₄, filtered, and concentrated. The crude product was purified by chromatography as described.

### General Procedure B.

To a 0.08-0.13 M solution of 3 in anhydrous DCE (1.0 equiv) was added AcOH (1.0 equiv) followed by the appropriate aryl piperazine (1.1 equiv). The mixture was stirred for 30 min, and NaBH(OAc)₃ (1.5 equiv) was added in one portion. After stirring overnight, the reaction was quenched with a saturated aqueous solution of NaHCO₃, and the aqueous layer was extracted with DCM (2×). The combined organic layers were washed with brine, dried over Na₂SO₄, filtered, and concentrated. The crude product was purified by chromatography as described.

### General Procedure C.

TFA (27-144 equiv) was added to a 0.03-0.15 M solution of the appropriate Boc-protected amine in DCM (1.0 equiv). The reaction mixture was stirred for ca. 4 h and then concentrated. The resulting residue was suspended in a 1.0 M aqueous solution of NaOH, and the aqueous layer was extracted with CHCl₃ (3×). The combined organic layers were dried over anhydrous Na₂SO₄, filtered, and concentrated to afford the corresponding amine.

### General Procedure D.

To a 0.07-0.10 M solution of the amine in DCM (1.0 equiv) was added DIPEA (1.5 equiv) followed by the appropriate carbamoyl chloride or chloroformate (1.25 equiv) over 5 min. After stirring overnight, the reaction was quenched with a saturated aqueous solution of NaHCO₃, and the aqueous layer was extracted with CHCl₃ (3×). The combined organic layers were dried over anhydrous Na₂SO₄, filtered, and concentrated. The crude product was purified by chromatography as described.

### General Procedure E.

A 0.03-0.05 M mixture of the appropriate carboxylic acid (1.1 equiv) in CHCl₃ was cooled to 0 °C, and EDCI (1.3 equiv) followed by HOBt (1.2 equiv) were each added in one portion. After 30 min, the amine (1.0 equiv) was added followed by DIPEA (1.4 equiv). The mixture was allowed to warm to room temperature and stirred overnight. Then, a saturated aqueous solution of NaHCO₃ was added, and the aqueous layer was extracted with CHCl₃ (3×). The combined organic layers were dried over Na₂SO₄, filtered, and concentrated. The crude product was product was purified by chromatography as described.

### General Procedure F.

To a 0.10 M mixture of HCTU (1.2 equiv) in DCM was added the appropriate carboxylic acid (1.1 equiv) in one portion. After stirring the mixture for 10 min, a solution of the amine (1.0 equiv) and DIPEA (1.2 equiv) in DCM (0.04 M with respect to the limiting reagent) was added over 5 min. The reaction was stirred overnight, quenched with a saturated aqueous solution of NaHCO₃, and the aqueous layer was extracted with DCM (3×). The combined organic layers were dried over Na₂SO₄, filtered, and concentrated. The crude product was product was purified by chromatography as described to afford the corresponding amide.

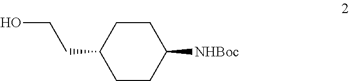 [SMILES: CC(C)(C)OC(=O)N[C@H]1CC[C@H](CCO)CC1]

### tert-Butyl (trans-4-(2-hydroxyethyl)cyclohexyl)carbamate (compound 2)

Using oven-dried glassware under an argon atmosphere, a solution of 2-(trans-4-((tert-butoxycarbonyl)amino)cyclohexyl)acetic acid (6.01 g, 23.4 mmol) in anhydrous THE (30 mL) was cooled to 0 C. Then, borane dimethylsulfide complex (3.4 mL, 36 mmol) was added dropwise over 5 min. The solution was allowed to warm to room temperature and stirred for 15 h. Afterward, the reaction was cooled back to 0 °C, quenched with a saturated aqueous solution of NaHCO₃ (60 mL), and the aqueous layer was extracted with EtOAc (2×60 mL). The organic layer was dried over anhydrous Na₂SO₄, filtered, and concentrated to afford 2 (5.18 g, 21.3 mmol, 91% yield) as a white solid. An analytical sample was prepared by recrystallization using Et₂O-hexanes (slow evaporation of solvent mixture). R_{f}=0.4 (50% EtOAc/hexanes); ¹H NMR (400 MHz, CDCl₃) δ 4.44-4.27 (m, 1H), 3.71-3.64 (m, 2H), 3.43-3.28 (m, 1H), 2.04-1.96 (m, 2H), 1.81-1.73 (m, 2H), 1.51-1.26 (m, 4H), 1.43 (s, 9H), 1.15-0.96 (m, 4H); ¹³C NMR (101 MHz, CDCl₃) δ 155.4, 79.2, 60.9, 50.0, 39.8, 33.6 (3C), 32.0 (2C), 28.6 (3C); IR (neat) 3425, 3365, 1682, 1517 cm⁻¹; mp 102-103 °C (Et₂O-hexanes); HRMS (MALDI) m/z [M+Na]⁺ calcd for C₁₃H₂₅NNaO₃ 266.1727, found 266.1730.

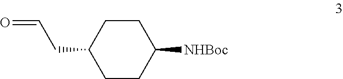 [SMILES: CC(C)(C)OC(=O)N[C@H]1CC[C@H](CC=O)CC1]

### tert-Butyl (trans-4-(2-oxoethyl)cyclohexyl)carbamate (compound 3)

General Procedure A was followed using compound 2 (5.18 g, 21.3 mmol). After work-up, the crude product was purified by chromatography (120 g of silica gel, 0-40% EtOAc/hexanes) to afford compound 3 (3.04 g, 12.6 mmol, 59% yield) as a white solid. R_{f}=0.5 (40% EtOAc/hexanes); ¹H NMR (400 MHz, CDCl₃) δ 9.76 (s, 1H), 4.47-4.29 (m, 1H), 3.45-3.26 (m, 1H), 2.35-2.29 (m, 2H), 2.06-1.95 (m, 2H), 1.90-1.74 (m, 3H), 1.44 (s, 9H), 1.20-1.03 (m, 4H); ¹³C NMR (101 MHz, CDCl₃) δ 202.3, 155.3, 79.3, 50.8, 49.6, 33.3 (2C), 31.9 (2C), 31.8, 28.6 (3C); IR (neat) 3377, 1715, 1686 cm⁻¹; mp 90-91

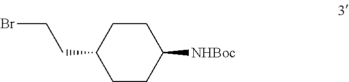 [SMILES: CC(C)(C)OC(=O)N[C@H]1CC[C@H](CCBr)CC1]

### tert-Butyl (trans-4-(2-bromoethyl)cyclohexyl)carbamate (compound 3′)

A solution of compound 2 (1.00 g, 4.12 mmol) and PPh₃ (1.63 g, 6.20 mmol) in DCM (41 mL) was cooled to 0 °C, and CBr₄ (2.06 g, 6.21 mmol) was added in one portion. After stirring at 0 °C for 1 h, the reaction mixture was allowed to warm to rt, stirred for an additional 1 h, and then concentrated. The crude product was purified by chromatography (40 g of silica gel, 0-10% EtOAc/hexanes) to afford compound 3′ (0.549 g, 1.79 mmol, 44% yield) as a white solid. R_{f}=0.2 (10% EtOAc/hexanes); ¹H NMR (400 MHz, CDCl₃) δ 4.46-4.29 (m, 1H), 3.42 (t, J=7.0 Hz, 2H), 3.39-3.28 (m, 1H), 2.05-1.96 (m, 2H), 1.82-1.71 (m, 4H), 1.49-1.38 (m, 1H), 1.43 (s, 9H), 1.16-0.94 (m, 4H); ¹³C NMR (101 MHz, CDCl₃) δ 155.4, 79.3, 49.9, 39.7, 35.4, 33.4 (2C), 31.8, 31.3 (2C), 28.6 (3C); IR (film) 3337, 1688 cm⁻¹; mp 82-83 °C; HRMS (ESI) m/z [M+H]⁺ calcd for C₁₃H₂₄BrNNaO₂ 328.0883, found 328.0883.

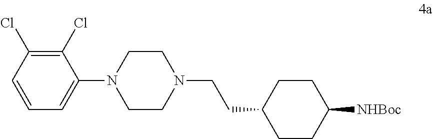 [SMILES: CC(C)(C)OC(=O)N[C@H]1CC[C@H](CCN2CCN(c3cccc(Cl)c3Cl)CC2)CC1]

### tert-Butyl (trans-4-(2-(4-(2,3-dichlorophenyl)piperazin-1-yl)ethyl)cyclohexyl)carbamate (compound 4a)

General procedure B was followed using compound 3 (2.00 g, 8.29 mmol) and 1-(2,3-dichlorophenyl)piperazine·HCl (2.44 g, 9.12 mmol) in DCE (56 mL). After work-up, the crude product was purified by chromatography (80 g of silica gel, 0-50% EtOAc/hexanes) to afford compound 4a (1.79 g, 3.92 mmol, 47% yield) as a white solid. R_{f}=0.2 (50% EtOAc/hexanes); ¹H NMR (400 MHz, CDCl₃) δ 7.16-7.08 (m, 2H), 6.98-6.91 (m, 1H), 4.44-4.24 (m, 1H), 3.44-3.27 (m, 1H), 3.14-2.97 (m, 4H), 2.71-2.52 (m, 4H), 2.46-2.36 (m, 2H), 2.05-1.92 (m, 2H), 1.81-1.71 (m, 2H), 1.49-1.37 (m, 2H), 1.44 (s, 9H), 1.29-1.15 (m, 1H), 1.14-0.95 (m, 4H); ¹³C NMR (101 MHz, CDCl₃) δ 155.7, 151.8, 135.5, 128.0, 127.9, 125.0, 119.0, 79.5, 57.1, 53.9, 51.8, 50.4, 35.9, 34.4, 33.9 (2C), 32.5 (2C), 28.9 (3C); IR (neat) 3366, 1677 cm⁻¹; mp 147-148 °C; HRMS (MALDI) m/z [M+H]⁺ calcd for C₂₃H₃Cl₂N₃O₂ 456.2179, found 456.2174; t_{R}=39.4 min (HPLC, basic). The oxalate salt was precipitated from a 0.03 M solution of the free base in 50% CHCl₃/acetone using oxalic acid (1.25 equiv). Dec>185 °C. Anal. calcd for C₂₃H₃₅Cl₂N₃O₂·1.25C₂H₂O₄: C, 53.83; H, 6.64; N, 7.39. Found: C, 54.06; H, 6.67; N, 7.48.

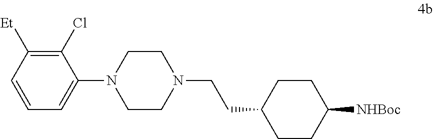 [SMILES: CCc1cccc(N2CCN(CC[C@H]3CC[C@H](NC(=O)OC(C)(C)C)CC3)CC2)c1Cl]

### tert-Butyl (trans-4-(2-(4-(2-chloro-3-ethylphenyl)piperazin-1-yl)ethyl)cyclohexyl)carbamate (compound 4b)

General procedure B was followed using compound 3 (0.710 g, 2.94 mmol) and 1-(2-chloro-3-ethylphenyl)piperazine (0.810 g, 3.60 mmol) in DCE (35 mL). After work-up, the crude product was purified by chromatography (silica gel, 0-70% EtOAc/hexanes) to afford compound 4b (0.990 g, 2.20 mmol, 75% yield) as a white solid. ¹H NMR (400 MHz, CDCl₃) δ 7.15 (t, J=7.7 Hz, 1H), 6.96-6.91 (m, 2H), 4.41-4.31 (m, 1H), 3.45-3.29 (m, 1H), 3.16-2.95 (m, 4H), 2.77 (q, J=7.6 Hz, 2H), 2.72-2.48 (m, 4H), 2.46-2.38 (m, 2H), 2.02-1.95 (m, 2H), 1.82-1.73 (m, 2H), 1.48-1.40 (m, 2H), 1.44 (s, 9H), 1.27-1.19 (m, 1H), 1.22 (t, J=7.5 Hz, 3H), 1.13-0.99 (m, 4H).

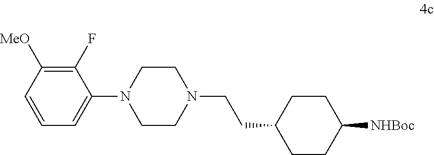 [SMILES: COc1cccc(N2CCN(CC[C@H]3CC[C@H](NC(=O)OC(C)(C)C)CC3)CC2)c1F]

### tert-Butyl (trans-4-(2-(4-(2-fluoro-3-methoxyphenyl)piperazin-1-yl)ethyl)cyclohexyl)carbamate (compound 4c)

General procedure B was followed using 3 (0.725 g, 3.00 mmol) and 1-(2-fluoro-3-methoxyphenyl)piperazine·HCl (0.816 g, 3.31 mmol) in DCE (25 mL). After work-up, the crude product was purified by chromatography (40 g of silica gel, 0-80% EtOAc/hexanes) to afford compound 4c (0.695 g, 1.60 mmol, 53% yield) as a light orange solid. R_{f}=0.4 (80% EtOAc/hexanes); ¹H NMR (400 MHz, CDCl₃) δ 6.95 (dt, J=8.3, 1.9 Hz, 1H), 6.66-6.53 (m, 2H), 4.43-4.26 (m, 1H), 3.86 (s, 3H), 3.43-3.25 (m, 1H), 3.19-2.98 (m, 4H), 2.68-2.47 (m, 4H), 2.43-2.35 (m, 2H), 2.03-1.92 (m, 2H), 1.82-1.69 (m, 2H), 1.48-1.36 (m, 2H), 1.43 (s, 9H), 1.29-1.15 (m, 1H), 1.12-0.95 (m, 4H); ¹³C NMR (101 MHz, CDCl₃) δ 155.4, 148.7 (d, J=10 Hz), 145.8 (d, J=245 Hz),141.2 (d, J=6 Hz), 123.6 (d, J=5 Hz), 111.2 (d, J=2 Hz), 107.2, 79.2, 56.8, 56.6, 53.6 (2C), 50.8 (d, J=3 Hz, 2C), 50.0, 35.6, 34.0, 33.6 (2C), 32.1 (2C), 28.6 (3C); IR (neat) 3370, 1685 cm⁻¹; mp 118-119 °C; HRMS (ESI) m/z [M+H]⁺ calcd for C₂₄H₃₉FN₃O₃ 436.2970, found 436.2964.

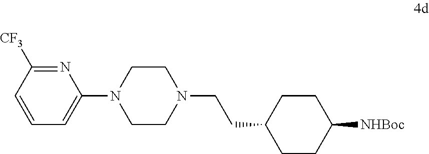 [SMILES: CC(C)(C)OC(=O)N[C@H]1CC[C@H](CCN2CCN(c3cccc(C(F)(F)F)n3)CC2)CC1]

### tert-Butyl (trans-4-(2-(4-(6-(trifluoromethyl)pyridin-2-yl)piperazin-1-yl)ethyl)cyclohexyl)carbamate (compound 4d)

General procedure B was followed using 3 (1.00 g, 4.16 mmol) and 1-(6-(trifluoromethyl)pyridin-2-yl)piperazine (1.06 g, 4.58 mmol) in DCE (28 mL). Note: compound 3 was added to a solution of the aryl piperazine. After work-up, the crude product was purified by chromatography (40 g of silica gel, 0-80% EtOAc/hexanes) to afford compound 4d (1.23 g, 2.69 mmol, 65% yield) as a white solid. R_{f}=0.3 (80% EtOAc/hexanes); ¹H NMR (400 MHz, CDCl₃) δ 7.55 (t, J=8.0 Hz, 1H), 6.92 (d, J=7.3 Hz, 1H), 6.75 (d, J=8.7 Hz, 1H), 4.44-4.28 (m, 1H), 3.60 (t, J=5.1 Hz, 4H), 3.43-3.27 (m, 1H), 2.51 (t, J=5.1 Hz, 4H), 2.42-2.35 (m, 2H), 2.03-1.93 (m, 2H), 1.81-1.68 (m, 2H), 1.50-1.38 (m, 2H), 1.43 (s, 9H), 1.30-1.16 (m, 1H), 1.14-0.97 (m, 4H); ¹³C NMR (101 MHz, CDCl₃) δ 159.0, 155.4, 146.6 (q, J=34 Hz), 138.3, 121.8 (q, J=274 Hz), 109.5, 108.9 (q, J=3 Hz), 79.2, 56.8, 53.1 (2C), 50.0, 44.9 (2C), 35.6, 34.0 (2C), 33.6, 32.1 (2C), 28.6 (3C); IR (neat) 3369, 1678 cm⁻¹; mp 131-132 °C; HRMS (ESI) m/z [M+H]⁺ calcd for C₂₃H₃₆F₃N₄O₂ 457.2785, found 457.2777.

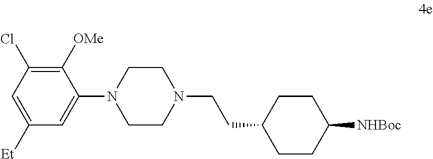 [SMILES: CCc1cc(Cl)c(OC)c(N2CCN(CC[C@H]3CC[C@H](NC(=O)OC(C)(C)C)CC3)CC2)c1]

### tert-Butyl (trans-4-(2-(4-(3-chloro-5-ethyl-2-methoxyphenyl)piperazin-1-yl)ethyl)cyclohexyl)carbamate (compound 4e)

General procedure B was followed using compound 3 (1.00 g, 4.16 mmol) and 1-(3-chloro-5-ethyl-2-methoxyphenyl)piperazine·HCl (1.33 g, 4.57 mmol) in DCE (27 mL). After work-up, the crude product was purified by chromatography (40 g of silica gel, 0-50% EtOAc/hexanes) to afford compound 4e (1.10 g, 2.29 mmol, 55% yield) as a yellow solid. R_{f}=0.5 (50% MeOH/DCM); ¹H NMR (400 MHz, CDCl₃) δ 6.84 (s, 1H), 6.61 (s, 1H), 4.44-4.26 (m, 1H), 3.83 (s, 3H), 3.44-3.26 (m, 1H), 3.23-3.02 (m, 4H), 2.68-2.49 (m, 6H), 2.45-2.35 (m, 2H), 2.03-1.94 (m, 2H), 1.81-1.71 (m, 2H), 1.50-1.37 (m, 2H), 1.43 (s, 9H), 1.30-1.15 (m, 4H), 1.14-0.97 (m, 4H); ¹³C NMR (101 MHz, CDCl₃) δ 155.1, 146.2, 146.0, 140.6, 128.0, 122.0, 116.5, 78.90, 58.8, 56.5, 53.7 (2C), 50.1 (2C), 49.7, 35.3, 33.7, 33.3 (2C), 31.8 (2C), 28.30, 28.27 (3C), 15.2; IR (neat) 3364, 1681 cm⁻¹; mp 86-88 0C; HRMS (MALDI) m/z [M+H]⁺ calcd for C₂₆H₄₃ClN₃O₃ 480.2987, found 480.2985. The oxalate salt was precipitated from a 0.02 M solution of the free base in 50% CHCl₃/acetone using oxalic acid (1.25 equiv). Mp 132-135 °C. Anal. calcd for C₂₆H₄₂ClN₃O₃·C₂H₂O₄: C, 58.99; H, 7.78; N, 7.37. Found: C, 58.70; H, 7.81; N, 7.30.

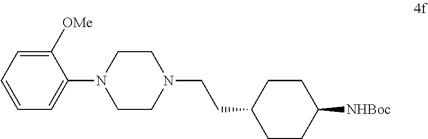 [SMILES: COc1ccccc1N1CCN(CC[C@H]2CC[C@H](NC(=O)OC(C)(C)C)CC2)CC1]

### tert-Butyl (trans-4-(2-(4-(2-methoxyphenyl)piperazin-1-yl)ethyl)cyclohexyl)carbamate (compound 4f)

General procedure B was followed using compound 3 (0.726 g, 3.01 mmol) and 1-(2-methoxyphenyl)piperazine (0.636 g, 3.31 mmol) in DCE (10.0 mL). After work-up, the crude product was purified by chromatography (40 g of silica gel, 0-70% EtOAc/hexanes) to afford compound 4f (1.01 g, 2.43 mmol, 81% yield) as a yellow solid. R_{f}=0.3 (70% EtOAc/hexanes); ¹H NMR (400 MHz, CDCl₃) δ 7.03-6.88 (m, 3H), 6.87-6.83 (m, 1H), 4.44-4.27 (m, 1H), 3.86 (s, 3H), 3.45-3.28 (m, 1H), 3.25-2.94 (m, 4H), 2.81-2.52 (m, 4H), 2.47-2.36 (m, 2H), 2.05-1.92 (m, 2H), 1.84-1.72 (m, 2H), 1.52-1.38 (m, 2H), 1.44 (s, 9H), 1.32-1.17 (m, 1H), 1.14-0.97 (m, 4H); ¹³C NMR (101 MHz, CDCl₃) δ 155.4, 152.4, 141.5, 123.0, 121.2, 118.4, 111.4, 79.2, 56.9, 55.5, 53.7 (2C), 50.8 (2C), 50.1, 35.7, 34.0, 33.6 (2C), 32.2 (2C), 28.6 (3C); IR (neat) 3345, 1704 cm⁻¹; mp 139-140 0C; HRMS (ESI) m/z [M+H]⁺ calcd for C₂₄H₄₀N₃O₃ 418.3064, found 418.3057.

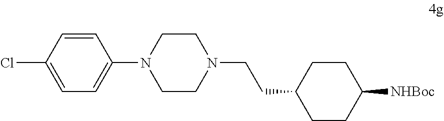 [SMILES: CC(C)(C)OC(=O)N[C@H]1CC[C@H](CCN2CCN(c3ccc(Cl)cc3)CC2)CC1]

### tert-Butyl (trans-4-(2-(4-(4-chlorophenyl)piperazin-1-yl)ethyl)cyclohexyl)carbamate (compound 4g)

General procedure B was followed using compound 3 (0.300 g, 1.24 mmol) and 1-(4-chlorophenyl)piperazine·HCl (0.290 g, 1.25 mmol) in DCE (4.0 mL). After work-up, the crude product was purified by chromatography (12 g of silica gel, 0-50% EtOAc/hexanes) to afford compound 4g (0.188 g, 0.447 mmol, 36% yield) as a yellow solid. R_{f}=0.2 (50% EtOAc/hexanes); ¹H NMR (400 MHz, CDCl₃) δ 7.19 (d, J=9.0 Hz, 2H), 6.83 (d, J=9.0 Hz, 2H), 4.44-4.26 (m, 1H), 3.45-3.26 (m, 1H), 3.21-3.07 (m, 4H), 2.63-2.49 (m, 4H), 2.43-2.33 (m, 2H), 2.05-1.91 (m, 2H), 1.83-1.69 (m, 2H), 1.50-1.34 (m, 2H), 1.44 (s, 9H), 1.30-1.15 (m, 1H), 1.13-0.96 (m, 4H); ¹³C NMR (101 MHz, CDCl₃) δ 155.4, 150.1, 129.1 (2C), 124.6, 117.3 (2C), 79.2, 56.7, 53.3 (2C), 50.1, 49.3 (2C), 35.6, 34.0, 33.6 (2C), 32.1 (2C), 28.6 (3C); IR (film) 3353, 1681 cm⁻¹; mp 166-167 °C (dec); HRMS (ESI) m/z [M+H]⁺ calcd for C₂₃H₃₇ClN₃O₂ 422.2569, found 422.2565.

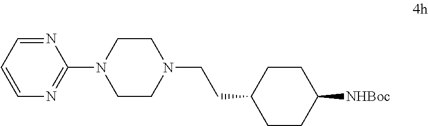 [SMILES: CC(C)(C)OC(=O)N[C@H]1CC[C@H](CCN2CCN(c3ncccn3)CC2)CC1]

### tert-Butyl (trans-4-(2-(4-(pyrimidin-2-yl)piperazin-1-yl)ethyl)cyclohexyl)carbamate (compound 4h)

General procedure B was followed using compound 3 (0.300 g, 1.25 mmol) and 2-(piperazin-1-yl)pyrimidine (0.206 g, 1.26 mmol) in DCE (4.0 mL). After work-up, the crude product was purified by chromatography (12 g of silica gel, 0-70% EtOAc/hexanes) to afford compound 4h (0.317 g, 0.814 mmol, 65% yield) as a tan solid. R_{f}=0.1 (70% EtOAc/hexanes); ¹H NMR (400 MHz, CDCl₃) δ 8.31-8.26 (m, 2H), 6.49-6.44 (m, 1H), 4.45-4.27 (m, 1H), 3.89-3.71 (m, 4H), 3.43-3.26 (m, 1H), 2.52-2.42 (m, 4H), 2.41-2.33 (m, 2H), 2.03-1.92 (m, 2H), 1.83-1.69 (m, 2H), 1.50-1.35 (m, 2H), 1.43 (s, 9H), 1.30-1.15 (m, 1H), 1.13-0.96 (m, 4H); ¹³C NMR (101 MHz, CDCl₃) δ 161.9, 157.8 (2C), 155.4, 109.9, 79.2, 56.9, 53.4 (2C), 50.1, 43.9 (2C), 35.6, 34.0, 33.6 (2C), 32.1 (2C), 28.6 (3C); IR (film) 3349, 1682 cm⁻¹; mp 147-149 °C (dec); HRMS (ESI) m/z [M+H]⁺ calcd for C₂₁H₃₆N₅O₂ 390.2864, found 390.2859.

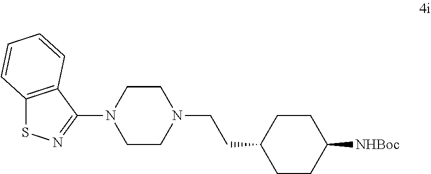 [SMILES: CC(C)(C)OC(=O)N[C@H]1CC[C@H](CCN2CCN(c3nsc4ccccc34)CC2)CC1]

### tert-Butyl (trans-4-(2-(4-(benzo[d]isothiazol-3-yl)piperazin-1-yl)ethyl)cyclohexyl)carbamate (compound 4i)

General procedure B was followed using compound 3 (0.302 g, 1.25 mmol) and 3-(piperazin-1-yl)benzo[d]isothiazole (0.301 g, 1.37 mmol) in DCE (4.0 mL). After work-up, the crude product was purified by chromatography (12 g of silica gel, 0-60% EtOAc/hexanes) to afford compound 4i (0.269 g, 0.605 mmol, 48% yield) as a yellow solid. R_{f}=0.3 (60% EtOAc/hexanes); ¹H NMR (400 MHz, CDCl₃) δ 7.90 (d, J=8.4 Hz, 1H), 7.80 (d, J=8.4 Hz, 1H), 7.50-7.42 (m, 1H), 7.38-7.31 (m, 1H), 4.47-4.28 (m, 1H), 3.64-3.50 (m, 4H), 3.45-3.27 (m, 1H), 2.74-2.56 (m, 4H), 2.52-2.39 (m, 2H), 2.06-1.92 (m, 2H), 1.86-1.72 (m, 2H), 1.52-1.37 (m, 2H), 1.44 (s, 9H), 1.33-1.17 (m, 1H), 1.15-0.97 (m, 4H); ¹³C NMR (101 MHz, CDCl₃) δ 164.1, 155.4, 152.9, 128.2, 127.6, 124.1, 124.0, 120.7, 79.2, 56.8, 53.3 (2C), 50.2 (3C), 35.6, 33.9, 33.6 (2C), 32.1 (2C), 28.6 (3C); IR (film) 3337, 1697 cm⁻¹; mp 129-130 0C; HRMS (ESI) m/z [M+H]⁺ calcd for C₂₄H₃₇N₄O₂S 445.2632, found 445.2625.

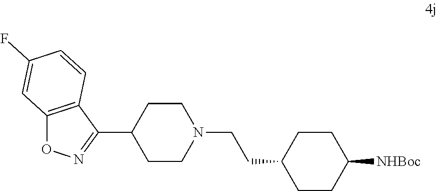 [SMILES: CC(C)(C)OC(=O)N[C@H]1CC[C@H](CCN2CCC(c3noc4cc(F)ccc34)CC2)CC1]

### tert-Butyl (trans-4-(2-(4-(6-fluorobenzo[d]isoxazol-3-yl)piperidin-1-yl)ethyl)cyclohexyl)carbamate (compound 4j)

General procedure B was followed using compound 3 (0.302 g, 1.25 mmol) and 6-fluoro-3-(piperidin-4-yl)benzo[d]isoxazole (0.276 g, 1.25 mmol) in DCE (4.0 mL). After work-up, the crude product was purified by chromatography (12 g of silica gel, 0-50% EtOAc/hexanes) to afford compound 4j (0.313 g, 0.703 mmol, 56% yield) as a white solid. R_{f}=0.1 (50% EtOAc/hexanes); ¹H NMR (400 MHz, CDCl₃) δ 7.73-7.67 (m, 1H), 7.25-7.20 (m, 1H), 7.08-7.00 (m, 1H), 4.45-4.29 (m, 1H), 3.44-3.28 (m, 1H), 3.14-2.96 (m, 3H), 2.44-2.35 (m, 2H), 2.16-1.93 (m, 8H), 1.85-1.70 (m, 2H), 1.51-1.34 (m, 2H), 1.44 (s, 9H), 1.30-1.16 (m, 1H), 1.14-0.95 (m, 4H); ¹³C NMR (101 MHz, CDCl₃) δ 164.0 (d, J=251 Hz), 163.9 (d, J=14 Hz), 161.1, 155.2, 122.6 (d, J=11 Hz), 117.3, 112. 2 (d, J=25 Hz), 97.4 (d, J=27 Hz), 79.0, 56.9, 53.6 (2C), 49.9, 35.5, 34.7, 34.0, 33.4 (2C), 32.0 (2C), 30.6 (2C), 28.4 (3C); IR (film) 3351, 1682 cm⁻¹; mp 144-145 0C; HRMS (ESI) m/z [M+H]⁺ calcd for C₂₅H₃₇FN₃O₃ 446.2813, found 446.2808.

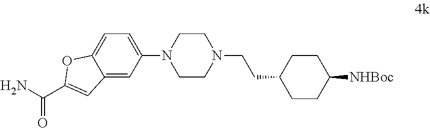 [SMILES: CC(C)(C)OC(=O)N[C@H]1CC[C@H](CCN2CCN(c3ccc4oc(C(N)=O)cc4c3)CC2)CC1]

### tert-Butyl (trans-4-(2-(4-(2-carbamoylbenzofuran-5-yl)piperazin-1-yl)ethyl)cyclohexyl)carbamate (compound 4k)

A mixture of compound 3′ (0.335 g, 1.10 mmol), 5-(piperazin-1-yl)benzofuran-2-carboxamide (0.295 g, 1.20 mmol), K₂CO₃ (0.303 g, 2.19 mmol), and TEA (0.31 mL, 2.2 mmol) in MeCN (11.0 mL) was heated to 100 °C in a pressure flask. After for 2 d, the reaction mixture was allowed to cool to rt and concentrated under a stream of Ar. The crude product was purified by chromatography (12 g of silica gel, 0-10% MeOH/DCM) to afford compound 4k (0.267 g, 0.567 mmol, 52% yield) as a white solid. R_{f}=0.2 (10% MeOH/DCM); ¹H NMR (400 MHz, CD₃CO₂D) δ 7.61-7.54 (m, 1H), 7.52-7.43 (m, 1H), 7.35-7.18 (m, 2H), 4.00-3.54 (m, 4H), 3.53-2.96 (m, 8H), 1.99-1.87 (m, 2H), 1.83-1.62 (m, 4H), 1.54-1.24 (m, 2H), 1.42 (s, 9H), 1.23-0.94 (m, 5H); ¹³C NMR (101 MHz, CD₃CO₂D) δ 164.1, 157.4, 152.3, 149.5, 148.0, 129.3, 121.2, 113.5, 113.3, 111.3, 80.6, 56.1, 52.5 (2C), 50.6, 49.3 (2C), 35.6, 33.5 (2C), 32.3 (2C), 31.1, 28.7 (3C); IR (neat) 3405, 3362, 3157, 1683, 1655, 1618 cm⁻¹; dec >240 °C; HRMS (ESI) m/z [M+H]⁺ calcd for C₂₆H₃₉N₄O₄ 471.2966, found 471.2961.

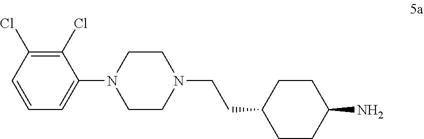 [SMILES: N[C@H]1CC[C@H](CCN2CCN(c3cccc(Cl)c3Cl)CC2)CC1]

### trans-4-(2-(4-(2,3-Dichlorophenyl)piperazin-1-yl)ethyl)cyclohexan-1-amine (compound 5a)

General procedure C was followed using compound 4a (1.33 g, 2.91 mmol) and TFA (9.0 mL, 0.12 mol) in DCM (20 mL). After work-up, compound 5a (1.00 g, 2.81 mmol, 96% yield) was isolated as a white solid. R_{f}=0.1 (10% MeOH/DCM); ¹H NMR (400 MHz, CDCl₃) δ 7.17-7.09 (m, 2H), 6.95 (dd, J=6.4, 3.1 Hz, 1H), 3.15-2.96 (m, 4H), 2.72-2.51 (m, 5H), 2.47-2.37 (m, 2H), 1.84 (d, J=12.2 Hz, 2H), 1.76 (d, J=12.3 Hz, 2H), 1.48-1.32 (m, 4H), 1.32-1.17 (m, 1H), 1.14-0.92 (m, 4H); ¹³C NMR (101 MHz, CDCl₃) δ 151.5, 134.2, 127.7, 127.6, 124.6, 118.7, 56.9, 53.6 (2C), 51.5 (2C), 50.9, 36.9 (2C), 35.8, 34.2, 32.3 (2C); IR (neat) 1578 cm⁻¹; mp 72-74 °C; HRMS (MALDI) m/z [M+H]⁺ calcd for C₁₈H₂₈Cl₂N₃ 356.1655, found 356.1660.

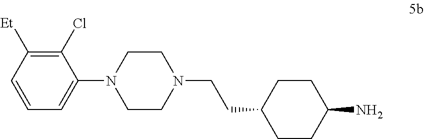 [SMILES: CCc1cccc(N2CCN(CC[C@H]3CC[C@H](N)CC3)CC2)c1Cl]

### trans-4-(2-(4-(2-Chloro-3-ethylphenyl)piperazin-1-yl)ethyl)cyclohexan-1-amine (compound 5b)

General procedure C was followed using compound 4b (0.990 g, 2.20 mmol) and TFA (5.0 mL, 60 mmol) in DCM (15 mL). After work-up, compound 5b (0.775 g, 2.20 mmol, 100% yield) was isolated as a beige solid. ¹H NMR (400 MHz, CDCl₃) δ 7.15 (t, J=7.8 Hz, 1H), 6.97-6.91 (m, 2H), 3.13-2.98 (m, 4H), 2.77 (q, J=7.5 Hz, 2H), 2.72-2.54 (m, 5H), 2.47-2.40 (m, 2H), 1.88-1.81 (m, 2H), 1.80-1.73 (m, 2H), 1.48-1.40 (m, 2H), 1.28-1.18 (m, 1H), 1.22 (t, J=7.5 Hz, 3H), 1.14-0.94 (m, 4H).

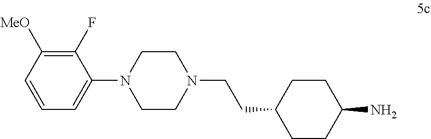 [SMILES: COc1cccc(N2CCN(CC[C@H]3CC[C@H](N)CC3)CC2)c1F]

### trans-4-(2-(4-(2-Fluoro-3-methoxyphenyl)piperazin-1-yl)ethyl)cyclohexan-1-amine (compound 5c)

General procedure C was followed using compound 4c (0.251 g, 0.577 mmol) and TFA (1.8 mL, 24 mmol) in DCM (4.0 mL). After work-up, compound 5c (0.191 g, 0.569 mmol, 99% yield) was isolated as a tan solid. R_{f}=0.1 (10% MeOH/DCM); ¹H NMR (400 MHz, CDCl₃) δ 6.95 (dt, J=8.3, 1.9 Hz, 1H), 6.66-6.53 (m, 2H), 3.85 (s, 3H), 3.18-3.01 (m, 4H), 2.69-2.52 (m, 5H), 2.44-2.37 (m, 2H), 1.88-1.80 (m, 2H), 1.79-1.70 (m, 2H), 1.48-1.16 (m, 5H), 1.12-0.91 (m, 4H); ¹³C NMR (101 MHz, CDCl₃) δ 148.7 (d, J=10 Hz), 145.8 (d, J=245 Hz), 141.2 (d, J=6 Hz), 123.6 (d, J=5 Hz), 111.2 (d, J=2 Hz), 107.1, 56.9, 56.6, 50.9, 50.8 (d, J=3 Hz, 2C), 36.9, 35.8, 34.2, 32.3; IR (film) 1611, 1576 cm⁻¹; mp 59-60 °C; HRMS (ESI) m/z [M+H]⁺ calcd for Cl₉H₃₁FN₃O 336.2446, found 336.2442.

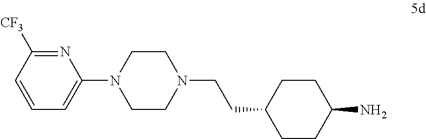 [SMILES: N[C@H]1CC[C@H](CCN2CCN(c3cccc(C(F)(F)F)n3)CC2)CC1]

### trans-4-(2-(4-(6-(Trifluoromethyl)pyridin-2-yl)piperazin-1-yl)ethyl)cyclohexan-1-amine (compound 5d)

General procedure C was followed using compound 4d (0.301 g, 0.659 mmol) and TFA (2.0 mL, 26 mmol) in DCM (4.0 mL). After work-up, compound 5d (0.234 g, 0.656 mmol, 100% yield) was isolated as a clear, colorless oil. R_{f}=0.1 (10% MeOH/DCM); ¹H NMR (400 MHz, CDCl₃) δ 7.55 (t, J=8.0 Hz, 1H), 6.92 (d, J=7.3 Hz, 1H), 6.75 (d, J=8.7 Hz, 1H), 3.60 (t, J=5.1 Hz, 4H), 2.63-2.55 (m, 1H), 2.52 (t, J=5.1 Hz, 4H), 2.42-2.35 (m, 2H), 1.88-1.81 (m, 2H), 1.79-1.71 (m, 2H), 1.47-1.38 (m, 2H), 1.34-1.16 (m, 3H), 1.13-0.93 (m, 4H); ¹³C NMR (101 MHz, CDCl₃) δ 159.0, 146.6 (d, J=34 Hz), 138.3, 121.8 (q, J=274 Hz), 117.7, 108.9 (q, J=3 Hz), 56.9, 53.1 (2C), 50.9, 44.9 (2C), 36.5 (2C), 35.7, 34.1, 32.2 (2C); IR (neat) 1604 cm⁻¹; HRMS (ESI) m/z [M+H]⁺ calcd for C₁₈H₂₅F₃N₄ 357.2261, found 357.2255.

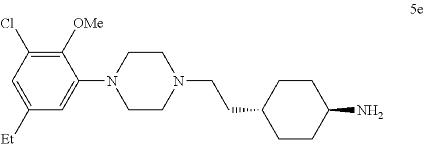 [SMILES: CCc1cc(Cl)c(OC)c(N2CCN(CC[C@H]3CC[C@H](N)CC3)CC2)c1]

### trans-4-(2-(4-(3-Chloro-5-ethyl-2-methoxyphenyl)piperazin-1-yl)ethyl)cyclohexan-1-amine (compound 5e)

General procedure C was followed using compound 4e (1.10 g, 2.29 mmol) and TFA (7.0 mL, 91 mmol) in DCM (16 mL). After work-up, compound 5e (0.87 g, 2.3 mmol, 100% yield) was isolated as a clear, orange oil. R_{f}=0.1 (10% MeOH/DCM); ¹H NMR (400 MHz, CDCl₃) δ 6.83 (d, J=1.9 Hz, 1H), 6.61 (d, J=2.0 Hz, 1H), 3.83 (s, 3H), 3.20-3.04 (m, 4H), 2.65-2.49 (m, 6H), 2.44-2.37 (m, 2H), 1.84 (d, J=11.9 Hz, 2H), 1.76 (d, J=12.0 Hz, 2H), 1.47-1.33 (m, 4H), 1.30-1.15 (m, 5H), 1.13-0.93 (m, 4H); ¹³C NMR (101 MHz, CDCl₃) δ 146.5, 146.3, 140.9, 128.3, 122.3, 116.8, 59.1, 57.0, 54.0 (2C), 50.9, 50.4 (2C), 36.9 (2C), 35.8, 34.2, 32.3 (2C), 28.6, 15.6; IR (neat) 1593, 1565 cm⁻¹; HRMS (MALDI) m/z [M+H]⁺ calcd for C₂₁H₃₅ClN₃O 380.2463, found 380.2467.

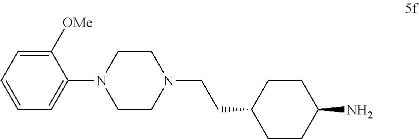 [SMILES: COc1ccccc1N1CCN(CC[C@H]2CC[C@H](N)CC2)CC1]

### trans-4-(2-(4-(2-Methoxyphenyl)piperazin-1-yl)ethyl)cyclohexan-1-amine (compound 5f)

General procedure C was followed using compound 4f (0.325 g, 0.778 mmol) and TFA (2.4 mL, 31 mmol) in DCM (7.8 mL). After work-up, compound 5f (0.242 g, 0.762 mmol, 98% yield) was isolated as a yellow solid. R_{f}=0.1 (10% MeOH/DCM); ¹H NMR (400 MHz, CDCl₃) δ 7.03-6.88 (m, 3H), 6.87-6.82 (m, 1H), 3.85 (s, 3H), 3.22-2.92 (m, 4H), 2.77-2.48 (m, 5H), 2.46-2.37 (m, 2H), 1.89-1.80 (m, 2H), 1.79-1.71 (m, 2H), 1.48-1.20 (m, 4H), 1.24-1.15 (m, 1H), 1.13-0.92 (m, 4H); ¹³C NMR (101 MHz, CDCl₃) δ 152.4, 141.6, 122.9, 121.1, 118.3, 111.4, 57.0, 55.5, 53.7 (2C), 51.0, 50.8 (2C), 36.9 (2C), 35.8, 34.2, 32.4 (2C); IR (film) 3350 cm⁻¹; mp 56-58 0C; HRMS (ESI) m/z [M+H]⁺ calcd for C₁₋₈H₃₂N₃O 318.2540, found 318.2538.

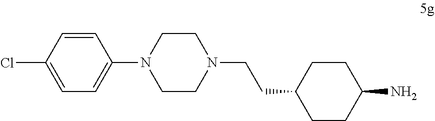 [SMILES: N[C@H]1CC[C@H](CCN2CCN(c3ccc(Cl)cc3)CC2)CC1]

### trans-4-(2-(4-(4-Chlorophenyl)piperazin-1-yl)ethyl)cyclohexan-1-amine (compound 5g)

General procedure C was followed using compound 4g (0.154 g, 0.365 mmol) and TFA (1.1 mL, 14 mmol) in DCM (2.5 mL). Afterwork-up, compound 5g (0.116 g, 0.361 mmol, 99% yield) was isolated as a yellow solid. R_{f}=0.1 (10% MeOH/DCM); ¹H NMR (400 MHz, CDCl₃) δ 7.19 (d, J=8.3 Hz, 2H), 6.83 (d, J=8.3 Hz, 2H), 3.21-3.07 (m, 4H), 2.65-2.51 (m, 5H), 2.43-2.34 (m, 2H), 1.99-1.80 (m, 2H), 1.79-1.70 (m, 2H), 1.48-1.30 (m, 4H), 1.28-1.17 (m, 1H), 1.13-0.92 (m, 4H); ¹³C NMR (101 MHz, CDCl₃) δ 150.1, 129.0 (2C), 124.5, 117.3 (2C), 56.9, 53.4 (2C), 50.9, 49.3 (2C), 36.9 (2C), 35.7, 34.2, 32.3 (2C); IR (film) 3332, 3271, 3183 cm⁻¹; mp 101-103 °C; HRMS (ESI) m/z [M+H]⁺ calcd for C₁₈H₂₉ClN₃ 322.2045, found 322.2040.

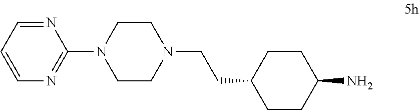 [SMILES: N[C@H]1CC[C@H](CCN2CCN(c3ncccn3)CC2)CC1]

### trans-4-(2-(4-(Pyrimidin-2-yl)piperazin-1-yl)ethyl)cyclohexan-1-amine (compound 5h)

General procedure C was followed using compound 4h (0.131 g, 0.337 mmol) and TFA (1.1 mL, 14 mmol) in DCM (2.4 mL). Afterwork-up, compound 5h (92.1 mg, 0.318 mmol, 94% yield) was isolated as an orange solid. R_{f}=0.1 (10% MeOH/DCM); ¹H NMR (400 MHz, CDCl₃) δ 8.29 (d, J=4.7 Hz, 2H), 6.46 (t, J=4.7 Hz, 1H), 3.87-3.73 (m, 4H), 2.63-2.52 (m, 1H), 2.51-2.43 (m, 4H), 2.41-2.33 (m, 2H), 1.87-1.79 (m, 2H), 1.78-1.70 (m, 2H), 1.46-1.37 (m, 2H), 1.35-1.15 (m, 3H), 1.12-0.92 (m, 4H); ¹³C NMR (101 MHz, CDCl₃) δ 161.8, 157.8 (2C), 109.9, 57.0, 53.4 (2C), 50.9, 43.9 (2C), 36.9 (2C), 35.7, 34.2, 32.3 (2C); IR (film) 3346 cm⁻¹; mp 56-58 0C; HRMS (ESI) m/z [M+H]⁺ calcd for Cl₆H₂₈N₅ 290.2339, found 290.2334.

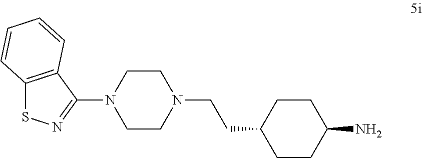 [SMILES: N[C@H]1CC[C@H](CCN2CCN(c3nsc4ccccc34)CC2)CC1]

### trans-4-(2-(4-(Benzo[d]isothiazol-3-yl)piperazin-1-yl)ethyl)cyclohexan-1-amine (compound 5i)

General procedure C was followed using compound 4i (0.152 g, 0.341 mmol) and TFA (1.0 mL, 13 mmol) in DCM (2.4 mL). Afterwork-up, compound 5i (0.108 g, 0.315 mmol, 92% yield) was isolated as a white solid. R_{f}=0.1 (10% MeOH/DCM); ¹H NMR (400 MHz, CDCl₃) δ 7.90 (d, J=8.1 Hz, 1H), 7.80 (d, J=8.1 Hz, 1H), 7.48-7.43 (m, 1H), 7.37-7.32 (m, 1H), 3.61-3.51 (m, 4H), 2.70-2.55 (m, 5H), 2.47-2.41 (m, 2H), 1.89-1.81 (m, 2H), 1.80-1.72 (m, 2H), 1.50-1.34 (m, 4H), 1.30-1.18 (m, 1H), 1.13-0.94 (m, 4H); ¹³C NMR (101 MHz, CDCl₃) δ 164.1, 152.9, 128.2, 127.6, 124.1, 124.0, 120.7, 57.0, 53.3 (2C), 51.0, 50.3 (2C), 36.9 (2C), 35.8, 34.1, 32.4 (2C); IR (film) 3350 cm⁻¹; mp 84-86 °C; HRMS (ESI) m/z [M+H]⁺ calcd for C₁₈H₂₈N₄S 345.2107, found 345.2104.

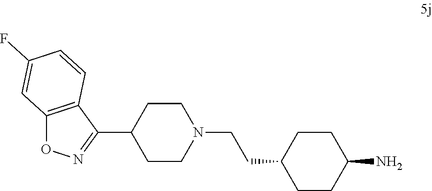 [SMILES: N[C@H]1CC[C@H](CCN2CCC(c3noc4cc(F)ccc34)CC2)CC1]

### trans-4-(2-(4-(6-Fluorobenzo[d]isoxazol-3-yl)piperidin-1-yl)ethyl)cyclohexan-1-amine (compound 5j)

General procedure C was followed using compound 4j (0.177 g, 0.396 mmol) and TFA (1.2 mL, 16 mmol) in DCM (2.8 mL). After work-up, compound 5j (0.134 g, 0.386 mmol, 98% yield) was isolated as a white solid. R_{f}=0.1 (10% MeOH/DCM); ¹H NMR (400 MHz, CDCl₃) δ 7.70 (dd, J=8.8, 5.1 Hz, 1H), 7.24 (d, J=8.6 Hz, 1H), 7.05 (t, J=8.8 Hz, 1H), 3.15-2.98 (m, 3H), 2.66-2.44 (m, 1H), 2.46-2.36 (m, 2H), 2.18-2.00 (m, 6H), 1.90-1.81 (m, 2H), 1.80-1.72 (m, 2H), 1.49-1.17 (m, 5H), 1.15-0.93 (m, 4H); ¹³C NMR (101 MHz, CDCl₃) δ 164.2 (d, J=250 Hz), 164.0 (d, J=14 Hz), 161.3, 122.8 (d, J=11 Hz), 117.5, 112.4 (d, J=25 Hz), 97.6 (d, J=27 Hz), 57.2, 53.8 (2C), 51.0, 36.9 (2C), 35.8, 34.9, 34.3, 32.4 (2C), 30.8 (2C); IR (film) 3349 cm⁻¹; mp 95-97 °C; HRMS (ESI) m/z [M+H]⁺ calcd for C₂₀H₂₉FN₃O 346.2289, found 346.2286.

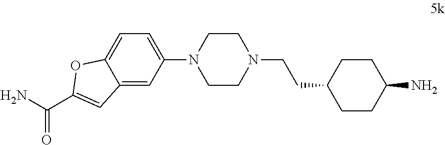 [SMILES: NC(=O)c1cc2cc(N3CCN(CC[C@H]4CC[C@H](N)CC4)CC3)ccc2o1]

### 5-(4-(2-(trans-4-Aminocyclohexyl)ethyl)piperazin-1-yl)benzofuran-2-carboxamide (compound 5k)

General procedure C was followed using compound 4k (0.254 g, 0.539 mmol) and TFA (1.7 mL, 22 mmol) in DCM (5.2 mL). After work-up, compound 5k (0.177 g, 0.479 mmol, 89% yield) was isolated as a white solid. Note: The aq layer was extracted with 10% MeOH/DCM. R_{f}=0.1 (10% MeOH containing 10% NH₄OH/DCM); ¹H NMR (400 MHz, CD₃CO₂D) δ 7.58-7.52 (m, 1H), 7.50-7.42 (m, 1H), 7.30-7.19 (m, 2H), 4.03-3.52 (m, 4H), 3.51-2.90 (m, 8H), 2.14-2.02 (m, 2H), 1.90-1.77 (m, 2H), 1.76-1.61 (m, 2H), 1.52-1.19 (m, 4H), 1.13-0.96 (m, 2H); ¹³C NMR (101 MHz, CD₃CO₂D) δ 164.8, 153.1, 150.3, 148.8, 130.0, 121.9, 114.3, 114.0, 112.0, 56.7, 53.3 (2C), 52.4, 50.1 (2C), 35.7, 32.1 (2C), 31.8 (2C), 31.7; IR (neat) 3413, 3285, 3165, 1655, 1608 cm⁻¹; dec >200 0C; HRMS (ESI) m/z [M+H]⁺ calcd for C₂₁H₃₁N₄O₂ 371.2442, found 371.2440.

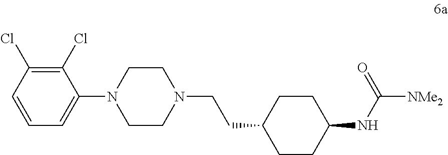 [SMILES: CN(C)C(=O)N[C@H]1CC[C@H](CCN2CCN(c3cccc(Cl)c3Cl)CC2)CC1]

### 3-(trans-4-(2-(4-(2,3-Dichlorophenyl)piperazin-1-yl)ethyl)cyclohexyl)-1,1-dimethylurea (compound 6a; cariprazine)

General procedure D was followed using compound 5a (1.00 g, 2.81 mmol) and N,N-dimethylcarbamoyl chloride (0.33 mL, 3.6 mmol) in DCM (28 mL). After work-up, the crude product was purified by chromatography (40 g of silica gel, 0-60% EtOAc/hexanes) to afford cariprazine (1.02 g, 2.39 mmol, 85% yield) as a white solid. R_{f}=0.5 (10% MeOH/DCM); ¹H NMR (400 MHz, CDCl₃) δ 7.17-7.10 (m, 2H), 6.95 (dd, J=6.3, 3.2 Hz, 1H), 4.11 (d, J=7.6 Hz, 1H), 3.64-3.52 (m, 1H), 3.15-2.98 (m, 4H), 2.88 (s, 6H), 2.71-2.53 (m, 4H), 2.47-2.38 (m, 2H), 2.07-1.96 (m, 2H), 1.83-1.70 (m, 2H), 1.49-1.39 (m, 2H), 1.30-1.18 (m, 1H), 1.16-1.01 (m, 4H); ¹³C NMR (101 MHz, CDCl₃) δ 158.0, 151.5, 134.2, 127.7, 127.6, 124.6, 118.7, 56.8, 53.6 (2C), 51.5 (2C), 50.0, 36.3 (2C), 35.8, 34.2 (2C), 34.1, 32.3 (2C); IR (neat) 3338, 1622 cm⁻¹; mp 212-213 °C (dec); HRMS (MALDI) m/z [M+H]⁺ calcd for C₂₁H₃₃Cl₂N₄O 427.2026, found 427.2023; t_{R}=32.1 min (HPLC, basic). The HCl salt was precipitated from 0.03 M solution of the free base in 50% CHCl₃/acetone using a 2.0 M solution of HCl in Et₂O (5.0 equiv). Dec>200 °C. Anal. calcd for C₂₁H₃₂Cl₂N₄O·2HCl: C, 50.41; H, 6.85; N, 11.20. Found: C, 50.43; H, 6.84; N, 11.01.

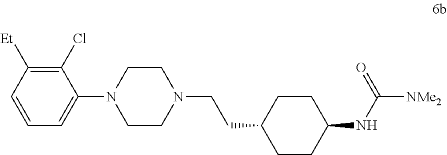 [SMILES: CCc1cccc(N2CCN(CC[C@H]3CC[C@H](NC(=O)N(C)C)CC3)CC2)c1Cl]

### 3-(trans-4-(2-(4-(2-Chloro-3-ethylphenyl)piperazin-1-yl)ethyl)cyclohexyl)-1,1-dimethylurea (compound 6b)

General procedure D was followed using compound 5b (0.500 g, 1.43 mmol) and N,N-dimethylcarbamoyl chloride (0.16 mL, 1.8 mmol) in DCM (20 mL). After work-up, the crude product was purified by chromatography (silica gel, 0-10% MeOH/CHCl₃) to afford compound 6b (0.414 g, 0.983 mmol, 69% yield) as a yellow solid. ¹H NMR (400 MHz, CDCl₃) δ 7.15 (t, J=7.7 Hz, 1H), 6.97-6.89 (m, 2H), 4.11 (d, J=7.6 Hz, 1H), 3.63-3.53 (m, 1H), 3.14-2.98 (m, 4H), 2.88 (s, 6H), 2.77 (q, J=7.6 Hz, 2H), 2.72-2.54 (m, 4H), 2.46-2.38 (m, 2H), 2.07-1.97 (m, 2H), 1.83-1.72 (m, 2H), 1.48-1.40 (m, 2H), 1.30-1.18 (m, 1H), 1.22 (t, J=7.5 Hz, 3H), 1.15-1.01 (m, 4H); ¹³C NMR (101 MHz, CDCl₃) δ 158.0, 149.8, 143.4, 128.8, 127.0, 124.1, 118.1, 56.9, 53.7 (2C), 51.7 (2C), 50.0, 36.3 (2C), 35.8, 34.2 (2C), 34.1, 32.3, 27.6, 14.2; t_{R}=20.3 min (HPLC, acidic). The HCl salt was precipitated from 50% acetone/CHCl₃ using a 2.0 M solution of HCl in Et₂O. Dec>200 °C. Anal. calcd for C₂₃H₃₇ClN₄O·HCl·H₂O: C, 58.10; H, 8.48; N, 11.78. Found: C, 58.32; H, 8.24; N, 11.56.

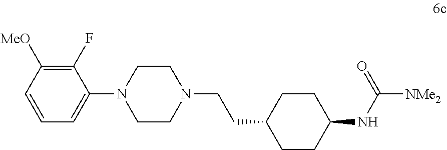 [SMILES: COc1cccc(N2CCN(CC[C@H]3CC[C@H](NC(=O)N(C)C)CC3)CC2)c1F]

### 3-(trans-4-(2-(4-(2-Fluoro-3-methoxyphenyl)piperazin-1-yl)ethyl)cyclohexyl)-1,1-dimethylurea (compound 6c)

General procedure D was followed using compound 5c (0.189 g, 0.565 mmol) and N,N-dimethylcarbamoyl chloride (66 μL, 0.72 mmol) in DCM (5.6 mL). After work-up, the crude product was purified by chromatography (12 g of silica gel, 0-10% MeOH/DCM) to afford compound 6c (0.179 g, 0.440 mmol, 78% yield) as a white solid. R_{f}=0.5 (10% MeOH/DCM); ¹H NMR (400 MHz, CDCl₃) δ 6.95 (dt, J=8.3, 1.8 Hz, 1H), 6.66-6.52 (m, 2H), 4.12 (d, J=7.6 Hz, 1H), 3.85 (s, 3H), 3.63-3.50 (m, 1H), 3.18-3.01 (m, 4H), 2.87 (s, 6H), 2.68-2.50 (m, 4H), 2.44-2.35 (m, 2H), 2.05-1.94 (m, 2H), 1.82-1.70 (m, 2H), 1.47-1.38 (m, 2H), 1.30-1.16 (m, 1H), 1.14-0.99 (m, 4H); ¹³C NMR (101 MHz, CDCl₃) δ 157.9, 148.7 (d, J=10 Hz), 145.8 (d, J=245 Hz), 141.2 (d, J=6 Hz), 123.6 (d, J=5 Hz), 111.2 (d, J=2 Hz), 107.1, 56.8, 56.6, 53.6 (2C), 50.8 (d, J=3 Hz, 2C), 50.0, 36.3 (2C), 35.8, 34.2 (2C), 34.1, 32.2 (2C); IR (film) 3342, 1626 cm⁻¹; mp 159-160 °C (dec); HRMS (ESI) m/z [M+H]⁺ calcd for C₂₂H₃₆FN₄O₂ 407.2817, found 407.2811; t_{R}=22.2 min (HPLC, basic). The HCl salt was precipitated from a 0.03 M solution of the free base in 50% CHCl₃/acetone using a 2.0 M solution of HCl in Et₂O (5.0 equiv). Mp 175-177 °C (dec). Anal. calcd for C₂₂H₃₅FN₄O₂·2HCl·1.25H₂O: C, 52.64; H, 7.93; N, 11.16. Found: C, 52.50; H, 7.79; N, 11.01.

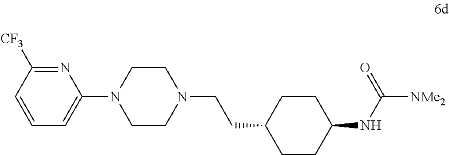 [SMILES: CN(C)C(=O)N[C@H]1CC[C@H](CCN2CCN(c3cccc(C(F)(F)F)n3)CC2)CC1]

### 1,1-Dimethyl-3-(trans-4-(2-(4-(6-(trifluoromethyl)62yridine-2-yl)piperazin-1-yl)ethyl)cyclohexyl)urea (compound 6d)

General procedure D was followed using compound 5d (0.232 g, 0.651 mmol) and N,N-dimethylcarbamoyl chloride (80 μL, 0.81 mmol) in DCM (9.5 mL). After work-up, the crude product was purified by chromatography (silica gel, 0-10% MeOH/CHCl₃) to afford compound 6d (0.107 g, 0.249 mmol, 38% yield) as a white solid. ¹H NMR (400 MHz, CDCl₃) δ 7.55 (t, J=8.2 Hz, 1H), 6.92 (d, J=7.7 Hz, 1H), 6.75 (d, J=8.5 Hz, 1H), 4.11 (d, J=7.7 Hz, 1H), 3.65-3.52 (m, 5H), 2.88 (s, 6H), 2.57-2.46 (m, 4H), 2.43-2.35 (m, 2H), 2.05-1.96 (m, 2H), 1.83-1.72 (m, 2H), 1.47-1.39 (m, 2H), 1.32-1.18 (m, 1H), 1.16-1.00 (m, 4H); ¹³C NMR (101 MHz, CDCl₃) δ 159.0, 158.0, 146.6 (q, J=34 Hz), 138.3, 121.8 (q, J=274 Hz), 109.5, 108.9 (q, J=3 Hz), 56.8, 53.1 (2C), 50.0, 44.9 (2C), 36.3 (2C), 35.8, 34.2 (2C), 34.0, 32.2 (2C); HRMS (MALDI) m/z [M+H]⁺ calcd for C₂₁H₃₃F₃N₅O 428.2632, found 428.2629; t_{R}=18.5 min (HPLC, acidic).

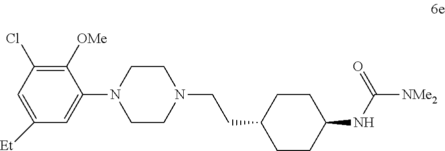 [SMILES: CCc1cc(Cl)c(OC)c(N2CCN(CC[C@H]3CC[C@H](NC(=O)N(C)C)CC3)CC2)c1]

### 3-(trans-4-(2-(4-(3-Chloro-5-ethyl-2-methoxyphenyl)piperazin-1-yl)ethyl)cyclohexyl)-1,1-dimethylurea (compound 6e)

General procedure D was followed using compound 5e (0.275 g, 0.723 mmol) and N,N-dimethylcarbamoyl chloride (82 μL, 0.90 mmol) in DCM (10 mL). Afterwork-up, the crude product was purified by chromatography (silica gel, 0-10% MeOH/CHCl₃) to afford compound 6e (0.215 g, 0.477 mmol, 66% yield) as an off-white solid. ¹H NMR (400 MHz, CDCl₃) δ 6.87 (d, J=1.9 Hz, 1H), 6.63 (d, J=2.0 Hz, 1H), 4.13 (d, J=7.6 Hz, 1H), 3.82 (s, 3H), 3.64-3.49, (m, 1H), 3.46-3.21 (m, 4H), 3.06-2.77 (m, 4H), 2.87 (s, 6H), 2.76-2.60 (m, 2H), 2.54 (q, J=7.6 Hz, 2H), 2.08-1.97 (m, 2H), 1.83-1.72 (m, 2H), 1.67-1.54 (m, 2H), 1.39-1.25 (m, 1H), 1.19 (t, J=7.6 Hz, 3H), 1.16-1.02 (m, 4H); ¹³C NMR (101 MHz, CDCl₃) δ 158.0, 146.6, 146.4, 140.9, 128.4, 122.3, 116.8, 66.0, 59.1, 56.9, 54.0 (2C), 50.4 (2C), 50.0, 36.3 (2C), 35.8, 34.2 (2C), 32.2 (2C), 28.6, 15.4. The HCl salt was precipitated from 50% CHCl₃/acetone using a 2.0 M solution of HCl in Et₂O. Dec>189 °C. Anal. calcd for C₂₄H₃₉ClN₄O₂·2HCl: C, 55.02; H, 7.89; N, 10.69. Found: C, 54.96; H, 7.88; N, 10.54.

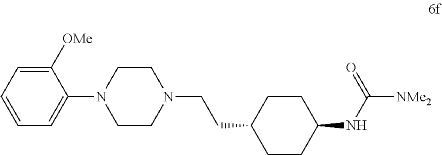 [SMILES: COc1ccccc1N1CCN(CC[C@H]2CC[C@H](NC(=O)N(C)C)CC2)CC1]

### 3-(trans-4-(2-(4-(2-Methoxyphenyl)piperazin-1-yl)ethyl)cyclohexyl)-1,1-dimethylurea (compound 6f)

General procedure D was followed using compound 5f (0.210 g, 0.663 mmol) and N,N-dimethylcarbamoyl chloride (79 μL, 0.86 mmol) in DCM (6.5 mL). After work-up, the crude product was purified by chromatography (12 g of silica gel, 0-10% MeOH/DCM) to afford compound 6f (0.172 g, 0.444 mmol, 67% yield) as a white, amorphous solid. R_{f}=0.4 (10% MeOH/DCM); ¹H NMR (400 MHz, CDCl₃) δ 7.02-6.88 (m, 3H), 6.87-6.82 (m, 1H), 4.11 (d, J=7.6 Hz, 1H), 3.85 (s, 3H), 3.63-3.51 (m, 1H), 3.22-2.95 (m, 4H), 2.87 (s, 6H), 2.75-2.51 (m, 4H), 2.46-2.36 (m, 2H), 2.07-1.94 (m, 2H), 1.82-1.71 (m, 2H), 1.49-1.39 (m, 2H), 1.31-1.16 (m, 1H), 1.15-0.99 (m, 4H); ¹³C NMR (101 MHz, CDCl₃) δ 157.9, 152.4, 141.5, 123.0, 121.1, 118.3, 111.2, 56.9, 55.5, 53.7 (2C), 50.8 (2C), 50.0, 36.3 (2C), 35.8, 34.2 (2C), 34.1, 32.2 (2C); IR (film) 3339, 1628 cm⁻¹; mp 137-138 °C (dec); HRMS (ESI) m/z [M+H]⁺ calcd for C₂₂H₃₇N₄O₂ 389.2911, found 389.2903; t_{R}=19.7 min (HPLC, basic).

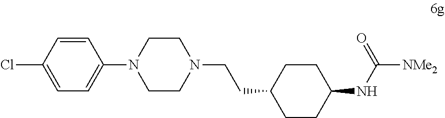 [SMILES: CN(C)C(=O)N[C@H]1CC[C@H](CCN2CCN(c3ccc(Cl)cc3)CC2)CC1]

### 3-(trans-4-(2-(4-(4-Chlorophenyl)piperazin-1-yl)ethyl)cyclohexyl)-1,1-dimethylurea (compound 6g)

General procedure D was followed using compound 5g (0.112 g, 0.348 mmol) and N,N-dimethylcarbamoyl chloride (42 μL, 0.46 mmol) in DCM (3.4 mL). After work-up, the crude product was purified by chromatography (12 g of silica gel, 0-10% MeOH/DCM) to afford compound 6g (0.100 g, 0.255 mmol, 73% yield) as a white, amorphous solid. R_{f}=0.4 (10% MeOH/DCM); ¹H NMR (400 MHz, CDCl₃) δ 7.19 (d, J=8.9 Hz, 2H), 6.83 (d, J=8.9 Hz, 2H), 4.11 (d, J=7.6 Hz, 1H), 3.63-3.51 (m, 1H), 3.20-3.10 (m, 4H), 2.87 (s, 6H), 2.62-2.53 (m, 4H), 2.43-2.35 (m, 2H), 2.06-1.95 (m, 2H), 1.82-1.70 (m, 2H), 1.48-1.39 (m, 2H), 1.29-1.17 (m, 1H), 1.15-1.00 (m, 4H); ¹³C NMR (101 MHz, CDCl₃) δ 158.0, 150.1, 129.0 (2C), 124.5, 117.3 (2C), 56.8, 53.3 (2C), 50.0, 49.3 (2C), 36.3 (2C), 35.8, 34.2 (2C), 34.1, 32.2 (2C); IR (film) 3363, 1626 cm⁻¹; mp 196-197 °C (dec); HRMS (ESI) m/z [M+H]⁺ calcd for C₂₁H₃₄ClN₄O 393.2416, found 393.2408; t_{R}=23.6 min (HPLC, basic).

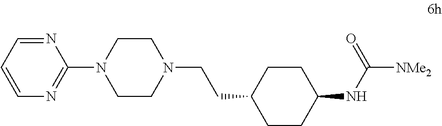 [SMILES: CN(C)C(=O)N[C@H]1CC[C@H](CCN2CCN(c3ncccn3)CC2)CC1]

### 1,1-Dimethyl-3-(trans-4-(2-(4-(pyrimidin-2-yl)piperazin-1-yl)ethyl)cyclohexyl)urea (compound 6h)

General procedure D was followed using compound 5h (82.8 mg, 0.286 mmol) and N,N-dimethylcarbamoyl chloride (34 μL, 0.37 mmol) in DCM (2.8 mL). After work-up, the crude product was purified by chromatography (12 g of silica gel, 0-10% MeOH/DCM) to afford compound 6h (81.4 mg, 0.226 mmol, 79% yield) as a white solid. R_{f}=0.3 (10% MeOH/DCM); ¹H NMR (400 MHz, CDCl₃) δ 8.29 (d, J=4.8 Hz, 2H), 6.47 (t, J=4.5 Hz, 1H), 4.11 (d, J=7.6 Hz, 1H), 3.88-3.77 (m, 4H), 3.64-3.50 (m, 1H), 2.87 (s, 6H), 2.52-2.44 (m, 4H), 2.41-2.34 (m, 2H), 2.05-1.95 (m, 2H), 1.81-1.71 (m, 2H), 1.47-1.39 (m, 2H), 1.29-1.17 (m, 1H), 1.15-1.01 (m, 4H); ¹³C NMR (101 MHz, CDCl₃) δ 161.8, 158.0, 157.8 (2C), 109.9, 56.9, 53.4 (2C), 50.0, 43.9 (2C), 36.3 (2C), 35.8, 34.2 (2C), 34.0, 32.2 (2C); IR (film) 3347, 1627 cm⁻¹; mp 172-173 °C (dec); HRMS (ESI) m/z [M+H]⁺ calcd for C₁₈H₃₃N₆O 361.2710, found 361.2702; t_{R}=18.5 min (HPLC, basic).

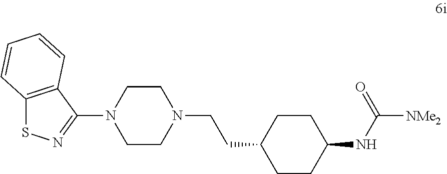 [SMILES: CN(C)C(=O)N[C@H]1CC[C@H](CCN2CCN(c3nsc4ccccc34)CC2)CC1]

### 3-(trans-4-(2-(4-(Benzo[d]isothiazol-3-yl)piperazin-1-yl)ethyl)cyclohexyl)-1,1-dimethylurea (compound 6i)

General procedure D was followed using compound 5i (96.7 mg, 0.281 mmol) and N,N-dimethylcarbamoyl chloride (34 μL, 0.37 mmol) in DCM (2.8 mL). After work-up, the crude product was purified by chromatography (12 g of silica gel, 0-10% MeOH/DCM) to afford compound 6i (0.108 g, 0.261 mmol, 93% yield) as a white solid. R_{f}=0.5 (10% MeOH/DCM); ¹H NMR (400 MHz, CDCl₃) δ 7.90 (d, J=8.2 Hz, 1H), 7.80 (d, J=8.2 Hz, 1H), 7.50-7.41 (m, 1H), 7.38-7.30 (m, 1H), 4.17-4.06 (m, 1H), 3.65-3.47 (m, 5H), 2.87 (s, 6H), 2.71-2.61 (m, 4H), 2.48-2.39 (m, 2H), 2.08-1.96 (m, 2H), 1.84-1.71 (m, 2H), 1.50-1.39 (m, 2H), 1.31-1.20 (m, 1H), 1.16-1.00 (m, 4H); ¹³C NMR (101 MHz, CDCl₃) b 164.1, 158.0, 152.9, 128.2, 127.6, 124.1, 124.0, 120.7, 56.9, 53.3 (2C), 50.3 (2C), 50.0, 36.3 (2C), 35.8, 34.2 (2C), 34.0, 32.3 (2C); IR (film) 3341, 1629 cm⁻¹; mp 177-178 °C (dec); HRMS (ESI) m/z [M+H]⁺ calcd for C₂₂H₃₄N₅OS 416.2479, found 416.2470; t_{R}=23.2 min (HPLC, basic).

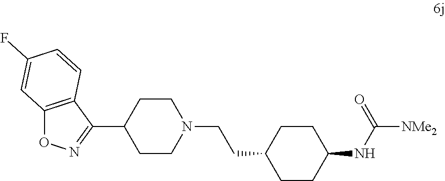 [SMILES: CN(C)C(=O)N[C@H]1CC[C@H](CCN2CCC(c3noc4cc(F)ccc34)CC2)CC1]

### 3-(trans-4-(2-(4-(6-Fluorobenzo[d]isoxazol-3-yl)piperidin-1-yl)ethyl)cyclohexyl)-1,1-dimethylurea (compound 6j)

General procedure D was followed using compound 5j (0.122 g, 0.353 mmol) and N,N-dimethylcarbamoyl chloride (42 μL, 0.46 mmol) in DCM (3.4 mL). After work-up, the crude product was purified by chromatography (12 g of silica gel, 0-10% MeOH/DCM) to afford compound 6j (95.3 mg, 0.229 mmol, 65% yield) as a white solid. R_{f}=0.3 (10% MeOH/DCM); ¹H NMR (400 MHz, CDCl₃) δ 7.70 (dd, J=8.7, 5.1 Hz, 1H), 7.23 (dd, J=8.5, 2.1 Hz, 1H), 7.04 (dt, J=2.2, 8.9 Hz, 1H), 4.12 (d, J=7.6 Hz, 1H), 3.64-3.50 (m, 1H), 3.14-2.98 (m, 3H), 2.87 (s, 6H), 2.46-2.34 (m, 2H), 2.20-1.95 (m, 8H), 1.83-1.71 (m, 2H), 1.50-1.39 (m, 2H), 1.30-1.16 (m, 1H), 1.15-1.01 (m, 4H); ¹³C NMR (101 MHz, CDCl₃) δ 164.2 (d, J=251 Hz), 164.0 (d, J=14 Hz), 161.2, 158.0, 122.8 (d, J=11 Hz), 117.5, 112.4 (d, J=25 Hz), 97.5 (d, J=27 Hz), 57.0, 53.8 (2C), 50.0, 36.3 (2C), 35.8, 34.8, 34.2 (2C), 34.1, 32.2 (2C), 30.7 (2C); IR (film) 3341, 1616 cm⁻¹; mp 155-156 °C (dec); HRMS (ESI) m/z [M+H]⁺ calcd for C₂₃H₃₄FN₄O₂ 417.2660, found 417.2651; t_{R}=23.5 min (HPLC, basic).

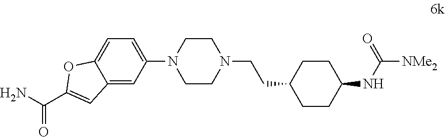 [SMILES: CN(C)C(=O)N[C@H]1CC[C@H](CCN2CCN(c3ccc4oc(C(N)=O)cc4c3)CC2)CC1]

### 5-(4-(2-(trans-4-(3,3-Dimethylureido)cyclohexyl)ethyl)piperazin-1-yl)benzofuran-2-carboxamide (compound 6k)

General procedure D was followed using compound 5k (0.167 g, 0.451 mmol) and N,N-dimethylcarbamoyl chloride (54 μL, 0.59 mmol) in DCM (13.5 mL). After work-up, the crude product was purified by chromatography (12 g of silica gel, 0-10% MeOH/DCM) to afford compound 6k (92.2 mg, 0.209 mmol, 46% yield) as a white solid. R_{f}=0.1 (10% MeOH/DCM); ¹H NMR (400 MHz, CD₃CO₂D) δ 7.60 (s, 1H), 7.51 (d, J=9.1 Hz, 1H), 7.33 (s, 1H), 7.28 (d, J=9.1 Hz, 1H), 3.97-3.55 (m, 5H), 3.52-3.09 (m, 6H), 2.89 (s, 6H), 2.00-1.89 (m, 2H), 1.84-1.68 (m, 4H), 1.47-1.16 (m, 4H), 1.15-1.01 (m, 2H); ¹³C NMR (101 MHz, CD₃CO₂D) δ 164.1, 160.3, 152.4, 149.4, 147.9, 129.2, 121.2, 113.5, 113.3, 111.4, 56.2, 52.5 (2C), 51.0, 49.3 (2C), 36.6 (2C), 35.6, 33.9 (2C), 32.4 (2C), 31.1; IR (neat) 3348, 3286, 3143, 1678, 1659, 1628 cm⁻¹; dec >225 °C; HRMS (ESI) m/z [M+H]⁺ calcd for C₂₄H₃₆N₅O₃ 442.2813, found 442.2804; t_{R}=18.0 min (HPLC, basic).

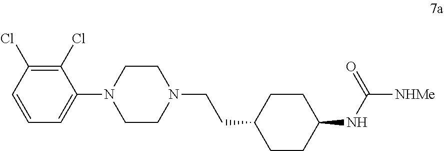 [SMILES: CNC(=O)N[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)-3-methylurea (compound 7a)

General procedure D was followed using compound 5a (0.250 g, 0.702 mmol) and N-methylcarbamoyl chloride (83.0 mg, 0.456 mmol) in DCM (10 mL). After work-up, the crude product was purified by chromatography (silica gel, 0-10% MeOH/CHCl₃) to afford compound 7a (0.141 g, 0.341 mmol, 49% yield) as a white solid. ¹H NMR (400 MHz, CDCl₃) δ 7.19-7.13 (m, 2H), 6.97 (dd, J=5.7, 3.9 Hz, 1H), 5.11-5.05 (m, 1H), 5.00 (d, J=8.0 Hz, 1H), 3.53-3.40 (m, 1H), 3.18-2.94 (m, 4H), 2.72 (d, J=4.7 Hz, 3H), 2.68-2.55 (m, 4H), 2.47-2.38 (m, 2H), 2.04-1.92 (m, 2H), 1.82-1.70 (m, 2H), 1.49-1.39 (m, 2H), 1.30-1.19 (m, 1H), 1.14-1.00 (m, 4H); ¹³C NMR (101 MHz, CDCl₃) δ 158.6, 151.1, 133.6, 127.3, 127.1, 124.3, 118.5, 56.4, 53.1 (2C), 51.1 (2C), 48.9, 35.3, 33.7 (3C), 31.9 (2C), 26.5. The HCl salt was precipitated from a 0.02 M solution of the free base in 50% CHCl₃/acetone using a 2.0 M solution of HCl in Et₂O (35 equiv). Dec>175 °C. Anal. calcd for C₂₀H₃₀Cl₂N₄O·HCl·1.25H₂O: C, 50.85; H, 7.15; N, 11.86. Found: C, 50.86; H, 6.86; N, 11.60.

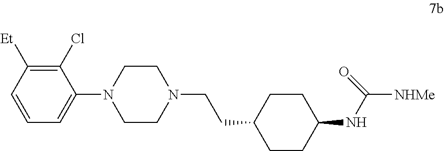 [SMILES: CCc1cccc(N2CCN(CC[C@H]3CC[C@H](NC(=O)NC)CC3)CC2)c1Cl]

### 1-(trans-4-(2-(4-(2-Chloro-3-ethylphenyl)piperazin-1-yl)ethyl)cyclohexyl)-3-methylurea (compound 7b)

General procedure D was followed using compound 5b (0.172 g, 0.491 mmol) and N-methylcarbamoyl chloride (0.139 g, 1.47 mmol) in DCM (15 mL). After work-up, the crude product was purified by chromatography (silica gel, 0-10% MeOH/CHCl₃) to afford compound 7b (0.150 g, 0.368 mmol, 75% yield) as a white solid. ¹H NMR (400 MHz, CDCl₃) δ 7.15 (t, J=7.7 Hz, 1H), 6.96-6.90 (m, 2H), 4.28-4.21 (m, 1H), 4.15 (d, J=8.0 Hz, 1H), 3.51-3.39 (m, 1H), 3.16-2.94 (m, 4H), 2.81-2.73 (m, 5H), 2.72-2.52 (m, 4H), 2.46-2.36 (m, 2H), 2.06-1.94 (m, 2H), 1.85-1.69 (m, 2H), 1.49-1.41 (m, 2H), 1.22 (t, J=7.5 Hz, 3H), 1.31-1.17 (m, 1H), 1.15-1.01 (m, 4H); ¹³C NMR (101 MHz, CDCl₃) δ 158.3, 149.8, 143.4, 128.8, 127.0, 124.1, 118.1, 56.8, 53.7 (2C), 51.7 (2C), 49.8, 35.7, 34.09, 34.06 (2C), 32.2 (2C), 27.6, 27.4, 14.2. The HCl salt was precipitated from a 0.03 M solution of the free base in 50% CHCl₃/acetone using a 2.0 M solution of HCl in Et₂O (29 equiv). Dec>199 °C. Anal. calcd for C₂₂H₃₅ClN₄O·HCl·1.25H₂O: C, 56.71; H, 8.33; N, 12.02. Found: C, 56.54; H, 7.92; N, 11.94.

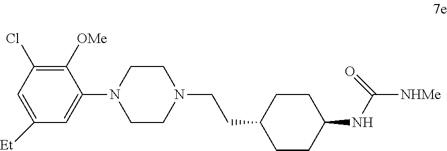 [SMILES: CCc1cc(Cl)c(OC)c(N2CCN(CC[C@H]3CC[C@H](NC(=O)NC)CC3)CC2)c1]

### 1-(trans-4-(2-(4-(3-Chloro-5-ethyl-2-methoxyphenyl)piperazin-1-yl)ethyl)cyclohexyl)-3-methylurea (compound 7e)

General procedure D was followed using compound 5e (0.420 g, 1.11 mmol) and N-methylcarbamoyl chloride (0.130 g, 1.38 mmol) in DCM (15 mL). After work-up, the crude product was purified by chromatography (silica gel, 0-10% MeOH/CHCl₃) to afford compound 7e (0.416 g, 0.952 mmol, 86% yield) as a white solid. ¹H NMR (400 MHz, CDCl₃) δ 6.84 (d, J=2.0 Hz, 1H), 6.61 (d, J=2.0 Hz, 1H), 4.16 (d, J=5.1 Hz, 1H), 4.08 (d, J=8.0 Hz, 1H), 3.83 (s, 3H), 3.52-3.40 (m, 1H), 3.22-3.05 (m, 4H), 2.77 (d, J=4.9 Hz, 3H), 2.66-2.49 (m, 6H), 2.44-2.36 (m, 2H), 2.06-1.95 (m, 2H), 1.84-1.73 (m, 2H), 1.48-1.40 (m, 2H), 1.31-1.23 (m, 1H), 1.20 (t, J=7.6 Hz, 3H), 1.15-1.01 (m, 4H); ¹³C NMR (101 MHz, CDCl₃) δ 158.3, 146.5, 146.3, 141.0, 128.4, 122.3, 116.8, 59.2, 56.9, 54.0 (2C), 50.4 (2C), 49.9, 35.7, 34.1 (2C), 34.0, 32.2 (2C), 28.6, 27.4, 15.6. The HCl salt was precipitated from a 0.04 M solution of the free base in 50% CHCl₃/acetone using a 2.0 M solution of HCl in Et₂O (27 equiv). Dec>220 °C. Anal. calcd for C₂₃H₃₇ClN₄O₂·2HCl·1.25H₂O: C, 51.88; H, 7.86; N, 10.52. Found: C, 51.89; H, 7.60; N, 10.36.

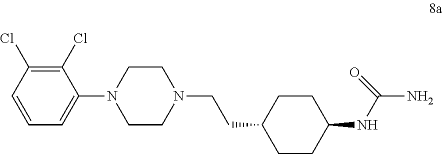 [SMILES: NC(=O)N[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)urea (compound 8a)

To a solution of compound 5a (0.108 g, 0.303 mmol) and KOCN (0.256 g, 3.16 mmol) in H₂O (1.5 mL) and THE (3.0 mL) was added a 1.0 M aqueous solution of HCl (1.5 mL, 1.5 mmol) over 3 min. After stirring overnight, the reaction was quenched with a saturated aqueous solution of NaHCO₃ (20 mL), and the aqueous layer was extracted with EtOAc (2×20 mL). The combined organic layers were dried over Na₂SO₄, filtered, and concentrated. The crude product was purified by chromatography (12 g of silica gel, 0-10% MeOH/DCM) to afford compound 8a (71.3 mg, 0.179 mmol, 59% yield) as a white solid. R_{f}=0.3 (10% MeOH/DCM); ¹H NMR (400 MHz, CDCl₃) δ 7.18-7.10 (m, 2H), 6.95 (dd, J=6.5, 3.1 Hz, 1H), 4.38-4.23 (m, 3H), 3.50-3.37 (m, 1H), 3.16-2.97 (m, 4H), 2.72-2.52 (m, 4H), 2.42 (t, J=8.0 Hz, 2H), 2.08-1.96 (m, 2H), 1.85-1.64 (m, 3H), 1.51-1.39 (m, 2H), 1.17-0.99 (m, 4H); ¹³C NMR (101 MHz, CDCl₃) δ 157.8, 151.5, 134.2, 127.7, 127.6, 124.7, 118.7, 56.7, 53.6 (2C), 51.5 (2C), 50.2, 35.6, 34.1, 33.8 (2C), 32.1 (2C); IR (neat) 3477, 3329, 1645 cm⁻¹; mp 212-213 °C (dec); HRMS (MALDI) m/z [M+H]⁺ calcd for Cl₉H₂₉Cl₂N₄O 399.1713, found 399.1711; t_{R}=24.9 min (HPLC, basic).

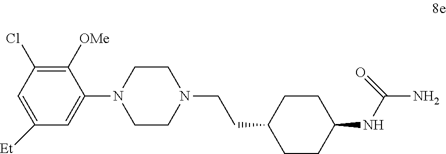 [SMILES: CCc1cc(Cl)c(OC)c(N2CCN(CC[C@H]3CC[C@H](NC(N)=O)CC3)CC2)c1]

### 1-(trans-4-(2-(4-(3-Chloro-5-ethyl-2-methoxyphenyl)piperazin-1-yl)ethyl)cyclohexyl)urea (compound 8e)

The same procedure as the one described for compound 8a was followed starting from 5e (0.203 g, 0.534 mmol). After work-up, the crude product was purified by chromatography (12 g of silica gel, 0-10% MeOH/DCM) to afford compound 8e (93.0 mg, 0.220 mmol, 41% yield) as a tan solid. R_{f}=0.3 (10% MeOH/DCM); ¹H NMR (400 MHz, CDCl₃) δ 6.83 (d, J=2.0 Hz, 1H), 6.61 (d, J=2.0 Hz, 1H), 4.58-4.33 (m, 3H), 3.83 (s, 3H), 3.49-3.34 (m, 1H), 3.26-2.99 (m, 4H), 2.68-2.48 (m, 6H), 2.45-2.34 (m, 2H), 2.06-1.94 (m, 2H), 1.85-1.70 (m, 2H), 1.48-1.38 (m, 2H), 1.30-1.22 (m, 1H), 1.19 (t, J=7.6 Hz, 3H), 1.16-1.00 (m, 4H); ¹³C NMR (101 MHz, CDCl₃) δ 158.2, 146.5, 146.3, 140.9, 128.3, 122.3, 116.8, 59.1, 56.8, 54.0 (2C), 50.4 (2C), 50.0, 35.6, 34.0, 33.8 (2C), 32.1 (2C), 28.6, 15.6; IR (neat) 3492, 3348, 1647 cm⁻¹; mp 163-166 °C (dec); HRMS (MALDI) m/z [M+H]⁺ calcd for C₂₂H₃₆ClN₄O₂ 423.2521, found 423.2518; t_{R}=32.0 min (HPLC, basic).

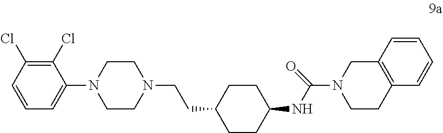 [SMILES: O=C(N[C@H]1CC[C@H](CCN2CCN(c3cccc(Cl)c3Cl)CC2)CC1)N1CCc2ccccc2C1]

### N-(trans-4-(2-(4-(2,3-Dichlorophenyl)piperazin-1-yl)ethyl)cyclohexyl)-7,8-dihydro-1,6-naphthyridine-6(5H)-carboxamide (compound 9a)

Using oven-dried glassware under an argon atmosphere, a solution of NEt₃ (0.21 mL, 1.5 mmol) in anhydrous THF (3.5 mL) was cooled to 0 °C, and a 15 wt % solution of phosgene in toluene (0.40 mL, 0.56 mmol) was added over 3 min. Next, a solution of compound 5a (0.179 g, 0.502 mmol) in anhydrous THF (3.5 mL) was added over 3 min, and the reaction was stirred for 1 h. Then, a solution of 5,6,7,8-tetrahydro-1,6-napthyridine (80.5 mg, 0.600 mmol) in anhydrous THF (3.5 mL) was added over 3 min, and the reaction was allowed to warm to rt. After stirring for 19 h, the reaction was quenched with a saturated aqueous solution of NaHCO₃ (40 mL), and the aqueous layer was extracted with EtOAc (40 mL). The organic layer was dried over anhydrous Na₂SO₄, filtered, and concentrated. The crude product was purified by chromatography (12 g of silica gel, 0-10% MeOH/DCM) to afford compound 9a (35.1 mg, 68.0 μmol, 14% yield) as a white solid. R_{f}=0.4 (10% MeOH/DCM); ¹H NMR (400 MHz, CDCl₃) δ 8.42 (d, J=4.2 Hz, 1H), 7.42 (d, J=7.6 Hz, 1H), 7.17-7.10 (m, 3H), 6.95 (dd, J=6.4, 3.2 Hz, 1H), 4.57 (s, 2H), 4.32 (d, J=7.6 Hz, 1H), 3.71-3.57 (m, 3H), 3.14-2.96 (m, 6H), 2.76-2.52 (m, 4H), 2.47-2.39 (m, 2H), 2.08-2.00 (m, 2H), 1.83-1.75 (m, 2H), 1.48-1.39 (m, 2H), 1.30-1.20 (m, 1H), 1.18-1.03 (m, 4H); ¹³C NMR (101 MHz, CDCl₃) δ 156.9, 155.0, 151.4, 148.0, 134.2, 134.1, 129.1, 127.62, 127.56, 124.6, 121.7, 118.7, 56.8, 53.5 (2C), 51.5 (2C), 50.1, 45.0, 41.6, 35.8, 34.08 (2C), 34.05, 32.2 (3C); IR (neat) 3326, 1620 cm⁻¹; mp 163-164 °C (dec); HRMS (MALDI) m/z [M+H]⁺ calcd for C₂₇H₃₆Cl₂N₅₀ 516.2291, found 516.2283; t_{R}=33.8 min (HPLC, basic).

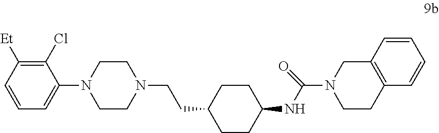 [SMILES: CCc1cccc(N2CCN(CC[C@H]3CC[C@H](NC(=O)N4CCc5ccccc5C4)CC3)CC2)c1Cl]

### N-(trans-4-(2-(4-(2-chloro-3-ethylphenyl)piperazin-1-yl)ethyl)cyclohexyl)-7,8-dihydro-1,6-naphthyridine-6(5H)-carboxamide (compound 9b)

The same procedure as the one described for compound 9a was followed starting from compound 5b (0.205 g, 0.586 mmol). After work-up, the crude product was purified by chromatography (12 g of silica gel, 0-10% MeOH/DCM) to afford compound 9b (0.192 g, 0.376 mmol, 64% yield) as a white solid. R_{f}=0.4 (10% MeOH/DCM); ¹H NMR (400 MHz, CDCl₃) δ 8.42 (d, J=4.7 Hz, 1H), 7.43 (d, J=7.7 Hz, 1H), 7.18-7.10 (m, 2H), 6.93 (t, J=7.2 Hz, 2H), 4.57 (s, 2H), 4.32 (d, J=7.5 Hz, 1H), 3.71-3.59 (m, 3H), 3.12-2.98 (m, 6H), 2.77 (q, J=7.5 Hz, 2H), 2.71-2.54 (m, 4H), 2.47-2.39 (m, 2H), 2.10-2.01 (m, 2H), 1.84-1.74 (m, 2H), 1.50-1.40 (m, 2H), 1.30-1.18 (m, 1H), 1.22 (t, J=7.5 Hz, 3H), 1.15-1.04 (m, 4H); ¹³C NMR (101 MHz, CDCl₃) δ 156.9, 155.1, 149.8, 148.0, 143.4, 134.2, 129.1, 128.8, 127.0, 124.1, 121.7, 118.1, 56.9, 53.7 (2C), 51.7 (2C), 50.2, 45.0, 41.6, 35.8, 34.1 (3C), 32.2 (3C), 27.6, 14.4; IR (neat) 3323, 1619 cm⁻¹; mp 58-60 °C; HRMS (MALDI) m/z [M+H]⁺ calcd for C₂₉H₄₁ClN₅O 510.2994, found 510.2988; t_{R}=35.2 min (HPLC, basic).

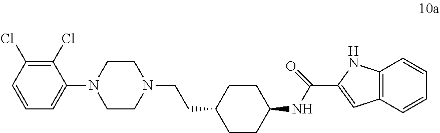 [SMILES: O=C(N[C@H]1CC[C@H](CCN2CCN(c3cccc(Cl)c3Cl)CC2)CC1)c1cc2ccccc2[nH]1]

### N-(trans-4-(2-(4-(2,3-Dichlorophenyl)piperazin-1-yl)ethyl)cyclohexyl)-1H-indole-2-carboxamide (compound 10a)

General procedure E was followed using compound 5a (0.101 g, 0.283 mmol) and 1H-indole-2-carboxylic acid (62.0 mg, 0.385 mmol) in CHCl₃ (8.0 mL). After work-up, the crude product was product was purified by chromatography (12 g of silica gel, 0-10% MeOH/DCM) to afford compound 10a (20.2 mg, 40.2 μmol, 14% yield) as a white solid. R_{f}=0.5 (10% MeOH/DCM); ¹H NMR (400 MHz, CDCl₃) δ 9.22 (s, 1H), 7.64 (d, J=8.0 Hz, 1H), 7.43 (d, J=8.3 Hz, 1H), 7.32-7.28 (m, 1H), 7.19-7.10 (m, 3H), 6.97 (dd, J=6.5, 3.1 Hz, 1H), 6.80 (s, 1H), 5.95 (d, J=8.2 Hz, 1H), 4.03-3.89 (m, 1H), 3.17-2.00 (m, 4H), 2.76-2.55 (m, 4H), 2.47 (t, J=7.9 Hz, 2H), 2.13 (d, J=11.8 Hz, 2H), 1.86 (d, J=12.4 Hz, 2H), 1.54-1.42 (m, 2H), 1.37-1.10 (m, 5H); ¹³C NMR (101 MHz, CDCl₃) δ 160.8, 151.4, 136.2, 134.2, 131.2, 127.9, 127.7, 127.6, 124.7, 124.6, 122.0, 120.8, 118.7, 112.0, 101.6, 56.7, 53.6 (2C), 51.5 (2C), 49.1, 35.6, 34.0, 33.4 (2C), 32.1 (2C); IR (neat) 3616, 3268, 1624 cm⁻¹; mp 240-241 °C (dec); HRMS (MALDI) m/z [M+H]⁺ calcd for C₂₇H₃₃Cl₂N₄O 499.2026, found 499.2025; t_{R}=37.8 min (HPLC, basic).

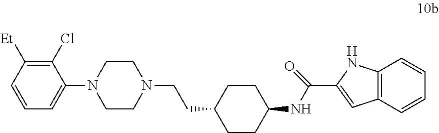 [SMILES: CCc1cccc(N2CCN(CC[C@H]3CC[C@H](NC(=O)c4cc5ccccc5[nH]4)CC3)CC2)c1Cl]

### N-(trans-4-(2-(4-(2-Chloro-3-ethylphenyl)piperazin-1-yl)ethyl)cyclohexyl)indole-2-carboxamide (compound 10b)

General procedure E was followed using compound 5b (0.100 g, 0.286 mmol) and 1H-indole-2-carboxylic acid (58.0 mg, 0.360 mmol) in CHCl₃ (10 mL). After work-up, the crude product was purified by chromatography (silica gel, 0-50% EtOAc/hexanes) to afford compound 10b (70 mg, 0.142 mmol, 50% yield) as an off-white solid. ¹H NMR (400 MHz, CDCl₃) δ 9.16 (s, 1H), 7.64 (d, J=8.0 Hz, 1H), 7.43 (d, J=8.4 Hz, 1H), 7.31-7.26 (m, 1H), 7.18-7.11 (m, 2H), 6.97-6.92 (m, 2H), 6.80 (dd, J=2.1, 0.9 Hz, 1H), 5.94 (d, J=8.2 Hz, 1H), 4.00-3.89 (m, 1H), 3.16-2.98 (m, 4H), 2.78 (q, J=7.5 Hz, 2H), 2.73-2.55 (m, 4H), 2.50-2.41 (m, 2H), 2.17-2.08 (m, 2H), 1.90-1.82 (m, 2H), 1.53-1.45 (m, 2H), 1.38-1.10 (m, 7H); ¹³C NMR (101 MHz, CDCl₃) δ 160.8, 149.8, 143.4, 136.2, 131.2, 128.9, 127.9, 127.0, 124.6, 124.1, 122.0, 120.8, 118.2, 112.0, 101.6, 56.8, 53.8 (2C), 51.8 (2C), 49.1, 35.7, 34.1, 33.4 (2C), 32.1 (2C), 27.6, 14.3. The HCl salt was precipitated from 50% CHCl₃/acetone using a 2.0 M solution of HCl in Et₂O. Anal. calcd for C₂₉H₃₇ClN₄O·HCl·0.75H₂O: C, 64.14; H, 7.33; N, 10.32. Found: C, 64.11; H, 7.09; N, 10.04.

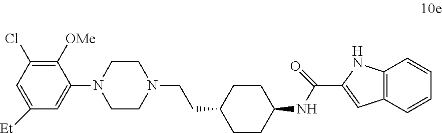 [SMILES: CCc1cc(Cl)c(OC)c(N2CCN(CC[C@H]3CC[C@H](NC(=O)c4cc5ccccc5[nH]4)CC3)CC2)c1]

### N-(trans-4-(2-(4-(3-Chloro-5-ethyl-2-methoxyphenyl)piperazin-1-yl)ethyl)cyclohexyl)indole-2-carboxamide (compound 10e)

General procedure E was followed using compound 5e (0.125 g, 0.329 mmol) and 1H-indole-2-carboxylic acid (58.3 mg, 0.362 mmol) in CHCl₃ (8.0 mL). After work-up, the crude product was purified by chromatography (silica gel, 0-10% MeOH/CHCl₃) to afford compound 10e (0.133 g, 0.254 mmol, 77% yield) as a white solid. ¹H NMR (400 MHz, CDCl₃) δ 9.33 (s, 1H), 7.64 (d, J=8.0 Hz, 1H), 7.44 (d, J=8.3 Hz, 1H), 7.33-7.24 (m, 1H), 7.14 (t, J=7.5 Hz, 1H), 6.85 (s, 1H), 6.81 (s, 1H), 6.62 (s, 1H), 5.97 (d, J=8.2 Hz, 1H), 4.02-3.90 (m, 1H), 3.85 (s, 3H), 3.25-3.06 (m, 4H), 2.69-2.51 (m, 6H), 2.47-2.40 (m, 2H), 2.17-2.08 (m, 2H), 1.90-1.81 (m, 2H), 1.53-1.44 (m, 2H), 1.39-1.09 (m, 8H). The HCl salt was precipitated from a 0.06 M solution of the free base in 50% CHCl₃/acetone using a 2.0 M solution of HCl in Et₂O (20 equiv). Dec>220 °C. Anal. calcd for C₃₀H₃₉ClN₄O₂·2HCl·0.25H₂O: C, 60.00; H, 6.97; N, 9.33. Found: C, 60.16; H, 7.10; N, 9.12.

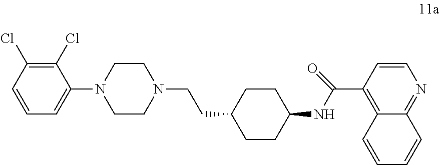 [SMILES: O=C(N[C@H]1CC[C@H](CCN2CCN(c3cccc(Cl)c3Cl)CC2)CC1)c1ccnc2ccccc12]

### N-(trans-4-(2-(4-(2,3-Dichlorophenyl)piperazin-1-yl)ethyl)cyclohexyl)quinoline-4-carboxamide (compound 11a)

General procedure E was followed using compound 5a (0.221 g, 0.620 mmol) and quinoline-4-carboxylic acid (0.118 g, 0.680 mmol) in CHCl₃ (15 mL). Note: The reaction mixture was washed with a 1.0 M aqueous solution of NaOH. After work-up, the crude product was product was purified by chromatography (24 g of silica gel, 0-10% MeOH/DCM) to afford compound 11a (0.231 g, 0.451 mmol, 73% yield) as a white solid. R_{f}=0.5 (10% MeOH/DCM); ¹H NMR (400 MHz, CDCl₃) δ 8.89 (d, J=4.3 Hz, 1H), 8.18 (d, J=8.4 Hz, 1H), 8.12 (d, J=8.4 Hz, 1H), 7.75 (ddd, J=8.4, 6.8, 1.2 Hz, 1H), 7.60 (ddd, J=8.3, 6.9, 1.2 Hz, 1H), 7.38 (d, J=4.3 Hz, 1H), 7.17-7.11 (m, 2H), 6.96 (dd, J=6.4, 3.1 Hz, 1H), 5.96 (d, J=8.2 Hz, 1H), 4.10-3.98 (m, 1H), 3.18-2.94 (m, 4H), 2.75-2.51 (m, 4H), 2.50-2.38 (m, 2H), 2.24-2.12 (m, 2H), 1.92-1.81 (m, 2H), 1.54-1.43 (m, 2H), 1.37-1.11 (m, 5H); ¹³C NMR (101 MHz, CDCl₃) δ 166.7, 151.4, 150.0, 148.8, 142.5, 134.2, 130.1, 130.0, 127.8, 127.64, 127.57, 125.3, 124.7, 124.6, 118.7, 118.4, 56.7, 53.6 (2C), 51.5 (2C), 49.6, 35.6, 34.0, 33.2 (2C), 32.0 (2C); IR (neat) 3274, 1633 cm⁻¹; mp 222-223 °C (dec); HRMS (ESI) m/z [M+H]⁺ calcd for C₂₈H₃₃Cl₂N₄O 511.2026, found 511.2024; t_{R}=36.0 min (HPLC, basic). The HCl salt was precipitated from 1 0.03 M solution of the free base in 50% CHCl₃/acetone using a 2.0 M solution of HCl in Et₂O (14 equiv). Dec>270 °C. Anal. calcd for C₂₈H₃₂Cl₂N₄O·2HCl·0.5H₂O: C, 56.67; H, 5.95; N, 9.44. Found: C, 56.68; H, 5.81; N, 9.34.

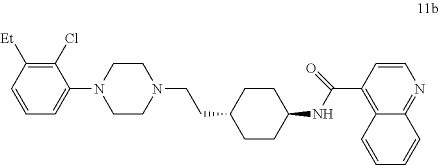 [SMILES: CCc1cccc(N2CCN(CC[C@H]3CC[C@H](NC(=O)c4ccnc5ccccc45)CC3)CC2)c1Cl]

### N-(trans-4-(2-(4-(2-Chloro-3-ethylphenyl)piperazin-1-yl)ethyl)cyclohexyl)quinoline-4-carboxamide (compound 11b)

General procedure E was followed using compound 5b (0.125 g, 0.358 mmol) and quinoline-4-carboxylic acid (68.0 mg, 0.393 mmol) in CHCl₃ (9.0 mL). After work-up, the crude product was purified by chromatography (silica gel, 0-8% MeOH/CHCl₃) to afford compound 11b (0.185 g, mmol, -100% yield) as a white solid, which was purified further by HCl salt formation. ¹H NMR (400 MHz, CDCl₃) δ 8.90 (d, J=4.3, 1H), 8.19 (d, J=8.5 Hz, 1H), 8.13 (d, J=8.5 Hz, 1H), 7.75 (t, J=7.7 Hz, 1H), 7.60 (t, J=7.7 Hz, 1H), 7.39 (d, J=4.3, 1H), 7.18-1.7.12 (m, 1H), 6.94 (t, J=7.6 Hz, 2H), 5.94 (d, J=8.3 Hz, 1H), 4.11-3.98 (m, 1H), 3.19-2.95 (m, 4H), 2.77 (q, J=7.3 Hz, 2H), 2.72-2.51 (m, 4H), 2.50-2.41 (m, 2H), 2.23-2.15 (m, 2H), 1.91-1.83 (m, 2H), 1.54-1.45 (m, 2H), 1.41-1.11 (m, 8H); ¹³C NMR (101 MHz, CDCl₃) δ 166.7, 150.0, 149.8, 148.8, 143.4, 142.5, 130.2, 130.1, 128.8, 127.8, 127.0, 125.3, 124.6, 124.1, 118.5, 118.1, 56.8, 53.7 (2C), 51.7 (2C), 49.6, 35.6, 34.0, 33.2 (2C), 32.0 (2C), 27.6, 14.3. The HCl salt was precipitated from 50% CHCl₃/acetone using a 2.0 M solution of HCl in Et₂O. Dec>250 °C. Anal. calcd for C₃₀H₃₇ClN₄O·2HCl·2H₂O: C, 58.68; H, 7.06; N, 9.12. Found: C, 58.74; H, 6.97; N, 8.94.

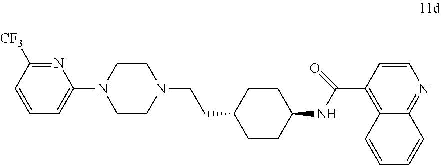 [SMILES: O=C(N[C@H]1CC[C@H](CCN2CCN(c3cccc(C(F)(F)F)n3)CC2)CC1)c1ccnc2ccccc12]

### N-(trans-4-(2-(4-(6-(trifluoromethyl)pyridin-2-yl)piperazin-1-yl)ethyl)cyclohexyl)quinoline-4-carboxamide (compound 11d)

General procedure E was followed using compound 5d (0.234 g, 0.656 mmol) and quinoline-4-carboxylic acid (0.125 g, 0.720 mmol) in CHCl₃ (16 mL). Note: The reaction mixture was washed with a 1.0 M aqueous solution of NaOH. After work-up, the crude product was product was purified by chromatography (24 g of silica gel, 0-10% MeOH/DCM) to afford compound 11d (0.234 g, 0.458 mmol, 70% yield) as a white solid. R_{f}=0.2 (5% MeOH/DCM); ¹H NMR (400 MHz, CDCl₃) δ 8.88 (d, J=4.3 Hz, 1H), 8.18 (d, J=8.5 Hz, 1H), 8.11 (d, J=8.4 Hz, 1H), 7.74 (ddd, J=8.4, 6.9, 1.4 Hz, 1H), 7.63-7.52 (m, 2H), 7.37 (d, J=4.3 Hz, 1H), 6.93 (d, J=7.3 Hz, 1H), 6.76 (d, J=8.7 Hz, 1H), 5.98 (d, J=8.2 Hz, 1H), 4.10-3.97 (m, 1H), 3.61-3.53 (m, 4H), 2.60-2.47 (m, 4H), 2.46-2.35 (m, 2H), 2.23-2.14 (m, 2H), 1.91-1.82 (m, 2H), 1.53-1.43 (m, 2H), 1.38-1.10 (m, 5H); ¹³C NMR (101 MHz, CDCl₃) δ 166.7, 159.0, 150.0, 148.8, 146.5 (q, J=34 Hz), 142.5, 138.4, 130.1, 130.0, 127.8, 125.3, 124.6, 121.8 (q, J=274 Hz), 118.4, 109.5, 109.0 (q, J=3 Hz), 56.7, 53.1 (2C), 49.6, 44.9 (2C), 35.5, 33.9, 33.2 (2C), 32.0 (2C); IR (neat) 3328, 1635 cm⁻¹; mp 187-188 °C (dec); HRMS (ESI) m/z [M+H]⁺ calcd for C₂₈H₃₃F₃N₅O 512.2632, found 512.2627; t_{R}=34.3 min (HPLC, basic). The HCl salt was precipitated from a 0.03 M solution of the free base in 50% CHCl₃/acetone using a 2.0 M solution of HCl in Et₂O (14 equiv). Mp 292-294 °C (dec). Anal. calcd for C₂₈H₃₂F₃N₅O·2HCl·0.25H₂O: C, 57.10; H, 5.90; N, 11.89. Found: C, 57.06; H, 5.93; N, 11.78.

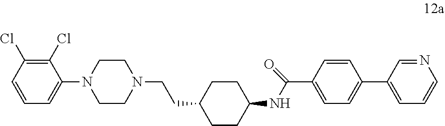 [SMILES: O=C(N[C@H]1CC[C@H](CCN2CCN(c3cccc(Cl)c3Cl)CC2)CC1)c1ccc(-c2cccnc2)cc1]

### N-trans-4-(2-(4-(2,3-Dichlorophenyl)piperazin-1-yl)ethyl)cyclohexyl)-4-(pyridin-3-yl)benzamide (compound 12a)

General procedure E was followed using compound 5a (0.101 g, 0.284 mmol) and 4-(pyridin-3-yl)benzoic acid-HCl (73.1 mg, 0.310 mmol) in CHCl₃ (8.0 mL). After work-up, the crude product was product was purified by chromatography (12 g of silica gel, 0-10% MeOH/DCM) to afford compound 12a (0.120 g, 0.224 mmol, 79% yield) as a white solid. R_{f}=0.5 (10% MeOH/DCM); ¹H NMR (400 MHz, CDCl₃) δ 8.88-8.84 (m, 1H), 8.63 (d, J=4.9 Hz, 1H), 7.92-7.83 (m, 3H), 7.67-7.61 (m, 2H), 7.39 (dd, J=7.9, 4.8 Hz, 1H), 7.18-7.10 (m, 1H), 6.96 (dd, J=6.3, 3.2 Hz, 1H), 5.98 (d, J=8.1 Hz, 1H), 4.02-3.90 (m, 1H), 3.16-2.97 (m, 4H), 2.74-2.53 (m, 4H), 2.49-2.40 (m, 2H), 4.02-3.90 (m, 2H), 2.18-2.09 (m, 2H), 1.89-1.80 (m, 2H), 1.54-1.42 (m, 2H), 1.37-1.00 (m, 5H); ¹³C NMR (101 MHz, CDCl₃) δ 166.3, 151.5, 149.3, 148.5, 140.9, 135.7, 134.7, 134.5, 134.5, 134.2, 127.8 (2C), 127.7, 127.6, 127.4 (2C), 123.8, 118.7, 56.7, 53.6 (2C), 51.5 (2C), 49.4, 35.7, 34.1 (2C), 33.3, 32.1 (2C); IR (neat) 3269, 1629 cm⁻¹; mp 257-258 °C (dec); HRMS (MALDI) m/z [M+H]⁺ calcd for C₃₀H₃₅Cl₂N₄O 537.2182, found 537.2185; t_{R}=36.9 min (HPLC, basic).

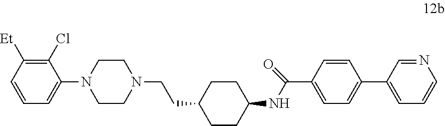 [SMILES: CCc1cccc(N2CCN(CC[C@H]3CC[C@H](NC(=O)c4ccc(-c5cccnc5)cc4)CC3)CC2)c1Cl]

### N-(trans-4-(2-(4-(2-Chloro-3-ethylphenyl)piperazin-1-yl)ethyl)cyclohexyl)-4-(75yridine-3-yl)benzamide (compound 12b)

General procedure E was followed using compound 5b (101 mg, 0.289 mmol) and 4-(pyridine-3-yl)benzoic acid·HCl (74.8 mg, 0.317 mmol) in CHCl₃ (8.0 mL). After work-up, the crude product was purified by chromatography (silica gel, 0-10% MeOH/CHCl₃) to afford compound 12b (0.150 g, 0.282 mmol, 98% yield) as a white solid. ¹H NMR (400 MHz, CDCl₃) δ 8.86 (s, 1H), 8.63 (s, 1H), 7.94-7.81 (m, 3H), 7.69-7.61 (m, 2H), 7.43-7.34 (m, 1H), 7.19-7.11 (m, 1H), 6.98-6.90 (m, 2H), 5.95 (d, J=8.0 Hz, 1H), 4.03-3.90 (m, 1H), 3.19-2.96 (m, 4H), 2.78 (q, J=7.6 Hz, 2H), 2.73-2.52 (m, 4H), 2.51-2.40 (m, 2H), 2.19-2.08 (m, 2H), 1.92-1.81 (m, 2H), 1.54-1.45 (m, 2H), 1.37-1.11 (m, 8H); ¹³C NMR (101 MHz, CDCl₃) δ 166.3, 149.8, 149.3, 148.5, 143.4, 140.9, 135.8, 134.7, 134.6, 128.9, 127.8, 127.4, 127.0, 124.1, 123.8, 118.2, 56.8, 53.8 (2C), 51.7 (2C), 49.4, 35.7, 34.1, 33.4 (2C), 32.1 (2C), 27.6, 14.3. The HCl salt was precipitated from a 0.02 M solution of the free base in 50% CHCl₃/acetone using a 2.0 M solution of HCl in Et₂O (44 equiv). Dec>223 °C. Anal. calcd for C₃₂H₃ClN₄O·2HCl·2.25H₂O: C, 59.63; H, 7.12; N, 8.69. Found: C, 59.24; H, 6.82; N, 8.54.

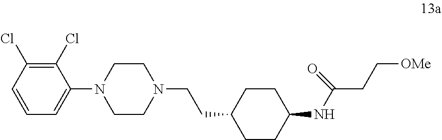 [SMILES: COCCC(=O)N[C@H]1CC[C@H](CCN2CCN(c3cccc(Cl)c3Cl)CC2)CC1]

### N-(trans-4-(2-(4-(2,3-dichlorophenyl)piperazin-1-yl)ethyl)cyclohexyl)-3-methoxypropanamide (compound 13a)

General procedure F was followed using compound 5a (0.85 g, 2.4 mmol). After work-up, the crude product was purified by chromatography (40 g of silica gel, 0-5% MeOH/DCM) to afford compound 13a (1.04 g, 2.35 mmol, 99% yield) as a white solid. R_{f}=0.5 (10% MeOH/DCM); ¹H NMR (400 MHz, CDCl₃) δ 7.17-7.11 (m, 2H), 6.95 (dd, J=6.4, 3.2 Hz, 1H), 5.95 (d, J=8.2 Hz, 1H), 3.77-3.66 (m, 1H), 3.62 (t, J=5.8 Hz, 2H), 3.36 (s, 3H), 3.17-2.98 (m, 4H), 2.73-2.52 (m, 4H), 2.47-2.37 (m, 4H), 2.04-1.92 (m, 2H), 1.82-1.72 (m, 2H), 1.47-1.38 (m, 2H), 1.31-1.18 (m, 1H), 1.17-1.01 (m, 4H); ¹³C NMR (101 MHz, CDCl₃) δ 170.7, 151.4, 134.2, 127.65, 127.57, 124.7, 118.7, 69.0, 58.9, 56.7, 53.6 (2C), 51.5 (2C), 48.6, 37.4, 35.6, 34.0, 33.2 (2C), 32.0 (2C); IR (neat) 3279, 1635 cm⁻¹; mp 180-181 °C (dec); HRMS (MALDI) m/z [M+H]⁺ calcd for C₂₂H₃₄Cl₂N₃O₂ 442.2023, found 442.2023; t_{R}=30.3 min (HPLC, basic). The HCl salt was precipitated from a 0.03 M solution of the free base in 50% CHCl₃/acetone using a 2.0 M solution of HCl in Et₂O (5.0 equiv). Dec>235 °C. Anal. calcd for C₂₂H₃₃Cl₂N₃O₂·HCl·0.75H₂O: C, 53.66; H, 7.27; N, 8.53. Found: C, 53.54; H, 6.95; N, 8.49.

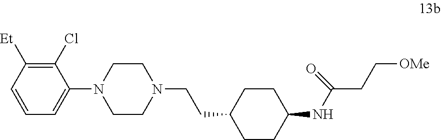 [SMILES: CCc1cccc(N2CCN(CC[C@H]3CC[C@H](NC(=O)CCOC)CC3)CC2)c1Cl]

### N-(trans-4-(2-(4-(2-Chloro-3-ethylphenyl)piperazin-1-yl)ethyl)cyclohexyl)-3-methoxypropanamide (compound 13b)

General procedure F was followed using compound 5b (0.197 g, 0.563 mmol). After work-up, the crude product was purified by chromatography (silica gel, 0-5% MeOH/CHCl₃) to afford compound 13b (0.245 g, 0.562 mmol, 100% yield) as an off-white solid. ¹H NMR (400 MHz, CDCl₃) δ 7.15 (t, J=7.8 Hz, 1H), 6.96-6.91 (m, 2H), 5.93 (d, J=8.2 Hz, 1H), 3.77-3.66 (m, 1H), 3.63 (t, J=5.8 Hz, 2H), 3.36 (s, 3H), 3.13-2.98 (m, 4H), 2.77 (q, J=7.5 Hz, 2H), 2.71-2.53 (m, 4H), 2.46-2.39 (m, 4H), 2.03-1.95 (m, 2H), 1.82-1.73 (m, 2H), 1.48-1.41 (m, 2H), 1.32-1.25 (m, 1H), 1.22 (t, J=7.5 Hz, 3H), 1.17-1.02 (m, 4H); ¹³C NMR (101 MHz, CDCl₃) δ 170.7, 149.8, 143.4, 128.9, 127.0, 124.1, 118.2, 69.0, 58.9, 56.8, 53.7 (2C), 51.8 (2C), 48.6, 37.4, 35.7, 34.1, 33.2 (2C), 32.0 (2C), 27.6, 14.3. The HCl salt was precipitated from a 0.04 M solution of the free base in 50% CHCl₃/acetone using a 2.0 M solution of HCl in Et₂O (22 equiv). Dec>240 °C. Anal. calcd for C₂₈H₃₈ClN₃O₂·HCl: C, 61.01; H, 8.32; N, 8.89. Found: C, 60.82; H, 8.21; N, 8.81.

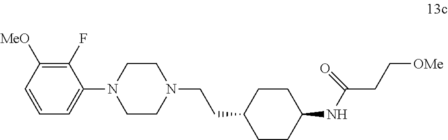 [SMILES: COCCC(=O)N[C@H]1CC[C@H](CCN2CCN(c3cccc(OC)c3F)CC2)CC1]

### N-(trans-4-(2-(4-(2-Fluoro-3-methoxyphenyl)piperazin-1-yl)ethyl)cyclohexyl)-3-methoxypropanamide (compound 13c)

General procedure F was followed using compound 5c (0.243 g, 0.724 mmol). After work-up, the crude product was purified by chromatography (24 g of silica gel, 0-5% MeOH/DCM) to afford compound 13c (0.159 g, 0.377 mmol, 52% yield) as a tan solid. R_{f}=0.2 (5% MeOH/DCM); ¹H NMR (400 MHz, CDCl₃) δ 6.96 (td, J=8.3, 1.8 Hz, 1H), 6.66-6.54 (m, 2H), 5.95 (d, J=8.2 Hz, 1H), 3.86 (s, 3H), 3.76-3.65 (m, 1H), 3.61 (t, J=5.8 Hz, 2H), 3.35 (s, 3H), 3.15-3.03 (m, 4H), 2.66-2.53 (m, 4H), 2.45-2.35 (m, 4H), 2.03-1.92 (m, 2H), 1.81-1.72 (m, 2H), 1.48-1.38 (m, 2H), 1.30-1.18 (m, 1H), 1.16-1.00 (m, 4H); ¹³C NMR (101 MHz, CDCl₃) δ 170.6, 148.7 (d, J=10 Hz), 145.8 (d, J=245 Hz), 141.2 (d, J=6 Hz), 123.6 (d, J=5 Hz), 111.2 (d, J=2 Hz), 107.2, 69.0, 58.9, 56.8, 56.6, 53.6 (2C), 50.8 (d, J=3 Hz, 2C), 48.5, 37.4, 35.6, 34.0, 33.2 (2C), 32.0 (2C); IR (neat) 3275, 1635 cm⁻¹; mp 156-157 °C (dec); HRMS (ESI) m/z [M+H]⁺ calcd for C₂₃H₃₇FN₃O₃ 422.2813, found 422.2807; t_{R}=21.6 min (HPLC, basic). The HCl salt was precipitated from a 0.03 M solution of the free base in 50% CHCl₃/acetone using a 2.0 M solution of HCl in Et₂O (5.0 equiv). Mp 211-213 °C (dec). Anal. calcd for C₂₃H₃₆FN₃O₃·2HCl·2H₂O: C, 52.07; H, 7.98; N, 7.92. Found: C, 51.96; H, 7.64; N, 7.78.

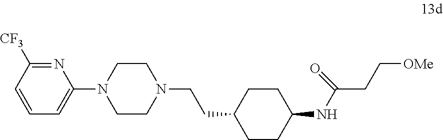 [SMILES: COCCC(=O)N[C@H]1CC[C@H](CCN2CCN(c3cccc(C(F)(F)F)n3)CC2)CC1]

### 3-Methoxy-N-(trans-4-(2-(4-(6-(trifluoromethyl)pyridin-2-yl)piperazin-1-yl)ethyl)cyclohexyl)propenamide (compound 13d)

General procedure F was followed using compound 5d (0.424 g, 1.19 mmol). After work-up, the crude product was purified by chromatography (40 g of silica gel, 0-5% MeOH/CHCl₃) to afford compound 13d (0.289 g, 0.654 mmol, 55% yield) as a white solid. R_{f}=0.2 (5% MeOH/DCM); ¹H NMR (400 MHz, CDCl₃) δ 7.56 (t, J=8.1 Hz, 1H), 6.93 (d, J=6.8 Hz, 1H), 6.76 (d, J=8.6 Hz, 1H), 5.95 (d, J=8.2 Hz, 1H), 3.77-3.66 (m, 1H), 3.65-3.54 (m, 6H), 3.36 (s, 3H), 2.58-2.47 (m, 4H), 2.44-2.33 (m, 4H), 2.04-1.92 (m, 2H), 1.83-1.73 (m, 2H), 1.48-1.39 (m, 2H), 1.32-1.21 (m, 1H), 1.17-1.01 (m, 4H); ¹³C NMR (101 MHz, CDCl₃) δ 170.6, 159.0, 146.6 (q, J=35 Hz), 138.4, 121.8 (q, J=274 Hz), 109.5, 108.9, 69.0, 58.9, 56.8, 53.1 (2C), 48.5, 44.9 (2C), 37.4, 35.6, 33.9, 33.2 (2C), 32.0 (2C); IR (film) 3277, 1634 cm⁻¹; mp 167-168 °C (dec); HRMS (ESI) m/z [M+H]⁺ calcd for C₂₂H₃₄F₃N₄O₂ 443.2628, found 443.2625; t_{R}=14.5 min (HPLC, acidic). The HCl salt was precipitated from a 0.04 M solution of the free base in 50% CHCl₃/acetone using a 2.0 M solution of HCl in Et₂O (3.0 equiv). Note: Et₂O was added to initiate precipitation. Dec>229 °C. Anal. calcd for C₂₂H₃₃F₃N₄O₂·HCl·0.5H₂O: C, 54.15; H, 7.23; N, 11.48. Found: C, 53.96; H, 6.99; N, 11.50.

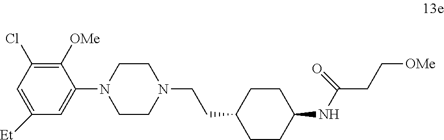 [SMILES: CCc1cc(Cl)c(OC)c(N2CCN(CC[C@H]3CC[C@H](NC(=O)CCOC)CC3)CC2)c1]

### N-(trans-4-(2-(4-(3-Chloro-5-ethyl-2-methoxyphenyl)piperazin-1-yl)ethyl)cyclohexyl)-3-methoxypropanamide (compound 13e)

General procedure F was followed using compound 5e (0.87 g, 2.3 mmol). After work-up, the crude product was purified by chromatography (40 g of silica gel, 0-5% MeOH/DCM) to afford compound 13e (0.89 g, 1.9 mmol, 83% yield) as a white solid. R_{f}=0.2 (5% MeOH/DCM); ¹H NMR (400 MHz, CDCl₃) δ 6.85 (d, J=2.0 Hz, 1H), 6.61 (d, J=2.0 Hz, 1H), 6.02 (d, J=8.1 Hz, 1H), 3.83 (s, 3H), 3.75-3.65 (m, 1H), 3.62 (t, J=5.8 Hz, 2H), 3.36 (s, 3H), 3.24-3.10 (m, 4H), 2.77-2.62 (m, 4H), 2.58-2.46 (m, 4H), 2.41 (t, J=5.8 Hz, 2H), 1.97 (d, J=9.7 Hz, 2H), 1.78 (d, J=10.2 Hz, 2H), 1.15-1.42 (m, 2H), 1.33-1.22 (m, 1H), 1.19 (t, J=7.6 Hz, 3H), 1.15-1.01 (m, 4H); ¹³C NMR (101 MHz, CDCl₃) δ 170.6, 146.4, 146.0, 140.8, 128.2, 122.3, 116.7, 68.8, 59.0, 58.7, 56.6, 53.8 (2C), 50.0 (2C), 48.4, 37.2, 35.4, 33.6, 33.0 (2C), 31.8 (2C), 28.4, 15.4; IR (neat) 3303, 1634 cm⁻¹; mp 118-120 °C; HRMS (MALDI) m/z [M+H]⁺ calcd for C₂₅H₄₁ClN₃O₃ 466.2831, found 466.2824; t_{R}=35.1 min (HPLC, basic). The HCl salt was precipitated from a 0.03 M solution of the free base in 50% CHCl₃/acetone using a 2.0 M solution of HCl in Et₂O (5.0 equiv). Dec>240 °C. Anal. calcd for C₂₅H₄₀ClN₃O₃·2HCl·0.25H₂O: C, 55.25; H, 7.88; N, 7.73. Found: C, 55.06; H, 7.69; N, 7.62.

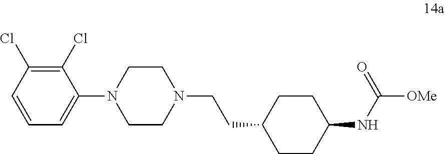 [SMILES: COC(=O)N[C@H]1CC[C@H](CCN2CCN(c3cccc(Cl)c3Cl)CC2)CC1]

### Methyl (trans-4-(2-(4-(2,3-dichlorophenyl)piperazin-1-yl)ethyl)cyclohexyl)carbamate (compound 14a)

General procedure D was followed using compound 5a (0.202 g, 0.566 mmol) and methyl chloroformate (56 μL, 0.72 mmol) in DCM (5.6 mL). After work-up, the crude product was purified by chromatography (12 g of silica gel, 0-5% MeOH/DCM) to afford compound 14a (0.204 g, 0.492 mmol, 87% yield) as a white solid. R_{f}=0.2 (5% MeOH/DCM); ¹H NMR (400 MHz, CDCl₃) δ 7.18-7.09 (m, 2H), 6.99-6.91 (m, 1H), 4.56-4.42 (m, 1H), 3.65 (s, 3H), 3.50-3.31 (m, 1H), 3.16-2.94 (m, 4H), 2.75-2.49 (m, 4H), 2.47-2.34 (m, 2H), 2.06-1.94 (m, 2H), 1.83-1.68 (m, 2H), 1.49-1.38 (m, 2H), 1.32-1.18 (m, 1H), 1.17-0.97 (m, 4H); ¹³C NMR (101 MHz, CDCl₃) δ 156.2, 151.3, 134.0, 127.5, 127.4, 124.5, 118.5, 56.5, 53.4 (2C), 51.8, 51.3 (2C), 50.3, 35.4, 33.8, 33.4 (2C), 31.9 (2C); IR (film) 3309, 1686 cm⁻¹; mp 152-153 °C (dec); HRMS (ESI) m/z [M+H]⁺ calcd for C₂₀H₃₀Cl₂N₃O₂ 414.1710, found 414.1705; t_{R}=34.0 min (HPLC, basic).

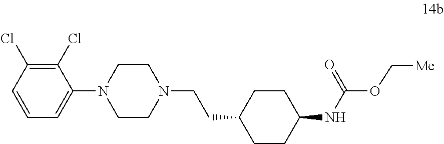 [SMILES: CCOC(=O)N[C@H]1CC[C@H](CCN2CCN(c3cccc(Cl)c3Cl)CC2)CC1]

### Ethyl (trans-4-(2-(4-(2,3-dichlorophenyl)piperazin-1-yl)ethyl)cyclohexyl)carbamate (compound 14b)

General procedure D was followed using compound 5a (0.151 g, 0.424 mmol) and ethyl chloroformate (52 μL, 0.55 mmol) in DCM (4.2 mL). After work-up, the crude product was purified by chromatography (12 g of silica gel, 0-5% MeOH/DCM) to afford compound 14b (0.167 g, 0.390 mmol, 92% yield) as a white solid. R_{f}=0.2 (5% MeOH/DCM); ¹H NMR (400 MHz, CDCl₃) δ 7.17-7.09 (m, 2H), 6.98-6.90 (m, 1H), 4.53-4.39 (m, 1H), 4.19-4.02 (m, 2H), 3.49-3.30 (m, 1H), 3.17-2.92 (m, 4H), 2.74-2.49 (m, 4H), 2.46-2.36 (m, 2H), 2.06-1.92 (m, 2H), 1.84-1.71 (m, 2H), 1.49-1.38 (m, 2H), 1.31-1.17 (m, 4H), 1.16-0.98 (m, 4H); ¹³C NMR (101 MHz, CDCl₃) δ 156.0, 151.5, 134.2, 127.7, 127.6, 124.7, 118.7, 60.7, 56.7, 53.6 (2C), 51.5 (2C), 50.4, 35.6, 34.0, 33.6 (2C), 32.1 (2C), 14.8; IR (film) 3303, 1678 cm⁻¹; mp 169-170 0C; HRMS (ESI) m/z [M+H]⁺ calcd for C₂₁H₃₂Cl₂N₃O₂ 428.1866, found 428.1857; t_{R}=35.2 min (HPLC, basic).

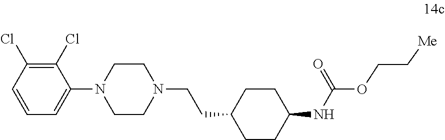 [SMILES: CCCOC(=O)N[C@H]1CC[C@H](CCN2CCN(c3cccc(Cl)c3Cl)CC2)CC1]

### Propyl (trans-4-(2-(4-(2,3-dichlorophenyl)piperazin-1-yl)ethyl)cyclohexyl)carbamate (compound 14c)

General procedure D was followed using compound 5a (0.201 g, 0.565 mmol) and propyl chloroformate (1.0 M solution in 1,4-dioxane, 0.73 mL, 0.73 mmol) in DCM (5.6 mL). After work-up, the crude product was purified by chromatography (12 g of silica gel, 0-5% MeOH/DCM) to afford compound 14c (0.234 g, 0.529 mmol, 94% yield) as a white solid. R_{f}=0.3 (5% MeOH/DCM); ¹H NMR (400 MHz, CDCl₃) δ7.20-7.07 (m, 2H), 6.95 (dd, J=6.3, 3.0 Hz, 1H), 4.55-4.40 (m, 1H), 4.08-3.91 (m, 2H), 3.49-3.32 (m, 1H), 3.17-2.96 (m, 4H), 2.74-2.51 (m, 4H), 2.46-2.34 (m, 2H), 2.08-1.94 (m, 2H), 1.84-1.71 (m, 2H), 1.69-1.55 (m, 2H), 1.49-1.37 (m, 2H), 1.31-1.18 (m, 1H), 1.17-0.98 (m, 4H), 0.93 (t, J=7.3 Hz, 3H); ¹³C NMR (101 MHz, CDCl₃) δ 156.1, 151.4, 134.1, 127.61, 127.57, 124.7, 118.7, 66.4, 56.7, 53.5 (2C), 51.5 (2C), 50.3, 35.6, 34.0, 33.5 (2C), 32.0 (2C), 22.5, 10.5; IR (film) 3294, 1674 cm⁻¹; mp 163-164 °C (dec); HRMS (ESI) m/z [M+H]⁺ calcd for C₂₂H₃₄Cl₂N₃O₂ 442.2023, found 442.2020; t_{R}=39.4 min (HPLC, basic).

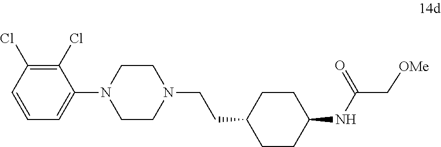 [SMILES: COCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)-2-methoxyacetamide (compound 14d)

General procedure F was followed using compound 5a (0.202 g, 0.566 mmol) and 2-methoxyacetic acid (52 μL, 0.68 mmol). After work-up, the crude product was purified by chromatography (12 g of silica gel, 0-5% MeOH/DCM) to afford compound 14d (62.3 mg, 0.145 mmol, 26% yield) as a white solid. R_{f}=0.2 (5% MeOH/DCM); ¹H NMR (400 MHz, CDCl₃) δ 7.18-7.10 (m, 2H), 7.00-6.92 (m, 1H), 6.32 (d, J=8.4 Hz, 1H), 3.86 (s, 2H), 3.82-3.70 (m, 1H), 3.41 (s, 3H), 3.16-2.95 (m, 4H), 2.74-2.50 (m, 4H), 2.47-2.40 (m, 2H), 2.03-1.94 (m, 2H), 1.85-1.75 (m, 2H), 1.50-1.40 (m, 2H), 1.34-1.23 (m, 1H), 1.22-1.03 (m, 4H); ¹³C NMR (101 MHz, CDCl₃) δ 168.5, 151.3, 134.0, 127.5, 127.4, 124.5, 118.5, 72.0, 59.0, 56.5, 53.4 (2C), 51.3 (2C), 47.9, 35.4, 33.9, 33.0 (2C), 31.8 (2C); IR (film) 3290, 1647 cm⁻¹; mp 156-157 °C; HRMS (ESI) m/z [M+H]⁺ calcd for C₂₁H₃₂Cl₂N₃O₂ 428.1866, found 428.1862; t_{R}=29.8 min (HPLC, basic).

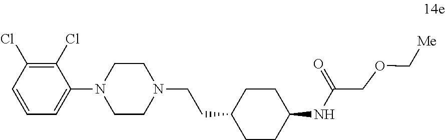 [SMILES: CCOCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)-2-ethoxyacetamide (compound 14e)

General procedure F was followed using compound 5a (0.152 g, 0.427 mmol) and 2-ethoxyacetic acid (48 μL, 0.51 mmol). After work-up, the crude product was purified by chromatography (12 g of silica gel, 0-10% MeOH/DCM) followed by recrystallization from MeCN (slow cooling of hot solvent) to afford compound 14e (87.9 mg, 0.199 mmol, 46% yield) as a white solid. R_{f}=0.4 (10% MeOH/DCM); ¹H NMR (400 MHz, CDCl₃) δ 7.18-7.08 (m, 2H), 7.00-6.90 (m, 1H), 6.37 (d, J=8.5 Hz, 1H), 3.89 (s, 2H), 3.82-3.70 (m, 1H), 3.55 (q, J=6.9 Hz, 2H), 3.16-2.95 (m, 4H), 2.75-2.51 (m, 4H), 2.47-2.39 (m, 2H), 2.02-1.93 (m, 2H), 1.84-1.75 (m, 2H), 1.49-1.40 (m, 2H), 1.33-1.02 (m, 5H), 1.23 (t, J=6.9 Hz, 3H); ¹³C NMR (101 MHz, CDCl₃) δ 169.1, 151.5, 134.2, 127.7, 127.6, 124.7, 118.7, 70.1, 67.2, 56.7, 53.6 (2C), 51.5 (2C), 48.1, 35.6, 34.1, 33.2 (2C), 32.0 (2C), 15.2; IR (film) 3298, 1647 cm⁻¹; mp 143-144 °C; HRMS (ESI) m/z [M+H]⁺ calcd for C₂₂H₃₄Cl₂N₃O₂ 442.2023, found 442.2013; t_{R}=36.3 min (HPLC, basic). The HCl salt was precipitated from a 0.03 M solution of the free base in 33% CHCl₃/acetone using a 2.0 M solution of HCl in Et₂O (8.1 equiv). Mp 247-248 °C (dec). Anal. calcd for C₂₂H₃₃Cl₂N₃O₂·HCl·0.5H₂O: C, 54.16; H, 7.23; N, 8.61. Found: C, 54.25; H, 7.08; N, 8.50.

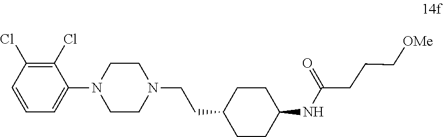 [SMILES: COCCCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)-4-methoxybutanamide (compound 14f)

General procedure F was followed using compound 5a (0.200 g, 0.562 mmol) and 4-methoxybutanoic acid (75 μL, 0.67 mmol). After work-up, the crude product was purified by chromatography (12 g of silica gel, 0-5% MeOH/DCM) to afford compound 14f (0.160 g, 0.351 mmol, 62% yield) as a white solid. R_{f}=0.1 (5% MeOH/DCM); ¹H NMR (400 MHz, CDCl₃) δ 7.18-7.10 (m, 2H), 6.99-6.92 (m, 1H), 5.49 (d, J=5.5 Hz, 1H), 3.78-3.64 (m, 1H), 3.45-3.37 (m, 2H), 3.32 (s, 3H), 3.16-2.94 (m, 4H), 2.74-2.50 (m, 4H), 2.47-2.38 (m, 2H), 2.27-2.19 (m, 2H), 2.03-1.94 (m, 2H), 1.93-1.84 (m, 2H), 1.83-1.68 (m, 2H), 1.48-1.39 (m, 2H), 1.32-1.17 (m, 1H), 1.16-1.01 (m, 4H); ¹³C NMR (101 MHz, CDCl₃) δ 171.9, 151.5, 134.2, 127.7, 127.6, 124.7, 118.7, 71.9, 58.7, 56.7, 53.6 (2C), 51.5 (2C), 48.6, 35.7, 34.1, 33.8, 33.3 (2C), 32.0 (2C), 25.8; IR (film) 3276, 1639 cm⁻¹; mp 186-187 °C (dec); HRMS (ESI) m/z [M+H]⁺ calcd for C₂₃H₃₆Cl₂N₃O₂ 456.2179, found 456.2176; t_{R}=29.9 min (HPLC, basic).

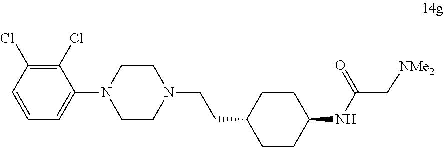 [SMILES: CN(C)CC(=O)N[C@H]1CC[C@H](CCN2CCN(c3cccc(Cl)c3Cl)CC2)CC1]

### N-(trans-4-(2-(4-(2,3-dichlorophenyl)piperazin-1-yl)ethyl)cyclohexyl)-2-(dimethylamino)acetamide (compound 14g)

General procedure F was followed using compound 5a (0.202 g, 0.566 mmol) and dimethylglycine (71.1 mg, 0.689 mmol). After work-up, the crude product was purified by chromatography (24 g of silica gel, 0-10% MeOH containing 10% NH₄OH/DCM) to afford compound 14g (0.171 g, 0.386 mmol, 68% yield) as a white solid. R_{f}=0.4 (10% MeOH containing 10% NH₄OH/DCM); ¹H NMR (400 MHz, CDCl₃) δ 7.17-7.10 (m, 2H), 6.99-6.91 (m, 2H), 3.80-3.67 (m, 1H), 3.18-2.96 (m, 4H), 2.91 (s, 2H), 2.72-2.52 (m, 4H), 2.46-2.38 (m, 2H), 2.27 (s, 6H), 2.00-1.92 (m, 2H), 1.83-1.74 (m, 2H), 1.49-1.40 (m, 2H), 1.32-1.23 (m, 1H), 1.22-1.02 (m, 4H); ¹³C NMR (101 MHz, CDCl₃) δ 169.7, 151.5, 134.2, 127.7, 127.6, 124.7, 118.7, 63.5, 56.7, 53.6 (2C), 51.5 (2C), 48.0, 46.1 (2C), 35.6, 34.1, 33.2 (2C), 32.1 (2C); IR (film) 3272, 1651 cm⁻¹; mp 150-151 °C (dec); HRMS (ESI) m/z [M+H]⁺ calcd for C₂₂H₃₅Cl₂N₄O 441.2182, found 441.2184; t_{R}=32.8 min (HPLC, basic).

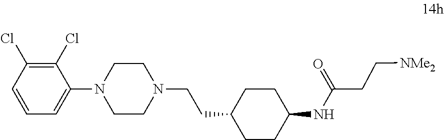 [SMILES: CN(C)CCC(=O)N[C@H]1CC[C@H](CCN2CCN(c3cccc(Cl)c3Cl)CC2)CC1]

### N-(trans-4-(2-(4-(2,3-dichlorophenyl)piperazin-1-yl)ethyl)cyclohexyl)-3-(dimethylamino)propenamide (compound 14h)

General procedure F was followed using compound 5a (0.202 g, 0.568 mmol) and 3-(dimethylamino)propanoic acid (80.3 mg, 0.685 mmol). After work-up, the crude product was purified by chromatography (24 g of silica gel, 0-10% MeOH containing 10% NH₄OH/DCM) to afford compound 14h (85.5 mg, 0.188 mmol, 33% yield) as a white solid. R_{f}=0.2 (10% MeOH containing 10% NH₄OH/DCM); ¹H NMR (400 MHz, CDCl₃) δ 8.05 (d, J=8.1 Hz, 1H), 7.17-7.10 (m, 2H), 6.95 (dd, J=6.4, 3.3 Hz, 1H), 3.73-3.62 (m, 1H), 3.16-2.95 (m, 4H), 2.73-2.54 (m, 4H), 2.53-2.47 (m, 2H), 2.45-2.38 (m, 2H), 2.38-2.28 (m, 2H), 2.24 (s, 6H), 2.00-1.92 (m, 2H), 1.80-1.71 (m, 2H), 1.48-1.39 (m, 2H), 1.32-1.19 (m, 1H), 1.17-1.01 (m, 4H); ¹³C NMR (101 MHz, CDCl₃) δ 171.8, 151.5, 134.2, 127.7, 127.6, 124.7, 118.7, 56.7, 55.7, 53.6 (2C), 51.5 (2C), 48.0, 44.7 (2C), 35.6, 34.1, 33.4, 33.2 (2C), 32.0 (2C); IR (film) 3293, 1633 cm⁻¹; mp 169-170 °C (dec); HRMS (ESI) m/z [M+H]⁺ calcd for C₂₃H₃₇Cl₂N₄O 455.2339, found 455.2333; t_{R}=30.8 min (HPLC, basic).

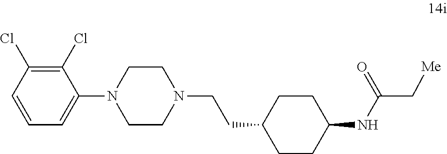 [SMILES: CCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)propionamide (compound 14i)

To a solution of compound 5a (0.151 g, 0.424 mmol) and TEA (0.18 mL, 1.3 mmol) in DCM (4.2 mL) was added propionic anhydride (81 μL, 0.63 mmol). After stirring at rt overnight, the reaction was quenched with a saturated aq solution of NaHCO₃ (10 mL), and the aq layer was extracted with DCM (3×10 mL). The combined organic layers were dried over anhydrous Na₂SO₄, filtered, and concentrated. The crude product was purified by chromatography (12 g of silica gel, 0-10% MeOH/DCM) to afford compound 14i (0.116 g, 0.282 mmol, 67% yield) as a white solid. R_{f}=0.4 (10% MeOH/DCM); ¹H NMR (400 MHz, CDCl₃) δ 7.17-7.10 (m, 2H), 6.99-6.91 (m, 1H), 5.23 (d, J=8.1 Hz, 1H), 3.79-3.62 (m, 1H), 3.18-2.90 (m, 4H), 2.78-2.48 (m, 4H), 2.46-2.37 (m, 2H), 2.16 (q, J=7.6 Hz, 2H), 2.03-1.91 (m, 2H), 1.84-1.71 (m, 2H), 1.49-1.38 (m, 2H), 1.30-1.18 (m, 1H), 1.17-1.00 (m, 7H); ¹³C NMR (101 MHz, CDCl₃) δ 173.8, 152.3, 135.0, 128.5, 128.4, 125.5, 119.5, 57.5, 54.4 (2C), 52.3 (2C), 49.4, 36.5, 34.9, 34.1 (2C), 32.9 (2C), 30.9, 10.9; IR (film) 3287, 1634 cm⁻¹; mp 204-205 °C (dec); HRMS (ESI) m/z [M+H]⁺ calcd for C₂₁H₃₂Cl₂N₃O 412.1917, found 412.1912; t_{R}=28.7 min (HPLC, basic).

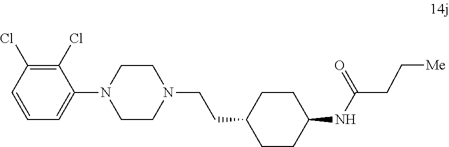 [SMILES: CCCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)butyramide (compound 14j)

General procedure F was followed using compound 5a (0.282 g, 0.791 mmol) and butyric acid (87 μL, 0.95 mmol). After work-up, the crude product was purified by chromatography (24 g of silica gel, 0-10% MeOH/DCM) followed by recrystallization from MeCN (slow cooling of hot solvent) to afford compound 14j (0.220 g, 0.517 mmol, 65% yield) as a white solid. R_{f}=0.5 (10% MeOH/DCM); ¹H NMR (400 MHz, CDCl₃) δ 7.17-7.09 (m, 2H), 6.99-6.91 (m, 1H), 5.24 (d, J=8.2 Hz, 1H), 3.79-3.64 (m, 1H), 3.20-2.98 (m, 4H), 2.76-2.47 (m, 4H), 2.46-2.36 (m, 2H), 2.10 (t, J=7.6 Hz, 2H), 2.02-1.91 (m, 2H), 1.84-1.72 (m, 2H), 1.70-1.58 (m, 2H), 1.48-1.38 (m, 2H), 1.30-1.17 (m, 1H), 1.16-1.01 (m, 4H), 0.93 (t, J=7.5 Hz, 3H); ¹³C NMR (101 MHz, CDCl₃) δ 172.2, 151.5, 134.2, 127.7, 127.6, 124.6, 118.7, 56.7, 53.6 (2C), 51.5 (2C), 48.6, 39.1, 35.7, 34.1, 33.4 (2C), 32.1 (2C), 19.4, 13.8; IR (film) 3282, 1637 cm⁻¹; mp 193-194 °C; HRMS (ESI) m/z [M+H]⁺ calcd for C₂₂H₃₄Cl₂N₃O 426.2073, found 426.2068; t_{R}=26.0 min (HPLC, basic).

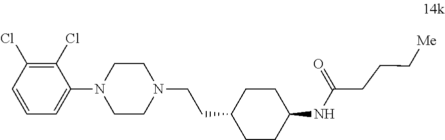 [SMILES: CCCCC(=O)N[C@H]1CC[C@H](CCN2CCN(c3cccc(Cl)c3Cl)CC2)CC1]

### N-(trans-4-(2-(4-(2,3-Dichlorophenyl)piperazin-1-yl)ethyl)cyclohexyl)pentanamide (compound 14k)

General procedure F was followed using compound 5a (0.251 g, 0.704 mmol) and pentanoic acid (92 μL, 0.84 mmol). After work-up, the crude product was purified by chromatography (24 g of silica gel, 0-10% MeOH/DCM) followed by recrystallization from MeCN (slow cooling of hot solvent) to afford compound 14k (0.227 g, 0.516 mmol, 73% yield) as a white solid. R_{f}=0.4 (10% MeOH/DCM); ¹H NMR (400 MHz, CDCl₃) δ 7.18-7.10 (m, 2H), 6.99-6.92 (m, 1H), 5.22 (d, J=8.2 Hz, 1H), 3.78-3.65 (m, 1H), 3.18-2.93 (m, 4H), 2.77-2.48 (m, 4H), 2.46-2.36 (m, 2H), 2.13 (t, J=7.6 Hz, 2H), 2.03-1.92 (m, 2H), 1.84-1.71 (m, 2H), 1.65-1.54 (m, 2H), 1.49-1.40 (m, 2H), 1.39-1.28 (m, 2H), 1.27-1.19 (m, 1H), 1.15-1.01 (m, 4H), 0.91 (t, J=7.4 Hz, 3H); ¹³C NMR (101 MHz, CDCl₃) δ 172.3, 151.5, 134.2, 127.7, 127.6, 124.7, 118.7, 56.7, 53.6 (2C), 51.5 (2C), 48.6, 37.0, 35.7, 34.1, 33.4 (2C), 32.1 (2C), 28.1, 22.5, 14.0; IR (film) 3280, 1637 cm⁻¹; mp 189-190 °C (dec); HRMS (ESI) m/z [M+H]⁺ calcd for C₂₃H₃₆Cl₂N₃O 440.2230, found 440.2226; t_{R}=37.5 min (HPLC, basic).

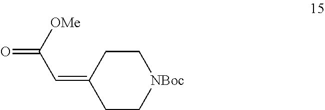 [SMILES: COC(=O)C=C1CCN(C(=O)OC(C)(C)C)CC1]

### tert-Butyl 4-(2-methoxy-2-oxoethylidene)piperidine-1-carboxylate (compound 15)

Using oven-dried glassware under an argon atmosphere, a mixture of 90 wt % NaH (0.521 g, 19.5 mmol) in anhydrous THF (76 mL) was cooled to 0 °C, and methyl 2-(dimethoxyphosphoryl)acetate (3.2 mL, 20 mmol) was added over 10 min. After stirring the mixture vigorously for 30 min, a solution of tert-butyl 4-oxopiperidine-1-carboxylate (3.01 g, 15.1 mmol) in anhydrous THF (38 mL) was added over 10 min. The reaction was allowed to warm to room temperature and stirred for an additional 18 h. Then, H₂O (50 mL) was added, and the aqueous layer was extracted with EtOAc (2×50 mL). The combined organic layers were washed with brine (50 mL), dried over Na₂SO₄, filtered, and concentrated. The crude product was purified by chromatography (80 g of silica gel, 0-20% EtOAc/hexanes) to afford compound 15 (3.69 g, 14.5 mmol, 96% yield) as a white solid. R_{f}=0.4 (20% EtOAc/hexanes); ¹H NMR (400 MHz, CDCl₃) δ 5.71 (s, 1H), 3.69 (s, 3H), 3.54-3.43 (m, 4H), 2.97-2.89 (m, 2H), 2.31-2.23 (m, 2H), 1.47 (s, 9H); ¹³C NMR (101 MHz, CDCl₃) δ 166.9, 158.4, 154.7, 115.0, 80.0, 51.2, 44.7 (2C), 36.6, 29.7, 28.6 (3C); IR (neat) 1711, 1680, 1655 cm⁻¹; mp 64-65 °C; HRMS (MALDI) m/z [M+Na]⁺ calcd for C₁₃H₂₁NNaO₄ 278.1363, found 278.1367.

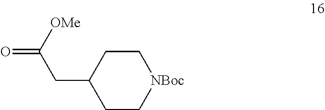 [SMILES: COC(=O)CC1CCN(C(=O)OC(C)(C)C)CC1]

### tert-Butyl 4-(2-methoxy-2-oxoethyl)piperidine-1-carboxylate (compound 16)

Using a Parr shaker, a mixture of compound 15 (2.00 g, 7.84 mmol), 10 wt % Pd/C (0.839 g, 0.787 mmol), and MeOH (60 mL) was agitated under H₂ (50 psi) for 5 h. Afterward, the mixture was passed through a pad of Celite (ca. 30 g) using EtOAc (3×50 mL), and the filtrate was concentrated. The crude product was purified by chromatography (40 g of silica gel, 0-20% EtOAc/hexanes) to afford compound 16 (1.81 g, 7.03 mmol, 90% yield) as a clear, colorless oil. R_{f}=0.3 (20% EtOAc/hexanes); ¹H NMR (400 MHz, CDCl₃) δ 4.21-3.96 (m, 2H), 3.67 (s, 3H), 2.71 (t, J=12.9 Hz, 2H), 2.24 (d, J=7.1 Hz, 2H), 1.98-1.86 (m, 1H), 1.68 (d, J=13.1 Hz, 2H), 1.44 (s, 9H), 1.22-1.07 (m, 2H); ¹³C NMR (101 MHz, CDCl₃) δ 173.0, 154.9, 79.5, 51.6, 43.8 (2C), 41.0, 33.2, 32.0 (2C), 28.6 (3C); IR (neat) 1737, 1688 cm⁻¹; HRMS (MALDI) m/z [M+Na]⁺ calcd for C₁₃H₂₃NNaO₄ 280.1519, found 280.1525.

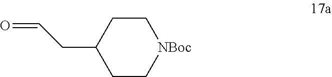 [SMILES: CC(C)(C)OC(=O)N1CCC(CC=O)CC1]

### tert-Butyl 4-(2-oxoethyl)piperidine-1-carboxylate (compound 17a)

Using oven-dried glassware under an argon atmosphere, a solution of compound 16 (1.10 g, 4.27 mmol) in anhydrous DCM (33 mL) was cooled to −78 °C, and a 1.0 M solution of DIBALH in toluene (5.4 mL, 5.4 mmol) was added over 5 min. The reaction was stirred for 3 h and allowed to warm to rt. Then, a saturated aqueous solution of Rochelle salt (60 mL) was added, and the aqueous layer was extracted with DCM (3×30 mL). The combined organic layers were dried over Na₂SO₄, filtered, and concentrated. The crude product was purified by chromatography (80 g of silica gel, 0-40% EtOAc/hexanes) to afford compound 17a (0.492 g, 2.17 mmol, 51% yield) as a clear, colorless oil. R_{f}=0.5 (40% EtOAc/hexanes); ¹H NMR (400 MHz, CDCl₃) δ 9.78 (s, 1H), 4.21-3.96 (m, 2H), 2.73 (t, J=12.8 Hz, 2H), 2.38 (d, J=6.7 Hz, 2H), 2.11-1.98 (m, 1H), 1.68 (d, J=13.2 Hz, 2H), 1.45 (s, 9H), 1.24-1.09 (m, 2H); ¹³C NMR (101 MHz, CDCl₃) δ 201.6, 154.9, 79.6, 50.3, 43.9 (2C), 32.1 (2C), 30.8, 28.6 (3C); IR (neat) 1722, 1686 cm⁻¹; HRMS (MALDI) m/z [M+Na]⁺ calcd for C₁₂H₂₁NNaO₃ 250.1414, found 250.1416.

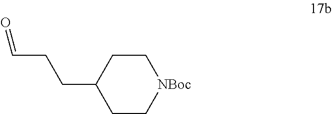 [SMILES: CC(C)(C)OC(=O)N1CCC(CCC=O)CC1]

### tert-Butyl 4-(3-oxopropyl)piperidine-1-carboxylate (compound 17b)

General procedure A was followed using tert-butyl 4-(3-hydroxypropyl)piperidine-1-carboxylate (1.02 g, 4.18 mmol). After work-up, the crude product was purified by chromatography (40 g of silica gel, 0-30% EtOAc/hexanes) to afford compound 17b (0.769 g, 3.19 mmol, 76% yield) as a clear, colorless oil. R_{f}=0.4 (30% EtOAc/hexanes); ¹H NMR (400 MHz, CDCl₃) δ 9.77 (t, J=1.3 Hz, 1H), 4.22-3.94 (m, 2H), 2.65 (t, J=13.1 Hz, 2H), 2.46 (td, J=7.5, 1.7 Hz, 2H), 1.68-1.54 (m, 4H), 1.44 (s, 9H), 1.42-1.33 (m, 1H), 1.15-1.02 (m, 2H); ¹³C NMR (101 MHz, CDCl₃) δ 202.4, 154.9, 79.4, 44.0 (2C), 41.3, 35.6, 32.0 (2C), 28.58 (3C), 28.55; IR (neat) 1724, 1686 cm⁻¹; HRMS (MALDI) m/z [M+Na]⁺ calcd for C₁₃H₂₃NNaO₃ 264.1570, found 264.1575.

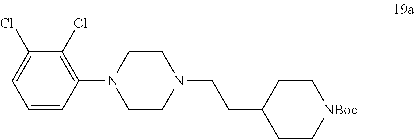 [SMILES: CC(C)(C)OC(=O)N1CCC(CCN2CCN(c3cccc(Cl)c3Cl)CC2)CC1]

### tert-Butyl 4-(2-(4-(2,3-dichlorophenyl)piperazin-1-yl)ethyl)piperidine-1-carboxylate (compound 19a)

General procedure B was followed using compound 17a (0.486 g, 2.14 mmol) and 1-(2,3-dichlorophenyl)piperazine·HCl (0.688 g, 3.22 mmol) in DCE (14 mL). After work-up, the crude product was purified by chromatography (40 g of silica gel, 0-80% EtOAc/hexanes) to afford compound 19a (0.61 g, 1.4 mmol, 64% yield) as a white solid. R_{f}=0.3 (80% EtOAc/hexanes); ¹H NMR (400 MHz, CDCl₃) δ 7.18-7.10 (m, 2H), 6.95 (dd, J=6.5, 3.1 Hz, 1H), 4.18-3.94 (m, 2H), 3.15-2.96 (m, 4H), 2.78-2.52 (m, 6H), 2.49-2.40 (m, 2H), 1.67 (d, J=13.0 Hz, 2H), 1.52-1.39 (m, 3H), 1.45 (s, 9H), 1.20-1.05 (m, 2H); ¹³C NMR (101 MHz, CDCl₃) δ 155.0, 151.4, 134.2, 127.7, 127.6, 124.7, 118.7, 79.4, 56.3, 53.6 (2C), 51.5 (2C), 44.2 (2C), 34.7, 33.7, 32.4 (2C), 28.6 (3C); IR (neat) 1682 cm⁻¹; mp 116-117 °C; HRMS (MALDI) m/z [M+H]⁺ calcd for C₂₂H₃₄Cl₂N₃O₂ 442.2023, found 442.2021.

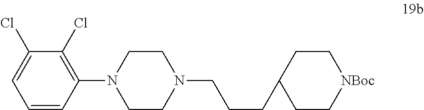 [SMILES: CC(C)(C)OC(=O)N1CCC(CCCN2CCN(c3cccc(Cl)c3Cl)CC2)CC1]

### tert-Butyl 4-(3-(4-(2,3-dichlorophenyl)piperazin-1-yl)propyl)piperidine-1-carboxylate (compound 19b)

General procedure B was followed using compound 17b (0.764 g, 3.17 mmol) and 1-(2,3-dichlorophenyl)piperazine·HCl (0.933 g, 3.49 mmol) in DCE (31 mL). After work-up, the crude product was purified by chromatography (40 g of silica gel, 0-100% EtOAc/hexanes) to afford compound 19b (0.884 g, 1.94 mmol, 61% yield) as a clear, orange oil. R_{f}=0.2 (EtOAc); ¹H NMR (400 MHz, CDCl₃) δ 7.19-7.07 (m, 2H), 6.95 (dd, J=6.4, 3.1 Hz, 1H), 4.19-3.95 (m, 2H), 3.17-2.96 (m, 4H), 2.78-2.48 (m, 6H), 2.43-2.35 (m, 2H), 1.71-1.62 (m, 2H), 1.61-1.49 (m, 2H), 1.45 (s, 9H), 1.42-1.33 (m, 1H), 1.30-1.22 (m, 2H), 1.15-1.01 (m, 2H); ¹³C NMR (101 MHz, CDCl₃) δ 155.0, 151.4, 134.2, 127.7, 127.6, 124.7, 118.7, 79.3, 59.0, 53.5 (2C), 51.5 (2C), 44.2 (2C), 36.2, 34.5, 32.4 (2C), 28.6 (3C), 24.2; IR (film) 1692 cm⁻¹; HRMS (ESI) m/z [M+H]⁺ calcd for C₂₃H₃Cl₂N₃O₂ 456.2179, found 456.2175.

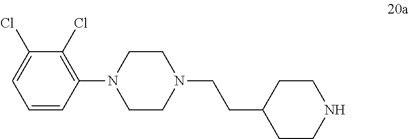 [SMILES: Clc1cccc(N2CCN(CCC3CCNCC3)CC2)c1Cl]

### 1-(2,3-Dichlorophenyl)-4-(2-(piperidin-4-yl)ethyl)piperazine (compound 20a)

General procedure C was followed using compound 19a (0.250 g, 0.566 mmol) and TFA (7.0 mL, 91 mmol) in DCM (37 mL). After work-up, compound 20a (0.191 g, 0.558 mmol, 99% yield) was isolated as a tan solid. R_{f}=0.1 (10% MeOH/DCM); ¹H NMR (400 MHz, CDCl₃) δ 7.16-7.10 (m, 2H), 6.95 (dd, J=6.4, 3.2 Hz, 1H), 3.14-2.94 (m, 6H), 2.73-2.49 (m, 6H), 2.47-2.38 (m, 2H), 1.73-1.55 (m, 3H), 1.51-1.34 (m, 3H), 1.00-1.06 (m, 2H); ¹³C NMR (101 MHz, CDCl₃) δ 151.5, 134.1, 127.64, 127.56, 124.6, 118.7, 56.3, 53.6 (2C), 51.5 (2C), 47.0 (2C), 35.0, 34.5, 34.0 (2C); IR (neat) 1577 cm⁻¹; mp 78-80 °C; HRMS (MALDI) m/z [M+H]⁺ calcd for C₁₇H₂₆Cl₂N₃ 342.1498, found 342.1500.

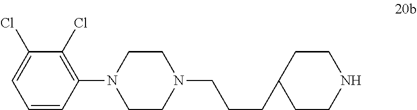 [SMILES: Clc1cccc(N2CCN(CCCC3CCNCC3)CC2)c1Cl]

### 1-(2,3-Dichlorophenyl)-4-(3-(piperidin-4-yl)propyl)piperazine (compound 20b)

General procedure C was followed using compound 19b (0.244 g, 0.535 mmol) and TFA (1.6 mL, 21 mmol) in DCM (3.7 mL). After work-up, compound 20b (0.188 g, 0.527 mmol, 99% yield) was isolated as a clear, yellow oil. R_{f}=0.1 (10% MeOH/DCM); ¹H NMR (400 MHz, CDCl₃) δ 7.16-7.08 (m, 2H), 6.98-6.90 (m, 1H), 2.17-2.92 (m, 6H), 2.74-2.49 (m, 6H), 2.42-2.31 (m, 2H), 1.95-1.77 (m, 1H), 1.72-1.60 (m, 2H), 1.59-1.43 (m, 2H), 1.42-1.16 (m, 3H), 1.15-0.98 (m, 2H); ¹³C NMR (101 MHz, CDCl₃) δ 151.5, 134.2, 127.7, 127.6, 124.7, 118.7, 59.0, 53.5 (2C), 51.5 (2C), 46.0 (2C), 35.8, 34.7, 32.2 (2C), 24.1; IR (neat) 1577 cm⁻¹; HRMS (ESI) m/z [M+H]⁺ calcd for Cl₈H₂₈Cl₂N₃ 356.1655, found 356.1652.

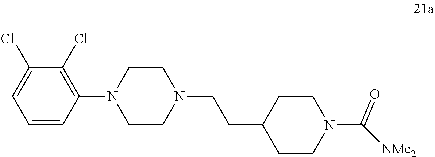 [SMILES: CN(C)C(=O)N1CCC(CCN2CCN(c3cccc(Cl)c3Cl)CC2)CC1]

### 4-(2-(4-(2,3-Dichlorophenyl)piperazin-1-yl)ethyl)-N,N-dimethylpiperidine-1-carboxamide (compound 21a)

General procedure D was followed using compound 20a (0.184 g, 0.538 mmol) and N,N-dimethylcarbamoyl chloride (65 μL, 0.71 mmol) in DCM (5.4 mL). After work-up, the crude product was purified by chromatography (12 g of silica gel, 0-10% MeOH/DCM) to afford compound 21a (0.159 g, 0.385 mmol, 71% yield) as a tan solid. R_{f}=0.5 (10% MeOH/DCM); ¹H NMR (400 MHz, CDCl₃) δ 7.17-7.10 (m, 2H), 6.95 (dd, J=6.4, 3.1 Hz, 1H), 3.68-3.60 (m, 2H), 3.14-2.94 (m, 4H), 2.80 (s, 6H), 2.72 (td, J=12.7, 2.3 Hz, 2H), 2.67-2.52 (m, 4H), 2.48-2.40 (m, 2H), 1.73-1.65 (m, 2H), 1.53-1.40 (m, 3H), 1.28-1.13 (m, 2H); ¹³C NMR (101 MHz, CDCl₃) δ 165.3, 151.4, 134.1, 127.64, 127.57, 124.7, 118.7, 56.3, 53.6 (2C), 51.5 (2C), 47.3 (2C), 38.7 (2C), 34.9, 33.8, 32.4 (2C); IR (neat) 1640 cm⁻¹; mp 96-97 0C; HRMS (MALDI) m/z [M+H]⁺ calcd for C₂₀H₃₁Cl₂N₄O 413.1869, found 413.1870; t_{R}=32.6 min (HPLC, basic).

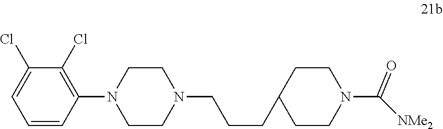 [SMILES: CN(C)C(=O)N1CCC(CCCN2CCN(c3cccc(Cl)c3Cl)CC2)CC1]

### 4-(3-(4-(2,3-Dichlorophenyl)piperazin-1-yl)propyl)-N,N-dimethylpiperidine-1-carboxamide (compound 21b)

General procedure D was followed using compound 20b (0.188 g, 0.527 mmol) and N,N-dimethylcarbamoyl chloride (65 μL, 0.71 mmol) in DCM (5.5 mL). After work-up, the crude product was purified by chromatography (12 g of silica gel, 0-10% MeOH/DCM) to afford compound 21b (0.176 g, 0.410 mmol, 78% yield) as a tan, amorphous solid. R_{f}=0.5 (10% MeOH/DCM); ¹H NMR (400 MHz, CDCl₃) δ 7.17-7.10 (m, 2H), 6.95 (dd, J=6.3, 3.3 Hz, 1H), 3.68-3.59 (m, 2H), 3.16-2.96 (m, 4H), 2.80 (s, 6H), 2.75-2.51 (m, 6H), 2.43-2.34 (m, 2H), 1.73-1.69 (m, 2H), 1.60-1.48 (m, 2H), 1.45-1.34 (m, 1H), 1.31-1.22 (m, 2H), 1.21-1.08 (m, 2H); ¹³C NMR (101 MHz, CDCl₃) δ 165.3, 151.4, 134.1, 127.63, 127.56, 124.7, 118.7, 59.0, 53.5 (2C), 51.5 (2C), 47.3 (2C), 38.7 (2C), 36.4, 34.6, 32.3 (2C), 24.2; IR (film) 1643 cm⁻¹; mp 71-73 °C; HRMS (ESI) m/z [M+H]⁺ calcd for C₂₁H₃₃Cl₂N₄O 427.2026, found 427.2021; t_{R}=35.5 min (HPLC, basic).

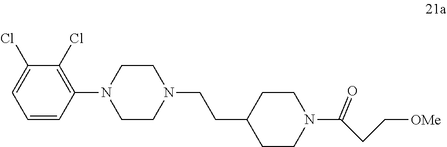 [SMILES: COCCC(=O)N1CCC(CCN2CCN(c3cccc(Cl)c3Cl)CC2)CC1]

### 1-(4-(2-(4-(2,3-Dichlorophenyl)piperazin-1-yl)ethyl)piperidin-1-yl)-3-methoxypropan-1-one (compound 22a)

General procedure F was followed using compound 20a (0.192 g, 0.560 mmol). After work-up, the crude product was purified by chromatography (12 g of silica gel, 0-10% MeOH/DCM) to afford compound 22a (0.121 g, 0.283 mmol, 50% yield) as a tan solid. R_{f}=0.5 (10% MeOH/DCM); ¹H NMR (400 MHz, CDCl₃) δ 7.19-7.11 (m, 2H), 6.96 (dd, J=7.0, 2.6 Hz, 1H), 4.61 (d, J=13.3 Hz, 1H), 3.87 (d, J=13.5 Hz, 1H), 3.70 (t, J=6.7 Hz, 2H), 3.36 (s, 3H), 3.18-2.95 (m, 5H), 2.82-2.64 (m, 4H), 2.60 (t, J=6.7 Hz, 2H), 2.57-2.44 (m, 3H), 1.74 (t, J=12.1 Hz, 2H), 1.64-1.45 (m, 3H), 1.23-1.07 (m, 2H); ¹³C NMR (101 MHz, CDCl₃) δ 169.2, 151.3, 134.2, 127.7, 127.6, 124.8, 118.7, 69.0, 59.0, 56.2, 53.5 (2C), 51.3 (2C), 46.1, 42.0, 34.7, 33.7, 33.4, 33.0, 32.1; IR (neat) 1644 cm⁻¹; mp 88-90 °C; HRMS (MALDI) m/z [M+H]⁺ calcd for C₂₁H₃₂Cl₂N₃O₂ 428.1866, found 428.1860; t_{R}=27.4 min (HPLC, basic).

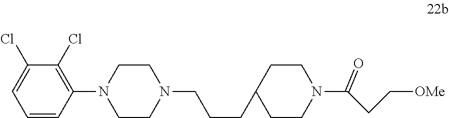 [SMILES: COCCC(=O)N1CCC(CCCN2CCN(c3cccc(Cl)c3Cl)CC2)CC1]

### 1-(4-(3-(4-(2,3-Dichlorophenyl)piperazin-1-yl)propyl)piperidin-1-yl)-3-methoxypropan-1-one (compound 22b)

General procedure F was followed using compound 20b (0.183 g, 0.513 mmol). After work-up, the crude product was purified by chromatography (24 g of silica gel, 0-5% MeOH/DCM) to afford compound 22b (0.175 g, 0.396 mmol, 77% yield) as a clear, orange oil. R_{f}=0.2 (5% MeOH/DCM); ¹H NMR (400 MHz, CDCl₃) δ 7.21-7.11 (m, 2H), 7.01-6.94 (m, 1H), 4.69-4.55 (m, 1H), 3.95-3.83 (m, 1H), 3.76-3.64 (m, 2H), 3.36 (s, 3H), 3.21-3.04 (m, 4H), 3.03-2.93 (m, 1H), 2.85-2.66 (m, 4H), 2.65-2.42 (m, 5H), 1.82-1.68 (m, 2H), 1.66-1.42 (m, 3H), 1.36-1.21 (m, 2H), 1.18-1.01 (m, 2H); ¹³C NMR (101 MHz, CDCl₃) δ 169.2, 151.1, 134.2, 127.7, 127.6, 125.0, 118.8, 69.0, 59.0, 58.9, 53.5 (2C), 51.1 (2C), 46.1, 42.1, 36.2, 34.2, 33.7, 32.9, 32.0, 23.8; IR (neat) 1634 cm⁻¹; HRMS (ESI) m/z [M+H]⁺ calcd for C₂₂H₃₄Cl₂N₃O₂ 442.2023, found 442.2019; t_{R}=17.8 min (HPLC, acidic). The HCl salt was prepared from a 0.03 M solution of the free base in 50% CHCl₃/acetone using a 2.0 M solution of HCl in Et₂O (15 equiv). The solution was concentrated, and the resulting residue was sonicated with hexanes to obtain a yellow solid, which was recrystallized from CHCl₃-hexanes (slow evaporation of solvent mixture). Mp 193-194 °C (dec). Anal. calcd for C₂₂H₃₃Cl₂N₃O₂·HCl·0.25H₂O: C, 54.66; H, 7.19; N, 8.69. Found: C, 54.37; H, 7.04; N, 8.52.

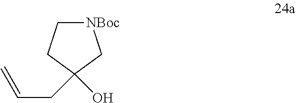 [SMILES: C=CCC1(O)CCN(C(=O)OC(C)(C)C)C1]

### tert-Butyl 3-allyl-3-hydroxypyrrolidine-1-carboxylate (compound 24a)

Using oven-dried glassware under an argon atmosphere, a solution of tert-butyl 3-oxopyrrolidine-1-carboxylate (1.52 g, 8.21 mmol) in anhydrous Et₂O (50 mL) was cooled to 0 °C. Then, a 1.0 M solution of allyl magnesium bromide in Et₂O (9.8 mL, 9.8 mmol) was added over 5 min, and the mixture was allowed to warm to rt. After stirring for 4 h, the reaction was quenched with a saturated aqueous solution of NH₄Cl (10 mL), and the aqueous layer was extracted with Et₂O (3×10 mL). The combined organic layers were washed with brine, dried over Na₂SO₄, filtered, and concentrated. The crude product was purified by chromatography (40 g of silica gel, 0-30% EtOA/hexanes) to afford compound 24a (1.11 g, 4.88 mmol, 60% yield) as a clear, yellow oil. R_{f}=0.2 (30% EtOAc/hexanes); ¹H NMR (400 MHz, CDCl₃, mixture of rotamers) δ 5.94-5.79 (m, 1H), 5.25-5.15 (m, 2H), 3.59-3.42 (m, 2H), 3.41-3.20 (m, 2H), 2.39 (d, J=7.5 Hz, 2H), 1.90-1.80 (m, 3H), 1.46 (s, 9H); ¹³C NMR (101 MHz, CDCl₃, mixture of rotamers) δ 154.8, 133.0, 119.9, 79.5, 79.0, 78.2, 57.7, 57.5, 44.9, 44.4, 43.5, 37.8, 37.3, 28.7 (3C); IR (film) 3412, 1697, 1670 cm⁻¹; mp 110-112 0C; HRMS (ESI) m/z [M+Na]⁺ calcd for C₁₂H₂₁NO₃Na 250.1414, found 250.1414.

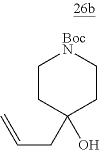 [SMILES: C=CCC1(O)CCN(C(=O)OC(C)(C)C)CC1]

### tert-Butyl 4-allyl-4-hydroxypiperidine-1-carboxylate (compound 24b)

The same procedure as the one described for compound 24a was followed starting from tert-butyl 4-oxopiperidine-1-carboxylate (2.00 g, 10.0 mmol). After work-up, the crude product was purified by chromatography (silica gel, 0-40% EtOA/hexanes) to afford compound 24b (1.44 g, 6.00 mmol, 60% yield) as a clear, colorless oil. ¹H NMR (400 MHz, CDCl₃) δ 5.93-5.79 (m, 1H), 5.25-5.10 (m, 2H), 4.00-3.60 (m, 2H), 3.28-3.04 (m, 2H), 2.23 (d, J=7.6 Hz, 2H), 1.74-1.34 (m, 5H), 1.45 (s, 9H).

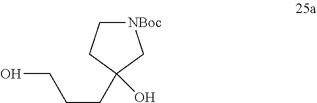 [SMILES: CC(C)(C)OC(=O)N1CCC(O)(CCCO)C1]

### tert-Butyl 3-hydroxy-3-(3-hydroxypropyl)pyrrolidine-1-carboxylate (compound 25a)

A 1.0 M solution of BH₃·THF in THF (15 mL, 15 mmol) was added over 5 min to a solution of compound 24a (1.75 g, 7.72 mmol) in THF (15 mL) at 0 °C. The reaction mixture was stirred for 3 h, and a 2.0 M aqueous solution of NaOH (24 mL, 47 mmol) was added followed by 30 wt % H₂O₂ in H₂O (24 mL, 0.23 mol). The resulting mixture was stirred for an additional 3 h and allowed to warm to rt. The mixture was diluted with H₂O (30 mL) and extracted with EtOAc (3×150 mL). The combined organic extracts were dried over anhydrous Na₂SO₄, filtered, and concentrated. The crude product was purified by chromatography (120 g of silica gel, 0-100% hexanes/EtOAc) to afford compound 25a (1.34 g, 5.48 mmol, 71% yield) as a clear, colorless oil. R_{f}=0.3 (EtOAc); ¹H NMR (400 MHz, CDCl₃, mixture of rotamers) b 3.79-3.61 (m, 2H), 3.55-3.32 (m, 3H), 3.26-3.16 (m, 1H), 1.97-1.89 (m, 1H), 1.86-1.66 (m, 5H), 1.45 (s, 9H); ¹³C NMR (101 MHz, CDCl₃, mixture of rotamers) δ 155.1, 154.9, 79.5, 79.4, 78.3, 63.1, 58.3, 57.9, 45.1, 44.5, 38.4, 37.7, 36.9, 36.6, 28.7 (3C), 28.1, 27.8; IR (film) 3374, 1668 cm⁻¹; HRMS (ESI) m/z [M+Na]⁺ calcd for C₁₂H₂₃NO₄Na 268.1519, found 268.1517.

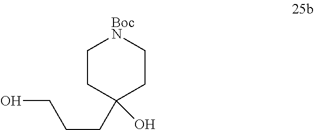 [SMILES: CC(C)(C)OC(=O)N1CCC(O)(CCCO)CC1]

### tert-Butyl 4-hydroxy-4-(3-hydroxypropyl)piperidine-1-carboxylate (compound 25b)

The same procedure as the one described for compound 25a was followed starting from compound 24b (2.60 g, 10.8 mmol). After work-up, the crude product was purified by chromatography (silica gel, 0-100% hexanes/EtOAc) to afford compound 25b (1.14 g, 4.40 mmol, 41% yield) of a clear, colorless oil. ¹H NMR (400 MHz, CDCl₃) 3.80 (d, J=12.9 Hz, 2H), 3.70 (t, J=6.1 Hz, 2H), 3.16 (t, J=12.3 Hz, 2H), 2.06-1.80 (m, 2H), 1.73-1.65 (m, 2H), 1.63-1.48 (m, 6H), 1.45 (s, 9H).

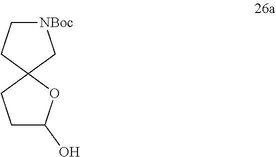 [SMILES: CC(C)(C)OC(=O)N1CCC2(CCC(O)O2)C1]

### tert-Butyl 2-hydroxy-1-oxa-7-azaspiro[4.4]nonane-7-carboxylate (compound 26a)

General procedure A was followed using compound 25a (1.34 g, 5.46 mmol). After work-up, the crude product was purified by chromatography (80 g of silica gel, 0-50% EtOAc/hexanes) to afford compound 26a (0.958 g, 3.94 mmol, 72% yield) as a white solid. R_{f}=0.3 (50% EtOAc/hexanes); ¹H NMR (400 MHz, CDCl₃, mixture of rotamers and diastereomers) 6 5.53 (s, 1H), 3.67-2.99 (m, 5H), 2.27-1.67 (m, 6H), 1.44 (s, 9H); ¹³C NMR (101 MHz, CDCl₃, mixture of rotamers and diastereomers) δ 154.7, 98.9, 89.2, 89.1, 88.4, 88.3, 79.4, 57.9, 57.5, 57.0, 56.4, 45.3, 44.8, 38.6, 38.1, 37.8, 37.1, 33.7, 33.6, 32.1, 32.0, 31.9, 31.4, 28.7 (3C); IR (film) 3406, 1695, 1675 cm⁻¹; HRMS (ESI) m/z [M+Na]⁺ calcd for C₁₂H₂₁NO₄Na 266.1363, found 266.1362.

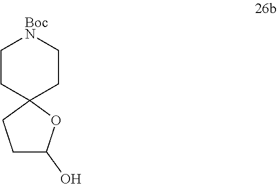 [SMILES: CC(C)(C)OC(=O)N1CCC2(CCC(O)O2)CC1]

### tert-Butyl 2-hydroxy-1-oxa-8-azaspiro[4.5]decane-8-carboxylate (compound 26b)

General procedure A was followed using compound 25b (1.14 g, 4.40 mmol). After work-up, the crude product was purified by chromatography (silica gel, 0-40% EtOAc/hexanes) to afford compound 26b (0.450 g, 1.75 mmol, 40% yield) as a white solid. ¹H NMR (400 MHz, CDCl₃) δ 5.55-5.48 (m, 1H), 3.69-3.48 (m, 2H), 3.44-3.26 (m, 2H), 2.56-2.50 (m, 1H), 2.01-1.85 (m, 2H), 1.85-1.64 (m, 3H), 1.62-1.39 (m, 3H), 1.45 (s, 9H).

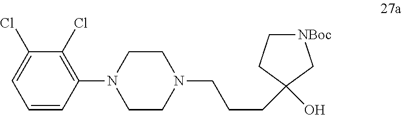 [SMILES: CC(C)(C)OC(=O)N1CCC(O)(CCCN2CCN(c3cccc(Cl)c3Cl)CC2)C1]

### tert-Butyl 3-(3-(4-(2,3-dichlorophenyl)piperazin-1-yl)propyl)-3-hydroxypyrrolidine-1-carboxylate (compound 27a)

General procedure B was followed using compound 26a (0.958 g, 3.94 mmol) and 1-(2,3-dichlorophenyl)piperazine·HCl (1.16 g, 4.33 mmol) in DCE (38 mL). After work-up, the crude product was purified by chromatography (silica gel, 0-10% MeOH/DCM) to afford compound 27a (0.754 g, 1.64 mmol, 42% yield) as a yellow solid. ¹H NMR (400 MHz, CDCl₃, mixture of rotamers) b 7.19-7.10 (m, 2H), 6.98-6.93 (m, 1H), 3.60-3.38 (m, 2H), 3.35-3.18 (m, 2H), 3.17-2.97 (m, 4H), 2.90-2.58 (m, 4H), 2.56-2.47 (m, 2H), 1.92-1.69 (m, 6H), 1.48-1.41 (m, 9H).

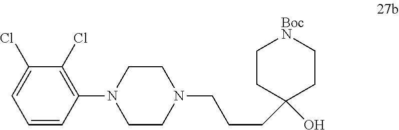 [SMILES: CC(C)(C)OC(=O)N1CCC(O)(CCCN2CCN(c3cccc(Cl)c3Cl)CC2)CC1]

### tert-Butyl 4-(3-(4-(2,3-Dichlorophenyl)piperazin-1-yl)propyl)-4-hydroxypiperidine-1-carboxylate (compound 27b)

General procedure B was followed using compound 26b (0.445 g, 1.73 mmol) and 1-(2,3-dichlorophenyl)piperazine·HCl (0.509 g, 1.90 mmol) in DCE (18 mL). After work-up, the crude product was purified by chromatography (silica gel, 0-10% MeOH/DCM) to afford compound 27b (0.810 g, 1.71 mmol, 99% yield) as a clear, yellow oil. ¹H NMR (400 MHz, CDCl₃) δ 7.19-7.12 (m, 2H), 6.96 (dd, J=7.1, 2.5 Hz, 1H), 3.89-3.64 (m, 2H), 3.24 (t, J=11.8 Hz, 2H), 3.16-2.96 (m, 4H), 2.83-2.57 (m, 4H), 2.53-2.46 (m, 2H), 1.74-1.63 (m, 4H), 1.56-1.38 (m, 5H), 1.45 (s, 9H).

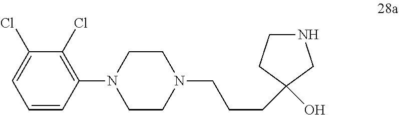 [SMILES: OC1(CCCN2CCN(c3cccc(Cl)c3Cl)CC2)CCNC1]

### 3-(3-(4-(2,3-Dichlorophenyl)piperazin-1-yl)propyl)pyrrolidin-3-ol (compound 28a)

General procedure C was followed using compound 27a (0.754 g, 1.64 mmol) and TFA (2.5 mL, 33 mmol) in DCM (12 mL). Afterwork-up, compound 28a (0.422 g, 1.18 mmol, 72% yield) was isolated and used immediately.

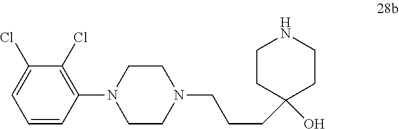 [SMILES: OC1(CCCN2CCN(c3cccc(Cl)c3Cl)CC2)CCNCC1]

### 4-(3-(4-(2,3-Dichlorophenyl)piperazin-1-yl)propyl)piperidin-4-ol (compound 28b)

General procedure C was followed using compound 27b (0.814 g, 1.72 mmol) and TFA (2.6 mL, 34 mmol) in DCM (12 mL). Afterwork-up, compound 28b (0.500 g, 1.34 mmol, 78% yield) was isolated and used immediately.

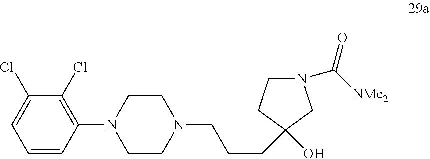 [SMILES: CN(C)C(=O)N1CCC(O)(CCCN2CCN(c3cccc(Cl)c3Cl)CC2)C1]

### 3-(3-(4-(2,3-Dichlorophenyl)piperazin-1-yl)propyl)-3-hydroxy-N,N-dimethylpyrrolidine-1-carboxamide (compound 29a)

General procedure D was followed using compound 28a (0.150 g, 0.419 mmol) and N,N-dimethylcarbamoyl chloride (48 μL, 0.52 mmol) in DCM (6.0 mL). After work-up, the crude product was purified by chromatography (silica gel, 0-5% MeOH/DCM) to afford compound 29a (68.0 mg, 0.158 mmol, 38% yield) as a clear, yellow oil. ¹H NMR (400 MHz, CDCl₃) δ 7.16-7.08 (m, 2H), 6.97-6.91 (m, 1H), 3.70-3.60 (m, 1H), 3.45-3.35 (m, 2H), 3.24-3.18 (m, 1H), 3.15-2.95 (m, 4H), 2.82-2.58 (m, 4H), 2.81 (s, 6H), 2.53-2.46 (m, 2H), 1.87-1.67 (m, 6H); HRMS (MALDI) m/z [M+H]⁺ calcd for C₂₀H₃₁Cl₂N₄O₂ 429.1819, found 429.1802. The HCl salt was precipitated from a 0.06 M solution of the free base in 50% acetone/CHCl₃ using a 2.0 M solution of HCl in Et₂O (4.0 equiv). Mp 184-186 °C. Anal. calcd for C₂₀H₃₀Cl₂N₄O₂·1.25HCl: C, 50.58; H, 6.63; N, 11.80. Found: C, 50.64; H, 6.39; N, 11.60.

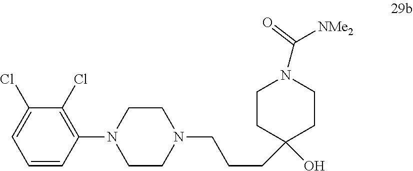 [SMILES: CN(C)C(=O)N1CCC(O)(CCCN2CCN(c3cccc(Cl)c3Cl)CC2)CC1]

### 4-(3-(4-(2,3-Dichlorophenyl)piperazin-1-yl)propyl)-4-hydroxy-N,N-dimethylpiperidine-1-carboxamide (compound 29b)

General procedure D was followed using compound 28b (0.804 g, 2.16 mmol) and N,N-dimethylcarbamoyl chloride (0.25 mL, 2.7 mmol) in DCM (42 mL). After work-up, the crude product was purified by chromatography (silica gel, 0-5% MeOH/DCM) to afford compound 29b (0.272 g, 0.613 mmol, 28% yield) as a clear, yellow oil. ¹H NMR (400 MHz, CDCl₃) δ 7.18-7.11 (m, 2H), 6.99-6.92 (m, 1H), 3.45-3.37 (m, 2H), 3.31-3.19 (m, 2H), 3.18-2.96 (m, 4H), 2.87-2.57 (m, 4H), 2.80 (s, 6H), 2.53-2.44 (m, 2H), 1.74-1.63 (m, 4H), 1.58-1.48 (m, 4H); HRMS (MALDI) m/z [M+H]⁺ calcd for C₂₁H₃₃Cl₂N₄O₂ 443.1975, found 443.1971. The HCl salt was precipitated from a 0.06 M solution of the free base in 50% acetone/CHCl₃ using a 2.0 M solution of HCl in Et₂O (2.5 equiv). Mp 189-192 °C. Anal. calcd for C₂₁H₃₂Cl₂N₄O₂·HCl·0.25H₂O: C, 52.07; H, 6.97; N, 11.57. Found: C, 51.98; H, 7.01; N, 11.22.

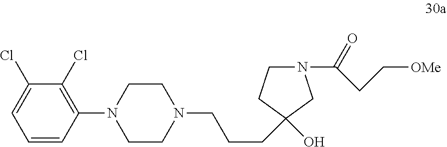 [SMILES: COCCC(=O)N1CCC(O)(CCCN2CCN(c3cccc(Cl)c3Cl)CC2)C1]

### 1-(3-(3-(4-(2,3-Dichlorophenyl)piperazin-1-yl)propyl)-3-hydroxypyrrolidin-1-yl)-3-methoxypropan-1-one (compound 30a)

General procedure F was followed using compound 28a (0.422 g, 1.18 mmol). After work-up, the crude product was purified by chromatography (silica gel, 0-5% MeOH/DCM) to afford compound 30a (0.137 g, 0.309 mmol, 26% yield) as a clear, yellow oil. ¹H NMR (400 MHz, CD₃OD) δ 7.31-7.24 (m, 2H), 7.16-7.10 (m, 1H), 3.76-3.60 (m, 4H), 3.59-3.45 (m, 2H), 3.35-3.32 (m, 3H), 3.28-3.15 (m, 4H), 3.14-2.96 (m, 4H), 2.94-2.82 (m, 2H), 2.68-2.51 (m, 2H), 2.04-1.70 (m, 6H); HRMS (MALDI) m/z [M+H]⁺ calcd for C₂₁H₃₂Cl₂N₃O₃ 444.1815, found 444.1814; t_{R}=16.9 min (HPLC, acidic).

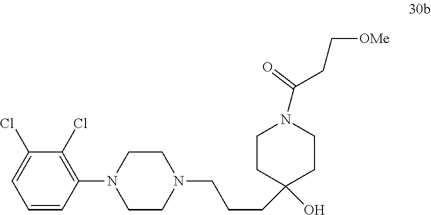 [SMILES: COCCC(=O)N1CCC(O)(CCCN2CCN(c3cccc(Cl)c3Cl)CC2)CC1]

### 1-(4-(3-(4-(2,3-Dichlorophenyl)piperazin-1-yl)propyl)-4-hydroxypiperidin-1-yl)-3-methoxypropan-1-one (compound 30b)

General procedure F was followed using compound 28b (0.500 g, 1.34 mmol). After work-up, the crude product was purified by chromatography (silica gel, 0-5% MeOH/CHCl₃) to afford compound 30b (0.313 g, 0.309 mmol, 50% yield) as a yellow solid. ¹H NMR (400 MHz, CDCl₃) δ 7.22-7.11 (m, 2H), 7.00-6.94 (m, 1H), 4.38-4.29 (m, 1H), 3.74-3.67 (m, 2H), 3.66-3.58 (m, 1H), 3.43 (t, J=12.6 Hz, 1H), 3.35 (s, 3H), 3.27-3.03 (m, 5H), 2.95-2.69 (m, 4H), 2.61 (t, J=6.9 Hz, 4H), 1.84-1.53 (m, 6H), 1.50-1.36 (m, 2H); HRMS (MALDI) m/z [M+H]+calcd for C₂₂H₃₄Cl₂N₃O₃ 458.1972, found 458.1962. The HCl salt was precipitated from a 0.08 M solution of the free base in 50% acetone/CHCl₃ using a 2.0 M solution of HCl in Et₂O (10 equiv). Dec>160 °C. Anal. calcd for C₂₂H₃₃Cl₂N₃O₃·HCl·H₂O: C, 51.52; H, 7.08; N, 8.19. Found: C, 51.62; H, 6.81; N, 8.14.

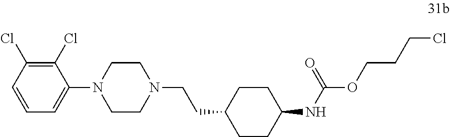 [SMILES: O=C(N[C@H]1CC[C@H](CCN2CCN(c3cccc(Cl)c3Cl)CC2)CC1)OCCCCl]

### 3-Chloropropyl (trans-4-(2-(4-(2,3-dichlorophenyl)piperazin-1-yl)ethyl)cyclohexyl)carbamate (compound 31 b)

General procedure D was followed using compound 5a (0.201 g, 0.564 mmol) and 2-chloroprpoyl chloroformate (88 μL, 0.73 mmol) in DCM (5.6 mL). After work-up, the crude product was purified by chromatography (24 g of silica gel, 0-5% MeOH/DCM) to afford compound 31b (0.252 g, 0.528 mmol, 94% yield) as a white solid. R_{f}=0.3 (5% MeOH/DCM); ¹H NMR (400 MHz, CDCl₃) δ 7.19-7.10 (m, 2H), 6.99-6.90 (m, 1H), 4.57-4.42 (m, 1H), 4.28-4.10 (m, 2H), 3.60 (t, J=6.6 Hz, 2H), 3.49-3.32 (m, 1H), 3.19-2.91 (m, 4H), 2.75-2.51 (m, 4H), 2.46-2.37 (m, 2H), 2.16-1.92 (m, 4H), 1.06-1.68 (m, 2H), 1.48-1.37 (m, 2H), 1.32-1.11 (m, 1H), 1.18-0.97 (m, 4H); ¹³C NMR (101 MHz, CDCl₃) b 155.7, 151.5, 134.2, 127.7, 127.6, 124.7, 118.7, 61.5, 56.7, 53.6 (2C), 51.5 (2C), 50.5, 41.5, 35.6, 34.0, 33.5 (2C), 32.3, 32.0 (2C); IR (film) 3318, 1682 cm⁻¹; mp 151-152 °C (dec); HRMS (ESI) m/z [M+H]⁺ calcd for C₂₂H₃₃Cl₃N₃O₂ 476.1633, found 476.1629.

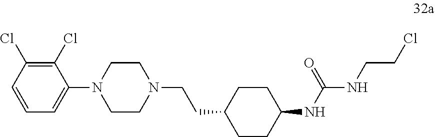 [SMILES: O=C(NCCCl)N[C@H]1CC[C@H](CCN2CCN(c3cccc(Cl)c3Cl)CC2)CC1]

### 1-(2-Chloroethyl)-3-(trans-4-(2-(4-(2,3-dichlorophenyl)piperazin-1-yl)ethyl)cyclohexyl)urea (compound 32a)

A solution of compound 5a (0.350 g, 0.981 mmol) in anhydrous DCM (9.8 mL) was cooled to 0 °C. Then, 1-chloro-2-isocyanatoethane (92 μL, 1.1 mmol) was added over 5 min. The reaction mixture was allowed to warm to rt, stirred for 2 h, and concentrated. The crude product was purified by recrystallization from EtOAc (slow cooling of hot solvent) to afford compound 32a (0.326 g, 0.707 mmol, 72% yield) as a white solid. R_{f}=0.4 (10% MeOH/DCM); ¹H NMR (400 MHz, CD₃CO₂D) δ 7.28-7.18 (m, 2H), 7.11-7.04 (m, 1H), 3.89-3.72 (m, 2H), 3.62-3.54 (m, 2H), 3.51-3.36 (m, 5H), 3.30-3.08 (m, 6H), 1.99-1.90 (m, 2H), 1.80-1.62 (m, 4H), 1.38-1.24 (m, 1H), 1.22-0.99 (m, 4H); ¹³C NMR (101 MHz, CD₃CO₂D) δ 160.1, 150.7, 134.9, 129.1, 128.3, 126.7, 120.3, 56.3, 53.1 (2C), 50.4, 49.3 (2C), 45.0, 43.0, 35.6, 33.8 (2C), 32.2 (2C), 31.2; IR (neat) 3378, 3294, 1632 cm⁻¹; mp 176-177 °C (dec); HRMS (ESI) m/z [M+H]⁺ calcd for C₂₁H₃₂Cl₃N₄O 461.1636, found 461.1627.

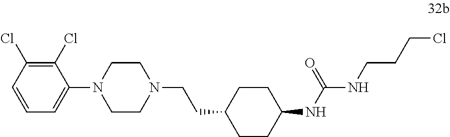 [SMILES: O=C(NCCCCl)N[C@H]1CC[C@H](CCN2CCN(c3cccc(Cl)c3Cl)CC2)CC1]

### 1-(3-Chloropropyl)-3-(trans-4-(2-(4-(2,3-dichlorophenyl)piperazin-1-yl)ethyl)cyclohexyl)urea (compound 32b)

The same procedure as the one described for compound 32a was followed using compound 5a (0.671 g, 1.88 mmol) and 1-chloro-3-isocyanatopropane (0.21 mL, 2.0 mmol) in anhydrous DCM (19 mL). After concentrating the reaction mixture, the crude product was purified by recrystallization from EtOAc (slow cooling of hot solvent) to afford compound 32b (0.409 g, 0.859 mmol, 46% yield) as a white solid. R_{f}=0.5 (10% MeOH/DCM); ¹H NMR (400 MHz, CD₃CO₂D) δ 7.27-7.18 (m, 2H), 7.11-7.01 (m, 1H), 3.86-3.68 (m, 2H), 3.59-3.52 (m, 2H), 3.49-3.35 (m, 3H), 3.31-3.06 (m, 8H), 1.98-1.86 (m, 4H), 1.79-1.61 (m, 4H), 1.37-1.24 (m, 1H), 1.22-0.97 (m, 4H); ¹³C NMR (101 MHz, CD₃CO₂D) δ 160.5, 150.6, 134.9, 129.1, 128.3, 126.7, 120.3, 56.3, 53.1 (2C), 50.4, 49.3 (2C), 43.2, 38.5, 35.6, 33.9 (2C), 33.6, 32.3 (2C), 31.2; IR (neat) 3379, 3296, 1630 cm⁻¹; mp 180-181 °C (dec); HRMS (ESI) m/z [M+H]⁺ calcd for C₂₂H₃₄Cl₃N₄O 475.1793, found 475.1790.

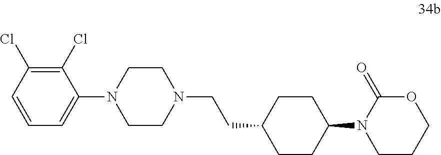 [SMILES: O=C1OCCCN1[C@H]1CC[C@H](CCN2CCN(c3cccc(Cl)c3Cl)CC2)CC1]

### 3-(trans-4-(2-(4-(2,3-Dichlorophenyl)piperazin-1-yl)ethyl)cyclohexyl)-1,3-oxazinan-2-one (compound 34b)

A mixture of compound 31b (0.178 g, 0.372 mmol), K₂CO₃ (0.159 g, 1.15 mmol), and anhydrous MeCN (7.5 mL) was heated to 100 °C in a pressure flask. After stirring for 2 days, the reaction mixture was allowed to cool to rt and concentrated under a stream of Ar. The residue was suspended in H₂O (10 mL), and the aq layer was extracted with DCM (3×10 mL). The combined organic layers were dried over anhydrous Na₂SO₄, filtered, and concentrated. The crude product was purified by chromatography (24 g of silica gel, 0-5% MeOH/DCM) to afford compound 34b (89.6 mg, 0.203 mmol, 55% yield) as a white solid. R_{f}=0.2 (10% MeOH/DCM); ¹H NMR (400 MHz, CDCl₃) δ 7.16-7.10 (m, 2H), 6.90-6.92 (m, 1H), 4.20 (t, J=5.4 Hz, 2H), 4.17-4.07 (m, 1H), 3.21 (t, J=6.2 Hz, 2H), 3.16-2.93 (m, 4H), 2.76-2.49 (m, 4H), 2.46-2.37 (m, 2H), 2.03-1.95 (m, 2H), 1.08-1.71 (m, 4H), 1.52-1.38 (m, 4H), 1.29-1.06 (m, 3H); ¹³C NMR (101 MHz, CDCl₃) δ 153.3, 151.5, 134.2, 127.7, 127.6, 124.7, 118.7, 66.0, 56.7, 55.8, 53.6 (2C), 51.5 (2C), 39.6, 35.7, 34.0, 32.3 (2C), 29.2 (2C), 22.6; IR (film) 1683 cm⁻¹; mp 149-150 °C (dec); HRMS (ESI) m/z [M+H]⁺ calcd for C₂₂H₃₂Cl₂N₃O₂ 440.1866, found 440.1862; t_{R}=34.7 min (HPLC, basic). The HCl salt was precipitated from a 0.03 M solution of the free base in 33% CHCl₃/acetone using a 2.0 M solution of HCl in Et₂O (8.1 equiv). Mp 246-247 °C (dec). Anal. calcd for C₂₂H₃₁Cl₂N₃O₂·HCl: C, 55.41; H, 6.76; N, 8.81. Found: C, 55.25; H, 6.66; N, 8.70.

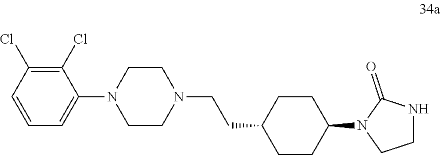 [SMILES: O=C1NCCN1[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)imidazolidin-2-one (compound 35a)

NaH (60 wt % dispersion in mineral oil, 0.177 g, 4.41 mmol) was added in one portion to a mixture of compound 32a (0.205 g, 0.443 mmol) in anhydrous THF (15 mL) under an Ar atmosphere. After stirring at rt overnight, the reaction was quenched with a saturated aq solution of NaHCO₃ (20 mL, and the aq layer was extracted with EtOAc (3×20 mL). The combined organic layers were dried over anhydrous Na₂SO₄, filtered, and concentrated. The crude product was purified by chromatography (24 g of silica gel, 0-10% MeOH/DCM) to afford compound 35a (66.6 mg, 0.157 mmol, 35% yield) as a white solid. R_{f}=0.3 (10% MeOH/DCM); ¹H NMR (400 MHz, CDCl₃) δ 7.18-7.09 (m, 2H), 7.00-6.91 (m, 1H), 4.57-4.45 (m 1H), 3.69 (t, J=12.6 Hz, 1H), 3.43-3.32 (m, 4H), 3.16-2.94 (m, 4H), 2.77-2.48 (m, 4H), 2.47-2.36 (m, 2H), 1.89-1.78 (m, 2H), 1.79-1.69 (m, 2H), 1.48-1.32 (m, 4H), 1.29-1.18 (m, 1H), 1.17-1.04 (m, 2H); ¹³C NMR (101 MHz, CDCl₃) δ 162.3, 151.5, 134.2, 127.7, 127.6, 124.6, 118.7, 56.7, 53.6 (2C), 51.5 (2C), 51.2, 40.8, 38.7, 35.7, 34.1, 32.3 (2C), 30.0 (2C); IR (film) 3219, 3087, 1686 cm⁻¹; mp 215-216 °C (dec); HRMS (ESI) m/z [M+H]⁺ calcd for C₂₁H₃₁Cl₂N₄O 425.1869, found 425.1864; t_{R}=23.8 min (HPLC, basic).

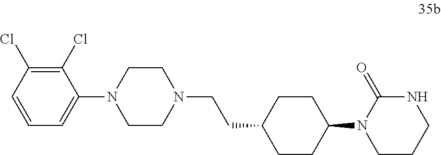 [SMILES: O=C1NCCCN1[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)tetrahydropyrimidin-2(1H)-one (compound 35b)

The same procedure as the one described for compound 35a was followed using NaH (60 wt % dispersion in mineral oil, 0.249 g, 6.23 mmol) and compound 32b (0.270 g, 0.566 mmol) in anhydrous THF (22 mL). After work-up, the crude product was purified by chromatography (12 g of silica gel, 0-10% MeOH/DCM) to afford compound 35b (0.137 g, 0.311 mmol, 55% yield) as a white solid. R_{f}=0.4 (10% MeOH/DCM); ¹H NMR (400 MHz, CDCl₃) δ 7.17-7.09 (m, 2H), 6.99-6.90 (m, 1H), 4.69-4.58 (m, 1H), 4.26 (t, J=12.5 Hz, 1H), 3.28-3.22 (m, 2H), 3.19-3.13 (m, 2H), 3.11-2.97 (m, 4H), 2.74-2.51 (m, 4H), 2.46-2.37 (m, 2H), 1.93-1.84 (m, 2H), 1.83-1.75 (m, 2H), 1.73-1.64 (m, 2H), 1.48-1.34 (m, 4H), 1.27-1.07 (m, 3H); ¹³C NMR (101 MHz, CDCl₃) δ 156.0, 151.5, 134.2, 127.7, 127.5, 124.6, 118.7, 56.8, 53.6 (2C), 52.8, 51.5 (2C), 40.4, 39.5, 35.8, 34.1, 32.5 (2C), 29.7 (2C), 22.7; IR (film) 3291, 3213, 3064, 1654 cm⁻¹; mp 238-239 °C (dec); HRMS (ESI) m/z [M+H]⁺ calcd for C₂₂H₃₃Cl₂N₄O 439.2026, found 439.2021; t_{R}=31.6 min (HPLC, basic).

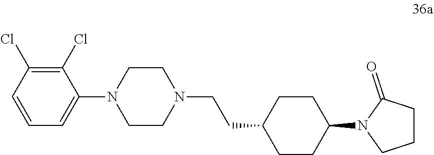 [SMILES: O=C1CCCN1[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)100yrrolidine-2-one (compound 36a)

To a solution of compound 5a (0.175 g, 0.492 mmol) and TEA (82 μL, 0.59 mmol) in DCM (5.0 mL) was added 4-chlorobutanoyl chloride (60 μL, 0.54 mmol) over 5 min. After stirring at rt overnight, the reaction was quenched with a saturated aq solution of NaHCO₃ (30 mL), and the aq layer was extracted with DCM (3×30 mL). The organic layer was dried over anhydrous Na₂SO₄, filtered, and concentrated. Using a pressure flask, the crude intermediate (33a) was dissolved in anhydrous THF (19 mL) under an Ar atmosphere, and NaH (60 wt % dispersion in mineral oil, 0.201 g, 5.03 mmol) was added in one portion. The reaction mixture was heated to 100 °C and stirred overnight. After cooling to rt, the reaction was quenched with a saturated aq solution of NaHCO₃ (25 mL), and the aq layer was extracted with EtOAc (3×25 mL). The combined organic layers were dried over anhydrous Na₂SO₄, filtered, and concentrated. The crude product was purified by chromatography (24 g of silica gel, 0-10% MeOH/DCM) to afford compound 36a (0.134 g, 0.317 mmol, 64% yield) as a white solid. R_{f}=0.5 (10% MeOH/DCM); ¹H NMR (400 MHz, CDCl₃) δ 7.17-7.10 (m, 2H), 6.95 (dd, J=6.4, 3.2 Hz, 1H), 3.98-3.88 (m, 1H), 3.33 (t, J=7.0 Hz, 2H), 3.18-2.94 (m, 4H), 2.76-2.50 (m, 4H), 2.46-2.34 (m, 4H), 2.03-1.93 (m, 2H), 1.86-1.78 (m, 2H), 1.74-1.65 (m, 2H), 1.49-1.34 (m, 4H), 1.30-1.19 (m, 1H), 1.18-1.06 (m, 2H); ¹³C NMR (101 MHz, CDCl₃) δ 174.5, 151.5, 134.2, 127.7, 127.6, 124.7, 118.7, 56.7, 53.6 (2C), 51.5 (2C), 50.6, 43.0, 35.6, 34.1, 32.2 (2C), 31.8, 30.0 (2C), 18.3; IR (film) 1677 cm⁻¹; mp 156-157 °C (dec); HRMS (ESI) m/z [M+H]⁺ calcd for C₂₂H₃₂Cl₂N₃O 424.1917, found 424.1909; t_{R}=33.1 min (HPLC, basic). The HCl salt was precipitated from a 0.03 M solution of the free base in 50% CHCl₃/acetone using a 2.0 M solution of HCl in Et₂O (8.2 equiv). Mp 278-279 °C (dec). Anal. calcd for C₂₃H₃₃Cl₂N₃O·1.5HCl·0.5H₂O: C, 54.64; H, 6.88; N, 8.69. Found: C, 54.64; H, 6.66; N, 8.57.

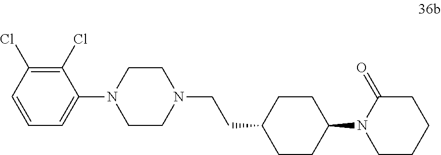 [SMILES: O=C1CCCCN1[C@H]1CC[C@H](CCN2CCN(c3cccc(Cl)c3Cl)CC2)CC1]

### 1-(trans-4-(2-(4-(2,3-Dichlorophenyl)piperazin-1-yl)ethyl)cyclohexyl)piperidin-2-one (compound 36b)

The same procedure as the one described for compound 36a was followed starting from compound 5a (0.175 g, 0.492 mmol) and 5-chloropentanoyl chloride (69 μL, 0.54 mmol), yielding compound (33b) as an intermediate. After work-up of the second step, the crude product was purified by chromatography (24 g of silica gel, 0-10% MeOH/DCM) to afford compound 36b (0.145 g, 0.330 mmol, 67% yield) as a white solid. R_{f}=0.5 (10% MeOH/DCM); ¹H NMR (400 MHz, CDCl₃) δ 7.18-7.10 (m, 2H), 7.00-6.90 (m, 1H), 4.54-4.43 (m, 1H), 3.29-3.14 (m, 2H), 3.13-2.93 (m, 4H), 2.80-2.49 (m, 4H), 2.48-2.33 (m, 4H), 1.86-1.78 (m, 2H), 1.77-1.70 (m, 4H), 1.69-1.61 (m, 2H), 1.51-1.37 (m, 4H), 1.28-1.08 (m, 3H); ¹³C NMR (101 MHz, CDCl₃) δ 169.4, 151.5, 134.2, 127.7, 127.6, 124.7, 118.7, 56.7, 53.6 (2C), 52.2, 51.5 (2C), 41.9, 35.8, 34.1, 32.8, 32.4 (2C), 29.2 (2C), 23.6, 21.1; IR (film) 1632 cm⁻¹; mp 167-168 °C (dec); HRMS (ESI) m/z [M+H]⁺ calcd for C₂₃H₃₄Cl₂N₃O 438.2073, found 438.2066; t_{R}=34.7 min (HPLC, basic). The HCl salt was precipitated from a 0.03 M solution of the free base in 50% CHCl₃/acetone using a 2.0 M solution of HCl in Et₂O (16 equiv). Mp 282-283 °C (dec). Anal. calcd for C₂₃H₃₃Cl₂N₃O·1.75HCl: C, 55.00; H, 6.97; N, 8.37. Found: C, 54.88; H, 6.82; N, 8.20.

The following examples further illustrate the invention but, of course, should not be construed as in any way limiting its scope.

### EXAMPLES

Fifty-six compounds in addition to cariprazine (compound 6a) were designed and synthesized that bind with high D₃R affinities (Kᵢ=0.14-50 nM) and exhibit a range of D₃R/D₂R binding selectivities (2- to 360-fold). Cariprazine and two lead compounds, compounds 13a and 13e, decreased cocaine self-administration (FR2; 1-10 mg/kg, i.p.) in rats, suggesting that partial agonists with modest D₃R-selectivity can be effective in treating psychostimulant use disorders (PSUD).

According to the present disclosure, compounds that are moderately D₃R/D₂R selective and are partial agonists, especially at D₂R, can be more effective than highly D₃R selective antagonists for treatment of PSUD. Highly selective D₃R antagonists/partial agonists have been shown to block the reinforcing effects of opioids, such as oxycodone, but are often less effective for psychostimulants, such as cocaine or methamphetamine, especially when the drug is readily available (low fixed ratio schedule of reinforcement e.g., FR1 or FR2). Nevertheless, compounds that are D₂R antagonists are generally not well tolerated in this patient population.

A series of D₃R selective partial agonists were designed with varying selectivities and functional efficacies at D₂R and D₃R, depending on their PP, SP, and/or linker as set forth in Formula I. Compounds were designed with high D₃R affinities (Kᵢ<1 nM) and improved selectivities (>20-fold) over D₂R that have the desired partial agonist functional efficacies, as measured in BRET-based assays for each receptor subtype. Target activities (D₁R, D₄R, 5HT_{1A}, 5HT_{2A}, 5HT_{2B}, and 5HT_{2C}) have been obtained on a subset of these compounds that had desirable D₃R/D₂R selectivity profiles as well as MPO scores (>3).

Based on the in vitro data obtained, compounds 13a and 13e were selected as examples for further study, which were tested side-by-side with cariprazine in a rat model of cocaine self-administration. These two compounds had higher but still moderate D₃R/D₂R selectivities (~20-fold) as compared to cariprazine (3.6-fold) and were effective in blocking cocaine self-administration in the rats (FR2; 1-10 mg/kg, i.p.). Overall, these convincing behavioral data support the notion that moderately D₃R selective partial agonists can be more effective than highly selective (>200-fold) D₃R antagonists in reducing drug-seeking behavior. The compounds described herein demonstrate high suitability for treating PSUD, for example, PSUD that can be dual diagnosed with affective disorders. Additional testing can be performed on the compounds of the present disclosure, for example, compounds 13a and 13e. Such studies can include, for example, determination of extrapyramidal side effects and efficacy in animal models of affective disorders, such as BD-l.

### Example 1

### D2R and D3R Binding Affinities

To investigate the structure-activity relationships (SAR), affinities of compounds at D₂R and D₃R were determined. Competitive binding experiments using membrane preparations from stably transfected HEK293 cells expressing human D_{2L} and D₃ receptors were performed with [³H]N-methylspiperone serving as the radioligand. The Kᵢ values for each compound are presented in Tables 1A and 2 along with the corresponding multiparameter optimization (MPO) scores, which are a prediction of brain penetrability. Compounds with MPO scores >3 were considered as potentially drug-like.

Radioligand binding assays were conducted similarly as previously described^{45, 67}. HEK293 cells stably expressing human D₂LR or D₃R (hD2R and hD3R) were grown in a 50:50 mix of DMEM and Ham's F12 culture media, supplemented with 20 mM HEPES, 2 mM L-glutamine, 0.1 mM non-essential amino acids, 1× antibiotic/antimycotic, 10% heat-inactivated fetal bovine serum, and 200 μg/mL hygromycin (Life Technologies, Grand Island, NY) and kept in an incubator at 37 °C and 5% CO₂. Upon reaching 80p-90% confluence, cells were harvested using premixed Earle's balanced salt solution with 5 mM EDTA (Life Technologies) and centrifuged at 3000 rpm for 10 min at 21 °C. The supernatant was removed, and the pellet was resuspended in 10 mL hypotonic lysis buffer (5 mM MgCl₂, 5 mM Tris, pH 7.4 at 4 °C) and centrifuged at 14 500 rpm (~25000g) for 30 min at 4 °C. The pellet was then resuspended in binding buffer. Bradford protein assay (Bio-Rad, Hercules, CA) was used to determine the protein concentration. For [³H]-N-methylspiperone binding studies membranes were diluted to 500 μg/mL, in fresh EBSS binding buffer made from 8.7 g/L Earle's Balanced Salts without phenol red (US Biological, Salem, MA), 2.2 g/L sodium bicarbonate, pH to 7.4, and stored in a −80 °C freezer for later use. For [³H]-(R)-(+)-7-OH-DPAT binding studies, membranes were harvested and used fresh; the binding buffer was made from 50 mM Tris, 10 mM MgCl₂, 1 mM EDTA, pH 7.4. On the test day, each test compound was diluted into half-log serial dilutions using the 30% dimethyl sulfoxide (DMSO) vehicle. When it was appropriate to assist solubilization of the drugs at the highest tested concentration, 0.1% AcOH (final concentration v/v) was added alongside the vehicle. Membranes were diluted in fresh binding buffer. Radioligand competition experiments were conducted in 96-well plates containing 300 μL fresh binding buffer, 50 μL of the diluted test compound, 100 μL of membranes (for [³H]-N-methylspiperone assays: 10-20 pg/well total protein for hD_{2L}R and hD₃R; for [³H]-(R)-(+)-7-OH-DPAT assays: 40-80, and 20-40 pg/well total protein for hD_{2L}R, and hD₃R, respectively), and 50 μL of radioligand diluted in binding buffer ([³H]-N-methylspiperone: 0.4 nM final concentration for all the hD₂-like receptor subtypes; [³H]-(R)-(+)-7-OH-DPAT: 1.5 nM final concentration for hD_{2L}, and 0.5 nM final concentration for hD₃; Perkin Elmer). Aliquots of radioligands solution were also quantified accurately in each experiment replicate, to determine how much radioactivity was added, taking in account the experimentally determined counter efficiency. Nonspecific binding was determined using 10 μM (+)-butaclamol (Sigma-Aldrich, St. Louis, MO), and total binding was determined with the 30% DMSO vehicle. All compound dilutions were tested in triplicate, and the reaction incubated for 60 min ([³H]-N-methylspiperone assays) or 90 min ([³H]-(R)-(+)-7-OH-DPAT assays) at room temperature. The reaction was terminated by filtration through PerkinElmer Uni-Filter-96 GF/B, presoaked for the incubation time in 0.5% polyethylenimine, using a Brandel 96-Well Plates Harvester Manifold (Brandel Instruments, Gaithersburg, MD). The filters were washed thrice with 3 mL (3×~1 mL/well) of ice-cold binding buffer. PerkinElmer MicroScint 20 Scintillation Cocktail (65 μL) was added to each well, and filters were counted using a PerkinElmer MicroBeta Microplate Counter. IC₅₀ values for each compound were determined from dose-response curves, and K values were calculated using the Cheng-Prusoff equation (Yung-Chi et al., Biochem. Pharmacol. 1973, 22 (23), 3099-31080. K_{d} values were determined via separate homologous competitive binding experiments. When a complete inhibition could not be achieved at the highest tested concentrations, K values have been extrapolated by constraining the bottom of the dose-response curves (=0% residual specific binding) in the nonlinear regression analysis. These analyses were performed using GraphPad Prism version 8 for Macintosh (GraphPad Software, San Diego, CA). All results were rounded to the third significant figure. Kᵢ values were determined from at least three independent experiments, each performed in triplicate, and are reported as the mean±standard error of the mean (SEM).

A first series of compounds involved modifications of the secondary pharmacophore (SP) (Table 1A, compounds 4a, compound 6a, and 7a-13a). Compared to compound 6a, similar D₃R binding affinities and selectivities were observed for the primary metabolites, compounds 7a and 8a. When the SP was a heterocycle (compounds 9a-12a), D₃R affinity was retained (Kᵢ=0.31-0.66 nM), while selectivity over D₂R increased from 5- to 44-fold. One compound (compound 13a) exhibited similarly higher affinity (Kᵢ=0.14 nM) but better selectivity (20-fold) for D₃R than cariprazine (compound 6a), while simultaneously maintaining a desirable MPO score (3.6). Kᵢ values were derived from IC₅₀ values using the Cheng-Prusoff equation and calculated as the mean of at least three independent experiments. The radioligand used in these cell-based assays was [³H]N-methylspiperone.

TABLE 1A
D₃R and D₂R binding affinities

| | | | | Kᵢ ± SEM (nM) | | D₂R Kᵢ / |
|---|---|---|---|---|---|---|
| Compound | Ar | R⁶ | MPO | D₂R | D₃R | D₃R Kᵢ |
| 4a | 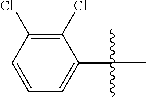 [SMILES: *c1cccc(Cl)c1Cl] | 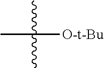 | 2.8 | 8.2 ± 2.6 | 2.13 ± 0.85 | 3.8 |
| 6a | | 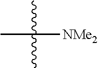 [SMILES: *N(C)C] | 3.6 | 0.78 ± 0.17 | 0.22 ± 0.06 | 3.6 |
| 7a | | 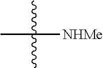 [SMILES: *NC] | 3.5 | 1.21 ± 0.06 | 0.21 ± 0.02 | 5.7 |
| 8a | | 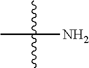 [SMILES: *N] | 3.8 | 1.49 ± 0.03 | 0.26 ± 0.10 | 5.7 |
| 9a | | 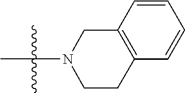 [SMILES: *N1CCc2ccccc2C1] | 2.8 | 1.86 ± 0.06 | 0.36 ± 0.05 | 5.1 |
| 10a | | 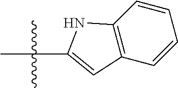 [SMILES: *c1cc2ccccc2[nH]1] | 2.5 | 6.5± 1.9 | 0.31 ± 0.10 | 21 |
| 11a | | 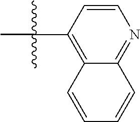 [SMILES: *c1ccnc2ccccc12] | 2.8 | 29 ± 13 | 0.66 ± 0.28 | 44 |
| 12a | | 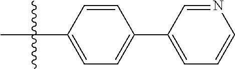 [SMILES: *c1ccc(-c2cccnc2)cc1] | 2.8 | 3.75 ± 0.65 | 0.59 ± 0.13 | 6.4 |
| 13a | | 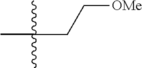 [SMILES: *CCOC] | 3.6 | 2.85 ± 0.63 | 0.14 ± 0.05 | 20 |
| 14a | | 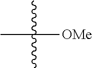 [SMILES: *OC] | 3.5 | 3.72 ± 0.02 | 0.323 ± 0.075 | 11.5 |
| 14b | | 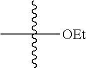 [SMILES: *OCC] | 3.2 | 8.40 ± 2.43 | 0.965 ± 0.129 | 8.7 |
| 14c | | 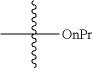 | 2.8 | 25.0 ± 2.1 | 1.74 ± 0.17 | 14.4 |
| 14d | | 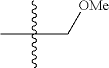 [SMILES: *COC] | 3.9 | 7.67 ± 0.69 | 0.268 ± 0.065 | 28.6 |
| 14e | | 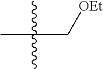 [SMILES: *COCC] | 3.5 | 41.3 ± 3.9 | 1.06 ± 0.32 | 39.0 |
| 14f | | 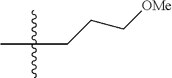 [SMILES: *CCCOC] | 3.3 | 21.7 ± 3.8 | 1.15 ± 0.29 | 18.9 |
| 14g | | 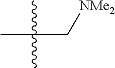 [SMILES: *CN(C)C] | 3.8 | 3.72 ± 0.46 | 0.398 ± 0.084 | 9.4 |
| 14h | | 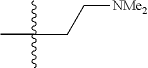 [SMILES: *CCN(C)C] | 3.5 | 1.25 ± 0.14 | 0.248 ± 0.046 | 5.0 |
| 14i | | 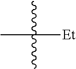 [SMILES: *CC] | 3.2 | 6.06 ± 1.07 | 0.447 ± 0.119 | 13.6 |
| 14j | | 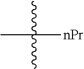 | 2.9 | 25.6 ± 5.7 | 0.875 ± 0.178 | 29.3 |
| 14k | | 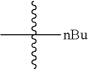 | 2.6 | 51.5 ± 18.6 | 2.37 ± 0.60 | 21.7 |
| 6b | 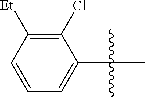 [SMILES: *c1cccc(CC)c1Cl] | —NMe₂ | 3.5 | 0.75 ± 0.07 | 0.26 ± 0.03 | 2.9 |
| 7b | | 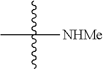 [SMILES: *NC] | 3.4 | 0.48 ± 0.11 | 0.18 ± 0.01 | 2.6 |
| 9b | | 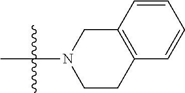 [SMILES: *N1CCc2ccccc2C1] | 2.8 | 0.87 ± 0.08 | 0.29 ± 0.08 | 3.0 |
| 10b | | 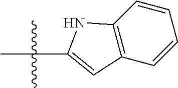 [SMILES: *c1cc2ccccc2[nH]1] | 2.5 | 7.8 ± 1.9 | 1.31 ± 0.46 | 6.0 |
| 11b | | 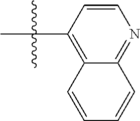 [SMILES: *c1ccnc2ccccc12] | 2.8 | 5.76 ± 0.93 | 0.34 ± 0.06 | 17 |
| 12b | | 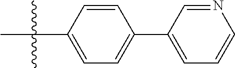 [SMILES: *c1ccc(-c2cccnc2)cc1] | 2.8 | 1.80 ± 0.34 | 0.58 ± 0.07 | 3.1 |
| 13b | | 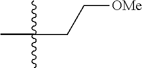 [SMILES: *CCOC] | 3.5 | 1.78 ± 0.18 | 0.25 ± 0.04 | 7.1 |
| 6c | 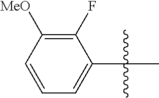 [SMILES: *c1cccc(OC)c1F] | 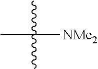 [SMILES: *N(C)C] | 5.2 | 134 ± 17 | 9.4 ± 1.2 | 14 |
| 13c | | 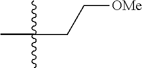 [SMILES: *CCOC] | 5.1 | 479 ± 26 | 1.31 ± 0.02 | 370 |
| 6d | 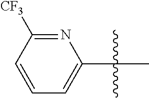 [SMILES: *c1cccc(C(F)(F)F)n1] | 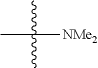 [SMILES: *N(C)C] | 4.7 | 26.7 ± 5.4 | 2.38 ± 0.54 | 11 |
| 11d | | 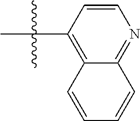 [SMILES: *c1ccnc2ccccc12] | 2.6 | 162 ± 48 | 1.00 ± 0.08 | 160 |
| 13d | | 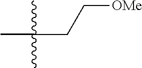 [SMILES: *CCOC] | 4.6 | 127 ± 14 | 1.06 ± 0.08 | 120 |
| 4e | 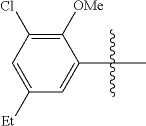 [SMILES: *c1cc(CC)cc(Cl)c1OC] | 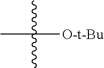 | 3.1 | 68.1 ± 4.3 | 38.8 ± 3.0 | 1.8 |
| 6e | | 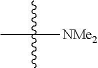 [SMILES: *N(C)C] | 3.4 | 4.12 ± 0.17 | 1.76 ± 0.11 | 2.3 |
| 7e | | 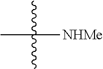 [SMILES: *NC] | 3.3 | 5.8 ± 1.3 | 0.64 ± 0.10 | 9.1 |
| 8e | | 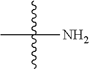 [SMILES: *N] | 3.5 | 5.3 ± 1.8 | 0.78 ± 0.28 | 6.8 |
| 10e | | 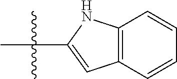 [SMILES: *c1cc2ccccc2[nH]1] | 2.3 | 114 ± 36 | 1.03 ± 0.26 | 110 |
| 13e | | 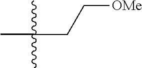 [SMILES: *CCOC] | 3.3 | 15.2 ± 2.0 | 0.73 ± 0.16 | 21 |
| 6f | 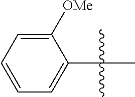 [SMILES: *c1ccccc1OC] | 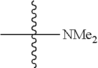 [SMILES: *N(C)C] | 5.0 | 6.07 ± 2.32 | 1.01 ± 0.32 | 6.0 |
| 6g | 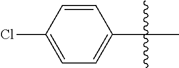 [SMILES: *c1ccc(Cl)cc1] | 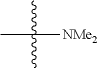 [SMILES: *N(C)C] | 4.5 | 337 ± 24 | 33.8 ± 0.8 | 10.0 |
| 6h | 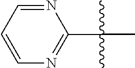 [SMILES: *c1ncccn1] | 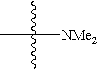 [SMILES: *N(C)C] | 5.7 | 3440 ± 464 | 600 ± 136 | 5.7 |
| 6i | 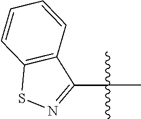 [SMILES: *c1nsc2ccccc12] | 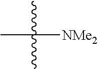 [SMILES: *N(C)C] | 4.6 | 4.43 ± 0.38 | 4.17 ± 0.33 | 1.1 |
| 6j | 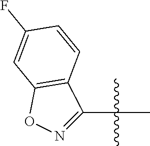 [SMILES: *c1noc2cc(F)ccc12] | 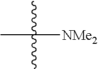 [SMILES: *N(C)C] | 4.4 | 5.26 ± 1.56 | 5.37 ± 0.72 | 1.0 |
| 6k | 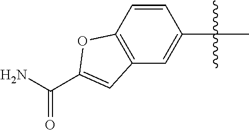 [SMILES: *c1ccc2oc(C(N)=O)cc2c1] | 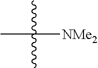 [SMILES: *N(C)C] | 4.2 | 61100 ± 35100 | 1460 ± 270 | 41.9 |
| | | | | Kᵢ ± SEM (nM) | | |
| Compound | Ar | 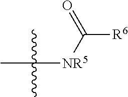 | MPO | D₂R | D₃R | D₂R Ki / D₃R Ki |
| 34b | 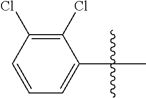 [SMILES: *c1cccc(Cl)c1Cl] | 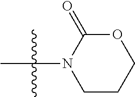 [SMILES: *N1CCCOC1=O] | 3.2 | 5.46 ± 2.14 | 0.248 ± 0.101 | 22.0 |
| 35a | | 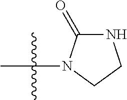 [SMILES: *N1CCNC1=O] | 3.9 | 5.66 ± 0.89 | 0.438 ± 0.053 | 12.9 |
| 35b | | 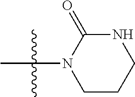 [SMILES: *N1CCCNC1=O] | 3.5 | 19.0 ± 4.7 | 2.97 ± 0.85 | 6.4 |
| 36a | | 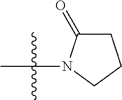 [SMILES: *N1CCCC1=O] | 3.0 | 4.49 ± 0.78 | 0.537 ± 0.167 | 8.4 |
| 36b | | 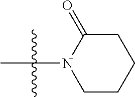 [SMILES: *N1CCCCC1=O] | 2.8 | 9.89 ± 2.77 | 1.74 ± 0.46 | 5.7 |

As is apparent from the results set forth in Table 1, compounds 13a, 13d, 13e, 14d, 14e, and 34b, amongst others, have high D₃R affinities (e.g., a Kᵢ of 1 nM or lower), desirable D2/D3 ratios (e.g., 20-120), and MPO scores (e.g., >3) that suggest drug-like properties.

The next several series of compounds explored different primary pharmacophores (PPs) in varying combinations with the aforementioned SPs. As shown in Table 1A, the 2,3-dichorophenyl was substituted with a 2-chloro-3-ethylphenyl (compounds 6b, 7b, and 9b-13b). Although high D₃R affinities were achieved (Kᵢ=0.18-1.31 nM), none of these compounds were substantially more selective than compound 6a (cariprazine) and most had poor MPO scores (<3). Conversely, the 2-fluoro-3-methoxyphenyl compound (6c) noticeably decreased D₃R and D₂R affinities (Kᵢ=9.4 and 134 nM, respectively). Of note, compound 13c showed much greater D₃R/D₂R selectivity than any of the compounds in this series, which was surprising. D₂R agonists can be sensitive to the radioligand they are displacing and typically show much lower affinities when tested against a D₂-like antagonist (e.g., [³H]N-methylspiperone). The 2-fluoro, 3-methoxy-substituted phenyl piperazine PP might be conferring agonist actions and thus binding affinities using the D₂-like agonist [³H]7-OH-DPAT were tested (Table 1 B). D₃R selectivities were found to be much lower in this binding assay (D₂R Kᵢ/D₃RKᵢ=0.9 and 15 for compounds 6c and 13c, respectively), suggesting these compounds were agonists at D₂R and possibly D₃R. In Table 1 B, Kᵢ values were derived from IC₅₀ values using the Cheng-Prusoff equation, and calculated as the mean of at least three independent experiments. The radioligand used in these cell-based assays was [³H]7-OH-DPAT.

TABLE 1B

| | Kᵢ ± SEM (nM) | | D₂R Kᵢ/ |
|---|---|---|---|
| compound | D₂R | D₃R | D₃R Kᵢ |
| 6c | 1.15 ± 0.14 | 1.31 ± 0.13 | 0.9 |
| 13c | 6.32 ± 0.51 | 0.43 ± 0.08 | 15 |

The 2-fluoro-3-methoxy-containing PP also caused a loss in affinities when the N,N-dimethylurea was replaced with the 3-methoxypropanamide (compound 13c), and further compounds were not pursued. The 2-trifluoromethyl substituted pyridine compounds (6d, 11d, and 13d) showed promising increases in D₃R selectivity. Specifically, SPs with the 4-quinoline (11d) and 3-methoxypropanamide (compound 13d) showed a 160- and 120-fold D₃R/D₂R selectivity, respectively. However, replacing the 2,3-dichlorophenyl substituent with the 2-methoxy-3-chloro-5-ethyl-phenyl group (compounds 4e, 6e-8e, 10e, and 13e) resulted in D₃R affinities and selectivities over D₂R that were only modestly different from the parent compound, unless the SP contained an indole (compound 10e). Nevertheless, the 3-methoxypropanamide SP in this series gave compound 13e with a 21-fold D₃R/D₂R selectivity, subnanomolar affinity for D₃R (Kᵢ=0.73 nM), and potentially drug-like MPO score (3.3).

For the last series of compounds, the terminal urea of compound 6a was incorporated into the trans-1,4-cyclohexane ring system of the linker. As shown in Table 2, compounds 21a and 22a retained the two-carbon chain between the 6-membered ring and the PP, which resulted in about a 20-fold D₃R selectivity over D₂R regardless of the SP. When the linking chain was increased by an additional methylene group, the D₃R binding affinity for compound 21b, which is a constitutional isomer of compound 6a, was reduced by about 9-fold compared to the parent drug. Interestingly, only the 3-methoxypropanamide containing compound (22b) retained the about 20-fold D₃R selectivity. If a hydroxyl group was appended to the linking chain between the PP and SP (compounds 29a, 30a, 29b, and 30b), the MPO scores noticeably improved to ≥5, but the D₃R affinities were substantially reduced (Kᵢ=18.7-50.7 nM). The D₃R/D₂R selectivities for these compounds were also undesirable (<10-fold), so the synthesis of additional compounds containing this hydroxyl group were not pursued further. Kᵢ values are derived from IC₅₀ values using the Cheng-Prusoff equation and calculated as the mean of at least three independent experiments. The radioligand used in these cell-based assays was [³H]N-methylspiperone.

TABLE 2
D3R and D2R binding affinities of linker modified compounds.

| | | | | Kᵢ ± SEM (nM) | | D2R Ki / |
|---|---|---|---|---|---|---|
| Compound | Linker | R^{21/22/29/30} | MPO | D₂R | D₃R | D3R Ki |
| 21a | 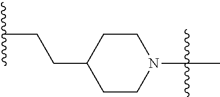 [SMILES: *CCC1CCN(*)CC1] | 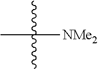 [SMILES: *N(C)C] | 3.9 | 20.5 ± 2.1 | 1.02 ± 0.10 | 20 |
| 22a | | 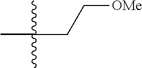 [SMILES: *CCOC] | 4.4 | 86 ± 28 | 3.5 ± 1.3 | 25 |
| 21b | 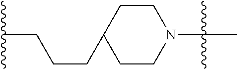 [SMILES: *CCCC1CCN(*)CC1] | 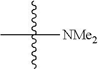 [SMILES: *N(C)C] | 3.4 | 7.2 ± 1.1 | 1.86 ± 0.10 | 3.9 |
| 22b | | 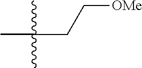 [SMILES: *CCOC] | 3.7 | 19.4 ± 2.6 | 0.96 ± 0.05 | 20 |
| 29a | 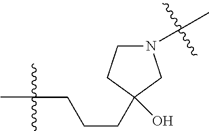 [SMILES: *CCCC1(O)CCN(*)C1] | 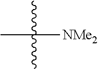 [SMILES: *N(C)C] | 5.3 | 89.8 ± 5.9 | 29.5 ± 3.7 | 3.0 |
| 30a | | 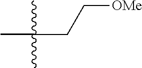 [SMILES: *CCOC] | 5.1 | 175 ± 19 | 18.7 ± 2.5 | 9.4 |
| 29b | 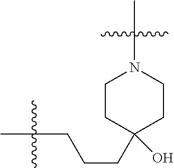 [SMILES: *CCCC1(O)CCN(*)CC1] | 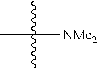 [SMILES: *N(C)C] | 5.0 | 38.4 ± 5.2 | 50.7 ± 5.7 | 0.76 |
| 30b | | 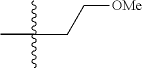 [SMILES: *CCOC] | 5.1 | 125 ± 27 | 27.5 ± 3.3 | 4.5 |

### Example 2

Characterization of Compounds in Assays of D₂R and D₃R Activation

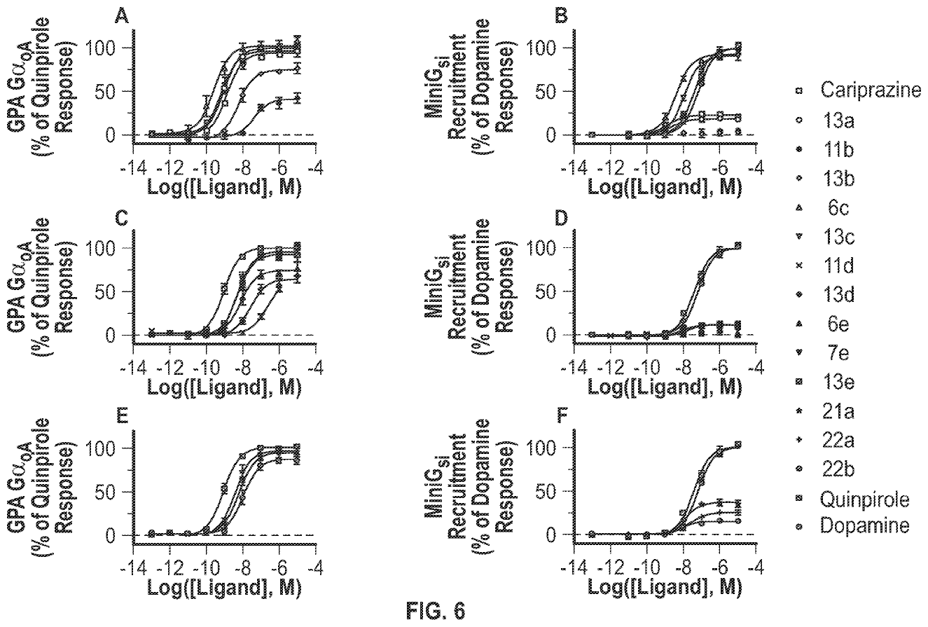

With the binding studies and SAR established, the abilities of fourteen selected ligands to signal through both D₂R and D₃R were characterized along with quinpirole and dopamine as reference agonists and haloperidol as reference antagonist. To measure D₂R activation, two BRET assays were used. The first consisted of a relatively amplified and sensitive G protein activation (GPA) assay that measures Gββγ release from Gα_{oA}, which allows detection of signals from less efficacious partial agonists (Table 3, FIG. 6, panels A, C, and E). The second measured recruitment of a truncated, soluble ‘mini’ Gi-protein fused with a Venus fluorescent protein (mGsi-Venus) to a D_{2L}R fused with a nanoluciferase (D_{2L}R-Nluc). This less amplified, proximal assay allows us to distinguish between full agonists and more efficaceous partial agonists (miniG assay, Table 3, FIG. 6, panels B, D, and F) that gave a maximal response relative to the reference agonist quinpirole in the GPA assay.

Bioluminescence Resonance Energy Transfer (BRET) studies of D_{2/3}R receptor signaling were performed as follows. All reagents were purchased from Sigma Aldrich-Merck unless otherwise stated. BRET experiments were performed in transiently transfected human embryonic kidney 293 T (HEK 293T) cells. Briefly, cells were grown and maintained at 37 °C in 5% CO₂ in Dulbecco's modified eagle medium (DMEM) supplemented with 10% (v/v) fetal bovine serum (FBS). Cells were seeded in 10 cm Petri dishes (2.5×10⁶ cells per dish) and allowed to grow overnight in media at 37 °C, 5% CO₂. The following day, cells were transiently transfected in full media supplemented with antibiotics (100 U/mL penicillin and 100 μg/mL streptomycin, Gibco) using a 1:6 total DNA to PEI (PolySciences Inc) ratio. BRET constructs were as follows: 2 μg of WT-Gα_{oA}, 1 μg of Gβ1-Venus(156-239), 1 μg of Gγ2-Venus(1-155), 1 μg of masGRK3ct-Rluc8 and 1 μg of receptor (D_{2L}R or D₃R) for GPA assays and 4 μg of Venus-mGₛᵢ and 1 μg of D₂LR-Rluc8 for mini G recruitment assays. Cells were then allowed to grow overnight at 37 °C, 5% CO₂. The next day, cells were plated in Greiner poly-D-lysine-coated 96-well plates (SLS) in media and allowed to grow overnight. On the day of the assay (48 h post-transfection), cells were washed once with D-PBS (Lonza, SLS) and incubated in D-PBS for 30 min at 37 °C, 5% CO₂. The Rluc substrate coelenterazine h (NanoLight) was added to each well (final concentration of 5 μM) and cells were incubated for 5 minutes at 37 °C. After 5 minutes, ligands (final concentration from 10 μM to 0.01 nM in D-PBS) were added to the plate and cells were incubated for a further 10 minutes at 37 °C. For the antagonist-mode assays that involve co-addition, quinpirole was added together with the ligands (final concentration of 3 nM) to generate 50-70% of the signal that can then be displaced by an antagonist. The plate was then read in a PHERAstar FSX microplate reader (Venus and Rluc emission signals at 535 and 475 nm respectively, BMG Labtech). The ratio of Venus:Rluc counts was used to quantify the BRET signal in each well. Data were normalized to the maximal response of dopamine/quinpirole or no drug for the 100% or 0% response, respectively and as indicated in the figure axes titles. All experiments were performed in duplicate and at least three times independently. All data points represent the mean value and error bars represent the standard error of the mean (SEM). Data were fitted using the built-in log(agonist) vs. response (three parameters) model in Prism 9.0 (GraphPad software Inc., San Diego, CA). For agonist-mode assays, data were fitted to a three-parameter concentration-response model where EC₅₀ is the concentration of the agonist needed to elicit half the maximal response of the particular agonist, defined as Eₘₐₓ. For the antagonist-mode assays, data points were fitted using a three-parameter concentration-response model where IC₅₀ is the concentration required to inhibit half the maximum response of the agonist used at a particular concentration. Values of pEC₅₀ or pIC₅₀ plus/minus error are given as the error has a gaussian distribution whereas the error associated with the antilog value does not.

As expected, the reference agonist quinpirole displayed 43-fold lower potency in the miniG assay as compared to the GPA assay consistent with a lower degree of signal amplification. Quinpriole and dopamine displayed equivalent maximal responses in the miniG assay (Table 3). All ligands tested retained D₂R agonism to various levels of intrinsic efficacy. The 2,3-dichorophenyl compounds (6a and 13a) are D₂R partial agonists (FIG. 6, panel B, Eₘₐₓ value of 22.5% and 18.4% of the dopamine maximal response respectively in miniGsi recruitment assay) but displayed a maximal effect similar to quinpirole in the GPA assay. The 2-chloro-3-ethylphenyl compounds (11b and 13b) are weak partial agonists at D₂R displaying lower efficacy than cariprazine with submaximal responses in the GPA assay (41% and 75% of quinpirole response, respectively) and no detectable effect in the miniG assay. The 2-fluoro-3-methoxyphenyl compounds (6c and 13c) displayed a maximal effect similar to that of dopamine and quinpirole in both assays D₂R agonists indicating they are full agonists. Indeed, these data corroborated the binding data obtained using [³H]7-OH-DPAT as the radioligand, as described above. The 2-trifluoromethyl substituted pyridine compounds (11d and 13d) were both weak partial agonists displaying lower efficacy than cariprazine in both assays. The 2-methoxy-3-chloro-5-ethylphenyl compounds (6e, 7e, and 13e) were also weak partial agonists displaying lower maximal effects than both quinpirole and cariprazine, but with higher potency than the 2-trifluoromethyl substituted pyridine compounds. Finally the modified linker compounds (21a, 22a, and 22b) all behaved as weak partial agonists in the miniG assay, displaying slightly higher (compound 21a, 38% of dopamine Eₘₐₓ) similar (compound 22a, 25%) and lower (compound 22b, 15%) efficacy as compared to cariprazine (23%). Thus, apart from the compounds with the 2-fluoro-3-methoxyphenyl PP, all compounds exhibited weak partial agonism at the D₂R like cariprazine but with subtle differences in maximal effects across the compound set. The efficacies of D₂R and D₃R compounds can be modified through modifications to PP, linker and SP. The range of potencies and efficacies in this series of compounds allows for determination of the optimal pharmacological profile including binding affinities, selectivities, and functional efficacies at both D₂R and D₃R. Compounds were tested in an amplified Gα_{oA} G protein (GPA Gα_{oA}) activation assay, shown on the left columns, and a less amplified miniG recruitment assay, shown on the right columns. ND indicates no agonist response detected, ‘-’ indicates compound not tested. Data represent the mean±SEM of three independent experiments performed in duplicate.

TABLE 3
The maximal effects (Eₘₐₓ) and potencies (EC₅₀) of compounds
determined in two assays of D₂R activation.

| | D₂R GPA Gαo_{A} | | | D₂R mini Gₛᵢ recruitment | | |
|---|---|---|---|---|---|---|
| | pEC₅₀ ± | EC₅₀ | Eₘₐₓ ± | pEC₅₀ ± | EC₅₀ | Eₘₐₓ ± |
| compound | SEM | (nM) | SEM (%) | SEM | (nM) | SEM (%) |
| 6a | 8.87 ± | 1.35 | 94.2 ± | 8.87 ± | 1.34 | 22.5 ± |
| | 0.08 | | 1.7 | 0.28 | | 1.7 |
| 13a | 8.77 ± | 1.70 | 94.7 ± | 8.65 ± | 2.26 | 18.4 ± |
| | 0.10 | | 2.2 | 0.27 | | 1.4 |
| 11b | 7.37 ± | 42.60 | 41.4 ± | ND | ND | ND |
| | 0.19 | | 3.0 | | | |
| 13b | 8.22 ± | 5.98 | 75.0 ± | ND | ND | ND |
| | 0.10 | | 2.3 | | | |
| 6c | 9.57 ± | 0.27 | 102.3 ± | 8.39 ± | 4.10 | 92.4 ± |
| | 0.15 | | 3.6 | 0.06 | | 1.6 |
| 13c | 9.13 ± | 0.75 | 96.4 ± | 7.96 ± | 11.02 | 90.6 ± |
| | 0.13 | | 3.3 | 0.06 | | 2.0 |
| 11d | <7 | >100 | <60% | ND | ND | ND |
| 13d | 7.57 ± | 27.12 | 64.3 ± | ND | ND | ND |
| | 0.15 | | 3.2 | | | |
| 6e | 8.14 ± | 7.18 | 74.3 ± | ND | ND | ND |
| | 0.14 | | 3.1 | | | |
| 7e | 8.30 ± | 5.00 | 95.9 ± | 8.15 ± | 7.08 | 11.1 ± |
| | 0.11 | | 2.9 | 0.43 | | 1.9 |
| 13e | 8.28 ± | 5.22 | 93.0 ± | 7.58 ± | 26.21 | 12.5 ± |
| | 0.11 | | 2.9 | 0.21 | | 0.9 |
| 21a | 8.40 ± | 3.97 | 95.9 ± | 7.93 ± | 11.86 | 36.7 ± |
| | 0.10 | | 2.8 | 0.12 | | 1.5 |
| 22a | 8.20 ± | 6.28 | 94.5 ± | 7.66 ± | 21.85 | 25.1 ± |
| | 0.06 | | 1.9 | 0.11 | | 0.9 |
| 22b | 7.96 ± | 10.89 | 86.7 ± | 8.00 ± | 9.92 | 15.4 ± |
| | 0.08 | | 2.4 | 0.18 | | 1.0 |
| Quinpirole | 9.03 ± | 0.93 | 100.1 ± | 7.40 ± | 39.78 | 100.0 ± |
| | 0.05 | | 1.3 | 0.08 | | 2.7 |
| Dopamine | — | — | — | 7.18 ± | 66.25 | 99.9 ± |
| | | | | 0.03 | | 1.1 |

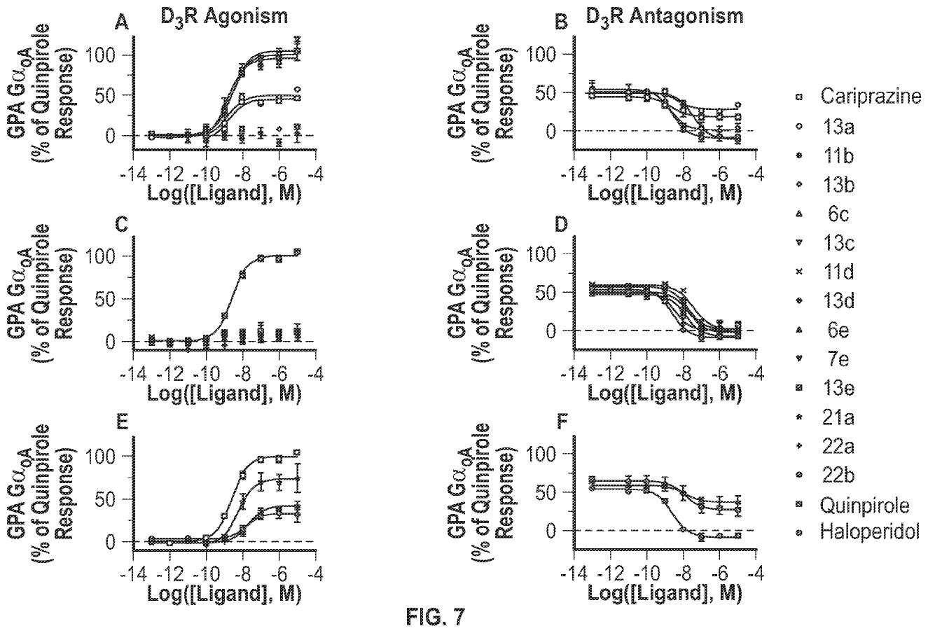

All fourteen selected compounds were then tested for D₃R signaling profiles, with the aim of selecting partial agonists or antagonists. The same GPA assay overexpressing Gα_{oA} was used to investigate D₃R agonism as the D₃R has been shown to selectively couple to this G protein α subunit (Table 4, FIG. 7, panels A, C, and E). The 2,3-dichorophenyl compounds (6a and 13a) both behaved as D₃R partial agonists in this assay with similar maximal effects (45% and 49% of quinpirole maximal effect), suggesting that this GPA assay would be suitable to detect D₃R partial agonism with a similar or lower level than that of cariprazine. The 2-fluoro-3-methoxyphenyl compounds did not display the desired weak partial agonist/antagonist properties at the D₃R but instead behaved as full agonists at D₃R in this assay, consistent with their action at the D₂R. The modified linkers compounds were all D₃R partial agonists with compound 21a exhibiting the strongest response with a higher efficacy than cariprazine (FIG. 7, panel E, Eₘₐₓ value of 73.2%). Finally, no agonist response was detected for the 2-chloro-3-ethylphenyl (compounds 11b and 13b), the 2-trifluoromethyl substituted pyridine compounds (11d and 13d), and the 2-methoxy-3-chloro-5-ethylphenyl compounds (6e, 7e, and 13e).

To quantify D₃R antagonism, a competitive assay was used where test compounds were added together with an EC₅₀ concentration of quinpirole to assess their ability to displace and antagonise the agonist response (Table 4, FIG. 7, panels B, D, and F). Haloperidol was used as a reference D₃R antagonist. Compounds that signaled as robust D₃R agonists (compounds 6c, 13c, and 21a) were not tested as antagonists. Out of the eleven remaining compounds, all showed some antagonist activity, with the partial agonists acting to reduce the quinpirole response down to their level of partial agonism (around 25% for compounds 6a and 13a, around 30% for compounds 22a and 22b). The most potent antagonists were compounds 13b, 13d, and 13a, showing similar potencies to haloperidol. In addition, compound 13a showed a very similar signaling profile to cariprazine (weak partial agonist with similar efficacy at both the D₂R and D₃R) but with improved selectivity for D₃R. Thus, this compound, like compound 13e, is a partial agonist at D₂R (with 2-fold lower maximal effect than cariprazine) in the miniG assay but acted as a D₃R antagonist. Furthermore, compound 13a displayed the highest affinity of those compounds found to be D₃R antagonists in the functional assay. Compounds were tested in an amplified Gα_{oA} G protein (GPA Gao) activation assay as agonists and antagonists. ND indicates no agonist response detected, ‘-’ indicates compound not tested. Data represent the mean±SEM of three independent experiments performed in duplicate.

TABLE 4
The maximal effects (Eₘₐₓ) and potencies (EC₅₀ and
IC₅₀) of compounds determined in an assay of D₃R activation.

| | D₃R GPA Gα_{oA} | | | D₃R GPA Gα_{oA} | |
|---|---|---|---|---|---|
| | agonism | | | antagonism | |
| | pEC₅₀ ± | EC₅₀ | Eₘₐₓ ± | pIC₅₀ ± | IC₅₀ |
| Compound | SEM | (nM) | SEM (%) | SEM | (nM) |
| 6a | 8.72 ± | 1.89 | 45.1 ± | 8.70 ± | 2.02 |
| | 0.14 | | 1.6 | 0.32 | |
| 13a | 8.80 ± | 1.60 | 49.3 ± | 8.34 ± | 4.62 |
| | 0.20 | | 2.3 | 0.23 | |
| 11b | ND | ND | ND | 7.44 ± | 36.00 |
| | | | | 0.25 | |
| 13b | ND | ND | ND | 8.73 ± | 1.87 |
| | | | | 0.16 | |
| 6c | 8.79 ± | 1.64 | 95.4 ± | — | — |
| | 0.14 | | 3.8 | | |
| 13c | 8.54 ± | 2.87 | 104.8 ± | — | — |
| | 0.16 | | 4.8 | | |
| 11d | ND | ND | ND | 7.47 ± | 33.63 |
| | | | | 0.15 | |
| 13d | ND | ND | ND | 8.49 ± | 3.21 |
| | | | | 0.24 | |
| 6e | ND | ND | ND | 7.87 ± | 13.41 |
| | | | | 0.13 | |
| 7e | ND | ND | ND | 7.70 ± | 19.85 |
| | | | | 0.19 | |
| 13e | ND | ND | ND | 7.76 ± | 17.45 |
| | | | | 0.13 | |
| 21a | 8.22 ± | 6.07 | 73.2 ± | — | — |
| | 0.20 | | 4.9 | | |
| 22a | 7.70 ± | 19.91 | 41.5 ± | 7.92 ± | 12.12 |
| | 0.13 | | 1.9 | 0.35 | |
| 22b | 7.80 ± | 16.02 | 33.0 ± | 7.89 ± | 12.93 |
| | 0.28 | | 2.9 | 0.37 | |
| Quinpirole | 8.59 ± | 2.59 | 100.1 ± | — | — |
| | 0.06 | | 1.7 | | |
| Haloperidol | — | — | — | 8.60 ± | 2.54 |
| | | | | 0.08 | |

### Example 3

### Off-Target Data for Compounds

Eleven compounds with a range of selectivities for D₃R over D₂R (between 7.1- to 370-fold) were then evaluated for off-target binding affinities (Table 5) and functional potencies/efficacies (Tables 6, 7, 8A, 8B, 9A, and 9B) against cariprazine (compound 6a). All tested compounds were selective for D₃R over other dopamine receptors (i.e., D₁R and D₄R), exhibiting markedly lower affinities for these two subtypes (Kᵢ ?3 mM and 200 nM, respectively). Like cariprazine, several compounds (for example, compounds 13a, 13b, and 13d) exhibited high binding affinities for the 5-HT₁ compound_{A} receptor (Kᵢ<10 nM), which is a key target in the treatment of schizophrenia. Interestingly, 5-HT_{1A} affinity decreased by 18-fold between compounds 13a and 13e (Kᵢ=6.0 vs. 108 nM, respectively) when 2,3-dichlorophenyl containing PP was replaced by a 2-methoxy-3-chloro-5-ethyl-phenyl group. In general, compounds were far more selective for D₃R over 5-HT_{2A} and 5-HT_{2C} (≥216- and ≥253-fold, respectively). The only exceptions to this trend were compounds 11b and 13b, which were not pursued further. The full subset of compounds also displayed high affinities for 5-HT_{2B} (Kᵢ<34.2 nM). Although activation of this serotonin receptor subtype has been associated with cardiotoxic effects,⁵⁶ all tested compounds behaved as 5-HT_{2B} antagonists in the functional assays, including cariprazine itself (see Table 9). Therefore, the studied compounds are not expected to induce cardiotoxicity from 5-HT_{2B} stimulation. Data set forth in Table 5 were obtained through the NIDA Addiction Treatment Discovery Program Contract (ADA12013) with Oregon Health & Science University. The radioligands used in these cell-based assays were ^{b}[³H]-SCH23390, ^{c}[³H]-spiperone, ^{d}[³H]-8-OH-DPAT, and ^{e}[³H]5-HT.

TABLE 5
Off-Target Binding Affinities

| | Kᵢ ± SEM (nM) | | | | | |
|---|---|---|---|---|---|---|
| Compound | D₁R^{b} | D₄R^{c} | 5-HT_{1A}^{d} | 5-HT_{2A}^{e} | 5-HT_{2B}^{e} | 5-HT_{2C}^{e} |
| 6a | 2920 ± 300 | 233 ± 20 | 1.81 ± 0.46 | 568 ± 72 | 0.151 ± 0.023 | 221.1 ± 8.0 |
| 11a | >3160 | 1390 ± 480 | 184 ± 25 | 1810 ± 640 | 1.99 ± 0.62 | 644 ± 58 |
| 13a | 4600 ± 400 | 756 ± 63 | 6.0 ± 1.8 | 54 ± 15 | 1.47 ± 0.36 | 252 ± 64 |
| 11b | 5390 ± 730 | 417 ± 40 | 75 ± 25 | 15.1 ± 4.8 | 1.27 ± 0.39 | 253 ± 86 |
| 13b | 6800 ± 480 | 342 ± 15 | 7.7 ± 1.8 | 20.8 ± 6.9 | 1.71 ± 0.43 | 34.0 ± 8.1 |
| 6c | >10000 | 1257 ± 23 | 33.8 ± 5.9 | >10000 | 34.2 ± 9.3 | >10000 |
| 13c | >10000 | 734 ± 16 | 69.9 ± 1.7 | >10000 | 29.6 ± 9.5 | >10000 |
| 11d | >3160 | 1790 ± 430 | 22.3 ± 6.4 | 1450 ± 340 | 18.8 ± 6.2 | 5800 ± 1300 |
| 13d | >10000 | 2520 ± 170 | 9.6 ± 2.6 | 4420 ± 340 | 11.3 ± 2.2 | 3170 ± 200 |
| 6e | >10,000 | 2434 ± 87 | 45.4 ± 9.0 | 680 ± 200 | 7.3 ± 1.9 | 1062 ± 86 |
| 7e | 8000 ± 1100 | 1346 ± 29 | 54.9 ± 4.2 | 207 ± 74 | 6.2 ± 1.5 | 162 ± 48 |
| 13e | >10,000 | 1870 ± 240 | 108 ± 11 | 158 ± 45 | 5.8 ± 1.7 | 800 ± 280 |

Table 6 shows agonist potencies and efficacies of compounds against other dopamine receptors. ND indicates not determined. Functional assays for dopamine receptors were not conducted if the K; value for the binding assay was greater than 500 nM.

TABLE 6
Agonist potencies and efficacies of compounds
against other dopamine receptors.

| | |
|---|---|
| | D₄R adenylate |
| D₁R cAMP | cyclase |

| | EC₅₀ ± | Eₘₐₓ ± | EC₅₀ ± | Eₘₐₓ ± |
|---|---|---|---|---|
| | SEM | SEM | SEM | SEM |
| compound | (nM) | (%) | (nM) | (%) |
| cariprazine | ND | ND | 80 ± 20 | 48.8 ± 2.2 |
| 11a | ND | ND | ND | ND |
| 13a | ND | ND | 18.1 ± 4.0 | 65.0 ± 3.8 |
| 11b | ND | ND | 21.2 ± 9.0 | 36.3 ± 4.1 |
| 13b | ND | ND | 15.6 ± 3.9 | 48.6 ± 3.4 |
| 6c | ND | ND | ND | ND |
| 13c | ND | ND | ND | ND |
| 11d | ND | ND | ND | ND |
| 13d | ND | ND | ND | ND |
| 6e | ND | ND | ND | ND |
| 7e | ND | ND | ND | ND |
| 13e | ND | ND | ND | ND |

Table 7 shows antagonist potencies and efficacies of compounds against other dopamine receptors. ND indicates not determined. Functional assays for dopamine receptors were not conducted if the Kᵢ value for the binding assay was greater than 500 nM.

TABLE 7
Antagonist potencies and efficacies of compounds
against other dopamine receptors.

| | |
|---|---|
| | D₄R adenylate |
| D₁R cAMP | cyclase |

| | IC₅₀ ± | Eₘₐₓ ± | IC₅₀ ± | Eₘₐₓ ± |
|---|---|---|---|---|
| | SEM | SEM | SEM | SEM |
| compound | (nM) | (%) | (nM) | (%) |
| cariprazine | ND | ND | 4860 ± 830 | 63.4 ± 7.6 |
| 11a | ND | ND | ND | ND |
| 13a | ND | ND | 6000 ± 1100 | 50 ± 17 |
| 11b | ND | ND | 5000 ± 1200 | 128 ± 18 |
| 13b | ND | ND | 2580 ± 460 | 98 ± 15 |
| 6c | ND | ND | ND | ND |
| 13c | ND | ND | ND | ND |
| 11d | ND | ND | ND | ND |
| 13d | ND | ND | ND | ND |
| 6e | ND | ND | ND | ND |
| 7e | ND | ND | ND | ND |
| 13e | ND | ND | ND | ND |

Tables 8A and 8B show agonist potencies and efficacies of compounds against different serotonin receptors. ND indicates not determined. Functional assays for serotonin receptors were not conducted if the Kᵢ value for the binding assay was greater than 250 nM.

TABLE 8A
Agonist potencies and efficacies of compounds
against different serotonin receptors.

| | 5-HT_{1A} [³⁵S]GTPγS | | 5-HT_{2A} IP-1 | |
|---|---|---|---|---|
| | EC₅₀ ± | Eₘₐₓ ± | EC₅₀ ± | Eₘₐₓ ± |
| compound | SEM (nM) | SEM (%) | SEM (nM) | SEM (%) |
| cariprazine | 5.5 ± 1.6 | 89.6 ± 3.6 | ND | ND |
| 11a | 286 ± 61 | 68.9 ± 4.0 | ND | ND |
| 13a | 24.4 ± 7.1 | 94.1 ± 7.9 | 66 ± 19 | 15.5 ± 1.6 |
| 11b | 152 ± 50 | 68.1 ± 5.5 | 150 ± 30 | 34.7 ± 4.1 |
| 13b | 6.0 ± 1.9 | 75.9 ± 2.5 | 36.3 ± 9.3 | 25.9 ± 1.9 |
| 6c | 55.9 ± 7.9 | 82.0 ± 3.8 | ND | ND |
| 13c | 121 ± 10 | 86.6 ± 3.5 | ND | ND |
| 11d | 110 ± 30 | 93.8 ± 6.5 | ND | ND |
| 13d | 27.8 ± 8.5 | 99.3 ± 6.8 | ND | ND |
| 6e | 88 ± 28 | 83.3 ± 6.4 | ND | ND |
| 7e | 146 ± 20 | 75.0 ± 3.5 | >10000 | <3 |
| 13e | 206 ± 27 | 78.1 ± 2.2 | >10000 | <1 |

TABLE 8B
Agonist potencies and efficacies of compounds
against different serotonin receptors.

| | 5-HT_{2B} IP-1 | | 5-HT_{2C} IP-1 | |
|---|---|---|---|---|
| | EC₅₀ ± | Eₘₐₓ ± | EC₅₀ ± | Eₘₐₓ ± |
| compound | SEM (nM) | SEM (%) | SEM (nM) | SEM (%) |
| cariprazine | >10000 | <4 | 215 ± 22 | 49.2 ± 6.6 |
| 11a | 10.0 ± 3.1 | 3.40 ± 0.56 | ND | ND |
| 13a | >10000 | <2 | 190 ± 18 | 77.8 ± 4.6 |
| 11b | >10000 | <2 | 3840 ± 660 | 52 ± 13 |
| 13b | >10000 | <1 | 43.5 ± 5.3 | 84.1 ± 5.7 |
| 6c | >10000 | <4 | ND | ND |
| 13c | >10000 | <1 | ND | ND |
| 11d | 34 ± 10 | 6.50 ± 0.34 | ND | ND |
| 13d | 37 ± 10 | 6.66 ± 0.91 | ND | ND |
| 6e | >10000 | <1 | ND | ND |
| 7e | >10000 | <1 | >10000 | <1 |
| 13e | >10000 | <1 | ND | ND |

Tables 9A and 9B show antagonist potencies and efficacies of compounds against different serotonin receptors. shows ND indicates not determined. Functional assays for serotonin receptors are not conducted if the K value for the binding assay is greater than 250 nM. If a compound is an agonist, it is not tested as an antagonist (−).

TABLE 9A
Antagonist potencies and efficacies of compounds
against different serotonin receptors.

| | 5-HT_{1A} [³⁵S]GTPγS | | 5-HT_{2A} IP-1 | |
|---|---|---|---|---|
| | IC₅₀ ± | Eₘₐₓ ± | IC₅₀ ± | Eₘₐₓ ± |
| compound | SEM (nM) | SEM (%) | SEM (nM) | SEM (%) |
| cariprazine | — | — | ND | ND |
| 11a | >10000 | <1 | ND | ND |
| 13a | — | — | 221 ± 57 | 72.1 ± 2.8 |
| 11b | — | — | 214 ± 38 | 48.2 ± 5.0 |
| 13b | — | — | 87 ± 22 | 62.7 ± 5.8 |
| 6c | — | — | ND | ND |
| 13c | — | — | ND | ND |
| 11d | — | — | ND | ND |
| 13d | — | — | ND | ND |
| 6e | — | — | ND | ND |
| 7e | — | — | 780 ± 220 | 9.81 ± 0.19 |
| 13e | ND | ND | 590 ± 160 | 100 ± 0 |

TABLE 9B
Antagonist potencies and efficacies of compounds
against different serotonin receptors.

| | 5-HT_{2B} IP-1 | | 5-HT_{2C} IP-1 | |
|---|---|---|---|---|
| | IC₅₀ ± | Eₘₐₓ ± | IC₅₀ ± | Eₘₐₓ ± |
| compound | SEM (nM) | SEM (%) | SEM (nM) | SEM (%) |
| cariprazine | 43 ± 13 | 99.0 ± 0.4 | >5000 | 9.2 ± 2.7 |
| 11a | 263 ± 71 | 83.2 ± 1.9 | ND | ND |
| 13a | 26.2 ± 1.3 | 9.73 ± 0.27 | — | — |
| 11b | 83.0 ± 4.7 | 96.8 ± 1.8 | >10000 | 3.9 ± 1.7 |
| 13b | 26.1 ± 1.2 | 100 ± 0 | — | — |
| 6c | 121 ± 23 | 9.93 ± 0.07 | ND | ND |
| 13c | 519 ± 81 | 82 ± 18 | ND | ND |
| 11d | 289 ± 57 | 92.1 ± 2.8 | ND | ND |
| 13d | 460 ± 100 | 95.6 ± 2.0 | ND | ND |
| 6e | 143 ± 17 | 100 ± 0 | ND | ND |
| 7e | 81 ± 23 | 100 ± 0 | 5380 ± 810 | 36.7 ± 4.2 |
| 13e | 158 ± 15 | 101 ± 0 | ND | ND |

### Example 4

### Effects of Compounds on Cocaine Self-Administration in Rats

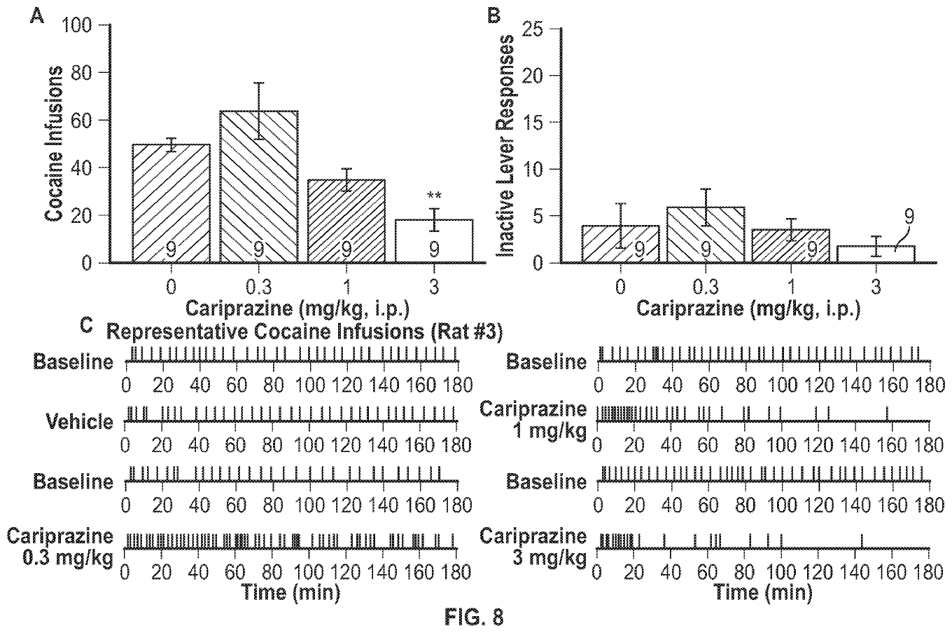

Cariprazine (compound 6a) was tested in a rat model of cocaine self-administration under a fixed-ratio 2 (FR2, i.e., two-active lever presses lead to one cocaine infusion) schedule of reinforcement. FIG. 8, panel A, shows dose-dependent biphasic effects of cariprazine on cocaine self-administration, specifically an increase at a very low dose (0.3 mg/kg) and a decrease at the higher doses (1, 3 mg/kg). One-way ANOVA with repeated measures (RM) over drug doses revealed a significant treatment main effect (FIG. 8, panel A, F_{(3, 24)}=9.616, p<0.001). Dunnett's post-hoc tests indicated a significant reduction in the number of cocaine infusions only after 3 mg/kg cariprazine administration compared to the vehicle control group (q′=3.497, p=0.005). In contrast, cariprazine failed to alter the number of inactive lever presses (FIG. 8, panel B, F_{(3,24)}=1.362, p>0.05), suggesting that cariprazine did not produce sedation or locomotor impairment. FIG. 8, panel C, shows the representative records of cocaine self-administration before and after the different doses of cariprazine administration, illustrating that at low doses (3 mg/kg, i.p.), cariprazine produced an increase in drug taking, which is possibly a compensatory response to a partial reduction in cocaine reward. However, at high doses (1, 3 mg/kg), cariprazine caused a typical extinction-like pattern of cocaine self-administration (i.e., an initial burst-like increase in drug intake followed by a cessation of cocaine intake), indicating a robust reduction in cocaine reward after high dose cariprazine administration. In total, this classical pattern of drug self-administration indicates that cariprazine pretreatment produces a dose-dependent reduction in cocaine's reinforcing effects (efficacy).

Male Long-Evans rats 275-325 g (Charles River Laboratories) were used in the cocaine self-administration experiments. All animals were housed individually in a climate-controlled animal room on a reversed light-dark cycle with free access to food and water. All experimental procedures were conducted in accordance with the Guide for the Care and Use of Laboratory Animals of the United States National Research Council and were approved by the National Institute on Drug Abuse Animal Care and Use Committee.

Rats used in cocaine self-administration experiments were implanted with a microrenathane intravenous (i.v.) catheter (Braintree Scientific Inc., Braintree, MA, USA). Each rat was first anaesthetized with ketamine (100 mg/kg i.p.) and xylazine (10 mg/kg, i.p.) and then a small incision was made to the right of the midline of the neck to expose the external jugular vein. One end of the i.v. catheter was next inserted into the vein with the catheter tip reaching the right atrium. The catheter was then secured to the vein with silk suture and the other end fed subcutaneously around the back of the neck to exit near the back of the skull, connected to a bent 24-gauge stainless steel cannula (Plastics One Inc., Roanoke, VA, USA) with a threaded head used to secure a dummy cannula and, during experimentation, an infusion line. The catheter and the guide cannula were secured to the skull with four stainless steel screws threaded into the skull and dental cement. After surgery, the catheters were flushed daily with gentamicin- and heparin-saline solutions (0.1 mg/ml gentamicin and 30 IU/ml heparin; ICN Biochemicals, Cleveland, OH, USA) as precaution against catheter clogging and infection. The animals were allowed to recover for at least seven days before behavioral training started.

Standard MED associates operant test chambers were used for self-administration experiments. Each box was equipped with two levers (active and inactive) and a house light set to on at the beginning of each session. Presses on the active lever triggered a compound light and tone cue. Data were collected and analyzed using MED PC software. A total of 48 animals underwent intravenous catheterization surgery. Following recovery, subjects were trained to lever press for cocaine (1 mg/kg/infusion) under an FR1 reinforcement schedule for five sessions. Then cocaine self-administration continued under FR2 reinforcement at 0.5 mg/kg per infusion for approximately two weeks until stable responding was observed (more than 20 infusions, <20% variability in responding across three consecutive sessions and a ratio of at least 2:1 active to inactive lever presses). Each session was 3 hours long with a cap of 50 infusions at a dose of 1 mg/kg/infusion to prevent overdose and a cap of 100 infusions at a dose of 0.5 mg/kg/infusion. Rats were randomly assigned to receive intraperitoneal injections of vehicle (25% 2-hydroxypropyl-beta-cyclodextrin; Onbio Inc., Ontario, Canada) or cariprazine (0.3, 1, and 3 mg/kg), compound 13a (0.3, 1, and 3 mg/kg), compound 13e (0.3, 1, 3, and 10 mg/kg), 30 min before cocaine self-administration testing. Each animal received 4 or 5 injections for the different drug doses. The sequence of the drug doses was counterbalanced. Between tests, subjects underwent an additional three to five sessions of cocaine self-administration until the baseline response was re-established. Behavioral experiments were analyzed with SigmaPlot 12.5 Software (Systat, Palo Alto, CA). All dose treatments groups were compared to vehicle group using One-way Repeated Measures ANOVA or One-way ANOVA tests followed by Dunnet's post-hoc test.

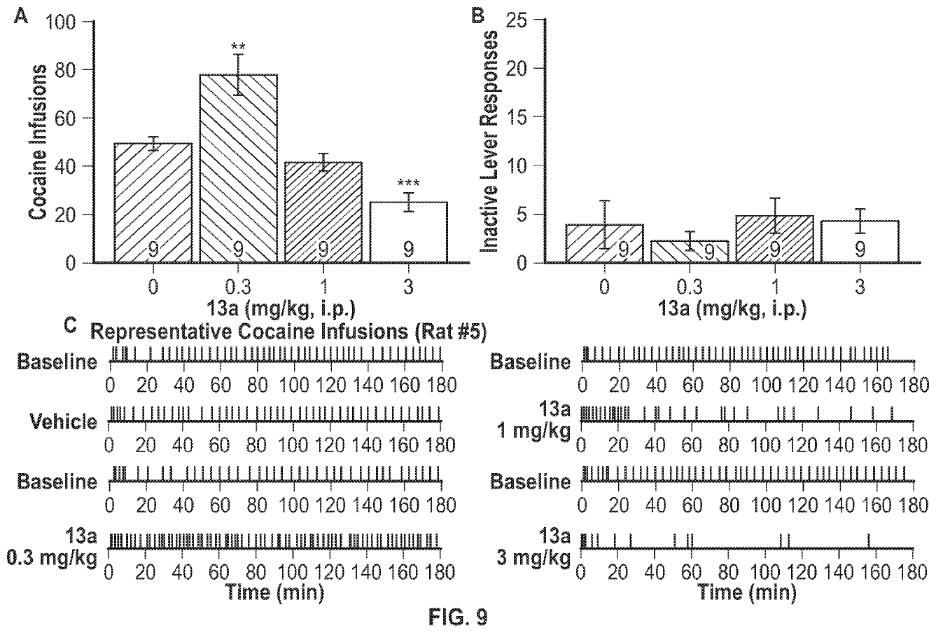

Based on D₃R binding affinities and selectivity profiles, as well as favorable MPO scores (>3) and partial agonist profiles at both D₃R and D₂R, compound 13a and compound 13e were selected for behavioral testing in comparison to the parent compound. Similar to cariprazine, compound 13a also produced a dose-dependent biphasic effect on cocaine self-administration. That is, at 0.3 mg/kg, compound 13a produced a significant increase, while at higher doses (1, 3 mg/kg), it produced a dose-dependent reduction in the number of cocaine infusions (FIG. 9, panel A, F_{(3, 24)}=23.822, p<0.001; Dunnett's post-hoc, q′=2.556, p=0.045 at 0.3 mg/kg; q′=2.873, p=0.022, at 1 mg/kg; q′=5.395, p<0.001, at 3 mg/kg). Like cariprazine, compound 13a pretreatment also failed to affect the number of inactive lever presses (FIG. 9, panel B; F_{(3,24)}=2.708, p>0.05).

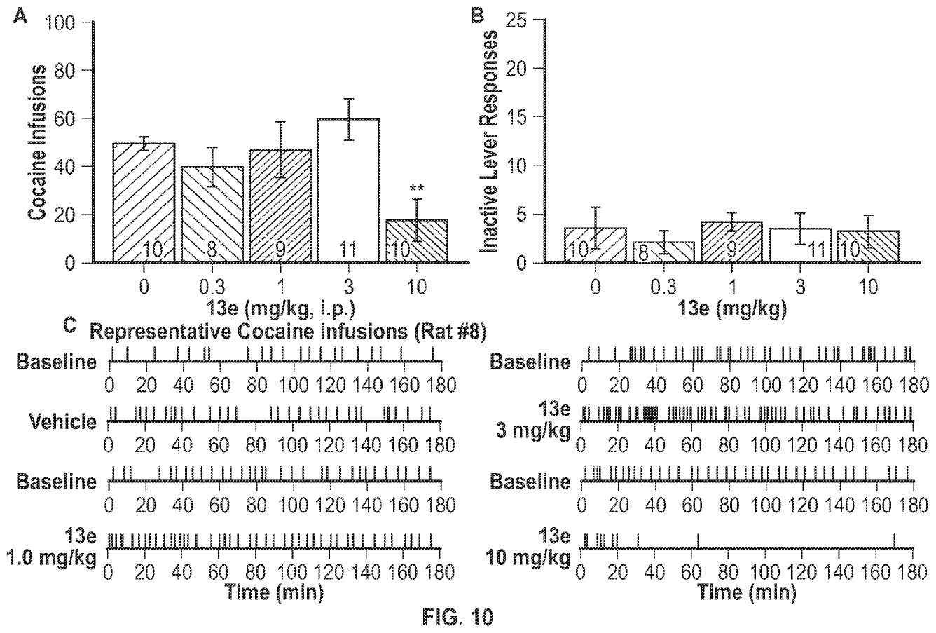

Compound 13e was tested and it was determined that higher doses (10 mg/kg) of compound 13e inhibited cocaine self-administration under FR2 reinforcement, in rats (FIG. 10). One-way ANOVA revealed a significant treatment main effect on cocaine infusions (FIG. 10, panel A, F_{(4,43)}=4.130, p=0.006; Dunnett's post-hoc, q′=3.283, p=0.007, at 10 mg/kg compared to vehicle). As observed with cariprazine and compound 13a, there was no effect on the number of inactive lever presses (FIG. 10, panel B; F_{(4,43)}=0.736, p>0.05).

This behavioral test provided several findings. Compounds 6a (cariprazine), 13a, and 13e appear to be more potent than highly selective D₃R antagonists in reducing cocaine self-administration under low-cost high-payoff (FR2) reinforcement conditions. In other words, considerably lower doses (0.3, 1, 3 mg/kg) of compounds 6a and 13a are able to alter cocaine self-administration. Compounds 6a and 13a produced unique biphasic effects on cocaine self-administration—increasing the number of cocaine infusions at low doses and decreasing drug intake at higher doses. These biphasic effects are usually seen after pretreatment with non-selective dopamine receptor antagonists, such as pimozide and (+)-butaclamol. In contrast, highly selective D₃R antagonists usually produced a monophasic reduction in cocaine or opioid self-administration, and much higher doses (10-30 mg/kg) are required to inhibit cocaine or oxycodone self-administration. The increase in cocaine self-administration after low doses of compounds 6a and 13a is a compensatory response to reduced cocaine reward, as the pattern of cocaine self-administration is comparable to the responses produced by lower doses of cocaine—evenly distributed drug intake with shorter infusion intervals. Thus, the increased behavioral performance reveals a reduction in cocaine's rewarding effects. Congruently, at higher doses, compounds 6a, 13a, and 13e all produced a classical extinction-like pattern of cocaine self-administration—initial burst-like increase in drug infusions followed by cessation of drug taking.

Taken together, these data show that compounds 6a, 13a, and 13e, produce a dose-dependent reduction in cocaine reward, which is consistent with their pharmacological profiles as partial D₃R agonists with moderate D₃R/D₂R selectivities (compound 6a, 3.6-fold; compounds 13a/13e, ~20-fold). Compound 13a displays similar pharmacological potency to cariprazine in attenuating cocaine reward, while compound 13e displays slightly lower potency as a higher dose (10 mg/kg) of compound 13e is required to inhibit cocaine self-administration. This behavioral potency difference is in line with their receptor binding profiles. Whereas compound 13a has a similar binding affinity to D₂R and D₃R as compound 6a (Table 1), compound 13e displays ~5-fold lower affinities for D₂R and D₃R compared to compound 13a (Table 3). Similarly, in vitro functional assays indicate that the EC₅₀ value of compound 13e in activating D₂R is ~10-fold higher than that of compound 13a. This is consistent with the effective dose of compound 13e (10 mg/kg, FIG. 7) in attenuating cocaine self-administration that is also ~3-fold higher than that of compound 13a (3 mg/kg, FIG. 6).

### Example 5

### Additional Behavioral Studies

### Surgery:

Right jugular vein intravenous catheterization surgery and cocaine self-administration procedures were conducted in Long-Evans rats as previously described (Xi et al., Neuropsychopharmacology, 33(7): 1735-45 (2008)). Briefly, animals were anaesthetized by an intraperitoneal (i.p.) injection of ketamine/xylazine (100/10 mg/kg), and catheters, constructed of micro-renathane (Braintree Scientific Inc., Braintree, MA, USA), were inserted into the right jugular vein. After being sutured into place, the catheter was passed subcutaneously to the top of the skull and exited and attached to a connector (a modified 24-g cannula; Plastics One, Roanoke, VA, USA). The connector was then mounted onto the skull using jeweler's stainless-steel screws and dental acrylic. To prevent clogging, the catheters were flushed daily with a gentamicin-heparin-saline solution (30 IU/ml heparin) (ICN Biochemicals, Cleveland, OH, United States).

### Multiple Doses of Cocaine Under Fixed-Ratio (FR2):

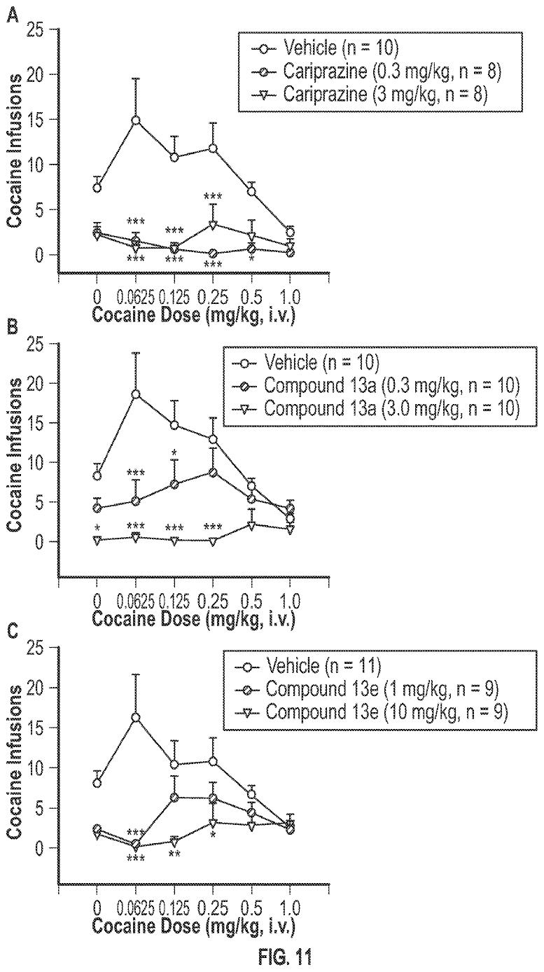

After 2 weeks of training on cocaine (1.0 mg/kg/infusion) dose, rats transitioned to a lower cocaine dose (0.5 mg/kg/infusion) and a FR2 schedule of reinforcement. Training continued until animals earned at least 20 cocaine infusions per session, exhibited less than 20% variability in responding, and reached an active/inactive lever response ratio of 2:1 or higher for three consecutive days. Once stable self-administration was achieved, animals transitioned to a multiple-dose self-administration program, during which ascending doses of cocaine were available for self-administration in 20-min intervals (0, 0.0625, 0.125, 0.25, 0.5, and 1 mg/kg/infusion), with 10-min intervals between each dose (Keck et al., 2013 Psychopharmacology, 229(2): 253-65 (2013)). Cocaine doses were achieved by adjusting the infusion volume and duration. Once stable self-administration was acquired following the criteria above, rats were randomly selected to receive systemic intraperitoneal injections of cariprazine (0.3 or 3 mg/kg), compound 13a (0.3 or 3 mg/kg), compound 13e (1 or 10 mg/kg), or vehicle (25% beta-cyclodextrin) 30 min. prior to the test session. The results are set forth in FIGS. 11A-11C. As is apparent from the results set forth in FIGS. 11A-11C, when the animals are pretreated with vehicle, they show a classic inverted “U” shaped response with increasing doses/infusion of cocaine. Both compound 13a and compound 13e, dose dependently shift these curves downward demonstrating that in the presence of these drugs, cocaine is no longer reinforcing and the animal stops pressing the lever.

### Self-Administration Under Progressive Ratio (PR):

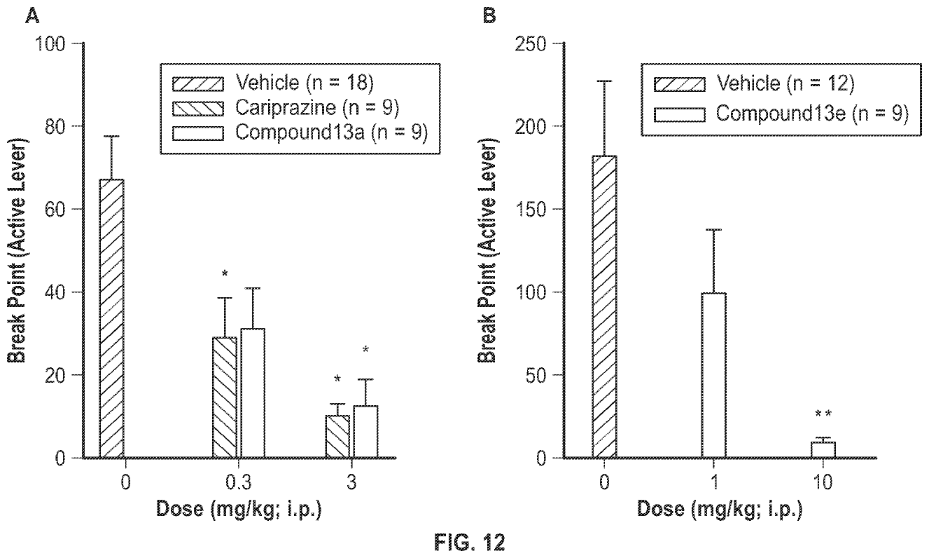

In rats, cocaine self-administration training under fixed ratio was conducted until stable levels of behavioral performance were obtained. Then, cocaine self-administration was continued under a progressive ratio (PR) reinforcement schedule following protocols previously described (Xi et al., Neuropsychopharmacology, 33(7): 1735-45 (2008)). During the PR schedule, the delivery of each successive reward was accompanied by an increasing number of lever presses in ascending order: 1, 2, 4, 6, 9, 12, 15, 20, 25, 32, 40, 50, 62, 77, 95, 118, 145, 178, 219, 268, 328, 402, 492 and 603 until the break-point was reached. The break-point was defined as the maximal workload (i.e., number of lever presses) completed for the last drug infusion prior to a 1-h period during which no infusions were obtained by the animal. Animals self-administered cocaine daily under the PR reinforcement conditions until day-to-day variability in break-points fell within 1-2 ratio increments for three consecutive days. After a stable break-point was established, subjects were assigned to different subgroups to determine the effects of cariprazine (0.3, 3 mg/kg, i.p.), compound 13a (0.3, 3 mg/kg, i.p.), compound 13e, or vehicle (30 min prior to test) on PR break-point for cocaine self-administration. Because it is difficult to re-establish a stable break-point level after each drug test, a between-subjects design was used to determine the dose-response effects of cariprazine, compound 13a, and compound 13e on break-point for cocaine. The results are set forth in FIGS. 12A and 12B. As is apparent from the results set forth in FIGS. 12A and 12B, when pretreated with vehicle, the rats will press the lever >60 times for each infusion. In contrast, in the presence of compound 13a and 13e, their lever pressing was significantly reduced, suggesting that these compounds, like the parent compound cariprazine, reduce the reinforcing efficacy of cocaine and reduce the rat's motivation to seek it.

### Drug-Induced Reinstatement of Cocaine Seeking:

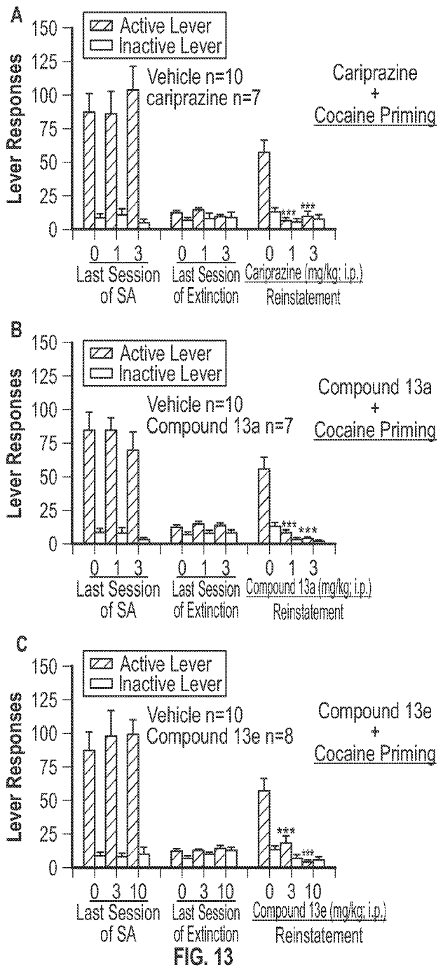

In this set of experiments, cariprazine, compound 13a, and compound 13e were assessed as to whether they reduce drug-induced reinstatement of cocaine-seeking behavior. Here seven different groups of rats (n=7-10 in each group) were trained to self-administer cocaine (0. 5 mg/kg/inf) under a FR2 schedule of reinforcement. After 2 weeks of self-administration, rats underwent extinction training during which responding on either lever produced no consequences. Once rats reached extinction criteria (<20 lever presses within their 3-hour session on 3 consecutive days), they were randomly divided into seven dose group to test the effects of cariprazine, compound 13a, and compound 13e on drug-induced reinstatement of cocaine seeking. Rats were injected with one of the cariprazine (1 or 3 mg/kg), compound 13a (1 or 3 mg/kg), compound 13e (3 or 10 mg/kg) doses or vehicle (25% beta-cyclodextrin) 30 minutes prior to the onset of the session, and injected with cocaine (10 mg/kg) 15 min before the beginning of the session to reinstate lever responding. The results are set forth in FIGS. 13A-13C. As is apparent from the results set forth in FIGS. 13A-13C, reinstatement to drug seeking behavior is completely and dose-dependently mitigated with compound 13a and compound 13e. These data suggest that both compound 13a and compound 13e may be useful in preventing relapse to cocaine seeking behavior.

Without wishing to be bound by any particular theory, it is believed that since compound 13a and compound 13e are more D3 receptor-selective than cariprazine, they will have less D2-mediated side effects, but are equally effective in these models of cocaine addiction and thus are promising as a therapeutic treatment for substance use disorders, and particularly psychostimulants such as cocaine (or methamphetamine).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present disclosure can include any combination of these various features or embodiments above and/or below as set forth in sentences and/or paragraphs. Any combination of disclosed features herein is considered part of the present disclosure. Further, when an amount, concentration, or other value or parameter is given as either a range, or a list of upper values and lower values, all ranges formed from any pair of any upper range limit or value and any lower range limit or value are also disclosed, regardless of whether ranges are separately disclosed. Where a range of numerical values is recited herein, unless otherwise stated, the range is intended to include the endpoints thereof, and all subranges, integers, and fractions within the range. The scope of the disclosure is not limited to the specific values recited in a range. All references cited in this specification are herein incorporated by reference as though each reference was specifically and individually indicated to be incorporated by reference. Each of the elements described herein, or two or more together, are also within the scope of the present disclosure.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound having Formula (I), an enantiomer thereof, a racemate thereof, a salt thereof, or any combination thereof: 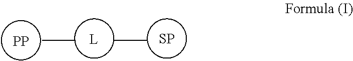
wherein
PP is primary pharmacophore of formula PP¹: 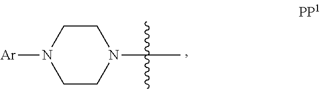
wherein Ar is a substituted or unsubstituted aromatic or heteroaromatic substituent;
L is a linker, covalently bound to the primary pharmacophore, of formula L¹, L^{2a}, L^{2b}, L^{2c}, or L^{2d}: 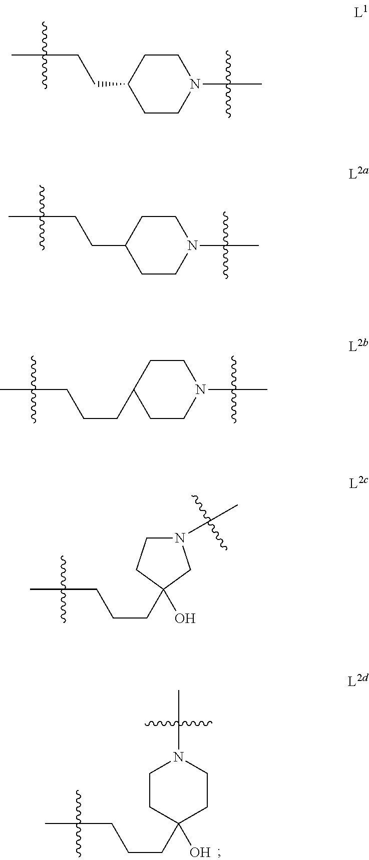
and
SP is a secondary pharmacophore, covalently bound to the linker, of formula SP¹ when L is L¹, or of formula SP² when L is L^{2a}, L^{2b}, L^{2c}, or L^{2d}: 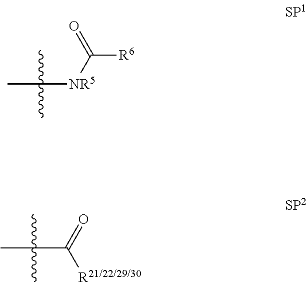
wherein R⁵ is hydrogen or C₁₋₈ alkyl,
R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
R⁵ and R⁶ combine to form a 5-, 6-, or 7-membered cyclic amide, cyclic carbamate, or cyclic urea, and
R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent; and
wherein the compound is a selective dopamine D3 receptor partial agonist.

2. The compound of claim 1, an enantiomer thereof, a racemate thereof, a salt thereof, or any combination thereof, wherein the compound is represented by primary pharmacophore PP¹, linker L¹, and secondary pharmacophore SP¹: 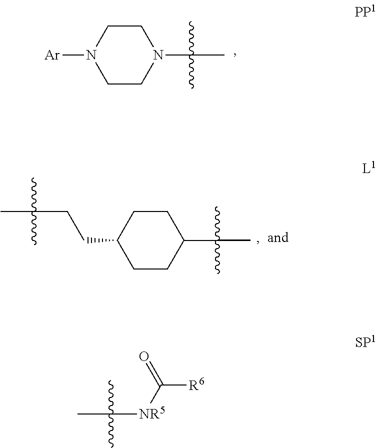
wherein
Ar is a substituted or unsubstituted aromatic or heteroaromatic substituent,
R⁵ is hydrogen or C₁₋₈ alkyl, and
R⁶ is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent, or
R⁵ and R⁶ combine to form a 5-, 6-, or 7-membered cyclic amide, cyclic carbamate, or cyclic urea.

3. (canceled)

4. The compound of claim 1, an enantiomer thereof, a racemate thereof, a salt thereof, or any combination thereof, wherein R⁶ is

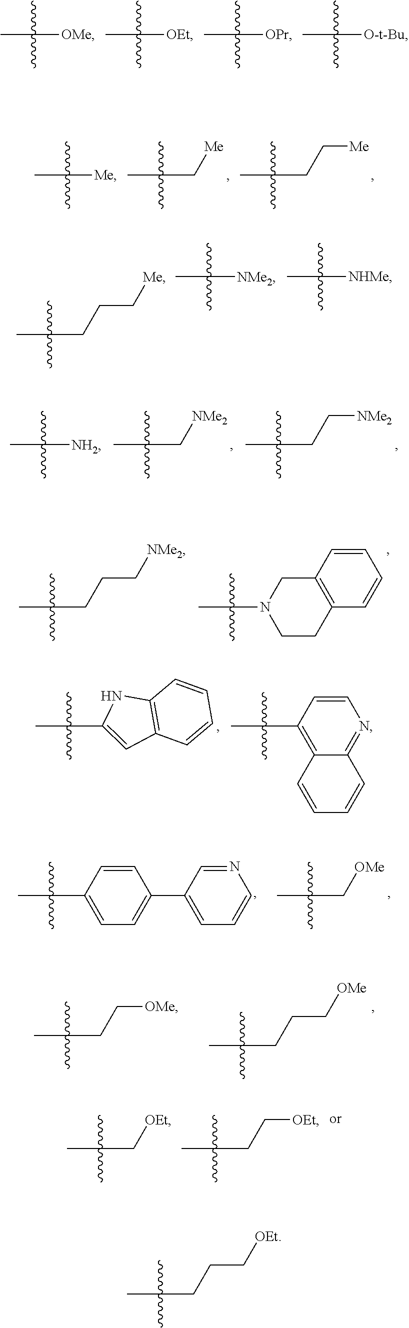

5. The compound of claim 1, an enantiomer thereof, a racemate thereof, a salt thereof, or any combination thereof, wherein R⁵ and R⁶ combine to form a 5-, 6-, or 7-membered cyclic amide, cyclic carbamate, or cyclic urea.

6. The compound of claim 5, an enantiomer thereof, a racemate thereof, a salt thereof, or any combination thereof, wherein SP¹ is

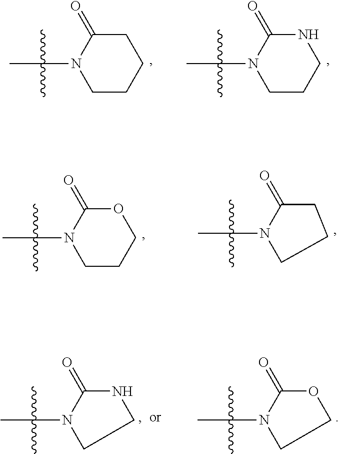 [SMILES: *N1CCCCC1=O ; *N1CCCNC1=O ; *N1CCCOC1=O ; *N1CCCC1=O ; *N1CCNC1=O ; *N1CCOC1=O]

7. The compound of claim 1, wherein Formula (I) is represented by Formula (II): 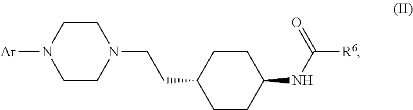
an enantiomer thereof, a racemate thereof, a salt thereof, or any combination thereof,
wherein
Ar is a substituted or unsubstituted aromatic or heteroaromatic substituent and
R⁶ is an amine, a heterocycle, or an ether-containing alkyl substituent.

8. The compound of claim 1, wherein Formula (I) is represented by Formula (II): 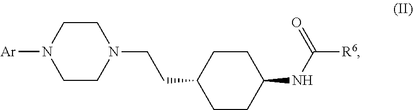
an enantiomer thereof, a racemate thereof, a salt thereof, or any combination thereof,
wherein
Ar is a substituted or unsubstituted aromatic or heteroaromatic substituent and
R⁶ is

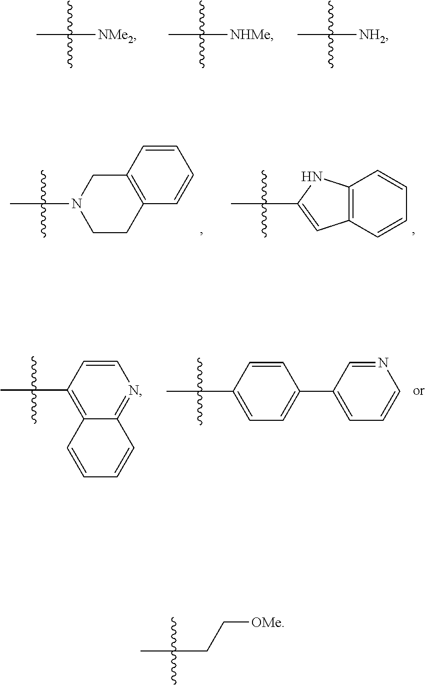

9. The compound of claim 1, an enantiomer thereof, a racemate thereof, a salt thereof, or any combination thereof, wherein the compound is represented by primary pharmacophore PP¹, linker L^{2a}, L^{2b}, L^{2c}, or L^{2d}, and secondary pharmacophore SP²: 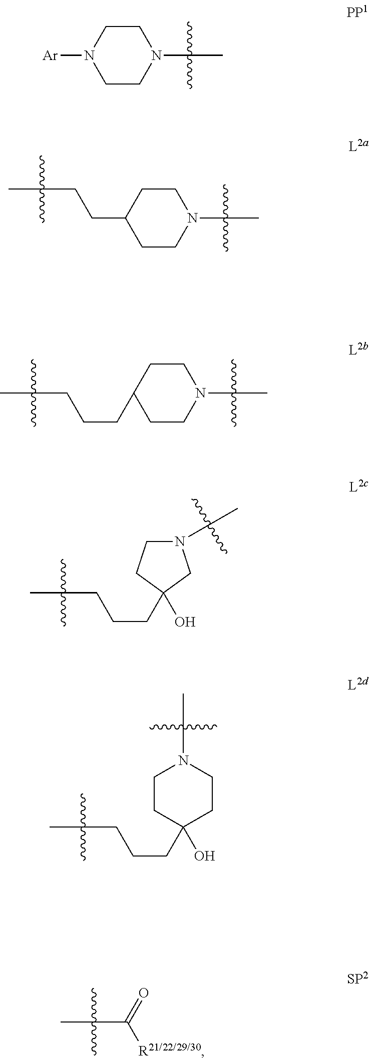
wherein Ar is a substituted or unsubstituted aromatic or heteroaromatic substituent, and
R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent.

10. The compound of claim 9, an enantiomer thereof, a racemate thereof, a salt thereof, or any combination thereof, wherein R^{21/22/29/30} is

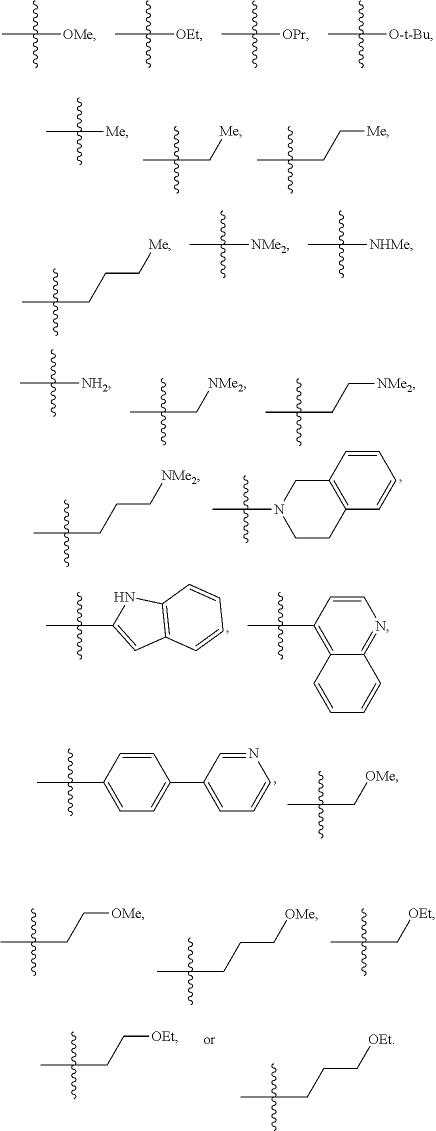

11. The compound of claim 1, an enantiomer thereof, a racemate thereof, a salt thereof, or any combination thereof, wherein Ar is

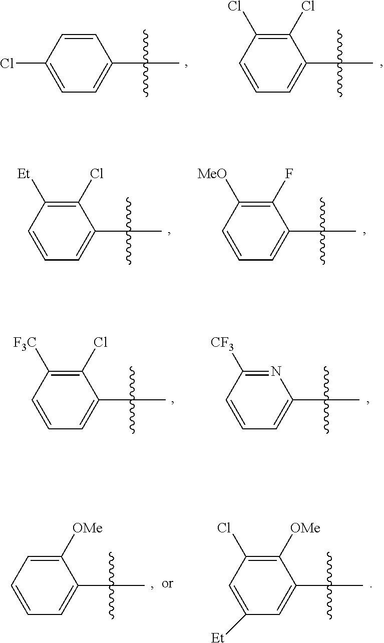

12. (canceled)

13. The compound of claim 1, an enantiomer thereof, a racemate thereof, a salt thereof, or any combination thereof, wherein the compound is represented by primary pharmacophore PP², linker L^{2a}, L^{2b}, L^{2c}, or L^{2d}, and secondary pharmacophore SP²: 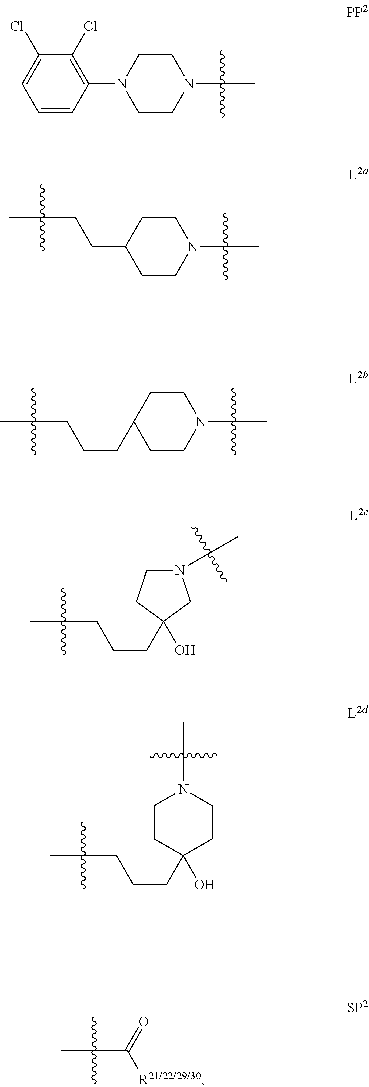
wherein R^{21/22/29/30} is an amine, C₁₋₈ alkyl amine, C₁₋₈ alkyl, C₁₋₈ alkoxy, a substituted or unsubstituted heterocycle, a substituted or unsubstituted aromatic, a substituted or unsubstituted heteroaromatic, or an ether-containing alkyl substituent.

14. The compound of claim 1, an enantiomer thereof, a racemate thereof, a salt thereof, or any combination thereof, wherein the compound is represented by primary pharmacophore PP², linker L^{2a}, L^{2b}, L^{2a}, or L^{2d}, and secondary pharmacophore SP²: 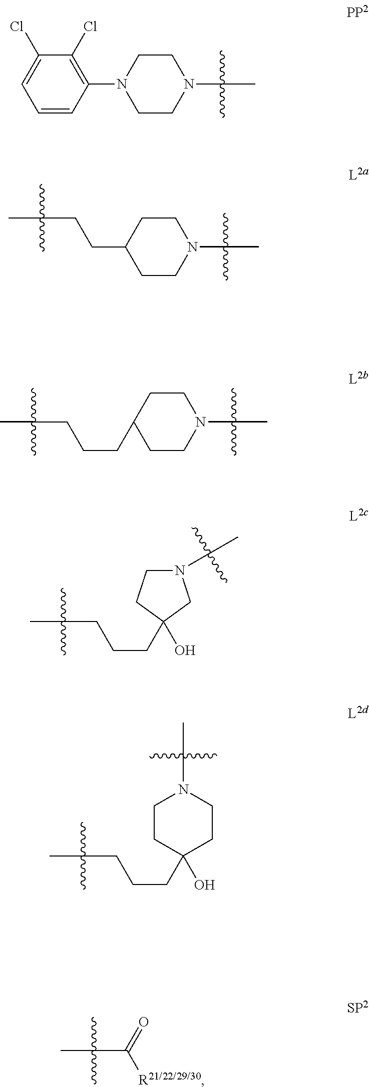
wherein R^{21/22/29/30} is an amine or an ether-containing alkyl.

15. (canceled)

16. The compound of claim 1, wherein the compound of Formula (I) is or a salt thereof.

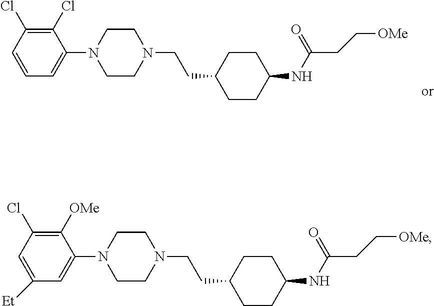 [SMILES: COCCC(=O)N[C@H]1CC[C@H](CCN2CCN(c3cccc(Cl)c3Cl)CC2)CC1 ; CCc1cc(Cl)c(OC)c(N2CCN(CC[C@H]3CC[C@H](NC(=O)CCOC)CC3)CC2)c1]

17. The compound of claim 1, wherein the compound of Formula (I) is 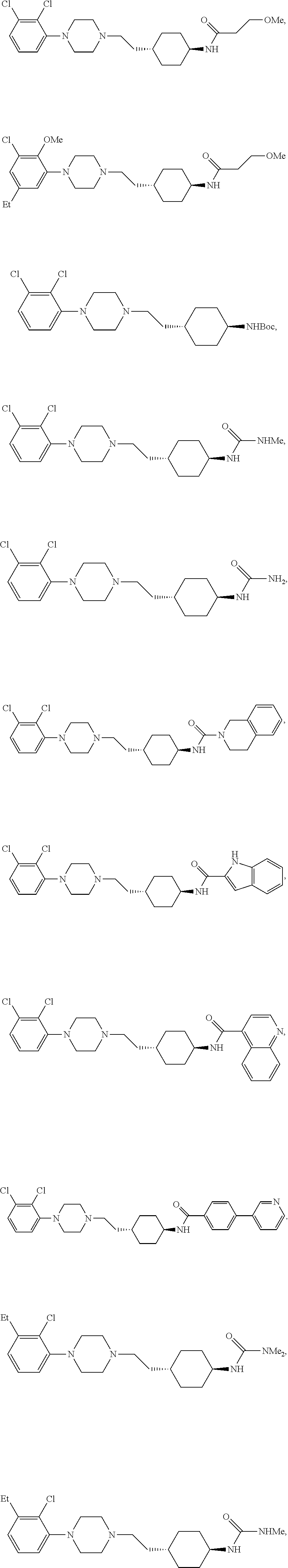 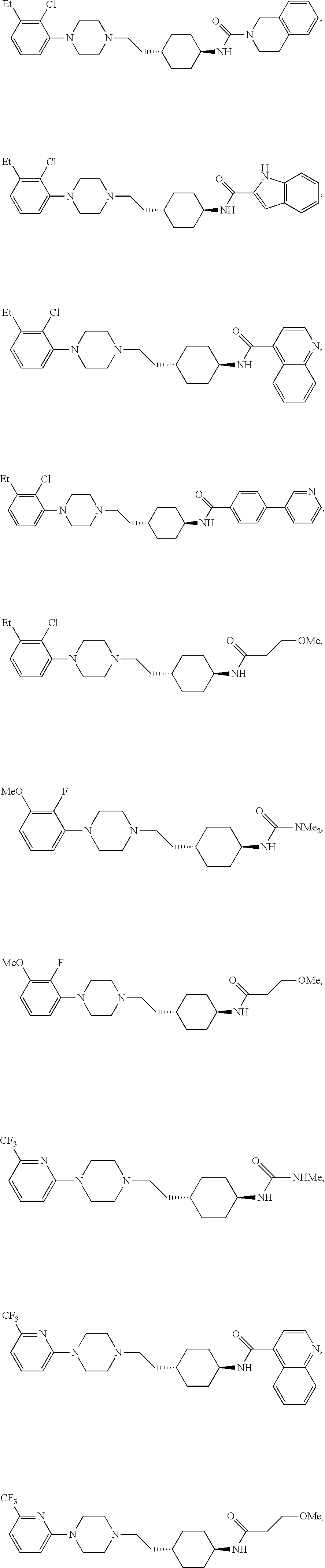 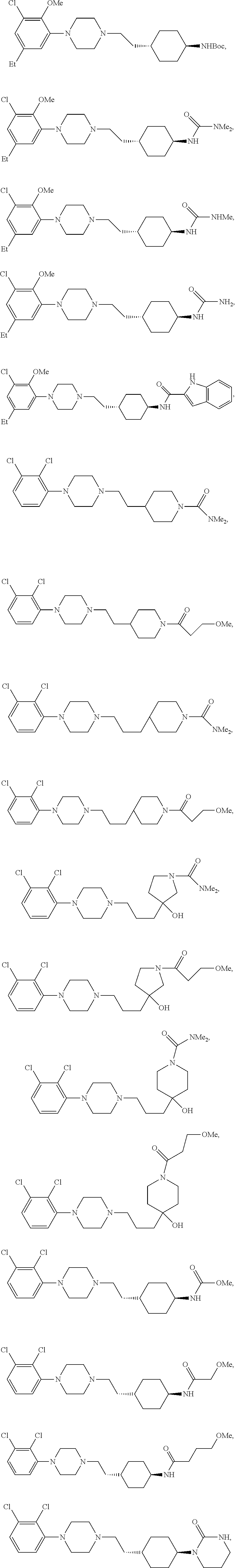 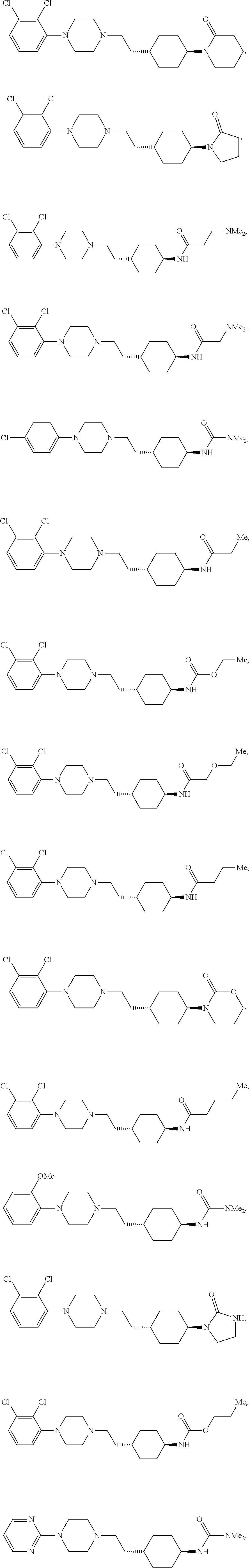 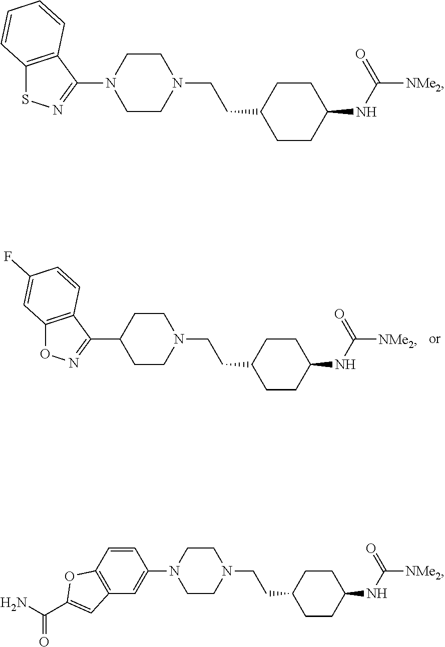
or an enantiomer thereof, a racemate thereof, a salt thereof, or any combination thereof.

18. The compound of claim 14, wherein Formula (I) is represented by Formula (IIIa): 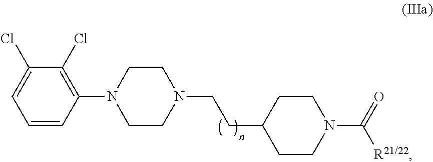
an enantiomer thereof, a racemate thereof, a salt thereof, or any combination thereof,
wherein
n is 1 or 2, and R^{21/22} is 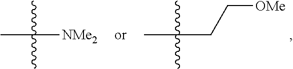
Formula (I) is represented by Formula (IIIb): 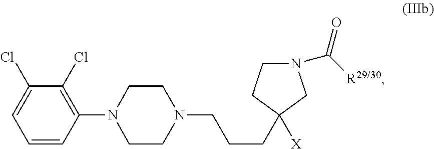
an enantiomer thereof, a racemate thereof, a salt thereof, or any combination thereof, wherein
X is OH and
R^{29/30} is

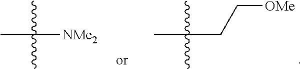

19.-22. (canceled)

23. A pharmaceutical composition comprising a therapeutically effective amount of a compound of claim 1, an enantiomer thereof, a racemate thereof, a salt thereof, or any combination thereof, together with a pharmaceutically acceptable carrier.

24. The pharmaceutical composition of claim 23, wherein the compound is a first therapeutic compound, and the pharmaceutical composition further comprises a second therapeutic compound, wherein the second therapeutic compound comprises an antidepressant, an antipsychotic, or a combination thereof.

25. (canceled)

26. A method of treating drug misuse or drug addiction, comprising administering to a patient in need thereof a composition comprising a therapeutically effective amount of a compound of claim 1, an enantiomer thereof, a racemate thereof, a salt thereof, or any combination thereof, optionally in combination with one or more additional active ingredients.

27. (canceled)

28. A method of treating a substance use disorder, comprising administering to a patient in need thereof a composition comprising a therapeutically effective amount of a compound of claim 1, an enantiomer thereof, a racemate thereof, a salt thereof, or any combination thereof, optionally in combination with one or more additional active ingredients.

29.-31. (canceled)

32. A method of treating one or more affective disorders, schizophrenia, or a combination thereof, comprising administering to a patient in need thereof a composition comprising a therapeutically effective amount of a compound of claim 1, an enantiomer thereof, a racemate thereof, a salt thereof, or any combination thereof, optionally in combination with one or more additional active ingredients.

33. (canceled)

34. (canceled)
